SEQUENCE LISTING (7 sequences)

SEQ ID NO: 1 - DNA, 17590 bp, Homo sapiens
  misc_feature 1..2000: 5′ regulatory region
aggacagtatctagcacaataccccaaatcgactaactcctccgtaaagaatagctaccactattgtgagagttttaagtcaagctgtgaataaaactcttgggtccacttaaaaatacctcccctggatgtaagcatccagggaaatcagggaatgccataagacagccctaatctaaaagcctacaagaagctcagtgggcttcaaggaagacactgctcttggtacgatgaggaaacctggccctctatttgcctcctgggccacagtaatattgataatagctgctgcttttagttgaggaccatgtacgtctgtgtcactgcactggccactttacttacactttcctgctttgtcctcacaaagatcctgtaaggtgtgtattggtcccatttagcaggtaagacaatgaagaccagaggtccagcaccttgcctaaaccacacctgctgggatttggattcaagtccaaccgtacagctcaaacgctcagccacttccctaaagtccacccccagctacattaagtaaaaaaatccagaaagatgccacctgggggtctggaactgcctcctccgagcacccggctctcccctccctgcggactcttctctggagaggatgtgatgcttcttacttttctcagatccctctccccaccctgcgagtgacgttgcgcctctgtgcctggtgggatagggatctgggagcttcgcctgttttttgcacactgccatcccctagtcttagggagcgagctctgtcccgcttttcacatctccgcgtctttccttgcactctacatcaccgctgggaatgtccccagacctgatcggggcatgcacactggggtgtgcgtgtgcgtgtggtgtgtgttcctgcgcgtgtgccgggctcgcggggcaggaaaaagcgcctaatccaggctctgcgtcactcccgcaattggttagaaatggagtttcctggtgtttaatcccgggagggcacttcgccttcgttgtttcccagagtccctgattttcctgcctcgcatgccagcgccccatagggcatccgtgcctcagttcacctcttgccatcctccaaggacggggagaaggggtaaggcgggggagagcaaggtggcttggtcgcccccggcccccgccccccatgttgtgtgcagtttccaccacgtctgtttcggagggagaagaggagggtgcagatgaggcgaggcgccttcgggagcgcggagagcgggcaggcagtgccacctgctgagagccactcaggccgagcaagcggcgggcagtgccacctgctataaataggccgccaaggacagggtgtgcgactgtacatcccgccacgagggcctgcatcacgcgcggggccccgcgcccccggctccccagggaaacgctgtgcccagatcctgcgcaggggtctggatggggcggcggcccgagtacttcccccctattccccccacagacactggctgaggatggcccgcgggcttgggggcggggggtggcaaggaggggagggaggccgcggcggacccgcagtgcagcagctgttgctcgcgtgtgactcgcccgtccgggccgtgctgcccaggcacagtcacacggcgcagtggggaggaggaggacaccgagtccccctcccagctccccggggaccgagtggggagatcccggctcctgtcttcccctcgcctccagcgcgctcgcccaggctgggaggaggaaaccagagccgcgcgcagacacctcctccttctcctcctcttcttcctcctcctcctcctcctcctcctcctcttcggctgctgctcctggtgccgccaccgtccgccggtgcctgttgctgccgccgccgcgggacctgctgtgtcctcagctgggtggagaagaggcgggcgccgagccgaggggagccccctccccgtccccccgcggcgggaagagcgcagccagccgggtgcgatggactccccgcccgcccaggccgtccccaggatgcccccaagcacctgcgcgtcccggcccggccccgggctctgagcgcgccgcggcacaggtaaggcgcttcctggggcttcgtcctggccaccctgctggctcctctcggggcgtcgcggccgccccctcccgcagcacgcccctgccccgcctggccgcggaggggaaggcatctggccgcccacggacgcgaggccagggtctctcgggggaggaagttcattgccatctcgttgccccccttacccccccacccccgccgcccttggacgaaagcgaaaccttaatgttgctagcgacccgagagctccgccggcttctcccccaacccccgccagctcactggtccgcgcatctctcccctcccccctcccgccaattatcctagcgtgtttgcaaggcgaccagattggaaagagtgtggtcagagtgaccccaagccacgctttaaaagttcagggtactttgcagtagtaactttggcagctccaccagtgcgcgcaacatttctttctatgggtacatcctgtaccagtcattttgaaaccctgcttcattgtttctagccgcttcctgatggctctgtgattatgagacccccctcaaacttcaccaggcattaaggttttgtttttgcttttttttcagagaggtatcatttcgtttgaaatccacctagatgtggcttttcctgttttgattttacttaacatagcttattctctggaagttgctttaaaaagaaattgaaagtgatggttgttccttccaccaaacagtttaattttcagggtgcctcatattaatggatatgttttcccttcatagatttctcattgtttcccttatgatgggatgatttcatttattaataaaatcagactttgaaagagcatttaaaaatgacctggtttaaataggtcacacccaagaaactcagctatctgtacaagttcaaacttctaaactttttcaatgagctaggggtggtggcacccacctgtagtcccagctacttgggaggctgaggcaggaggatcacttgagcccaggagttcgaggccatagtgagctatgactgtgccacctcactggagcctgggtgacaaagtgagatcccatctcttaaaaaaaaagagtttaggggacattttctgaagtgaacacaagtagagcattctaacactattgagtgcaaggagacctggaagggactaagtggttcaaagcaggaaataaaatcatcaggtgataattaaaataatttctttcctgtggatttgtccagccatttgcaaaccaggagaataggaaaaaaaatcactagtgtagttataaattattacattacgttttcaaaggaaaattttgcaaatgcgtctccttgtcatagtctattgttatctaccccactgagagtgctggggcttccccttttcaccacgacagcatttctggttgggtggcagtcatgcagtgttgacctggtgtcccataaggcacagtttgtcaaaacactagtgggtattaggaggaaacgtgcaactctgaagcaacagagcttgccccttcttcctcattatccagctggtgataatccctgtcccccacttccctagaagacagctttgaccaggaaggctgcaatgacaatgagatgtacccctatgcagagccagatgtgggcgggtggcttttttgtggtccagatcttctaggatcttctaggatgtaaccctggcaagcagtggggagcctgaatcaagcagcatggctgttacctcttctgtgttcacagcagcatcttcagttgtcttggtgcctggagcaggcaccacagctgcctgctctgttggccaccagctttctagagtagatggtagggaggagagcaaggggctcaagaggattctgtctttgaacatgcttttaantttgatctgacagaatggcagctccctgaagtccttcctactctctccacagcatttctctgtaggtccccagtttttgctcttttcagattcccagaggacntgaaaatgtatcacggcccatttggggacttcctgtatatgtgtgggtgcctcaggatcatttgttttgcccttttccagtctaccgtgctgcccttctcaagtttaatgnaccacgttagtttcaatattttatatatttctcagcagttttcatctcttggtcattaaacttgagaagtaaaatctgctcattaaaatgactgagtccatggccaggcatggtggctcatgcctgtaatcccagcactttgggagtccaaggcgggtggatcacttgaggtcaggagttcgagaccagcctggccagcatggcaaaaccctgtctctacaaaaatatagatctacaaaaactagccaggcatggtggcatgtgcctgtagtcccagctatttgggaggctgagacaggagaatcgcttgaagccaggaggcggaggttgcagtgaaacatgatcgtgccactgagtccattcagcagcagagtagtgttggggtttgtatccctgtagtgatgacgaaggatttaggttttcagtcagaactgttaccttacaatttccttcactgacttttcttcctttccaacaccacattccaataaaaaatatctttagaccagattcttcacgaaagacatgaaggttttcatgcttcaaggtttttgactttttttttttttttnaaaggagtcttgctgtgtcacccaggctggagtgcagtggcgtgatctcagctcactgcaacctccgcctcctgggttcaagtgattctcctgcctcagcctcccaagtagctgggactacaggcgtgctctaccacggccggctaaattttgtgtttttagtagaggcgaggtttcaccatcttggccaggctggtcttgaactcccgaccttgtgatccacccgccttggcctcccaaagtgctgggattacaggtgtgagccacggcgcccgaccagtttttgacatttctaagccaaaagttccatttgatgaggtcttagatgcaggggcaatgtgtcccttttcagatttcagatgtttagaaaaagatgtgtcatatttgggccaactgaaaaactcttgatatgtaggtttttatgaagctgtgcagaatgtaggaaatacattttagaaccaacaaagaggcatttaattttgagtgtgcctgtctcctttgagatgagcaacagctatttttctcttcaaaagacaatgcgtgtatttatcagcacattttatataatcagcaaatctaaacctctgaattaggtaagccctataggtttgttgccagaattagtgaatttatacatgcaaagtgcttagaacagtgcctggtacacagtgagcactcaatattatttattgctattattatgtttatttattttatacttttagagtataattttgatgttaggtttggattgctgaggccaagcaaaatttagatagaccaacccagctaatccactagaaagatatttgagggttattcccatctaaagatctatgggatctttggatatcatctgtgagaaacaacagaagtttgtagataagacagatatagattcaaatgccaccttcacaatttattagtgagggaaccctttggtaaaatgagcatgacaaaacctacttctcagctttgtgaacgtacagaagatcatgaatgtaaaatgtccatgaagtggtaggtggtcaacagatattgctctagcaaagtggttaagagcaagcaaactctggagccaaagggcctgggttcaaatcccgattctgccacttcttatagtatggccttgggcaggtgacctcacttttctgtgccactattcaatgataatattcctttattgtccaacgttttgtaagttaaatcagttaataaacacactatgataatgtgttggtaactattctttttacttttagcagaataacttaaaggaacactgcaggtaggagggttatacataatctctgagggccagctaggacactcgcccatttcccaccctttttcctgtgcaatgaagagtataagaagtgacagtgccattctaaaggactagccttgagttggctctaatttatatgactcgtgcctgtaatcccagcactttgggaggccgaggctggcagatcacnttgaggtcaggagttggagaccagcctnggcaaacatgacgaaatctcatctctaccaaaaatacaaagatcagccgggcatggtagcacacacctgtaatcccagcttctggggaggctgaggcagaagaattgtttgaacccagggggcggaggatgcagtgagctgagatcacgccactgcactacagcctgggtgacagagtgagattctgtctcaaataataataataatttatatgagaaagaagtcattcaaaagcatcattactttacatgtcaaattagaaaggcacaccccagtactaaagcatccttgatgatgaaaacatttagaaccactggtttcaggagctccatgcaatggtgaaacagcctctactccaagggttgttgctccctgtgggattcctgggtgaggaacacactgctcccgttggggtggaatcctgtggaggaagtggatgaagagtgtagccaagtcagtgagcctactgcatgggattagagtacttcaggttgcagtataattctgttcaggtgcatgctcactccatctggcgtaaagaacagagaaattaaaccattgattcacagagcaatatgagtagctgcctggggacttcctgtccattctggggtncccaacagccaatcaatattggccggttcctaatctgacctagagctagaggccactagcacccctccattcatcctttcttctctccctacccactcccaccagcattctgaggaggtgagggctgaagctgcagaggctgttgtactgtcagttactgtaaagtccccatcctgctctctgtagtttgctgtgaaggagtggagggggctgggaacaaagggggttcccaataagcagcttactaatacctacccttgcttctctcacttcctgatcaataattcccatcctttcttcatgtacctcccccacatttttgttctttaagggaagaagggagcagctttccatccccacacaatgttggggacatttggtattatacattatgaaaataagatttaccagattttagagatggaagaaaacttggggatgatcttgttccattctcttataagaacaaacaatatttggagaagctgagtaacttgctttttcaacttcacacttgaaagtgttttcatgaggaagttggggttctctgcagcacttggatgggagtcagggacttggattgtcccaattctgtcactaaatttggacaagccacttaacttttccagaatctagttgcctcatttcaaatattagggaaatttctaaatggcttaaaaggagcttgttagctttaaaattttatgattctaagtgtatgctgccagagatatgtagcatagcaggacacattaacaagattattgaaactgttctaataaaggacatctttgtgtcttgggtagctactatgtttaaagactgtgctaggtgggagttgtgcagaatacacaggtttgctgtagagggatagggcgtgtacacagacaactctactaacaagaacgttactagaagcttattggaatcacagtatttcttgctgagggtatgaaacataagagttctccttggaatatgaggttctatttggggcttaaagaatggtcaaaggttgagtgcaaataacatggattgagatggctttaaaaaataatcaaatggtttgttagtattaaactggtgcagaaataattgcagtttttgccattccttttaatggcaaaaattgcagttacttttaaaccaaatccctaatattatttgcatagtttatctctgttatggaagtttttattgacaagtaatgtagatattcacctgatctaagttaccctgaatcttatattagcagaatctgaattgcttataaataattatggctatgttggatgtagaacttattatttgatagtttatgaacagtgctaaggtctaatctactttttacagagaagctaagaacatgctacagctggttgaaaaacaaaaacttcaggcattgaaatgttttgtcaatgaaatggcaggactcatttgatgactgattattatcaactgatttaaatgactgaatttttggtactgtgtacatctatactctaagaaggaaattgaaagtaattctgctatgcttgttgccactatattaataactgcatcatctaaaataattgatagagctcagatttatcctttgtaataattctagtacttctttaaacatgttttgggattagcagctgtcaacagttagaacatgaaacagattctgttacaggagtagaagtcgatccagacatttaatgtcattttcacctgtgagagagagaataaagagaaagagagatcattatttatgggattatgtgaacttcaagtccgttttcattattaggagaagctgtgctttaaaggacagtcagggactttactttcatgaaatgcctgagctgtaaataaagtattgctttattttttatttcttgaacatttgaaataaaaaattagctatgagttatgttcaaattatattataaaaatttgctcttagcattgtgcatatatattatacagaaaaacacagagtaaaaagaatagacttcagttcctgttcagaaaaggtttaaaatttgaatactgattttggaaaccccaaaccttaagaattcaagaagcttacggtcttcttgagggacacctattcaaactcttaaatatggtgattgggtagaaagtgcagaaaagcctgctgatacatgccctaaaacaccttggaaaaaagaggtggtagttgcttgaggtaggacttaagtactagttggaaatagaagacaaggatggagactgttggtagatgactctccatgggtccttcctgtttctacacaccttgtaagcagggcattgagtgcctgtgttccaaactaccttttccatcatgtttctacagcaaacagtcatggaagatagaaatagagtcttcctctggagcaaagggcagacacgcttgcttcctgtacttcccactataagatattccggctccctaaactcagctgcctttcctgtaacccaccatgatacagatgtcacctgacctgtgggaattgggggtcagggaaccaagagaaatgctgactgtctggctactgtgactgccctgagtaataaattgtccttcgtctccaacccaggagtctcatgttttctaccagcaggataactgtggcrggctaacgtgttagtttgcaagtaaggtaaaatctcagaccctttgcagtttgtggcagggattatattctgaggagagaggaaccgtatgcaccatggctcagaggcatgagaaacggggaaccataactagttctctatcttcagagcctttaaaaggtgcaccaaggagggcattttaggggagaatataaagttggagatatagacacagccagattcctgagagaccttatatgccaggtagaagacttcagattgtatgggggaattattagagaatttttagcaggggtgtgatatgataaattttgtgttgattaagttactccaggaaatatgcgatgggtggattgaaggatggggcaccttttctctaggacgaaaaagaaagagtagttggtgaagtcagttagaggtagtaataggatgaagaagggatctgaatgaccccttggccattcagtgagtagtgatgctattcacctagatacagcacatagnngggaaangaaatnctgggaaggagggagatgagaccgagttagctttaaaataactaaattcaggcctaggagcctataggctatccagatagaaatatttaatngcctatatggatctggaactcaggaaggaggcttccgtgggagcagaacacttgggcaccattagggtgtatgtggtagatgcattcttgtgcagcagtcaaggggatgggatttagactcaagtgcaaattgccccccatctcctgtgataagtgactgaagctctccgggcttcagtttcctagttcatcatagtgggctctagcggataaatgttacaaaggttaaatgagacaacataggcaaagtgcgtggtactcaatagaagtcagctgctgtcatcagcagcaggatcaccagaatgtggtgcttgacaccaaaagattaggtgagattgcccaaaacagcaggtgaaatgaggggagaggatgnaagtcaaacacaggaagaaaagcctttgaagtatgtggaaagaaacaaccagaaaggtaagataagaaccagaagagattcaagaaggaaggtgtggccgggcgcggtggctcaagcctgtaatcccagcactttgggaggccgaggcgggcggaacacgaggtcaggagatcgagaccatcctggctaacacggtgaaaccccgtctgtactaaagatacaaaagaattagccgggcgcggtggcaggcgcctgtagtcccagctactcgggaggctgacgcgggagaatggcgcgaacccgggaggcggagcttgcagtgagccgagatcgcgccactgcacntcnagcctgggcgacagagcgaggagccgtctcaaaaaaaaaagaaaaaaaaaaaaaagtaaggaaggtgtggccaagattgagaaattcgtcagagcaaacaaggcagtcaggggctaaatagcctcctttaaattttacaaccttgaggacctcggcaactttaacagaatttcagtggatccctagggcaaaccaggccttacaaaccaggaatggatggtcaataggaagtggagacagtaagtgtagaccttaccttggagggaaggnaagagaaagagccatggccaagggaagtttgaaatcaaaggaaatatctttttttttttttttcgattggagagacctcagttattcttttaaaatacttattgagcccctcagttattcttttaaaatacgtattgagtccctactttgagtcaggcacnatggcagacacgagggngatagcagtgaatcagacagatgcaacgcctgccttcatggagtttcaccttagcatctgtccatatgctaggggagtggggcaggggcagggagctggatacaggagagactgaagatccagggagcaagtgagtaaagaatagggcttgagatcccacagacaactcagctttgaacaaaagggttttgtcatccaataggacaagaaggcgttaggatacatcaaacgtggttgttgaaaacagaaaagggctgggcactgtggctcatgcctataatcccagcactttgggaggccaaggtgggcagatcacttgaggccaggagttcgagaccagcctggccaacatggtgaaaccccatctctactaaaaatacaaaaattagccaggtgtggtggtgcatgcctgtaatcccagctacttggaaggctgaggcaggagaattgcttgaacccagggggtggaggttgcagtgagccacgatcgtgccactgcactccagcccgggcaacagagcgagactctgtctcaaaaaaaaaaaaaggaagaaagaacatagacagggaaatgtagttaaggnnagtttgggtttgggtttggtagaagcgttttctgttnnttgtttgtttgttttcagaaagagtctcactctgttgtccagactggagtgcagtggcacaatcttggcttgctgcagcctctgcctcctggattcaagcaattctcctgcctcagcctcctgagtagctgggattacagacacctaccaccacaccaggctaatttttgtatttttagtagagacggggtttcaccatgttggccaggctggtctcaaactcctgacctcaggtgatccacctatcttggcctctcaaagtgctgggattacaggtgtgagccactgcacctggcctaacattgatatctgttgatgagaagaagccaggtgttggagtgatagcttatagcacatgaactgaataaaacagtgtttaagacaatgtttgcaacataataggcactgaagacatgttaatggaaggtggatttgtgattcagaacctctagactacctgggcgagtcttttaaaatgtaagtaatatcttaagtgatattacttgtcccagatcagttgtttaaaactgaggtttaatgctgtcagagtagcactgtatcgtcttctatcatgggggcctttgttggctttaggaggtttgtgtttcatagtagtttcccagtgggctctttgttacctgtaatgagtgtgacagttatgccataaccaggttttatatggaatacaattttgagaaagttctttctaggcagagaagcttatttgaacctcttattatatttgggtttcaggcttttgagttcttctgaaataatagccctttgaaggtagctattgctatgacttcattaaattctaatgcctctggttttctcccccaggtttctgcatatgaagtgtgtaaaatagattgcttgatccaaaacagaaaaacagtgataactgttttgctgagttcccagacccttcccaagatggaaccaataacattcacagcaaggaaacatctgctttctaacgaggtctcggtggattttggcctgcagctggtgggctccctgcctgtgcattccctgaccaccatgcccatgctgccctgggttgtggctgaggtgcgaagactcagcaggcagtccaccagaaaggaacctgtaaccaagcaagtccggctttgcgtttcaccctctggactgagatgtgaacctgagccagggagaagtcaacagtgggatcccctgatctattccagcatctttgagtgcaagcctcagcgtgttcacaaactgattcacaacagtcatgacccaagttactttgcttgtctgattaaggaagacgctgtccaccggcagagtatctgctatgtgttcaaagccgatgatcaaacaaaagtaagtgagatggagatccaaaagactaaggtgtggctggctggtttttattgtatgggggtcaggatatttattttaagtatactgaaatgaataaggaattaatgctgcagttataaattgattacttagctgaatttttgttttatggtgatagtttatagttttaaagcacatttgaaaacagatacgagaaattatcagtttttgagttcaaaaattcaagagaaatcagtctaaaactactaattaagagcagaagtgttaagatgtacattatttcagatgaatgttctaaagccatgcctctcaaactgaaatgagcttgtgagtcacctggggatcttgttaaaatgtgaatcttgattcagtaggtctggggtggaccccaagactgcatttgtaacaagctgccaagaaatgctgatgctgcccttttgcaggttgcactttgagtggcaaagttctaaatctccacatttgtaatcctattaagaaaaatatagtcattcgtaaactgtgtaaaaatgctactggccagtttcccaaggcataatgttcacttaggcaaaggtcattgataagaacgctggatatgcatctaagttttgatgcgatcaggggttctttgtgtttttttctttcgcaaacctcaggtcagatctgattagcttgttattatcacatgatatggctgaaaaaaaatgtgagacatggtaaaagttctgctctttcctcgttcatttgtgcttgctttgttattagcattcgttgtagctctgggcaggactcatttgaagatgcttgncccattttatgaggattagcttagataaaattgaaaatataatgcaaatagcaactttctcagttgggctcagggctccacagctaaccccatggactgtggagtcttgccgttgttttgggtgccaagcaagccaagtcacatgtgattcaagctgtctgccacatgtacagggcgaggatgcgagtgtcaatccacctgttaactgtcagtgaagccttgaaagcttctcatattttcaaggttaaaatctggatagaaatgctaaagttttctctctgcactccattaggttattttatgtactctctagggtgtaaggaccttatttagaaattaatattcttggtatcaagtagatgcccttttgcttgttcatttgttggttcttctagtcattcagaattgctgttgcaggtactgttggagatgatattagcagaggcttgtaggaaggcaggagcatcagtggggaataggaccaggtgatctatgtataggacataatggaaggactgagaagggagcctaacacacacccaaagggtagagaaggctttgtgaaataaaggctaatatggagctcaaaaccaccatttcactcacagaatcaaactctcatattataaatcatttcatgttattgtccacacatctcaagtgggcacggcagcatcaggcttggagattcagagggactaacttcctgtactctaatcctacttctgcacccataaactgggtggcctcaggcaattgagtctgttttcttatctgttaaatggggataattacagtatttatccaatagagttgctggaaagactaaatgaggtagcactcgacctgaaacttagtaagcatttatagccataaaaacattttcattcaagaaaattttactagaggcagattatatgctaatttcatttcacgtcttaggtaaaaagaaacatgatacctagatgagtgccttcagctttcaaagatgagattctggtcatatttgaggaacattttaaaaactacacgtataacttaatggctcctattatttggacaaattccagaatgaaaatgagaggactgaacagcctgtacctcagtccagctctatatagtatttggactgaatttccttggggagagtttgtgcgtggaatcgttgttcagcattttacacatttgactctttcccaaaatcttttacggccatctgagaataggcttctggccagtcattcggatgcctgacaagagaaagagatttataaccaaattctgtaattgggacttccagtctttccccaagtagagaattggacttactctatatgctaaaaacccatggttgaaatatgaattagttcttaagtgatttttggcttgcataccatttttgcaaacacaaattgtcattactctgctcatttaataaaagaataatttgtagtataggtatatacctcaatcagtgattttgttgttggaaacagaacagtaaatcacactggccatgatgctaacagcgtgatagattttctgttcttgggacaccaatgtcactgtatctcatagcgaaggattatctgctgtaggagcattctcttgactacttataacatttgctgggtgaaataattctccaggttaaggcctcttctaaacagatgaggtcagcactaactgcatttgccagagaagacatatgcatttactgccagcatcataaacacaaaactacagtttgcgaggaaaccctttgaccagcatctaattaattcactgagtaatgtcttgggagaagaggcatgtaaaggaacaattttataagcatgccatgagattgttttcnnattgtatgttccatagaatatgaggaaacttcaaaacattttgtggaaaaattgaattaaaaagtaaaaaacacatatatacataagctttatttctcaagataaactttatcaagttcaagacacttttgtaagcaatgttaacagccattgagtcggtctctaaagaactgagggtcctgggaatttaaccatgtttatacagtcttttatacattattaactggagaaaaattggcgctctttaaagattttttaaaattgagaagcaaaaggacgtcagaaggagccaaattaggcctgnnaagtggatgcctaatgatttcccatggaaactcttgcaaaattgctcctgtttgatgagaggaatgagcaggaacattgtcatggtggacaaggactctggtgaagctttnncaggcgattttctgctaaagctttggctaactttctcaaaacactctcatgataaacagatgttatcattctttggccctccagaaagtcaacaaacaaaatgccttgggcatcccaaaaaactattgcaaccatttgcccttgaccagtccactttcgctttgactggaccacttctgctctcagtagccattgcttaaatttgtcttgatctttaggattgcgctggtaaaactatgtttcatcacctgttacaattctttgaagaaatgcttcaggatcttgatcccacccgtttaaaatttccattagaaactctgctcttgtctgcagctgatctgagggcaatggttttggcacccatctagtaaaacgtttgctcagtgttaatttttcatccaggattgtgtaagctgaaccagcagagatgtctatgatattggctagttggtcctcttcaatgagggcatgaacaagatgaatattttcctcaaacaattatctggatggtctgctgctgcaggcttcatcttcaatattgtctcgtcccttctttttcttttcccccccgcttgagacacagtcttgttctgttgcccaggttggagtgcagtggcccgatttcggctcactgcaacctctgcctcccgggctcaagcgattctcctgcctcagcccaccaagtagctgggattacaggtacacatgatcgtgcctggctaatttttgtatttttagtagagacagggtttcaccgtgttggccaggctggtctcgaactcctgacctcaagtaatccacctgccttggcctcccaaagtgctgggattataaacatgagccaccacacctggcctcatcctttcttaaaatgagttatacatttgtaagctgctgatttctttggacattgtgcctataaactttttgtaaagcatcagtgatttcaccattcttccacccaaacttcaccataagtttgatgtttcttcttgctttgattttagcaggattcatgtttctctgatagggggtcttttcaaactgatgtcttatccttcttagagcctcatcccagatcctgttcagacatgctacaagttaatacaagtttatttggtgccaaaaaatggaaatccatgcatagtttttaaataatatgcatttttcatgnactttttgaagaccccttgtatacttaaactgctccacatggaaaagcttccatgatcaaatgcagtaaggcagcatctcaaacat

SEQ ID NO: 2 - DNA, 99960 bp, Homo sapiens
  exon 4661..4789: exon A
ctcaagcttgaatacttgaatccaaactttcatgcttagagtttaccccatctgttgaaggatgtgcaatataatgactgcaatagaattcactgtggagcctccaaattagaaattattgtctgtgagggccaggcacggtggctcacgcctgtaatcctagcactttgggaggctgagatgggaggattgtttgaggccaggagtttgagaccagcttggtcaatatagcgagacccccatctctgttttttttttttaaagaaattattgtctaagaaccagtgtcatcttccaaggagaaacttctagatacttgttttaagataaataagaaacaagtcatttctaaatgtgaattattttttaaatgcaattttttaaacattttattttaattatggcaatagacgtggaaaagactcttttttgatagtaggggagagcagaagaaacattgaattaagtacacagagattcttcagacctgctttaaaaacacatgcatacaaatgcacttctgtctcttaggatctactaactgatgctgcttgctttagtcttttagctaatattttctttctttctttctttcttttttgttggagacagagtctcgctctgtcgccaggctagagtgcagcggcacaatcttggctcactgcaacctccgcctcccgggttcaagcgattctcctgcctcagcctcctgagtagctgggactataggcgtgcgccaccacgcccagctaatttttgtatatttagtagagacggggtttcactgtgttggatgggatgttctccgtctcttgtcctcgtgatccgcctgccttggcctcctaaagtgctgggattacaggcgtgagccactgcgcctggctcatattttctttatatatcaaaacaattcagcttgcttcacttttatgaaagctttattatgagtttgaaagcaattctgcattttcttaacattgtaactggtgttgagttgaaggcaggcccctgggagccctttgtgggcaattcccttcactctggaggctgcctcgagcctggacaggcacttacacttggtcagtgattgcacagaaccggttgcaacagattctgtgcacctccctgtggcgcgtagcatttagcaggcacttggtcactatttgctgagtgagtctgttaccttaggcgtgtatttcccgtggacctgcctggggatcattgctcattcactcattttgaacaagccaatattacatgtccagggtacgctctatagtgtgaaacacaaaggtaaatgatagttccccttctcaaaggaatttctaaggtagtagccattcttttgatgcatattctcattctcatagagagtccaattatggataattggacaaagctgaatgtcgcttttatgagaatccattctttctcttttatgcyttgaaaaatgtgtagcattcattagtgaattaggatttcattattcaaagaagacataaggtcttcgaacagcagatgactgaataaaataatacctaacagcagtagaatgaggggaggacatattcaaggaacattttatgcccattagattggcagaaatttttaaaaagtgacaataccgtataaaggtgaactttcctatactgatactgggaacatgaatttgtaccattcagggaagagaaacttgataatatctggtgtagtctgaaggggcacagtccctgtgacccagtgaggacattcctcattatttcccttgccaaacatttcacatgagtctataaggagctctatataagagaggtcactgcagcctcctttgtaagagcaagaaaaaaaagcaaataagtgtttaacaataggaacatagataaattaggttatgcagtgaatatttgcactctgactaaagtgagtgaatcaaaaaaaatttgtcaacaggaataaatctcaaaaataatattgaaagaagaaagctaatttacagaaggatgtgtacagtatgacaccattcatttagtttcaactacatatcttttatggacacatacatataaaagcagaaaacatgaattgataggataaacaccaaatatttctgcatatggccaggtgtggggaagtagtggtgattaagcttcaaagatgtctgcagtggttcccattaaaagtagaaagtaggctgggcacagtggctcacgcctgtaatcccagcactttgggaggccaaggcaggtggatcatttaaggccaggagttcgagaacagcctggtcaacatggcgaaaccccatctctacaaaaaaaatacaaaaattagccagatgtggtggcgcacacttgtagtcccagctactcgggaggctgaggcatgagaatcacttgagcccaggaggtagaggttgcagtaagccaagatcgtaccactgcactccagcctgggtgacagagtgagactccatcccaaaaaacaagcaaacaaaaaaagctcatagagtaggtaatagtcatgatatctgatgttttttgattgtctggtttacattttttatttttattttttgagacaagtctcacgctgtcacccaagctggagtgcggtggtgcgatgtcagctcactgcaatctctgcctcctgggttcgagcgattctcctgcctcagcctcccaagtagctgggattacaggcgtgcaccaccacacctggctaatttttatatttttaatagagacagggtttcaccatgttggccaggctggtctcgaactcctgacctcaagtgattcatctgcctcagcctcccaaagttctgggattacaggcatcagccactgcacctggccttggtatatgtgttttaatttgtattcattcatttaagcctcatgacagctctgcgaggaaagttcactatacgtcttcaggctgcaggtagaggacctgaaagggacaggaggtaacagtctggccaagaccacagagccagggaatagcagaggaacatttcacctgggcattgcactccagagctgggcttctcactgttctcaacccctggcaaatgctcacttgaacaaagccaggtggtgatacaaaggtatttgttatattagtctctacacttttctgtgtgcttgaaataactgcaacaaagaatatatcagtatttagagtaatgggggatttgcttgtgtgtgtttgtatttttgagatggagtctcgctctgtcgcccaggctggagtgcagtagcatgatcttggctcactgcaacctccggcttctgagttcaagcgattctcctgcctcagcctcctgagtaactgggattacaggtgtgcgccactacacccggctgttttttgtatttttagtagagacagggtttccccgtgttggccaggctgatctcaaactcccgacctcaggtggtccacccaccttggcctcccaaagtgctgagattacaggcatgagccactgcgcctggccgtttttttttctaacaaaattattttctaacagaaagcaatcaggtgagaatccacataagaaacaatttaattcagagatttttgttgcatattaaaaaaaaaatgtaccttcggctgggtgtggtagctcactcctgtaatcccagcactttgggaggctgaggcaggtagatcacttgagctcaggagtttgagancagcctggccaacatggtgaaaccccgtctctacaaaaactacaaaaaattagctgtgtgtagtcccagctactctgggggccgagggagaaggattgcttgaacctgggaggtcaagactgcagtgagccatgattgtggccctgtactccagcctgggcaacaaagtgagaccctggcaccctgtctcaaaaaaaaaaaaaaagtacctccttgtaaataagtaacactaagacttcatttagtggttgtcaagcaaactccattgtatttttattttcagtttttatggctagtagttaagggagagaagcttggttgcagagaagaatgaaaggatgatgggaaaataaaagtaggagagaggaaaacgcaagaaagcaagagatctgtagaaagggatgaaggaattgtataggcagagagaataggttctttaattgagaaatttatgttgtctcaccttctgaaatgcccccaaaggtaagttattgttttattttgaaaagctaatgatagctacctttctaccacgctgtgttcaatgttttacacactttacctgtttgagtctcacaacacagtgttatgattcgatcttgccattggtctcactttactgaagaggaagtttgaggctcagaaaagtaagaaactggccgaagaccacggttagtgaagacagatctctgatccagttgcagagtctgagcaataaactacttcaactgattggtttcaaagcacatttcgtcattttacttggggtaatcaaagcaactctctgaggcaaaattatttcctggacttgcagccatgtcactaaggagcagatgaggtgagatcacagacaggatcagaatgatggcctggtgccaaaaagatgtgtcctagagatttttcattcctttaagaagcagagaagggagcgataaatgacttttcgtttttcacttttttagacatcgcagatggcagcagagaatattggaagtgaattaccacccagtgccactcgatttaggctagatatgctgaaaaacaaagcaaagagatctttaacagagtctttagaaagtattttgtcccgggtaagtagcataatttctcctgatttaagttaaatcactttttaggagagtgtaagattgagttctatgcttttattccatcaatgttcatcataaaggtaaaagtataaaacctttttttatgttttctcaggcttataacagtattatctacattttaaattgtttttaatttggcctaggtttaaaaaaaatattccttactcttttgtattatatccaatgggatttttttgccgctccaaagaatatttgttagccagtccctataaagagcatgcattagatacactgaagtgtggcttctgttctccctactatcactatgtataacttaaaaaacagttactgtcagctgctggtgttagctatctaaaaggctatatagtaggggtcagcaaactatgcccatgggccaaattctacccacctcctatttttgtaaataaagttttgtygaaacaccgccacatccattcattttccagttatctaaggcttcttttttgcagacttcagcagttgccacaaacactatatgcctcacaaagcataagacacttactatctggccctttacagaaaaagtttgccaaatatagctctatagaaagaacaaagtacacatgtacatcaatctgggagttctttaagaaattatccctccctcccatgagtgtaaatagcctgatggcacgtctgagaaatcaaatctgattttccctcagagtttcacacctttctggagtgtgcagtatcttattatagttctttttgattttatggcacacttcttttgaaacatctgattttattttattttttaattaaggaaagttaaattttattttcttcgaagatgtttctgagaattttgcaatatcttctgagatcatgaaaaacagttgatttacaaaaccagagttgggaggggctgcatttgagagctcccaaagggatagagtgctgtccgagtgacatgcggctggccgttatgatgacttgtgacccaggggagggagttagttgctgagtgggcttgagcacttgattttccttatagacgaattgtcttgtcttcctgcctatcactcatgccaaattacttagccaccaggtgttttggaacgtttaggttagtgtcttatttattttttaaaaaaatgatggaaatgttgattattttaatgtacaaatatccttagtagcatttctcagtagataacatttttttcctgagcttatttaaatggaccaatctgcttctagctgatgcctttgcaaaagcctccagagtcataactcgactgccttttctttatgtagggtaataaagccagaggcctgcaggaacactccatcagtgtggatctggatagctccctgtctagtacattaagtaacaccagcaaagtaagcacatttctctttatrcgacaccctgaagaaaccaacaaataggtcttgctcatctcctgtctacatacctccaatcataaaacgtttgctgcttgcaaatttcttggcacaggtggaggactggtcatgcagttctatcataacataaaagttttacataaaagagcagatggggctgggtgcagtggctcaacgcctgtaatctcagcactttgagaggcggacgcgggcggatcatgaggtcaggagatcgagacgctcctggctagcacagtgaaaccccgtctctactaaaaatacaaaaaattaaaaattagccgggcgtggtggcgggcacccgtagtcccagctactcgggaggctgaggcaggagaatggcatgaacctgggaggcggagcttgcagtgagccaagatcacgccactgcactccagcctgtgtgacagagagagactctgtgtaaaaaaaaaaaagcagtagattttcctattaaaaaaataattaatattgggaaaacatcagaaagtggatttgtgaatttagagaagtatacagcttaaatttttctttttttaagaaaattttattttggatttgggggtacatgtgcatgtttattacctgggtatattgcatactggtggggattgggcttctagtgtacccatcacccaaatagtgaacattgtacccagtaggtaatttttcaaccttcacaccccctttcattctcccccacttgtggggaaattaaatttctgaaactttatcctgtagctggctctatgattataatgaaacattactgttttatttaaataagcaagtatctatgtccttcttttaataacttgctttctagacatttaatcatatttaagcctggtcagttcaactttataactcctgaaaagtgggtttgggttttgtgctagggaggccagctttcccttctgctaccagaggactctctttggcagtagtgagggagggagtgtttgtggaggccagctccttaccacaggcagggtttacagtcctctgccatccctcctagacatatggctttcagaatttttctaacctacagtaagaagcacatttaacattgtggcgtagttcacaaacacacatacctacacattcacacacaaaattaaaagttcacaaaacaatatttactgtgaacaacatacaatacatactgatattttgttctattttatttttaaaatgctcatggcaaactactcagttgtaccacctactaacatgatagagggagcagtttgagaaacacttccttagatggatgagtgcttctcaaatttcaggtgctccgcctcccgggttcaggccattctcttgcctcagcctcctgagtagctgagactggttaaagtgcagattctggttcagtaggcagggcggggggagccctgaaatgctgcatttctgacaagctccaaggcaatgctgctgctcctggtctgcagaccgcctctggggagtgaggtcctagacagcagtcttgtaaatgtgagtttctgagttaaaatccaggggaacatagtgtcgtccagcctccatctaatacacactgatcccaccctgcaattcattgcaagtgtgggaaggctatttgcttatttgttgtgtacagatgaaccacacaccgcccctttcatgtaggaagttacctaggaggagagagatgacagatacagaaacagccccagcatcaagcagagtgtggtaggagcccagaagttacaaataagagacattggtaacttcagtgtcagaagagcaaggggaagggaagttaggtttggtcagtggaaccagggaaaaggtgaggggtgagggggcagtgatgcttttggactaaatcttggtgtaggaattgtgcctatggaagtgaacagagaagaaggcattctagacagaccagtgtcaatagacataccatgaagacattcatgtcactcagtgggctttccagtaagccttattgcttgctttttatttttttccaaaaggcagatctaggaatatatacatattcattcttcaggactcgatagttgtgaagattcttttaaaaggatttaaaagtctgtctaagattgcaatttctagagtcattctaagagagatgcaacttttcagaagctgcttgtatgtattgtatatgtttaagtgtactttacatctttcttttattcatcttgaattgagaaactactatattctattttatgtaattggatcccttctaaaaaattgatcacctaggagttgcaaagaaaccaaatagccctgaaacttgacaaatgaaaatggccctttcagttgtccaattaagctaagggttagctctttgatatgatttggagggatattagtaagaatttagatcaacaggtttgcatgatggagattgtgttctgtgatgtattgtcttagagagacttttaaatccttaaaagaatcttcacaactgttgagtcctgaagaatgaaaaacttcagttatgaaagtaatcaatatttcatagtatgttgggaattttttcctaattcttatacaattaaatgtatgtaacttctccctttggtaaacacatttctttttttttttttttcaaattaaaaccctcaatacttgttacctaaaaggcactcaactgtgtaaatgaacaggtagaattcagagtctccagtccactgttagatgcattcattcttgtttactctattcctgttgatttattttttctcttccaacaatttcaataggagcaagctgctacaattcctctttttgaatattttgaatatattaaaaatatattggccactagccacgtcctgggtgcagtgttaaacatcagtttgcttgagtggtagtagttcattcctttgaaaaagcgtgcatcgtgaaggcatacaactttaaaatattgtcatgattctcaacaaatgtttgagcactcactccatagatttattgcatacctaataaaacaataacttatgtttgtgtaacattttacaacataaaaagtacttttggttgtatcatcttgctttgttcttgaaactcagatacatttttactttaccctcttacagaagaaattgaggtgcagaaagaaattatttgccctgaattgcagcagtaagtgcctacagagtgattttccatattctaagaatattgatacagttcttaatctcaaattatgaagtcgaatctcaacagtagatcagattcggagagagccttaaaatgtgggtttaacatgagtgaacacatgtggcaaagataaagaacttggttaagcagtggagacaagttctccagcactcacaccccttagaagctgcagtaaacagtcctgttttctagagagagggcactattcatggcgttgttcagaacgttacagattgtggcttatgtccttcactcctgcacttggccagtctccccatttctccagcaagccagcagtgtgtccttgaggagcgggcatttatttaatggaccttcattttcttctgcttttggtggtggcttctagatggcattataatcagaacacatacttagatactgcaatgtttgcccgtgcaggaactagagatttataaatcccacatattccccatggtgtgtctgatctgctgtgtgtttgctcccaggagccatctgtgtgtgaaaaggaggccttgcccatctctgagagctcctttaagctcctcggctcctcggaggacctgtccagtgactcggagagtcatctcccagaagagccagctccgctgtcgccccagcaggccttcaggaggcgagcaaacaccctgagtcacttccccatcgaatgccaggaacctccacaacctgcccgggggtccccgggggtttcgcaaaggaaacttatgaggtatcactcagtgagcacagagacgcctcatgaacgaaagtaagatttgtttaaatttgttgcataaatagctggggcatatctgtgactagccaggtatgtgcatcccaggtatgtttattgagtgagagaaatgagtcaggctttactcttggtttggagataaaactggaagcagtgacatgttcgttcgagctgcttgtgagtatacaagcaatgggtacttgtattgtcaggaagcaagtgaaagtgagcaaaaatggtacctaacatgcatagtcattactcctcaaacaaagtaagagacgttgttgactgtggaactttgctgctgtgaggaagagggcaagcggatgagtctccccatctgaagccctggagcagggttataatgggagggagaggcgctgatccttacaggcagagcaagagaggtatgctggcctcatagggtgacaggggtgcttcagcttctggtcctagctctgccgtgaactaattgtgacctggacgaatttgctaaattctctgaataacaaaattggagtagatgttttctaaaatctctcactgtaagaattctagattcttctaacaagatttattcattgtaatagttgggttcctgtgaccagttagaatcgtctggttatggagaagagtaatcagaagttccccccattccttccaagtgtcccttagtgattcatttaattctgtgtgccagagactataaatggacacagttatctttaaaaacaactttaaacaattttaaaaatctctcacctaatatgaatcaaggtcacacctgtgtacagtcgctgccttcttctgaccagcagccgcagaagtcccaggacctatgtgttcgtgtagttcatacacggatcattgagagtgtgagttagtacagaagtgtttggaatgttctgagtaaagaagtgtgagcattaacagtcctggatgatggagcagagcctcccagctttgttttctgtcagccattggaaagagttcttggttctttggaattcagcggggtagtggtgatcccaaaagcaggggacatgtcagaaggtactgcttaataaatacacgcttttagagacacacatcgttgggttgtagctgtgtaagtttcttgtgtttaacaccctgtctgcacattacttctgtgctgcctcaccactgcctgcccactcctctgttgttggcgttttcagtgatcattgaaacattcctgtctggagagtcctagttctcttgtgaagtctgctgtttctcaaaagccagagttgataggacttagtatcagtacttttcctttctccatgaagaatgtagctttataatagatgatgtcacacatccgtaatgggagggatgaggagatgcctgtctgtctgcctctctagcatggcccattctgctttctttcccccttgtgagctcttttccgatttatctacaggaaataagacattgaaattcagggcaggatattgttcattttaaagggaaatgtattttttaaagttcagtttttttttgcttttgtttatactttaattaaaaattttttttcctgccagttcctgaaaaagaaaatagagaaagaaatattattgttcctgggcgaagtggctcactcctgtaatcccagagctttgggagactgaggtgggaggttgcttgaggccaagagttcaaggtcacagtcagctgtgatcgtgctaccgcactccagcctgggtgatagagtgagacctattaaaaaaaaaaagtattgttgggagcataaacacgtgggaaatggtcaagaacggccgtcaatatactctgtttttcactgaaaactacctttgccagagagcgagcagagatgaggaaaaggagtggaagaagtcctccactctgatagtgttactggaacaacgagacaaaagcggtgtgctccttccacctgtttgctccgtgtccctgtcggcgccccctctcctgctaacccccccgtgctttctctgattgctgtttagtgtggatccttcacctgtgggtgagtctaagcaccgcccaggtcagtcttcagctcctgctcctccacctcgtcttaacccctccgcctcctcgccaaacttttttaagtacctaaaacataattccagtggagaacaaagtgggaatgctgtgccaaagaggtgagcacactcacgtggcaagtttggtgttgtctgttttcctggggagttcacactgatgaggatgtgctgaatggggggaatgtccatgcaggaagcagagccactgtgtgtgtgtgtgtgtgtgtgtgtgtgtgtgtgtgtgcgcgcgcgcgcgtgtgtctttgtttatattttgtcttattttcagctgtcatttgaaccaagttaattttactattgatgacttttnttaagattattatgaaaacagatcttaatggcagattggtttgtgtttgtgtttgttttttttttttttgagacagggtctcactctgttccccaggctggagtgcagtggcgtgatctcggctcactgcagcttctgccttgtgggttcaagcagttctcctgcctcagcctcccgagtagctgggactacaggcacacgccaccatgcccggctaatatttttatttttttttgtagagatggggtttcaccatgttgaccaggttgttcttgaactcctaacctcaagtgatccgcctgcctcagtctcccaaagtgctgggattacaggtgtgagccactgcaccctgctgcaaattgtttttttatacttattttcacatttccttgccctagtggacacttacatgcatgcgtatatacacacacacgcgcgcgcngtgcgcgcacacacacacacacacacacacacacacacacacacacacacaggataacatctgtgtttgatcatgtacactgcaatttgtgccatatcagaaacttcctgattgatttaggggaattatttttcccagtttgaaaggaagagttatttggaaaatggatggattttcttttttaaaaaattattgatcccattcatttaaaatcaaattttattggtgaaaatgaaaattaaatctcgttcgtgaactacttttaatttcttacctagttttcttttcttagcattagaacaaaaatgtttcttttattttgaagcttatattttatactttgtgtttttatgtttctttatcctaaactcttttttcaaccaaactcttagcatctcctactgtaatgccctgcggaaaaaacttcattcttcttcctctgtgccaaattttctaaaatttctggctcctgtagatgaaaataacacctctgattttatgaacacaaaaaggtagggcttaatttagatatatcaagcctgggtgttactaagtgttgaatatcattagatatacaagggtgttttaattactattttgccatttaaaaaatcatttcagctaaatctgttgtatcttctttcttatacttttttcttactgaatgccatttttaaaaatgtgcaaccaacctgttctctagttttgacgaggattagtttaagtgttgtcttaagaaaagtctttgccaagtctctgagaccagtgtttctggttagtgagcatatgtctgtttcaaatcaggatgtctgatctgttcaggacgtctaatctgtaagttgaggggattgcttacttacaggtacataacttgggtataaattggaagggccttctcaggttgtcctgtgaataggagaaaacatttatgattgtgtttatatattgataactgtattttgtagtttaaaaaatacacacgttaaaacaattatcatcatcaagtgactgcatagttattgccttgctggttctgtgtaattaaattgcaagttttttcattttttgtgggaatccttggagacatgggcctgtgctgagcagatattcccatgcacagaagagggcagaatggggccccttggcatcaccccctttccccctttaggcagtttctctttatcaaagtggcaccaagagaggcccaattggaactatgatatgtggaacatgtttcttaatctctgttacaatcgaaatcacttaagggcatgtaatctttctcttttcatgaaaagaattctgtaagaaagcagttctttaggaatgatgacccactgtgagcttgatataacttctgtgattgattatttgtttatacaaagatagttgataatttagtgatttgtttaaaaaaatgttaagctaacaaaatcccgtgaattcctccccactagtcataaatcaatcatcttataattttagggactttgaatccaaagcaaaccatcttggtgattctggtgggactcctgtgaagacccggaggcattcctggaggcagcagatattcctccgagtagccaccccgcagaaggcgtgcgattcttccagcagatatgaaggtaaggccggtacctgaaatgaaacctcaaagagagcacgctgacagaggaccctgggagccycatcatattggtaagaaagcagagcgccgtcctcttcagtattggcaggtctgaggcaatcacaaaggtaactagggagggaatttagaggttaccctccatttcttagggaaggaatttaaagctaatttagggtaacctctccataaacaggagcagagctctgatgtttagagtggtcacagtgttaaccagcggtgaatccagacaggtctgcggcaacctcacttcttgcctcctaggacataaggcaaaaggagagactgaggcaagttttagagcagcagtgaaagtttattaaaaacttcagagcaggaatgaaaggacgtcaagtacactttgaaggtggttaggcgggcaacttgagagatgaagtgtgagatttggccttttgacctggggttttatatgctgccatacttccggggtcttgcgttccttcttctctgattcttcccttggggtgggctgtccgcatgtgcattggcgtgctagcacacgggggttgtgggggagcgtgcgcagggtgtttactggagttgtaggcgtgctcacttgaggcgttcttccctgtccagtctagcattcctagaggaacgtcatgcaccaggtaaattccgccatgttgcctcttaatgcgcatgcttgagcccactcgcccagctcccgagatcttattgggaagctgcagctccccagttttaggtgttttctatctactgggagcccgcccttccttggtgcccgctgtgaccaacgatcactttagagaaacagttgacaactgcctgaccaacacctgatggtcgcctgacattgctggtgcatatctggaaagggccctctcctgccgtcctcatgtctgacgagctacccgctgtaaccaaagcgtgggcttcggagtctgctttcaaatcccagcttttccccttaggagctgtgaactagaataaactgtctaaagttaccacctataacctgggattaattatgcctgttgccacactgatagagacaaggcagcatgatatcattactgatacattttttttaaagcattcaaaattcatagtactggaaagaaaatcagtgatgcgaatgtttccagggtaatgtcacctcccatgctgtggaagtccttcgggtgagcctggccccttgcttcttttgccccagcctttctatgtgggggcaccatggagctgccactcaccagcaccttttttccctcaagtagtttgtacctataaagtattcctgccgtgggtggcccctcggtggagctgctgagcctagccagggtttgatttctcttcctgccagtgtgagccagatggccacatctctcttcccctgccccgtggagaggtctgcttaccgcaaagaagggctcttcctcccaggtcctgtagcaccctgttagagggtgtggagtggagcagtgggaaccagagccaccagagggaggccctggaggaggaacgaagctgattcatgtctgaaaggggtgccagaacccaagtttcggtgtttaataaagagtgcctcggtgttgcggtggccatacctcacagggcatggtcgcttggaaatttctgctcggaaatgctttgtgcagtggccaggatgcgttaggggccacagatgactgcttgctccatcatagaacagttccaagttttcaaacgagcattcacagactgagccgcatcctgcctccctgtcctctgattcctggcttcttctctggtctctgaagccacacggaaatgtgtttgcatctgtttcctgcccttcagatgacagaggaccatggaagctgctgcctcctttagctctcttctccaggggaattgccctcgtcactgtttgggaacccctggtccgagtcctgtcctccgaagagcctctgcccctcctggagtcctgagttgaacttggtgttcacttggcctctggctctggcagtgtgttgctccttccgttgacctgccactgctctgttaatgcagattgatcttcataatctgtttctgctttaagtgattaactcaaacattcttggctcttattctatcttgtcctttgggatatgaaccattatttaaatttggactggtttcctggcttggcacagttgaccatgcctgtaagctcagtgctttgggaggccaaggcaggaggatccctggaggccaggagttcgaggccacccttggcaacatagtgagaccctgtctctacaaaaaaataaaaattagctgagcgtggtgttgtgaagctgtagtcctagctacttgggagactgaggcaggaggattacttgagcccaggagtttgagtttacagtgagctgtgatcacacccctgcactccagcctgggcaacagagtgagaccttgtctcgcggggtgggggagtcatgtctatacttgagaagtttttttccctcgcatagtttgtacctataaagtattcatcagttttgagcagtccttttgcgattgttttgagtcttgattctggtgaccagtaagttgtatatatttgcctgtcaagtggacaaacatggcctttgtgcctttaagtaatggctaaaagtaccaaacagaacagggcctggcatagatgctgctcctcctgttcctaggccgtaatcacccctgattcatcagaccccaaacaagtcagctcctctccctcctgtgccccaccacccaatctcctgcaggaagatgtctggagacccctgtcagcgctaggcagagatcaccatccatgtccacctttcctctgatgcaggctcccactagcccctctggcttgtgccatgccagccatgaactcaccctcatgccccacccgagccctggcacaggctattccctctgcctggaatgctcttcgtcagtatccccatggctccctccctccccttcccttgtatcctgactctcccatagcagctctctccctgtaacacatgttcacaggttcatcttcgtcacccatctccggcagctcctgcaggcttgatggctgctaaaggcaggcaagtcagtggctcagattcttgcaaacttagtgattagtgatttctcaactccctcctcatgcctcctctgtctctataggcacatattatttcttatctcttctcagaaccaagccgcctgaatttctgaataacattgtttaagtgttctgtgtatgcaaaagaaaaacgagaataaaaggattattaaggaagaattaatataataatagccacatattatgctctttttatactctgctaagtgctttacatgaattatttcctttaattagacaatcttaagaacatcgacatttttatgaagcccattttacaggtgggtgagtggaggctgggagtggcttaaatcactttccccaaaccagggagttagtgggagccagaggcaggacctgagctcgcgggtctgagctccaaagctcattctctgaactgtgcacagcactgggctgcagccagagatgcaggacgctgcgggaccctctggaggtggtcctgcctgtgcttccctcttcccacaggaagctccctataggcatctgtgttgggcgtggactctcagtgtacctgcatgtctccctgttggccagacaccaacactgaatggaaaacatgtttctgggcattttaatgtacgtacttgccttcagtcaatctcctccgcccccttccatcctgaccgcctccctaatagttagcagtgggactggagcttgaatggcaactgatttctgtctgagaggacaaatcaggcatctttgtcctctgccactgtctgttccccatccttaggatgcacgatgccagagccctccactgtggtctgtgaccactttgacccacactagcaggtctccatatgttccttccagctgagagacatcacatccaaagacagtttagagctctgaggtttctttccccagaggtccctgctttgtgcaaactgtctccagccaagcgtgcacaagactctgttcctgatttgcctgggcggctgagccatgggcagctgagcctgcagccgctggactcactgcattcccactctgactttggcatgaaagacacacaagtgtgcttgtgagaaatagatcttaacagtaccttttaacacctatttcaggtgctcaaaatgactgcctgttttacatttatattctggcagtgcaaacttcaattggacaggaaatcttacaacctctcttccaggtgraaaagcgaggcagggatgtttatacagttccatccatgtcatcccacttggaagatactagtaaaacacaccaacagtaatacaaaaaccatggtgtttgcaatagtgataatgttactagtgaaggaaaaatagaaactttctgtaatttggagattctaatttttataggtgggctaaaaaaaaaayctggagagaagggtgttaagtgagtaaggagtgtgtctctaactaaatatagtgtaaaaagagaagaaaatacaaagtcaggcacagtggatagaggtggatagtctaatctctaatagtataatgggcaaaattgtctcaaacaaaattagtctgcctcttgtttactcagggatgtgtgactgttttctatgcacaaaatccccatgaaataattaagttgcaagaatctgaactttatattttggaaacctatctgaggtaggtaggaagttaatttatatttagaaatttgcttgcatatgtctagtagctccaggacaaatattcccaaatcccagactatttttttttctttttaaattcaacagtgaccagttggtctcttgtaagaattacagccttaagttagcaaagtctaagagggctggttttaatcctgaacctcagagggtccctgcttctcaaatactaagtaggtcacgtgcacagcaggtactacattgaagggaaattgtatgataaataggaaatcagcgatttttacttggagacttggcaaggcaaatgtttttgtaataaaaatagatcgtgaaatagaatcctgaaagctgcctgtttaaatgtaaagcaaatggctttagtgatgctttaagtgtggcagtcacttctggctgccgcagaaactatagaaagtgcattctctcttggtgctgtgggttcttagggtgaatgccttgtgtgacgctgagtatgtggaaggaccattcattcttggtaactatacactaggcagagggtggcgttagcgaagctactgcaggttgggtgtgtttaagatttggatttatttttcttttaatttttatttttagttccagggtacatgtgcaggatgtgcaggtttgttacatggttaaacgtgtgccatggtggtttgctgtacctatcaacccatcacctaggtattaagtccagcatgtggttatttttcgtaatgctctccctgctccctgccgccccccaacaggctccagtgtttgttgttccctttcctatgtccatgtgttctcatgattcagctcccatctatgagcaaaaacatgtggtgtttggttttctgttcctgcgttagtttgctgaggataatggctttcagcttcatccatgtccctgcaaaggacatggtctcattcatttttatggctgcatagtagtccatggtgtatatgtaccacattttctttatctagtctatcattgatgggcatttgggttgattccatgtctttgctattgtgaatagtgctgcagtgaacatatgcatgcatgtatctttgtaacagagtggtttatattcctttggttatgtacccaggaatgggattgctgggtcaaatggtatttctagttctagatctttgaggaattgccacaccatcttctacaatgtttgaactaatttacattctcaccaacagtgtaaaagcattcttacttctccgcaacctcactagcatctgttgtttcttgactttttaataatcaccgttctgactggtgtgagacagtatctccttgtggttttgatttgcatttctctaatgatcagtgatgttgagctttttttcatgtttgttggctgtatgaatatcttcttttgagaagtgtctgttcatgagagagacatatttgctcctctgagtaaagggtaaggatgcttacgtctgtgtgacagccttctctctttttcagaacctcactgtggatcgccatcgttggcctgtactgaaggtaaagcagatagaggcagtctcatctgtcagatgaagacctcatacacctgttgattaagaggctttcttcagatcatggtttagagcggtgttttacaaacttgacgtgcttggagtcttctggaaatcttgttaaaaggcagactctgttgcagtaggtccgggtgggttctgaagttctaacaagctccccagtgaggctgatgtttcaggtccactgtgaggaggcagggcttagaataaacaaccgtgggaaatccagtccagatctttgatgcatcctaggttaggcctgtctgtcaggctgccctgggtctctagtgatggactccaggagtctctcaagtctcaaataagtctgagtcatcagggatattttttgagaagagttgtgctgtctgaagaagcaaagagtgagtgtgatggggaaaatgcagtgattaaaaacatggtaaggtttaaagaaagatttgaccatatgccaggtgaacccaaatgtatggtgctttgcgtctttcctgccctttggttttccagggaggcaaggccttatctcttatggagcacaggagacacagtgtgggcgtttgttttctcagccgtgggctctaacctaattgtcaagcctacaaaaaaaaaatgctgaaaatcaacttctgactagatatctggtagtacataatctccataattttctctctgggtgtattatgcaaaagataatcctttngttattaagaaacaatttttaaggcaactcccaactttgaaacggggaaaaatcattttatttacctctatgtgctagggaacaatattaaatttagttttatacttttcctttaagcatttcagattatattgtgcatttcaccaacaatagaagctttcagactttatatgtcttgtaaaaaaaagcctaatatagataagaataatttattgatttgaaacccattgtataagaaatagtccagtgaaacttaagttcaaagttttttttgtcttgtggatgtagctatgtcaatatgcctagtttatagtaacattaagtctagtggattagatattagatatcaattgagatgtaagcagtaataaacagtaatgcctaaactgaagtatataatctgaatctttatgatgaccaatttatattattgtgaaaaacttaggaactgatttgaaacatgatttacatgttttacatgaaacatgatttacatgtgtcatatatagttttcaataatttacgtaccagcaggaaattttagtggataagtggaataaactgcaggtgaaactttgctggaaaatacaagcatagtgacatctgtgcaccaaaagcacctggggagattttttaaaacatgggccagacatgccttcctggctgtctccctcactgtcagtgagtgtggggatggggtctgggcctgaattctttctttttagactcctcaggattctgattgctgccacgttgagagggttgacctcaattcggacctcagagggtgacttgagaaactgtcaccacttggtggcagtgttgctccccgcatcttgattgcccttgtttctttccaatcccggaaaagtgtgcttgtttttttttttttccctgcgtgtttttgtttttgatcttgctataatatttatattccttgctcatttgcaacttatttgaatggagagctactttctgaaatctagatgtttttctttttctacagggttttagggcatgggcaaaacacggaagaaaaaagttgtcttcagttggcagagacgtggatttttaagattgttcttaatttactttctgtataactttgcttttctgtggtgaacaaagaccaggttcaagataaaatattgcaagccaagaatctgattgttcatggatttctatggttaaagatacttgatcacctccccatccgccccctaccccacccaccctgcgccgcccccacaccccattgtgcttcttggcttgtcatttcaaaagtcaaggaagtcacagtgaatggcaagattttacctcgacttgctatttttgtgcctgttaacaattgtgagttaacactgactgagcttttcctagtgaacctccggcgtttaaacagccagtccataacactgtgtgagggctggagctaaggttattggtgacacaagatagcacctgagccagtgctgcttggtaggagggctgaggggaagagggctgagggcttggatgctgagatgctagagtcacatcgcctggatttgaatccctgccctcctgttctgataccagctgacccatgacgatgctacagcacctgacagcagattcctccttagggctggtctaactctagagtgtgtgcctgtgtgcctgcaggagaatgtccaaagtgggtgatcttgatctgttaacctttgaattttaacctataccagggagccattgaagagtttaaagcaagtgaatgacgagtagtttgaaaatatttccaggtggatagaatttgtggacatacatgaacatgagcagcctcaaaatcagggctgggactagagtgaggccagcacgtgtccagggtgcaaaatgtaaggaggcattcactttcagggcctggcaggtgtggaccctgaacttccaggaccttgagagtgagtgtctcctaaggattacaccctgggggcctatttgcctcatcctggtccctggtcctctgtgtaccctattgcctgcttcagtaaacaggcagccctgcaagggaaggaagggttggatcagctctgaggagggagtttttttagaaggatagatttgttttgtttaaaaaacagctttattgagatataattcacatcctatacagtttgttcatttaaaatgtacaattcaatgttgtgaggttattttttggtatatccacagagttgtgtgaacatgaccacaatctaattttttttatttttttttttttgagacggagtattgctctgtcgcccaggctggagtgcagtggtgcgatctcggctcattgcaacctctgcctcctgggttcaagtgattctcatgcctcagcgacctgagtagctgggattacaggcatgccccaccaagcctggctaatttttatatgtttactagagacggggtttcaccatgttggccagactggtctccaactcatggcctcaagtgatccttctgcctcagcctcccaaagtgttgggattacaggcgtgagcccgacccaccgcagtctaattttgaaacattttttgtccccctagaaaaaaacctgtagttgtcacttgccaatctactgccgtccacctctaaccatagacagcccctaatctactttctgtctctatagatttgcctattctgaacacttcatctaagtgcaatcatataatatgtggtcttttgtgtctggcttctttgatttaacatgttttcaaaattcattatgtcataatacataccagtaatccattcttttttaatgacttattaatattccgttgtatagagacatcacatatggtttatcctttaccagtcgagaggcatttggattgtttgcacttttggctgttacggataataccgctgtgaacattgatgtatgtgtttttgtgtgttgaatgtgagctggtgtggaaactcctcctccaggggggccttacctgtgattctacccacggggatggttaagccagcagggatgggaagggtttggtcctgctggccctaggctttcctgcaggctgccatgtgcctttcttctgcctaggctgaaacggaggctgccctggtttctggcactgccctcgtgagtgtgtgggaaggctgggggaagccaagtctccatggtgcctccatcagggaccctgcagctgggaggcagccagagggccacaggttggtagcattcacacagagctacatttcttttttttttttttttgagacaatcttgctctgtcgcccaggctggagtgcagtggtgcgatctccgctcactgccacctccacctcccaggttcaaggaattctcctgcctcagcctcccaagtagctgggactacaggcgtgcgctgccatgcccggctaattttttgtgtttttagtagagacggggtttcaccacgttgaccaggatggtcttcatctcccgacctcgcgattcacctgcctcggcctcccaaaaagtgctgggattacaggcgtgagccaccatgcccagcctacatttcttttttttttttctttgagatggagtcttgctctgtcacccaggctggagtgcaggggcaccatctctgctcactgcaacctctgcctcctgagttcaagtgattctcctgcctcagcctccggagtagctgggattacaggcaactgccaccacacctggctaatttttttatttttattttttaatagagacggagtttttccatgttgaccaggctggtctcgaactcctgacctcaagtggcctcaagaggccaatccgccttggcctccccaagtgctgggattataggtgtgagccactgcacccacccagcccgtagctacatttctgtcagctgtttgcaaactgtgccccagaatcccctggaggacttgtagaaccaccagttactgggttacgcccccaaatgtctgatgctggagatgaattatcttgggtggagccctcaagccgcagcagctgataagcatggggacctcctattctgataaaaattccaaaaaagtcctgagtgattaataaacagcacattgaaaattagaaatgagttctatggcaggggatgaaacaggcaacaaagcctattttctttgcaatgaagcgcatcagatattaataatagccattgtaattatctttatcatgtattaagcattttgtgtttttcacttttacacaattagatgatccccataggtattaccgccttttttttttttttttttttgagacagagtcttgctctatcccccaggctggagtgcagtggcacgatcttggctcactgcaacctctacctcccaggttcaagctattctcatgcttcaccctccttagtagctgggattacaggcgcctgccaccagacccagctaattttttgtatcttttttagtagagacagggtttcgccatgttgggcaggctggtctcgaactcctgacctcaggtgatccgcccacctcggcctcccaaagtgctgggattataggcgtgggtcaccacaactggacttactgcccatcttttaagagatgaggacagaaagattgagtgacacagttatgtctcctgcagctcttggttcacatagccaggattcgtatcaatctatttagctctaaatctagtctcttaatcacagtaatgaaccgttgacagttttacgagtaaattatcaagagttttgataggtttgctcacttaaattagtgcttgtacagtaatgggctgtgttagtgtgaaggaatgtatcttatgttggaagtactctagaattaaatgttaactcttgctaataaagcatacatttggggcattattagcaactttttttttttttttttagcaaaattagaggcttcctagttgagtggtttatgttatttatatttatttatttgtttgtttgtgacagggtcttgctctgtcacccaggctggagtacagtaagtagcacaatcatagctcactgcagcctcgacctcttgggctcaagcagtcccctgcctcagcctcctaagtgcctgggaccacaggtgcgcatcaccacgccctgctaaatgtttacagtttttgtagagacagggtctcaccatgttgcccaggctggtcttgaactcttgaattaaagcaatcctcttgcttcagactcccaacatgctgggattacaggttgtgccactgcgccaggcctccatgtatttgaatgaaagagcagacatctcctggaggtggcaaagctatgcatgccccccctggaggggagctgggggctctggggttacagtgatggcacattcagggagctctccgctttgtgagatcctgagataaagccaaaggatgcattaaactgcttctaaatgaactttttccaagtgaatttgttatatcacttctatataaatgaaaatatttgcagcatgagtactaacaagattttttttttcttttaccccgatggagtctcgctctgtcgccgggctggagtgcagtggtgcaatcttggctcactgcaacctccgcctcctgggttcaagcgattctcctgcctcagcctcccgagtagctgggattacaggtgcgcaccaccacgcccagctaatttttgtatttttagtaaagatggggtttcaccatgttaaccaggatggtctctatctcttgactttgtgatctgcccgcctcggcctcccaaagtgctgggattacaggtgtgagccacgctcccggccaagattttaaacattatttaccaaagtaggaacgtggtaattatggtcttatataattctgaaaatgatttctagtaccaaactatgaattttatacttgaaagaatgatgggtttttcacagaaagttgaagttattatggtttgtnttcctgttcanggtgtttttgctggagaatgttcgatgaacagcagttctggtgataagttatggatgtacacagctggtgtggtttttaggattttattttgcagcagcatcttcctcaaacagttgccaggggaaggctttccttcttcttactggtaccagcctttctcttgcagacaaggcagtatgggagggttgggagacaaaacagaagctgttggtttcttcagcctggcaaggattcagattgcaggttatagattggaggccgtcagtggggatacctttccggacaaagtggtgtttctgcctggcactgcttgccagagaagtttcagttcttcattctccgtcagagaaacccatatggaccacattctgatagttttcttctgtttccctaacaccgaaggctcagcccctggtgcaggtcccagtgtacagcaggctgcatacagttagaccagatgttcttgtagtacgaaaagtcaccgagtttccattcacttgtgggtggcaggtatggccctccttacctcccatggcccaggtttctctgtcctgccgttttcacattttccaggctttcacctccaggtaccaaaattcacatcatttagagattgtgtctgcctgccaatacgcggatgtaccagtgagggattgttctcgcctgacgagaggtctggatgatgagagagcagagctggccctggggctcagtggtgacaccctcgagcttggctgcttctgttcttctgcttcctctgcttggattccttcgcctttggcttcccctccagttccaagcagaacaaaacaggagatatcaaggaggaaagggtgacccctctatatctggagagcaaaactgtcgcggaaatccctagtgtacttccatttgtgtctcattatctgaaaccgagttacctggctggtcacgtgcagccaccagaggcaggaaggtagtgattctgcctgtgtggaatgttctagcattccctggtagcttttgtttcttcaggcagccatgactttgcatagatcatttccttttgcccaggacactcctgctcgttttctcccctcctcaccaaacccacagtgcattaacagcgacagacttctcctcatcctctcaggccacttggatgtcaccatttcttctctttacccctcaggcgtagtcagcctctctgtgcctgatgttttatggctttgtgtatgccccgatggagagcgtcttactgtgtcttcgggttatttatctcaacctcgcatctgtgctatcctgtacagtaaccaacagccacatatcactatttaaaattaaatacaaactaattatacttaaatgtaataaaaatgtagcccctcacattagcctcatttcaagagccacatgtggctaccgtattgtaagcagagctcaagaacattcagcaatattgtgagagtggctaccatattgagagcagagctctagaacattcccttatcccagaaagttctcttggacatgctgctcaaggtggtgaactctgagatctccagtccccccagctccgtcactcagaaccacaaatgtggcaccatcagccttcagggtggtgcttgtgttgtactgtctcctgactaaagaagtaaacttcaggcagtcaagattttctacaacccacactgctcctaaaactagtgttactggatatgtaaaagctattgagcccagtgctttcaaggaatcctaaaagcaagtggggactgtcatgtatccaggttccttgttttgcagaagaagaaatagaggctttaggggaaaggggtccactcaaggtcatacaggcaaggtcatacagtcagtggtagaatggactggaatcttgattgttcatctcactcccctttccattaagccataactgattgagtatcaccaacctgtttgttttccctgattatgttcccttctcrcctgtttaatcaggtttcagctccttgtgagaggaagttgttttcagcttcttagccccctacctgtaggcggtgctctgggaacgctcaggaagcagatgcatgtgagcctgtctcagccaatgattgcttagttgcaagaaacaaaaaaatgactcaaactagcttaatcaaaaggaggcttttaaacagcaagataagggtaaaggctgggaactaggaagttgtcaggaaccaaggctgtctctctggatctctctttgaggccatgtaatttttttttcctctcggtctctttattctgcacaccagctcacccagcttgcttgttactatatgcagtcatgccaacccccagatctgtatgacctgttagcctcaggggcacccacatagctggctgccaatctgtgttttcttccaggtttcgagagggagaaatgattggcccagctcagggtcacttacccagggagaggtgggggaagtatggaggcaccgtggtatcagggaacccctgggccaagcttgtccaacccgcggcctgctttctttcattttttctgttttttgttttgttttgttttgtttttttacagctcatcagctattgttagtgtattttatgtgtggcccaagacaattcttcttctgctgtggctcagggaagccaaaagattggccacccctgtcctaggccattatctgggctgtgggaggtgtggagcagggtcagagctggagggggagggcatagcctccagccaccataagttggtgtgttcttggtaattatattgcttgtcaaccgaaggcagaatcaggacaatgaaagtaatgagaatccctagctttgtaacagttagtggttatctaaaagtaggtgaaattgtacatgagtgagtggcatgaatttcttattactaaagtgctcagatagctggctaactttctgtcaaagatccctctgctaggatcaacatttgattaatatatttatcctgtaataagaatttgggattcttaaagcaaaatagttgtcatgtggctgactacacaaccaaagatggtccaggtgtcgctggaagaggagagactgaagagctgttgccaggttcccacgtggaccttcggcatgacccggccatggggaggcctcacacgctcctgcatcgcccacatcttgccaagccatggaaaacacttgggattcatatctaaatcctagtttaagcttggtgaggacagtggcctggtgcagagtttgggtcatagatggtgcttggtttcttttgtataaaggggtatatgattttggaatatttaccaaatgtgggcattttttctataaaaattattgtatctactgagattatagtatgtaaaaaaaacatacacatggagaaagaatacaaagagagcattgatattctacagaagtggcaagaagatgtggtgataggtgatatttttgccttttgtttcaattttgtattgtagtgacttttttggtagaaaaaactaantttctaattaagggagaaacatttgaagtacatttagtctttctagaaaacctcatcttctcatagaagtttaagatggagacatacttccattgtgaatcatgatgctaaccagtattcagatttgttggaaatggactcagttttaaaattgcttctctcttgtgggctggaactgcaaatgattgtttggggatttttccccttttcttctatggagttattcaacttggcatgaccagtgatttgagctgagaacatggaacccttgatttgcagaaatcaagcccccaaaggtacagatacagtggtcattgtctgaagggtttcttttgttcttggccctcctgtccctgctcttactgtggcagctgcagctgcaggtgcctctgaagccttgccatccatggtcacttcctgcctgctccccacccacccctgggaaagagcccccaagtgtccaaaagcactgtgttgcctaatgcttgttgagagtctacatttctctagatctagcagaagtaaaatttcagtttgttatatttatagtttcaggaatagtttgggaatggatttaataaaaaatttaaaagcccatcatttttatatctctttttcgatatttgatggtttaaaagacatcaaagttatcttctcccattactcatcctatacaattaaaacctgttttttgaagttgtaataggtaagttagccttaggtcaccccatatttatgtaaactccagcccactgccacagctactttgattgtgatctgtcattgtgttacccactgtagggcagaaatggttcctgcctcatgccgttgctgctttactcttcctgaagtggtgtggttctgtctctgtagtccttggcacactgtaggttctcagatggcagggtgaaaagttcttctgtttgcttaaatctctcataataccctgagtctgtggagctcaataaattctacttggtattattgatagattatttggagccttttatgttagaaaagggattcttaatccaatgctccgttttacagatgagaagactgaggctcaaagaccatacccccaggagccatgatttgcactgtatttaggaatagtgtctagggtcagcacctggtgttggccgactgcagagcagcctggttaggagccctggggttgggcgggtctgggctgctggtgccacagcagtctccctcccctgggactttgggcctgctacccacccctgttccttcctttgtgagatagggctagcagtaactgtcttgtttcatcagaggcagtattgcataatgaatgagagctggggcctaaattaggcacaagtgcaagccctcagaaaactatgtacacctagagagagagagagacacacgtctgtatgacagagaggcagggtttgggaatgttctgatttcatgttttgaattggtgtgacctttgggaggatatccttggaatcgcagagcttcgtttacatcatgactttcctgcccacccacattttctgagaagccagagttttaaatgtggaccccgtgagcttttctctgttgcctcattttggcctgtggccttttgttttcttggtatgtcatgaggcaaaataaaatgaaactcagtgctggttaataactcccatcataatgtatatttctgtgaatggctttttagccatttgagaggaaaaagggtcatgtaaatttcagaaaggcctgattggctggagagtcagtgtagtgtcacagttaagagtatagatttaaaaaaaattttttattgtggtaaaaaacataaacataatactaccatctaaaccatatttaagtataaagttcagtagtgttaagtatattcacattgttgtgcaatggatctgcagaatttttcatcttgttaaactgaaactctatgcccaataaacaactcctattccccctctcccagcccctggcaaccaccattctactttctgtttctctgagtttgactactgtagataactcatttaagtagagtcatatggtatttgtcttcttatttctggtttatttcgcttagcataatgtcctcaaggttcattcacgttgtagcatatgacaggatttctctcttttttttccgccttttttttgagttatattctgttgtatgtatatttaacattttcttcattcatctgttgacattcatctgcttccaccttccaccttttggctattgtgaagactgcagctatgaacatgggtgtgcaaatgtctcttcaagatcctgctttcagttctttcggatatgtacccagaagtgggtttgctggatcgatcatagtgtagttctgtgagtaaccctcatactgttttctgcagctgctgtaccattttacattcccaccaacagtgcccaagggctccagttcctctacaccctcacccacacttgtaatcttctggattgcagattttctggatcaatcttctggattacacttgattttctgtgttgggcctggatgtttagaacagtatccctcctttggagtggtaaatatgtaagtttttattataaaataatggccatcctagtgagcgtgaggtaatatctcattgtggttttgatttccttcatagttaatgtggttgggcatcatttcatgtcctggtcggccatttatgtttcatatttggggaaatgtcttttcaagtctcaagtcctttgcccattttttaattgagttatttgattttctactgttgagtatggattattaaatcagactggcctgaacttaaatcatggcccttccatttttgaccaaaagcagctgtgtgtcccatttgtgccttggcttcttcggtgtaatgccggcataatgatagccccaccttgtagttaagagtgttggggcagtcagtgaggaagcactcactccacaggagcttgttacgtaaggagaaggcagccggtccattcctaataggggtctgaaggaaggaagaagggctgaaggaagtaaaaagagcctcctccatgaatggcagccattcttgaaatccaccttggctgccttcatttttaatgtcagtggacttttaagacaaccaaaaggatgttcttggatgaccagagactgtggcagagggaggatggtcacattgccaaggatctctctcaacctcttggatagtgtgctgctggtagtttgcacaattgcttcagctttttggcaaagtacatgtaaaatcctgaagtcactgccagaggaaacctggttcctgagatagcagcttgatgctcctgccccatcccaggtgcacacctcactgggcagctctggctctgaattgagggacagcaaaaacctctaaccaaccatactgaaaagcaggcattgggggctttaggggaaggttcttttcaaaactcatgatggggagagaccaaagactgggaatcattgtaaagaagttagtcatagatgcttcactctttacaatcatcccaacacaaggttaaacaacatgcagttttcacgatgtcccagaaagcgacgagtgcagtgaggtgaaacgtggccatctgagcacacaatgaccaggcttggaaggatcgatttcccctgtgctggccctcagaatttaaggcacaacttttaagctgagtgtgcagcactcgattttctatgttgggcctggatgtttagaaaagtatctctccttcagagtggtaaatatgcaaattttttactgaattacttcatttaatcaaagcagccgacttctcctgcctcccctgtttctgtcttggggttgaatatttggtcccatgtaacaactcttgattcttaatgatgccacatggaagctgtgtgtgctgggatttgccatattcagttatggtcagtagagactttcttagtctctctctctttttttttttttttgagacaaattcttgctctgtcacccaggctggagtgcagtggcccaatcttggctcgctgcaacctctgcctccgggttcaaatgattctcctgcctcagcctcccgagtagctgggattgcaggcacgcgccaccatacttggctaatttttgtatttttagtagagacagggttttgccatgttgtccagactagtcttgaactcctgacctcgtcatccgcctgccttggtctcccaaagtgctgggattacaggcgtgagccaccgcgcctggccacagtcagtagagacttttgaaaggaaatattacctctttaatgatgtttttagtccaagtaaattgtggtaatgtttaagaaatttgcttaccacaaaaacagttttcaaggagcatttgaacttgtccactttaagtcataaaatggattaaagtgtttgaaatctattgggattgtaaatttatgtcagtgtactgactttcaagagatcttgatgatcatgtcgtctgttttcattttctactacatgagaacattgaagcctgaaacttaacacaaacccgagttccccacttgcctaagagtcatggatacctaaaaagtatgctacttcccaagttgatttctttcaggatatgggcccttcaaaggaaagcagtgaggctggggttttccaggtggaaaggtcacatttccacatataactcagcgaacattgtgttgggttgggagaagaattggttcactattttaaactttttgtttcatcttgaggacttccccatcccctctcctccgcaaagcacaaaagtatttcctaatttttaagtcatgggcttcctttaatggattctgaactcagatcacgtccagataagcattgtgtaatgggatgggtggggttagatattttagtcacagatgcatgagaggagggagggtggaggacagcaaagtttataactggagcctatagtagtttatctcttgtcatcggccaggtcacagagtctcacttcaggacagctgtgcaagcagaacccccatcacggttttcttgatgcctttgacagtcacctgtacatgcctctgggacctttcctcctcctttctctttttgttttttttcccttggtcacatgtttcattctactaaatgtctaaccagctcttctctgtaaattacagagctgtgatggcaccttgcttgttgattatttctggttgaatagtttccaatgggacttctctggagataagtcctgtattagtccgttctcacactgctaataaaaacatacctgagactgggtaatttataaaggaaagaggttgactcacagttcagcatggctggggagacctcaggaaacttacaatcgtggtggaaggggaagcaaacatgtccttcacatggcagcaggagagagaagtgccgagcaaaaggggggaaagccctttataaaaccatcagatctcatgagaactaactcactatcatgagaacaggatgggggaaactgcccccatgattaaattatctctgcctgttccctcccatgacatggggattatgagaactacaattcaagatgagaattggtggtgacatagccaaaccacattaaatcccaagtgcgcatgtctggccctgatccctttatgtgagactggggtcatgatcctcccgcacccgtcttctgagccctattcctacttgggcatgcttaggcacttcagcatctgcatcccattgatgtcttaagggtggttccagaccttggaggtacacacgacacactgctgatgaaaacctagaatatagaatggaagttacatttattcatagagtgaaaatccaaaaatagaccagagagaagatatgaaaatatcaagaatgcttatcttagggaggtaggattataggtaactttttttttccttagataaatatatagatagatatattagtgtttacagtttctctgccaccaaccaaaatatttttttcaggaggaaaaaaaaccccagccagccaacatacctaaaaaccatctcctgggcccgagagggaaaaattgggctccttttcttgaaattgccatttgtgccactgttgtattattttaccagtaactccagattccaggctcctgtatctgagttctctctccttccacagtggagctcataccttcctgtttcctggctgccactcagatttaggctccgtttttcagacctcagtggctgtaatagctgttccttctacctcttaggatggttctttctgtaatagcctttgtcatcacatcatcagaggatgatagctcttaatgaggatctaaaatttgcaggtaagatatccctgcctctgacatgagatagatgtattgcatgctatttaacatacaactatactgagtgtgcagttgtatgtaaaagcattgttctaggtattgggttgaaagtggatcaaatgctagacaaaggagcgtacaagtcttgtaaggaagacagctgccaagagagaagaaaggatggggaaatgctgcgtctactaagttcaaggttctgaattggaaagctgcagctattgaggagaagagtcttttaaaattcctaaagggtttttgttatcttttattgatgcaaatgctattttgtggcataaaccttaataattttggggttgaaactcttatcaggataaaatgatccttttctatcccaagcttaataaatattgtttaagtacaaattaaatatatgaaatctgcccatctatattataaatgtcatatggcagaaattataccttgacttttggttctttcacaaaaccttaattttttttttttttttttgccttcaatgaattttgtctgattttacattaaaagcctgtaatttctcaagtcttgagtctggggagccgtcgtcatccttttttcccctctcccttgtcttctggatgttcaagcgattttaattagatgttgggcttttatgtcaagtgctggcattgcactccatgataatccagggactcggaagcacatgttatgcgtcaccctgggttggtgcagtggaactggggtgggttggaagtagtattctaaatctgcttcctgcgatggggtaggtcaggttgtcctgtgttgacaaggaagaagtctgggtgaggaagcgggatgaaagcagaccagacgctagagtccactttcaagtccgatcccaggacctggcttaaagttaaagaacagcaaagatgaaaggtgccgcacagcagcacaggtcggtggccacgttaatgacatagaaagcaagtgctgtgaattcaaaagaaaggacagctctgagccagagtacttggtgactttgctcaaacaaatccctttctggcacccccaggccttccctcccgcttcaaaaaaattctgaattgtgccaatccattgaggctcagctcaaggccatcccatgcctttccatcgtaataaagccttgtttcctgggctttaaacatattccttttttcttaggtacagattgaacttttttaaaagggaagttgtcagaggctctgtaaaacgttaaatcaaacctgctttgttttagggatggggtagcttggaatcagatttgctcctgctatggactgaacatttgtgtccccccaaaattcctatgttgaagccctaatgcacagtgttatggtgtttgaagggaggcccttgggaggtgattaagtttagatgagattgtgtgagtgaagccctcatgaatgggattactgtcatcccaaaaagaggtagagaccccagagcttcctctctcttcaccctgtgaggatacagcaagaaggaagctctctgcaagtcaggaagagagagggctctcactagaatacacttgtactgccaccctgatcttggacttcccctccagaactgtgagaaacaaatgtgtgttgtttaagccacccagtccctatgattttattagagcagcccgagctccattctccactccctggcttcctgcatggactttgcaaccagagcttcacggggtatagtttaatagctgtttctctgtaacgtagccacttttctctttccaggtctagttttgaccctcataacactttgttaggggagatttgagggtgaggaagttggcttgcttttcttttcaccatgtctcagtagaaacagaagcagaaaggccctgagatactgagcccacctttctcagcagggtgtgacagcccggagtaccctgggctgaggaggccagggctggaggggaggctcccacggtggaggggttgaaagctgggttgtaatgagctgcttttctgtagatgcctaaatgatgtgggttgagaaatcgtgatcttagcttttagtagtatatttttctgtttatgttaggtgagtcatcagtctgtctctgactatgttcagatctggaagttttctggaaggaaatttgttattgctgtaatagtgtaggttgttgatctggattagcagggagcggccccttaatacattcttaagaaaatggtatttagttcagtctttggctttgaactttgcctttgacaaagatgaaagtgcgacttgactggtgtttgaaaaacatggtgatatggccaggtgtggtggctcatgcctgtatcccagcacgttgggaggccgaggcgggcagatcacctgagatcaggagttcgacacctgacttggtcaacgtggtgaaacctcatctctacttaaaatacaaaaaaattagccaggtgtggtggtgtgcacccataattgcagctacttgggaggctgaggcaagggaatcacttgaaccctggaaggcggaggttgcaatgagccaagattgtgccattgcagtccagcctgggcaacaagagcgagactccatctcaaaaaaaaaaagcaagttatattacattttaaaactctatttaatggtcaggtcatccatccataatgggtagagtcattgcttaattaatttaaaacaatgtatttaaaaggtacctttgttccctagtgtcacataacgtgaaatatccaattaaggtaactgtaatgtaaagtaagtggctaaaaaagtgctgaacgccaaaggccagagattcaaccttttgtgtgcattagaatttcccaattgttcaaatccaggttgctggatctaccccagagtttttgatccagtaggtttggggtgggaccaagaatttgcatttctaacaagctcccaggtggtgttgaggctgaagctcgtgtggggaccacattttgagaacttctcccgtagactgaactcatggtctaggttctgtcagctgtgacccctgtgctgctggagggagtggtcagatgtcctgacctctgtgcccacagtgaggtccaagctgagtaggtttgaccagcagctgtaatcacagagtgaacaatgtaaacgaccaatgttgggtggtctgacatcttttaaaaaaaatccacgtggatgagatcacagggttaagtgtgggcagcagtcagggtaactccatgtggttactgcccatgcactctctgctgtttttcacctcttcttcagagtgtggtcaggatggtggccttgcccagcacaggaggcccttttccttctgaccacctgacctgacccacctcttagcatctgcaggcactccctgtcccttcgctgggccccgtggggaactacttgcagtcatcaaattcatcatgctgctttcttttaattcccacacttgccaaggtgggactgccccgcatctccttcccagtcgtgtgtcagaactcagcactggacctttcccctttccccactcccacccctcctcaccccgacgaacgtctcacttgggatcatctcttctgaggttggacctgcacagccgccctctgcactctcgccaccttatgggctgcccttgaccccttggcacacagacctggaagttggcctgctcagctgtctccttaggggtggagcttggttttctttcatcactgttctgcgatgaattgaatgcatgattggtcacaggaaggtaggggagggataaacaccttatgatatgtttcttataaggttttatatgtagaaagttatatgaaagtgtcagatatctatatatgaagtatatgtgaagttttatgatagttttgcataatttaagaataaactctttaaaggagctgagtcccaatcccttgggtcgagagttgcgtggctcccggggcctgcttgtttccttccactctgcgtgttcgttgctggcccctcataggctgtcccagacctctttgacttctctcctttctgcccagtcttccctgagacgctccaggctccctggcctcctgcttctcggagcttctcttgtgtttgttttctgtgctcagggcgccatggtgctataggccacagaggaggcgtctggggtccctcggggcaggtgcagcaggaggaagccgtctccgagggcatgaccttggaactgagcattgacagaggagagtcagccagacaaagaaaggccaaaaccccacccctctcccaccctatttctacgtgaccatgggccctggacacagcaagacggtgaccccgggcctcctattgttgcgaggagcccctgggaaaatgttggcattttcttcatagaacaggtttctcttctccagtattcttcagtaaatcaactttcttttttatccccaaccccagtctgattgcgaagaagtctaagcaacagaaagattttgccaaatagattatcttttttagaacaaaatagatcatgatattaataggaattcagcacttactcttgtctaagtactgtttttaagtgctctcaaggatttttcatttaatccccacaacaaagctgtggggggtggatgctattattatcggtgatttatgaatgaggaaactgacacagaggggtggtcgaggagcttgcccatttcctggtagttagtaccagggctggcatcatcagttgcctgctccttttcctctttgcttttgtgtccattaccccaaggcattaggatgagccagccaagttctagtcctggattcaccacctaattagctctgtgtcccatgtcttgccgtggagggataaaaccaattcctagcttatccgttggtggtgaagatgaaatcagtggggtacttgtaaagcacactgcccagcacatagtaagtgcccagaaaatgtgacgtcggacctctttaagcttcagtttccacatctgggaagagagggggagttgagctaagtcattttccagtgtccctttcagctccatgttcctgtgagcactgacagtttccccacaatmctgaagaaagaaggaaaataagggcggggtggcgaaggtcgccactgtgacgtggctgctggtgggaagtccctggggaggcaaggcccagcttcccagacacagccctcaggtgctcatcctggtggcactgaccaggggccatggtgggcttttccaccccaccatgtctcataaaattacaagaaccacagttgaaaatcagtgttacagaaatggtaataggatagggcaaactgttacaaagatcagcacttaagattctggctgaggcggaatatttgtttctctttagttttgttgtctttaatcaagaactgagagccctgactttcagctcctcaaaaaatacagcttccttccccttgcagatgcaaaaacaaacgccacttctttccaagcataattttctcccatgcgttatctcctgtctacagctttttcttgatccttctccagctcctgtagacctcccatttagagccaccagccgcccatcactggggctgcgcagagctcttggtgctctgtgccctgggctcgcccacccaggcctgttctctgngcctcttcctggttctcttccctggacttcccactgccgtgtggncttcagtgctcctctgagctgttgtcatgacctctaaccagactgagtcaggacttttttcttcctcatctctaagtcatccttacacagccttggaagtttaccctaaatggctattttgggagggagtggggataaagatctgcaggcctcttgctcctggtccttgtttctgcttatcttggcttctgtttttaagtgtgtgtgcacctctttcctcatcacacccttcccctccgtatggctcccatctcaggcagagttaggtgctctgttctgtgtccatagctctttttcgagcccttcttctcactgtttggtagtggcctttcatgtgtgtctgatccactaggctgtgcactccctgcctgccaggatatggttaaagtgctaaagaatgtatatatgagatcacttttgcttaaaaaacccccaatcttctggaattcccaatttctaaccaattaatatgtggattgactagaccttaagcaaccaagagtcagccagccttgtcttctatattcaggcgcatactatctggtcgttagacaaaatgggtcattatcagtgatgagttaataattacctgcacatcttgtttatgctggttctttacctaaagtggctcccatcaattaaacctgtatggattttacctgttcttccagaaccaccccactttccacaaaaactgacaacaatgatggtaagaagaatggtagttgacattttattaaatgtttactgtgtgcaggcttgtttttttccacacatttacctacttaatgctcacaataatcctatgaactagtcagttttatgcagatttcgcagattaggaaactaaggtggcaagtgatcagataacctgtttgaggttgagtagctagatcatggtagagccaggttcaatcccagatacctggctccagggcccatgctcttgaccttataaacggctgaaattcatctttttttgctgaacttccagaacactttctttgtatttcccttattttggtagtcttgtacttctctgctaccctgattcatacttggatttctagcagcatgcctggcatgaggcaacaacttaacagtatttctttataccaaatgaatgttgtcttttttttttttttttttcttgagacagagtctcgctctgtcgcccaggctagagtgcagtggcactatcttggctcactgcaagctccgcctcccgggttcatgccattctcctgcctcagcctcccaagtagctgggactataggcgcctgccatggcgcccggctaattttttgtattagtagagacggggtttcaccgtgttagccaggatggtctcgatctcctgacctcatgatctgcccgcctcggcctcccaaagtgctgggattacaggtgtgagccaccatgcccggccatgaatgttgtctttaaaaaattctgttttcctctagctagactgtcatataatgcaactgtaggaaataatcaggttctctttggagtattttccataaaagatccacagaagtcatggcagggttgagagtggacttgggcaaatgaatctgttcattcattgaatattccatgcatatctgctgtttcccaggcatgggatatggcagggaacacagaaatctctgcctcctgggctctgctttctgttgtagtagaggtaaagctgctcatactttgtaaacaatatgacaacattaagtctacatggtcattttactttgtttttttctaagaaattttgagctgttcgtaacaacagacgctgcagatgttaatcccgttgttgttaacttttctccagagatttaatgttcaattttctcctttccagaatcgatttatgttgttcaaacagaggtttgagaataactggaatttttttaacttcttttttttttttcgcatggagttcagaattttcaagagggatgaagagagttataaaatgctctatggtgggtaacacacagaaaaagccagaaaattggagaataaggatctgtctactcgtttccttctagagctcctctttcttacagggcacttaacatgtgatttaatgtcgtgtctttaaaaggaggagaactgcagttcagaacttaatgtcagtgctttgtgaaagtgcaagaaagaagccctgtattctgcacttgagagagccagatactgggcagataggaggtggtgtgcacgttgctttttgtctttctcgatcatggcattgattctgttcataacaatgatgcaatgtcatcctcttccccacacatttgtgtgcagatagaaagaatgcaacagcacagagttgttggggaataatttggcatctaaaatatcgacataccagcatagatcatatttatgactctgttgggagtgtcacagcaatgatttaataggaggcagttgtctccaaggcctcctgaattatgactggttttaaaattcttagaacccattggaggctattgtttctgaaaggctacataatttaagtgctccacatccgtcattataggagatgtcagaatagtaaaatctaatcctggactaagttgttatcgcagccctttggtttggtggctttgccgactttataaatatgcctgtcagtgcctgtggtctctacagttgggcagtcggcggtgaatatcatttctcacattttacactgggggactggaacccagaaggcatatgttttcccaagaggcaccaacacagttggcccatgaggtagagcagcccctccttcggctcagcctccgctgcactgagccaagccaagcttcctacactggcctctgtgcagctgtctctcagcaagaatgcaagtcggggagagaagccggatccctgggattgttctagagagtagaaacctcagagtagccctccttagaccacctaacgcattgcatcgctgcatacatgtaagggactcaatgctggtaggattggcttaggaatgatgcaagtgaaaacagtgccccggtttatcattagaacaaggttcttagctgacagttgcctcagactttgattttgttctccttgacctgccactccactcgagtccacatctctcaagactgcacacgcctgaaggaggactgattacaaaccaaagccttgtgcccagtctggatctttttgcattgttgagaaagcagcttactttctttggactgattcagcaggccaaatttagaacaaagatttttaactatctccctttataaattactgagctattttgtagccaggctactcttaatatgaacaaaaaatattatacaaatttgttgttaatcgtaaactataaaaaaatcagtaattgttaccacgtgaaatgaatttggataaaagagatacgtttttgccccttcccagggtttaggagagacgaaatggtgagattttagctctgaatcagaggttcttattagaggtggttttgttcctcctgacccctaggggatatttagcaatgcctagaggcattgatggtgggcagatgctactatgccctctgctaaacattctacagtgtataaaactgttcctcctgacaaagaatcatccagccccaaaatgtcagtagtgctgaggttgagaaaccctcctttaaactcttgggtttatttgctgacctttacagtggatcagcttttatttagttcatgtagaggtgaaattaatactagtgctcaaatatgtctttgtattctggacttggcctggatcccccgaccaaatttgggacaagctcctgccatgtgttgaggacctgaattcaggcagctaacaacagtatttgaactgtgttttcagtggtgggagtgaaggagatgagccgacgtgctagcaagcgcatagggttgcatgaggaaatagagagtaaagctgcagcgtggagccctgctattcagagtgtgcttggagaaacagcagtggaggcattactggggagcttgatggaaatgctcccctcagacttgctgaatcaaaatctttaatttagcaagatccccagtgaggcttgtgcatgtagaagttagagaagcacggggtaaactcttcttttttttactttggaggaaaatacaccttttttcttattatggctctgacccttactagctgtgtgaccttggccaagttataaaacctcactgcaccttatttgttttagctggaaaatggagatcataatatcacctgtcctatgagattgttgtaagaatcaaacaagcttatttatgccaagaacccatatggtaaaagctcaacaaactgtcactagtgataataagaaaaagatcacaaaagtagaaaacattagggagacagcttaggtcttaaatctcacagttgtcgtccccaaacaatacttgtatttttgcagatccagtttctctgaatactaaaataaaaccggagtttcataaacttctatagacagtggtccttgtcagtagcccaagtggcagagagtacatggatctggggacaaacagcctctactgttaggaatgttccatcctcctggcctgagttacacctgctcattgtgattccgaatttgaaaggaacacagtaggaattttcaagaccctgggaagaggaaggctgtggtaaacaggaaggatgagattagaagaaggagtttaggtgaggtgagcccttgttttactagtagggtttaagaatatccaagtcagctggacatggtggctcacacctgtaattctagcactttgggaggccgaggtgggcagatcacctgaggtcaggagttcgagaccagtctggccaacatggtgaaaccccgtctcaactgaaaatacagaaattagcagggcatggtggcgcatgcctgtaattccaactactcactcgggagtttgaggcaggagaatcgcttgaacttgggaggtagaggttgcagtgagccaagattgggccaccacactcccacctgggcaacagaatgagattccgtctccaaaaaaaaaaaaaaaagaaaaaaaaaaaaagaatatccaggtcaaccccacctaaccctcagcggggctcccttctgttgcctgggtgggtcctgggttctcttgacgcacacgagattgtgagagtgtatggaaacactgccctcgctatcaggacagcgcctgccatgccagccagaacacatcataggaattgcaaaactcttttgcaaaccagtgagagatatgcttccaatgtgaggtaaagcagaactttaatcacagctgcagtgttccacagaattccaagagccaagatggtaaaagaataaaaaaaaagaaaggaaagggctcaaattaaagacttcaagctgcagaataagattaaataaaaggattcaattgaactgcatcatattcagtaatgactaatcctaagtatacagggtttgggggtgaaaggatttgtaagtgttttgcaggaaaatattttttccatctttcattttaattagaatagatttgcattattttttcttagtttttatttttaaaatatttattgccacaaatttagaaaatacagggaaaacataaataacagtacatgtaaaccaatattttgtcccttcttttgttcaacagctatttctcaggcacctgctgggtgtcagcagctgtgctcagtgtggtgaccaaaacccttgtcaacaaggcagcaaggttctaacctggttagggcttacagttgagtagctgaaattttgatttcttttctgtgcccctagtaaagatatgatagcaaacaataagagctattttttttattgtgttcttactctgtgttgggccctgttctcagtggtttatagcctattaactcagtctctttaccaccactctgaggggaggctctgtcatacccacttgacagatcgggaagtggaagcatcaggaggttaagcaacttgttaaagatcacaaaatcaataatgacagagttttgattagaatcccagcagcctgtctccagaacctgccctattaagtgcagtgcaactgtactgcctttcataatatgtatcaaattgagatgatactttataatttcaattcttgcttttctattgaacagtacacagtaacatcctcctataatgcatataaacccccaaaagatgtagaattttaatttattcatttgtctgataggctcataatgaaataagactctataaagctgtgtaatttagatataggaaacatttggattatagtggtatgtagtgggaacaaatggtcttctgaatcaggaagacatgagttagagtatgccggtgtacctccttactcactgtatgaccttgggcaagtttctgaactttagtttcctttccaggctaatatctgccttctggacttgtcatcaggattaaatgagtctacctatataaaatgcccagcgcagtgcccagcacgtggtagaaggtctgctagtggttactgttactgctggctattaaatacattttaatcttccttcagaatacctggccagatagcacagtggttaagaatgcacatgaaagccagactgttgggttccagtcctggctcgactccttcctagctatgtgacattaggcaacttacataaactccttgttcctcagtttgcatttctttaaaactgcatagttatcatacccatgtcttagagttttgtgagtgtaaattattgtatataaagctctgagaacagtttggtacacagtaggcactgtatgaacattttctgtaattatcaataatataattattaaataacattttcagaaggagataaaaatattacaccttaaaaagcaggtatctttaaattcttcctcagctactgaagttttgcttactatttgacatatcatttgtttcacgtttgtggctcagacgtggcttatgccaatgcatattaacacaggaattttaaatttggtgatattattatattttatctgaatgaacagaatttgctgatttgacactgtgtttgaatgtgcattttttgttgaaaaatgacaattctggaatgccgtctccctttccagattattcagagctgggagagcttcccccacgatctcctttagaaccagtttgtgaagatgggccctttggccccccaccagaggaaaagaaaaggacatctcgtgagctccgagagctgtggcaaaaggctattcttcaacagatactgctgcttagaatggagaaggaaaatcagaagctccaaggttggtttgccatcttgatattgaacaggcctggtcttatcttggctctgaagttaatcacatcagacataagcatgctgtcttaaaaatacagcagcacgatagtctaatgtatacatctatctatatctgtttactttttcagagtaatattaacactgtttactttctggtgatctaatgatagtttcaccaacaatattcattattcctctatggtcactgttagtacagtgtttagaacttctgagatccaagctttaaatctaagctctaacacgctgaaaggtgcttttcattttgttttgttttcccctctgtctctctctctctctctactttatcctcagccatggtctgtgcctgtgtgttaggtatgaacttttcttgtgtaagtcattaacatacgtaacttcactctgtgtgctttttcagtgatttgcaagtaatctgaaaaaaaagaattagctgagttctacctgtactgatatcaatagtgtcaaaatatgacatgaactttgaaagtttagattttgttcatttcctgtttccatgctgacactggaaccaattaatgttatcttcaaagtagcttaagatgcaaagtttacatactctttggaaagagcatgagtcttagggtatctagagaactgcccggtgataaagtagtgaagattttgagcaggaagtctgcataatctctttcaaagggaagatgtagcagatggttcagtcaccctgccattgccccagaacaattttggaattacagtacatttcattcagcatcattcttgattgcaaattttgatcttttaaaatgaccttgatgcttgtatagagctaaaaagtcattaagacaccaactctgaggaataagctcctgagaatgtgttgcatctgtgagtttcagttgcatagctagtgtcatagcgagtggatagacgttctctgtgcatgtccctacaatgcttgtgagttatgacaacactgtgtacgagcaacatagtttctgcagttgaaaagtacgaattcatagaatgtaaagagatagtgtctatatcttttgactgaaaacagaaaatgagatataaaggaataagacctttcgacatgaaagtaaccccacagttggaataggctagtaagctttccaacatgcagttttgaagctgagaaagacgggtcctctcatcagggtgctgtggaagatgatagcacactggggggcgtttagagcaggtgagtgctgttttcttccaacccagtttttctgccactttcttatgtttttgtgaaggtaattttaaaagcagatgtctaaaagatgtttggtagtgatggcattactgcatgtgtcatcagttaaatgacagctcgggagcacagcagttatgttcgtgtgtatcttgggatttttgttgaagaggaaaaaggcagttatgttcatcatgtaggtcaaactttaatgccaatactggccaatattcttgcaaatgacagccatgtaaaatcagggcatagctataaaatgggaacggtgctcacagctggcttctttgtggtgaggacagctataattggtgaggcaaaaccagtgtgccacaaaagcagaatacattctgctgtgcaagcaatgaccagacagactagaatgaaaaggcaagagtttcctaaggttacctggaaccccttgccaggtgttgcattaagtttactggcccttgccaacattcttctaatgcttcctcatttcatctggcttcttggcagtgttcagtttttgtggtcttttatttttactgtttgacttcatttctcttcttagctctgtaaagttccacatgtgtttatctttgtggtgaaaacacaataaacttgcttaatataatgttggaagtattaatccattgtattagtgtgtacaggacctggattgctgataaaaaaataactagcaataacagcctgattgcttaaaaatatttagtaagttttgtcggggtggattggggcagggcagaacttttacattaaatatagatgcaagatttgataagaatcagccagagtgtacagtaagtattcacttaatgttgccaataggttcatggaaactgcgaatttaagcaaaatgatgtataatgaaacaaattttactaagggtttattgataaaaacaagagttaagttcctatggcatatttctgggcacaaaaacatcaccaaacttctaaataaagacccaagacacttctaatattaaatattgatgtaaacgtgagatatgcaaacatttaagcaagattaatacaaatatgataattattggcttggcacagtggctcactcctgtaatcccagcactttgggaggctgagacagggagatcacctgaggtcaggggttcgagaccagcctggccaatgtggtgaaaccctgtctctactataattacaaaaaaaaattagccaggtgtggttgtgcacacctgtaatcccagctacttggaagagtgaggcaggagaatcgcttgaacccaggagacgaaggttgcagtgagccaagatggtgccactgcactccagcctgggcaacacagtgagactccatcttaaaaaaaaaaaaaaaaagaaagaagtaattatttttccacttattccacttcagggtctcagggggccagaacctatccctacagcttgggatgcaaggcaggaaccagccctggaccgaatgccattccatcttggggtgactcacacacacactcagactgggaccatgtagacatactgattaacctaatgtgcacatctttgagatgtgggaggaaactggagcacttggagaaaacccacacagacatgaagagaacacaaactccacacagataatggccccgggctaagaatccatttttttcttgtcaacattataagaaagcgacattgagcataaagacattatttgaggacctgctgtactatgtacttagagagataggcattctatcttgagttccttttttttctcccttcttgaaggaaggttaaattgcatctgagatggctcttgaaattgatcaggggttcaagctgacttgcatactctttgggaaagaatttagaaggatgtgtatgaggaagttcttatggttaagcctgtttcctgacttgaatagatgaatcaaatattttttactattctggaagcatcgcattctggaaagaaccatactatgtcatctcagtctacctcactccattgtaggcacttggaagctgaagttgtgatttctccaaaattagatagctaattttcattggtgttagaacaaaaagcgctgcctctctttgaagacaccagtcctccaccgtcctcctctgcaaggccgttttccccccccttttttttttttttgagacagagttttgctcttgttgcttaggctacagtacagtggcacaatctcggctcactgcaacctccgcctcctgggttcaagcgattctcctgtcttagcctccagagtagattacaggcacccaccaccacacccggctaatttttattattagtagtagtagtagtagtagagatggggtttcaccatgttggccagaatggtctcgaactcctgacctcaggtaatcctcccaccttggcctcccaaagttctgggattacaggcatgagccactgtgtccagccaatttttctgtatttttaaatgaagatgtgagcagcctaatgtaagatcacaacatgtgattcaatacagccgtggcttggtgttgacatgttattaccagttgagctaatccatgtaactcagcattttatgctttactaagattaaaatgatgtgataacattaaattttgaattacagttgatgttttttatttaaaaaacatttttcttagttaaataatacatgatggtttaaaaatcaaatattcagtgcaattcttctaaaatctctgcaagtgtgggggtcatttaattgctgagcctcccagcctattagctttccattctgagctttcaagagatggtggcagctggcaaggcagttttgtctgggaaagccattgttaacagagcagaattggggatggagcagccatagcccacccaccagagtaggcacaaatcagacctgaacgttatcacaaagtccaagttggctcagacatttgtgttaaatcataataaatattttagagaacttggttgcaaatttacatttgatctcagtcagtcctcttcccctatctctacaagcttacaaaccgcatgggtgtgtgggggtcttatttaatattgcgaacagctggttcctgtatctgaagttcttgccctggagcctgggtgtttgttgtagttctgcaccatctgccttggttgataaggcattttggaggccactgattttaggcagcagtgttgttaggatacggaaacagcaggatgtttgtggattgagccttttcagctgaatcttctggccagttctttctggctgtgtgaagttgtgtcgactacagagcaggatgctcatgttgcctgctgggctctgttagggtggccagacgtgcttgtagcagccttactgccagaggaacgtacgttggcatccagagtccagtgctgccgccagttgcagtgcagcaaggctagccccaaacctgatttgctgcaaggattagctcaactctagtgacatttattgtgttttctcatagcccaaatcacagccaaaaaaaaaaaaaaaaaaatctagggttgacatttttaaaaatttttttaaaaaacatttttcttggttaaataatacatgatggtttaaaaatcaaatattcattgcagttctaaaatctctgcaagtgtgggggtcatttaattgctgagcctcccagcctcttagctaaaaaatctagggttgacatttttaaaaatgtattcaacagagtacgagggaaaagattaaagatggtggatggaaaaccataaaagctgagaggaaggcagcactgggcttagagtcacttggcttccctctagctagtaaataaccagcaccaaatcacctgatcctcctgaacttcagtttctgtggccatgaaataagaggttgggtccaggaatcaatgtaaattgtcaatttaacatttccctttattgatattactcccccctgggcttgataatttagttataattcttcatgcagctttaggttgagtaagtttggtgggaaacagtagctctcttcatatatttgagagatgtcatttgaaaggggtagatttattcagtttaactccaagaagcagaaatgggacccatggtagaagctaccaaatggaggtttggctctaaataagaaaacgatctttggagtgcctctcctagtttagatgaaaaaaattgcatcaagttgtaaccatgctagtcattgggaattttattaacaacacgtagctcctgtcctggggaggctcatagtttgataggggtaagatggaaagaattgggcagatgtggattatgtcttagcagtagagccaacagagtatgttgggggtgaaggggtaagagaaatcacatacctcctaggtttttagcattttccaaaatgaggaaaatgggtagaggcatggacagtgacttatatttagacgcgttaagccagttgtaactgcttgacgtctcagcgggataacaagtaggcagccatgttgtgtaatggaaattccatagctgtagcctttactaatgcgctctggaatggtctattccagcctctgaaaatgatttgctgaacaagcgcctgaagctcgattatgaagaaattactccctgtcttaaagaagtaactacagtgtgggaaaagatgcttagcactccaggaagatcaaaaattaagtttgacatggaaaaaatgcactcggctgttgggcaaggtaagcttcattgggaagcatctagtcaacctcacccctcattggtgattggggagaagtgtggaattaaaaaaaagtcaagtctaattttagtggccatctcccttcttttcatcacatcttaatctatttccatataccttacttaatagacatgagtttcaccacctttcatgattcctcttaattaaaattcccagaaggccgggaaataggaagaagacagaaaaacccaagggttttgttgcctataaactagataatgatttgatgatatactttgaattaaattataaactagaaactaattgtatggcttgtctctgggtactctagggagacaacatagtgtggggagcacagacttcagacaggtggtcttgggcttaaatctcaggcctgccacttactttgcagtgtgatcttagacaaatgcctcaccctctctgagctccagtttctacaagtgtaagatgtgggtgctgacagtggatgttgtgaggagcacacagcatgtgtctgctatactgtaaggccttagagagcgggcaggattcactgttttttcagtgagatctgccagcccaaactgttactggtccaagaagagataagtacagaacttgaaactaagcttttggaaatgtttccagcaatgtgacacagtgatcctaattaaaaatgtggacttatattttgtccatctgtttttttttaaattttgtttttctactaatttatttttactgtatcgtataaaaatatcagcctgtagtagattggaaaatttttaaaaagaaaaaaaattgatgcttcacagatagtttgagaaccgctattttgaagcttaccttcagtcattattagtgttctagtcaaacaatgatttctttaaaaatatatgttaatgtcttctggcaagagtaaaagcctgagtctaatctgattctatgctactgagttctggttgagctcatcatgaataaccaggtgttctgaataagggtttcaagtatgtatagaatgggtttttcctgagtttatcagttgtgcagtgggaaaacgttgtatatgcactttttcttttttgagatgtagtttcactcttgttgcccaggctggagtgcaatggcgcgatctcagctcactgcaacccctgcctcccaggtttaagctattctcctgcctcagcctcctgactagctgggattacaggcgcccggcaccatgcctggctaattttttgtgggtttttttttttttttttaagacagagtcttgctctgtcgcccaggctggaatgcagtggcgtgatctcagctcactgcaagctctgcctcccgggttcacaccattctcctgcctcagcctcctgagtagctgggactacaggtgcccgccaccatgcccgggtaaattttttttgtatttttagtagagatggggtttcactatgttagccaggatggtctcgatctcctgacctcatgatccacccaccttggcctcccaaagtgctgggattacaggtgtgagccaccgtgcccggccaattttttgtgtttttactagagacgggttttcactgtgttggcaaggctggtcttgaactctggacctcaggtgatctgcctgcctcggcctcccaaagtgctgggattacagatgtgagccactgcacccggcctgcatatgcatttttcatctctaggagcataaatggaacaaagcagtgttttttactatagttttttaggcatttttaaccttttctgaattttgacatcaattttagtaatcatgggaagttattgtttgttacgcattttccctttctatggataaggaaacttgggcttagagcagttgaatagtggcttagggccacagagctgggttcacaccaccgtactgcactgcctcctgttgaacaggatctccaggtgcttatctcagaacacgtatgcagtggtgaagaccgaagttctggatggacaccagctttcagtgtgactttagcaggtaccctctttctgggctcttgcccccttactgatagaaggagagacttgcactgagtagaggatcttggagctgtcttggagttctaatattccttgcacctgtactttttcttgaggtttacctttacaccaaatgaccccaaattgctgttttgaaaagggagaaagcagagaaaagaatgagtctgttcttcccccattcacagttgcctagatgatcaccttcaggtgtctttgcttctgcgaaaggcaaattgcatgggtctgtgacagctattccaaatatttgagcttcttagaagcctggcacctggatatttgtttttcactgggcatattttgtgggggctaatagaaatactctaggaatctggaccctgggtagtgaaagttgggcacagatgattgagcattctgtatactggagtgagctaaggctgacctggaatttccttatgtgttgcctgactttgccacatcactttttactgcagaagctctaaccataaagggggctttgtcagtcaggtggttttaacacattaagatttaacaactccaaacaaatgagggcggtctattttgtggttcagaataaaaatgtgaatcaaaaaatttgagcctaaatttgaatcatatctttgacctttgaagtagaggccaactcacctcagagaccttgtaagagaggacagttgtgtggattaagaggcccttcctcatagtgacataaaagaccctgaagtgatggaaataaaggaatttataaaattttcccagttaaaattagatgaggggccaggattagggtatcaatttaggagaagataacataatcctatgacattatagataattcagtttagtacacatcaaaatgatttctctaaagatatctagatagaaccttataagctggaatgtcttttttaggaatgggattgcagaggggctgcctgggctgctgacagtaggggccagatgcaaactctgcttgcctttgacccggcaatgccatttataaaaacttactctagaaactaatcagccaaaaatgtactgcagtaagaatgcttattgtgacattgtttaatagtcaaaacaaaacaaaaaacccaacatgtgactatcccatgtcatattccttgaaaatgacaccataagtagatctgtatttactgacgtaaaagatgtctaagttgttaaatgaaaagagtacagcatggtctcctgtactgttgatatttccatgtgcgtatacatggaaaaacacccacgatgcagatgtccaggttatagacaggatgaccatagggcccaacctggcatagccctggtttatgactcctgtcctggcaaaattattaatagccttcctcctcttcactgtcaaaagcttcctgctttggatggtaaatatatgcttattctagttatgggtggtttttcactttcttctttatacctcttgcatttcagaggtttttttgcaccacttttaaacagtgagtgtatattattttttaagtgagtaagaagctatttacatgggggatggaggaatggcctcctgccctcccagaccctgcctgcaagccgtaggtgggctccactgccaggtttctcttgggttaggagtgaaggcagcaccatggtggggaagggcattccaggccattcttagcaaaaacattgggtccaacctgcatgatcctgtgctttaaatcacagaatctaagcttactcctgaataccacaatatctggtactgtccagtgacacagccaatattcttttctttcaaaaaataaaggtctgataagacaatgggaatgatttagtaataggaaattggacatttcataacttgggaaaatttcccagtttgagaaaaagtattttgtgaaaaaaagccccactataaatcacttatcatgctgactgttttctagcccacatttacttctcatcagcatttgaagtatttgtggggagggtgtgcgtgtgtgtgtatgtacccaggatatatctatgagctggaatagcagagggagacaagaaatagaataatagtagaaagcagagatcagggtatatttgcttcctgttgctaccataacaagttactacaaaaatagtgactaaagcaacagaaattcttctctcagtgttctggaggccatagctccaaaaccatgccgttagcttgtctgtacttggcctcttccagcttctggtgtctgtcagcttcctagacttgtggtcacggcactccagcctctgcctccttggtcacattgattccccttctcatctcctcctctgtatgtctattataagaatgcttgtcactggatatagggcccatctggataatccaggatgctctcctcctcccaaagtccttacttaattatatctgcaaagaaggtaacattcacaagctccagggattaggaagtgaacacatctcttttgaggggacaccattcaactcactctacagggtcattatattaatgctgagataaaattacagaaggtataggatgtggtcatggtttacaggggccctgtatttcttctacaggccaacttaaaaaaaatgatacgtgaaagggaaagaagaaagtacttactacacagtaagtatttccaagaggtggcccagtgagacttttgaatctgttaataaaatgattactatttggttcaaatccacagatggttattttatcattaattgcaagataggaacacaaaatattttttctctagtccccatttgagtagcagccttgtttgacatttctgacatggaggacaccaagagaaaatggcagtcagcatccctgggctgtcactcaccggcctaatgacctagggcaagggacctgttctcactgcctctcttttctttaccatgaggataatcatgtttcccttagagggttatgagtatggtatgggccaatacacataacgtgcatggaatggcgatggtgcatagtggcctcgcaatcagtgctatctgctgctgctacctgccagagcagaaacttttcccaaaggtggccagagacagaaaccagagaaaccatccttctggacaggctgtctgagtggcagggcagggtacaaagcggccactttttttcccggatggaaagaaagatcaatgcctaacttggaggcttcctttctcccaaaagacaagaaagacttggcatcttattcttcagtcttcttgctctccccctttccacctttttggccttgtaatagctgagtaatgagctaaagaattttggttcaaaactgtcaccttttaaaattaggtttgccctaaataacatccttgactttaagagaattttcttaagttttagacatttttaatcactgtgagtattcaaattaatcacatgcaaagcattagttagaggctcttggacattttctgtttttagagctttgttggatgctcacatggcaatgtctgtgcagtcagttcctacccagcctctgggctcttcttgcagcttatcttgcagaaagaagcctcatcagaattccagaatctcagctatgattagcttactccacctcagctcagaaacatgcatgattccctggagctaccaaacgtggggcaggtttcttgccgtcaattttgcctctcacaataacccttccagccttcttgccagctgctctcttccacatgcacccttgtgcctgaggcaaactgaatcactctcggttccctctctcttgtacttttctcttccttttccctcatccttaaggctcggctcaaatgaaggattctgtggaaccttgattgctcagttagaaatgagcaaactgtcgcaaggacagaaaaccaaacatcgcatgttctcactcataggtgggaattgaacaatgagaacacttggacacaggaaggggaacatcaaacactggggcctatcgtggggtggggggagcggggagggatagcattaggagatatacctaatgttaaatgacgagttaatgggtgcagcacaccaacatggcacacttatacatatgtaacaaacctgcacgttgtgcacatgtaccctaaaatttaaagtataataataataataataaaaagaaatgagccagcttctctttcatctgagctctacttccttttgattctctctgctttctgagatcacatcttacatgacaatttttcatacttggctttatttccctagaatgttgttaattggcaccaggttggagctcaggtcgtatactttattccttgcagagtctgacagggtcagaacatgataacacatttgagaagtgagaagaagggaggaaggggccagggaagtgaggggagaatagggggtggaagtaggggaagaagcaaatagggcaaggttttagttgcctcccttctgttcttatgctgttaattaataatggaaccagtggccaggcatgatggctcatccctgtaatcccaggactttggaggctgaggcaggagtatcgcttgagcccaggagtttgagacaagcctggacagcatagtgagaccctgtctctacaaaaataaaaaaaaaattagccaggcatggtggtgggcacctataatttcagctacttgggaggccgaggtgggaggatcattggagcccacaaggttgaggctgcagtgagatgtgattgtgcctctgcactgcagctcgggtgacaaagccagactctgtctcaaaaaaaaaaaaaaaggaacaagaatttggataaatggaacatgaaacacaattcatttttattattaagttgtattctgtgcataaattatttccatgtcttctctcccttttaaaggtgtgccacgtcatcaccgaggtgaaatctggaaatttctagctgagcaattccaccttaaacaccagtttcccagcaaacagcagccaaaggatgtgccatacaaagaactcttaaagcagctgacttcccagcagcatgcgattcttattgaccttggtaagtctgtgccatcgattggagatgacaatggaagtttcactcacatgaaaaatctgaagagactgtccaagttatgtattgacctgcctttaggtttagcaatcaaaatttactactgagacttttaatttaaaaagccctagggtaatcacaaatgtcatcttcaagcatataaaaatctctgtattttcactggggagcttgttaactttgcttggcatggagggagggtgttcattaaggctgcagtcataattgtggttcagtccagtaactcaaatattgataggaggtttttacagtcaaccgaaggaacatcctggaaaacgtatagatgttcagaaccgaggcttggtttaattacaggagccactccctcgtttttactgctcacaaacagaattcatcagaaaaattgtagaaagcagtttgtgtgtgtgccttgaatgattttattttggaaactgggtggcaccttgtctcttgaatagtttttaaaataagaagatgggaacaatatacagtcagccctccatatctatgggttctgaatttggggactcaaccaacctcagatggaaagtatttgggaagaaaaatcaatgaaaactaaacaataatatagattttaaaatatagtaactatctatgtagaatttacattgtattaggtgttataggtaatctagagatgatttaaggtgtgtgggaggatgtggccgggcacagtggctcacgcctgtaatctcagcactttgggaggccaaggctggtggatcatgaagtcaggagatcgagaccatcctggctgacacggtgaaaccctgtctctactaaaaatacaaaaaaattagccaggcatggtggtgggcgcctatagtcccagctactcaggaggccgaggcaggagaatggcgtgaacccaggaggcggagcttgcagtgagccaagatcatgccactacgctccagcctgggtgacagaatgagactctgtctcaaaaaaaaaaaaaaaaagtgtatgggaggatgtgtgtaggttatgtgcaaacatagcaccatcttatagaagggccttgagcaccgtggattttggtgntctgtggggactcctgcaacctatcccccgaggatgccaagggatgactgtattggatagatttgcagttgccactgtgaaggacttgttgaactggggtgtgattatgatgcacagagggccctcctgacttgtcagtggccatgcacagggccaggtggcaatgcactcccgtttgcctgccgcctatcacccaagctgctgtctctactggtggtgagctggctcgatgtggtaggagatgggccctgctgcttttagagcatgtggccctgcttccagaatacctgttctggttgcagctgctgctgctgaaggctccacagaacacacagtgctttggggccctgcggtggcccggttctctgattgttcctgcagccacgacagaggatgcagtgtgagccgcatcaggcagtatgaagtcctttcctctcaagccacgtagctagccttaaaggttaatttcataacccttaaggttattttttttttttaatttttttttttgagacggtgtctcgctctgtcgcccaggctagagtgcagtggtgtgatctcagctcactgcaagctccgtctcctgggttcacagcattctcctgcctcagtctcccaagtagctgggactacaggtgcccgccaccatgcctagctaattttttgtatttttagtagagacggggtttcaccgtgttagccaggatggtctcaatctcctgacctcgtgatccgcccgccttggcctctcaaagtgctgggattacaggcgtgagccaccacacccggccccacttaaggttattctttagcttgaacatcatctctgagaaactttccctgactgtggtctcctctcccacctcaagactggatgaggtgtcttgctaagccccctgtagcaccccacactctccccatggtgcgtatcacatttctcatcatcaccgttatctgcttattatcatcactgctgctgcctaacttcaccttgggccaaatgttgtgcaaagggacttaaactcctttctttaatccttacaacatgatcaggtagatgttgttctgtttctctttagagttgagaaaatagaaacagacaggttacgtaacttgctgaaagtgacacagccgatttgccgctaatcagtgtgacttcggaagctgcacttttttttcaacttttattttagattccaggattgcgtatgcaggtttcttacaaaggtgtattgtgtgatgctgaggattggagtgtgattgaacttgtcacccaggaaccaagcatggtacccaataggtagtttttcaacccttgccttcctccctccctctccaccccccaggagtccctggtgtctgttctcatctttatgtccatgtgtacccagtgttcagctctcatttctaagtgagaacatgtgatgcttggtttctgtttctgaattagtttacttagggtaatgacctgcagctgcatccatgttgctgcaaaggacatgattttgtccctttctatggctgcagagtattgcatggtgtccatatatcacattttctttatccggttcactgttactgggcacctgggttggttccatgtctttgcaattgtgaatagtgctgtgatgaacgtgtgagtacatgtgtctttttggtaggatgatttattttgttttgagtatatactcagtaatgggattgcagggtcgaatggtaattcagctcttagcagaacctgtatttcttactccacctcccccgcctgtccttagtatacagcagtggctctttattgcctttttcccttataggatacagccctctgcggactgggctggggctgtttggccattataccctcggcttctaggacagtggctgtgacacagcagatgctcaaagaatatctttaagattcagagtgtgagacactgcactagcaccgccatctcatgggccctcacaacagccctgggaaggtggcctgcaccctctctaagaaatgaagaaactgaggtcacatgttgaccatggtcacaaagtcacctgaggggaggtgacaggaactgaacccactgtcactctgtgtttccctgggaccctctgagcgcaggaggcccgtgttgctgtgcagtggcaggccaaggcaatgccttggtggagctggggcccatttggcccactgacctgaggaaagcagttttgtgaattggcagtagctgcatttgctgacatggtgagttacaggaaatgccatcatgttcctatcatgtgaaacaaagtgagaaataggttcagggtgggaggctgaaagggaggaatgcagacagccccgctccccacacttgctccaaggctgggnaggaggaacgggaaggtgtctcccctcctggattcagtcaccttcttctcttcattcccctgcagtatcccctcattcttccacggacacgatcagcccctgcttcttgttgctcagatgtcatcacttttctgcagagggaaaagaagagaccagatcagaacaagggcctcggcgtggctgtgcactccgaaggcactgtgtgtgcctgagccccaccacggcctcccctgcagggctcaggcagccttccttgagctggcatgaggtctgtgggagcccggtccactggcagggctggctgcattcaagtcctctccatccctgcctctccccaccctctccctctgnngcccccttctctgacagtgctgacccccctctctcttccccactctttcccatcctcgcctggcctccggtttggatgctgtccacacacttcccgagggcctgagaggacctccgtgtgaggcaatgcatttcccaggtcacctctgtgtctctctccaggctttttccagggactccccggggtcagtctcctctccccactggaacggggaaactgggattggcctagacccggcagtggagtcccaggtgccctgcctgcccggctgactccgcccagggaggcctcccacagaagctcctccagactccacctgttacctcccccactcctctcacccaaggctgtgctgtggccaagtcagttgtttagtctacactttctgtttagtctacaccatggctacctcaaggcccagtgaaggtgtgtagtataaagcaaaatcaaatccatatttcagttttccttaaaaagtgaccttcatattctggccagaagaacagaatggttggttggatatattttgagttttcatgggtttttgttttcctgcctcttgttatactttctgaaattggcttttagtctaaacaggtttttttttttttttttttttttttnggcaatgtgttttcctccaaagagtaagaataataggcctcatggctgggtcgtgttctacagtttgtgaatattttctcaacctttgtcaaatttcatctttacacatcctgtgtgaaattgggcacgtgccgttatttccaacttagaggggatgaatgagcccttaagagcttgagttctctgcccacattgcgagttactcagtgccagaaggagttctggaacccaggtctcctcagtctccataccacatcccttctagggcaccatgttgcttctgtgtttcttggctctgcccactccatgccagcacaactctccccacccctgctttggtggaatcatgttcctttggggtagatcacaccagccagaggcaactgctctcagcttagcagatggtactcatcacattattcttgaagccttgggtcaggagcctgccccacccatctgcatccatttgtccagccctcagacaattgccactgttttcatgtctattctttgactctctatcctgggtagacaacatggactgcccagcatcctgtcttctgtctggggctcccactgtcgtcctgaccacgctgggggctgccagtgacactgggaaactcccgagggacccccttcaggcttcacatcatctgctccctccctagcatcccagcctagaacactttccagccatcagctgcattccccagtgaggcgtgcagcctctcccatgataggagggcttcagccgaaagaacacttcaacaggcccagaaacccaggagcaccattagatcagaaagcagaagcaagaatgcatctaatctcccccacatcaattgctatagttttattaatctgcatattataggtcagtaaggggatggcacagtttataatccctgcaagagtctgatgatcttttggtgaccagaagtgccattttttgatgggcttctagagatcctccatcagggataccagacatgtttggcatgcctgtgctgccgcgagacgctaagcgtgtgtccagactacacgtgtgggtcatgggtccagcagcagagctgtcatattgattgtttgcttctactaaatgtataaagcctgcctggtgtccagaagaaaagaaactataatccaattttttagaatccataaaaggtaagaagtaggagaacatttagaatccacaaaagatgagaagtaggagaacrgttggattttttagaatccataaaagatgagaagwaggagaacctccaaaaggaaggaatcagctgagagtattgaagatgaccaagtacaaacaggcagaggggagcgcttccccttctcctctcccaggcggtgggctgcctcgctcggccaggacacacagagcagcatcgtgcrctttgaggggcaggtggagctgctcatcactagcaggggtgctggcggggaccacagtgttctcttccatctttgagttgaagtcctgtgtgagaaatgagaaaccttcatggcaaaagacagaaagggacctagaatgtaacattcagcagtcttgttatctcacgcacctgtctgtccagttggggacgttgctgtatggaggtcagttgaacaatcacagttgaggagcctaatgaattcttgcaccaccagccacacacattattctgaagagtgagccattgtctctgatcttatcaggatcacatcgtgggatcatatttatttggtcattctgaatataccctttaagtccaaagtgaaataactaaatgtcgttgataaaaggaaagaataaagtggggtatgatttcctttcacagaggtctggaatcttcctgcctttttcaagtcagtcggtggtgctggcaaatgtttaataaccagctcctctcacccctcagaggaagcccttggtgttcagtgtttgcagatttccattgtgcaactagtcctcccacaccccattttaaactacccacttgatgtcactggtcatggagttgggctcacagagccagtgggagtcaactggagcagccactggactcattcaagtgtttcccaaaacaatctgctcctagaaggactctcccttaatctcctaaccctgccattcaggatgattccctgcactctgggaagcacacgttctagtgggaagactgatactgggcaactgataaccaagtgacttaaacttctgagggttacaaagggtgtttgtatcctcagtgtctcatttcagattctgctcagagctaaatgcaacaatgtgagaagatgttagtatcccagatcttcatccaggaaggaatcttagagatcattaggttgtagggtttctcttctgcagaggagatagagggtcggtgtcagattgctggtttgccagtaccactccctggagaaaagagcaaaagaaagaaacttgttagtcaactgtgcagagccaccgtgagactgaatagctttgtgggtggccccgtgtttgctgcaagagacctctggcctcttgtagcagctgccacatggtaaacagagccgagatatcaggagtctcgctgaaaatgcagtcagatgggctctgaatagaggaaggcaggacactcttgagatgggatggggtttctcacagcaccgtacagggaccacctgcaagatctcttgaggggcttgtgaaaaacacatccctgaggtcaccattcttgacctgctgcttattgagtttctgatgcctgggatgtgcaggtttaacaagcccccagatgatcctaataggattcctgcctgaaaattgctgggtgaaggctcttccccctccaagtgataaagaaggaaaagattgatcctggaagaacatccgttagatgagcaaaattttgtggagcacttcatgaagaggaattactaggtcatttagaaatatgtttgaattgtggatcatcttgtaggcctttctggcatatttctccacttagatccacaagacacatcgaatgtctttttataaaggggttttttaatgcccatgtttgaccctctccacttaacagtcccattctcattttatatgtgaaggtaatctgctttacagaaaaatgtaaaggacctgcacttctctgctttgtggtaagttgtaaaatgcagtttaaagaggcaggcctcatatcctgatagatttgtaggaaggattgcacagttttacccagcttccctcgagtttggcagaaattagctttccctgagctggtgtcttcccgagctagcatgcttctcctatggggtgtgtggccttctctcctgtctttttgaggcagagcttcaatctagaatctgttcacaaactgaacaaatgcaacaaacagtaaacagtcttttgctcatagttaaggtgccttgagttgggtgtgaggggctgagtgtgttctcaggggtgctctgcccacggctccggccaactgctgcaggtgcgcatcatatgggtggtctttgtggaatgccatcagcactagcttagtacctcctaaatgggagctggagggctacagtgctcaacactggattatacgaatgtggattgtccaggaaatgcttttaatccccctcatccactctctacccacgtgacctgcctctccctctttacttggtgtttactcaggaatgtgggtgagttgtcgtgttagcctagaacagccattcccaaactttgatggaaggatgccattcactttgaaaattatcgagtagcccaaagagcttctgtttccatggataattcctatcaatatttactatattaccaattaattaaaactgagattagtatttatttgattcgtatttatttttacatagctataataaactcatacatataaaaaattattaaaaaatgactgttttccaaaataaaattagttagaaatgtgacattgtttctacattgaaaaaatctctttaatgtctgatttaatagaatccgctgaattttatttgcttcattcattctgttttgcttgttatttgaagcatataaagcaaaagctgaccttgcacagatctatagtaggaaaagcagggggagggcctcatggacccctaaaaggatctcagcgacctccaggggtcctcaggctgaccaaacattgagaactattgacctggaagaatgtaaaataggaaaacagtgtctcccccaatagaatttcgtgtaaaacgtggactgtgttacaaagtcagatgggtgcagttgtcctgcttaaccgctaatcaggagctgaaggccagagactcacagctgttccccagcctggtagtgaacccagaggcctgtcttgctgtgcagtgggacaggaagttgcatttgggagtctcatagaacacactggaagatgtgttttagcttggccaggttcatgcaggacagattttctgcataaagaaaatcaatgacagtttctgaaactgcatcctggaagccttgaccagtttgggtaataacaagagatttgaaagtgtggggtgtacaggtgttttgctgaatctaggtggtggtggtgattattattatttggaattcagctttcagttctacctgcttgtgagttccaaactttgtgaaaattagttgcttggacgaaacttttctttgcctctggaaggctgtcagaaagcgagatttcccagcttatgtgcagtgttatagttaatagagtaatggctctgcaaagttgttcctttactttaaatgtaatttattttgcatttgtgctacagaacggtcataagtgtgcccttttgtcctcttgtttggaaactgggtttttataatgtgtgtggtctatccgaagattattgcccattattgaacaccattcatagcaaccatttgcattaggcattgtacgtgtactctccactctgcaaactatgtgttctgtccctttttaaaaagaggaagctaaggttcagagaagctaggtagtccattctgagcttcacgtgccagaggccattttgtacttacttcaaatgccattgaaataaatgcacatcagagaattgttcttagcataaggggcgctacatgtaactttttattagtgaaatggatgatgttcaagggctgtggttgattagaaaggcgtccagaccctggctccagggactatggagcagaactcgaggccagtgcctgtcgagcgggtccccacactccatctgtgtgacctgactgtggatggcctggctctgccgttagattgccacggtgccctcctctggttgaacctttctcgagaagtgcttgttggaggcttgagtgcagagcctgtgagaagctctatgtggttcctattgcctgtcagcttgctgataaaggtcattggtttggcaaaatttggcccaaggtttgccttctcataacataccactcggtagcaaggctgggaggaaggtggctatagctatttctggaagctgcttagggggctgcctccccctaaattggtacataatttgcagggcctattgcaagatgaaaatgcagaaccctttcttgaaagattattaggaatttcaagacagagacaacagagcatgaagccttgtgcaaggtccttctaagcacagagccagtgtgaccgcacagaacacacacccgtgaagccagctctgcccccaccatctgaccactcttgagtggccaattagcataggtcactccccaccctgctaggcccaccctcttaggaatgttgtgaggcttaaataagaaatagccactctacaagcggtgtcaattagcatgggctctggtttctgtgtgaggtagtttgctaacatgagagggtatctgattagctaaaacgataacactgacagattaaattcagaataactaaaccttccctgtgttcctttatgccacatgactcctgcatattctgctaccagcacctgtttgataccagacggaggggtccatttgggatgggacaggagcatcagcagaaatgcagaagtggggaagtgctccatcttcttggaagctgagctggcaagggtaatggaatgaaagagattgtgaatatttttgagactatgaggaaaccagtacactggtgttgcccagtacagaagccacatgtggctgttaagcacttgagatgtggctactccaaattgaggcgtgctgtcagtataaagaacacactggatttcaaagacttggcatgaaaaaagaatgcctaatgtctcagtatttttatattgattatgttgaagtgatagtattttgtgtatgttgggtgtaacaaaatatctaattaaaattaacttcacctgttattttctaatgtgggtgctagaaactgttacatcctgcatgggggtcacattccagttcagttgcatgtgctgctacccattgttctacacacacacacacacacacacagctgcacacaacctagaggggtcagagaccccaggagcccctgcttctggtgcccaggctaagcgctggagtggaagataaagctgggagggtgggtaaggaggtgagtgcacggagctccaggctaacagagtggataatttgttctttgagcactggggagctatggattgcttactagcagcaaggtgacttgtgcagggtatatctgggggaggtttactgggggaagagatagaggaggcaagaagtgaatacagaacgagaaatcaggacagtggttaggagaccgtagctttcctcttgagtcaagttcagataacacatctggactgatgaaattctttttcaggaagctgaggaagagcccatgaaaatatgttcctccctgtgctgagaccgaataattgcagtgaacaattaacgtgtggcctagatccaccttttgccttcgctgatccaagcaggttcataattcttgcctgggcccaagcttggccctggctgccagctgcctggctccagatgtttcttaatcgtttcaagtacttctctgctccctggaaacaggcactcccatcagtcacattccagaggaggaggaagaggaacttgacaagtatcagctacaaaagcctcctgaacaaaagaaatcctttaagcctatttgaataacagttttttgtgaaaataatcaggatgttgagagcttttttttttttcttttaaactctttttggaaggtaacttttgtgaaaagaaaacacctgctgctcctcaggctgtttcaaaacactgcctatagtttgaaagtacggagatatgcatgtggtatgaagcatttgcaggcataatatgtgtagtctgggaaaagcagatccagagagtgcttgtagtaaggcgaggccttttagctgcatttagatgatgctgggattggggtgggtgcagggtgcagcagtggggaggaagaactgtgtgtgttcctcttgagaataggggttatgtctagaggattaacagttttcttttttcntttttttttttttttttggagttggagtttttctcttgtctcccaggctggagtgcagtggcatgatctcagctcactgccatctctgcctcccaggttcaagcaattctcctgcctcagcctcccgagtagctgggattacaggcacctgccaccacgcctgactaattttttctattcttagtagcgatggggtttcgccatgttgggcaggctggtctcgaactcctgacctcaggcgatcctcccgcctaggcctctgaaagtgctgggattacaggcatgagccaccacacctggccaacagttttcttttttcgattgaagttcagctatttgcaggaccgaaggtagttctgattactttcacctgtacttccaccaaaaaataaataaaacaaccatgagtaattgctgatttttaattgaaagcattattccaggaataactggtggacttcgtttgcagaggaagtggcaaagactgattgatattatgatccagcttctaaagattttgctgcttaatctgaagcacattggatttctggttcaataggctttctttttttgtttttattattacaactaatatgtattcttttcacagggcgaacctttcctacacacccatacttctctgcccagcttggagcaggacagctatcgctttacaacattttgaaggcctactcacttctagaccaggaagtgggatattgccaaggtctcagctttgtagcaggcattttgcttcttcatatgagtgaggaagaggcgtttaaaatgctcaagtttctgatgtttgacatggggctgcggaaacagtatcggccagacatgattattttacaggtatagagtgttccttatgtctttaatacaacaaaatgctaagaatgtttcttatccctctccagatgtgcctcaggagctttttcaccgtcaggtaacattgtaatagctgtcactgctgataaaggactctgtgctaggcattattccaagcgcttcatctgcacttccctctaataacaggaagacactgttcatcgtctcaatttgtagattggaaaactgagtctccaagagattataaattgggcccagtcacacagctagcaagtgtcagagctggactggaaacccaggcctctctgactctagggccttccctcttgcccccatcagccatcagatgatctcagacctacctcccagcctctgcatctgctcttcctctgcctcacccccacccttgtcatctcaggttcagctcaaatatcacatcctgggagaagctcattctgactaccctgatgttgtgttccccacttccaccttgggcacactgcgtcacgttatcctggctgatttcttttgcacaacacagccactgccagaaatgatcttgtttccataatcatctccctgtctattttctgatttttcatagcctgtgaactttaggagagggaagggatcttacgggtcttggagccgagttcctagtttctgaaacagtgcgtgggttgaagtaggcaccccataagtatttgttgaatgaacaattctgtcagagaaaaccaaacacagtagcgtattgcaaataccacgtgctgctcttgctgcctgtcagagggaaaactctggatcctgcttcaggaatattcctaaatgttgcagcacatgttgatatgttcatttactaccagtaagatactatgccttcagagctctagagagtatcctgggagggaatacattagagccaaggacttgctttgagagcaccaaattatgtgattcaaaatcttttcaccttgacctgtgaacatggaccacgtgaatgcaaatatcatagaaggaactcattcactgaaagattttgaccacataacactttccacatgtactgtgaggttcttcctacattccctttattaactttaaagacagtggtcaccaggcagtggaatttttgagttttctataatttatgtaacacacaactcttttggggtggtgcctttggttgattagacagtcttcgatatgggagagccacagctggtgcttatgggattatattatctgagcctctgaaaacggttttgttttctttctctcagtttagataggacatatccaacttggtggatcttagcggattctgaccctctgtaggttgttgtttctttaggctcaggccgtggcactgctcagatctgggctggctctcgggcctctgtgagcctgtaactcttggtggcactactaggaactggcatgagatttctgccagaatcatgtcattctgtgaagttggagttccactttagttggaaaaagtttttatttcatcttaagatgcacacttgtcttcttgttttaacttgccaggtatctggatattccatatattatacaccaaaagaaattatgcttctcctgcctattgagtaatttcaggggtccagagggaacttgctgagtgaacatgtacaatggattcctatggaatcataagatgcccctaattcagtcttagtaaagagactggcttcttatttctaattcctccaggcttgagttgtgcaaagagtatgtatttgtaagagaatttatgaaatgtttgcacaagacagatttttagatcttcttagtggaggaatacaagggaacaataaaaaggaagtggcagtagaagacccagcgttagcgtcctgggcctacacccagccagtgcctggcaccagcaggcacttgggaagcacttgttggatgaattagtagctgagctcagtggatcgcaagccaaatcgaatgtttaaagttctagtaagtcttctcttacacccaccctgtgagcagtaggcataactttattgctgtggcagatccctaattctcagcccttgtggctgtcttcctgcagatccagatgtaccagctctcgaggttgcttcatgattaccacagagacctctacaatcacctggaggagcacgagatcggccccagcctctacgctgccccctggttcctcaccatgtttgcctcacagttcccgctgggattcgtagccagagtctttggtgagcattagtaaatctgtttgccagaaccagccttctcttattagaggggaaacatttcctgtctctccrtggtgattcttatttttatacctgtagctcttaccagaacagggtattgtttgatagtctaagattagtcaggggtgggttttgtgactttggagtcctccttaacttctgataatcacggggcttcccttagatgccttcatcttgtgggatgtggatccgatccgtgtagatccgatcgctcaccatgagggtctccctagagcagacatttggaggacttggctgaggagccacaggtgtatgtttctcatgaattgccttcctcagccactctgggttgtgagtattgactgatgctgactgtgggcctctgggccctttctagattcccttggcatctcttcctcccctttctcttcttgccctgcccttggctctacactttctcccaagtcactgtcttggagaccagtgtcaggaccttgagtaacacctccgtgtggatggctcgctctccccgctcagccttgacacttcatgaaggcctcttgcccctgagcccacatgtcacagccactgccactcccgtgcccccgctgttaaccttgggtggttcacatgtaaaacctgcctttatattcttgatttactttttgagaacattgtcaaagttaggtgagtgttcatacacaaagccttcaacctgccttcatatgcagggatagggctgtccacgtgcgcatcaggaaccgagtggaatgttgtgagcatggtcagttcgggcacagtttgttttccctactgcagaataaaagtgatatttttgacaattcaggttcttttttttattgtaaaggaggaggctactaaaaaaatgatagttattatatatcaaatgtttttaagcatcacttgacagcttaaaaacatgtgatctttaaaaaatttgtttttatgattagagagcatcttaagggaaatgttcaaagacattgatactacttcagacatgctttgggtaaacatcttaaatatccaaattctagaaatcctaaaatttgctttttaatataagtgagcatttacccttcttctctcttttcctttccccccaaatactagatttttattattcacttttatctacaagaacctttaaagagtttcccattttgctttactataagaattcatattccttcttttctgtccctgaaaaaataaaatcactaaattaaaatagatacaaaaagctatctcctggttgagcatatctttagtgagagttcatgaaggtttataccatggttaaaaaaaaaaaaagattaactaaaagcctcaaaattgtgtgcttagtttattaacaaaagagttacagaaactaaaatctcaagctctaggctttaagctttcttgccaataacttctatgtttttgacttctctaacactggaaattaaaagaaaattattaatctaccttccttacattttctccacattttagctatgattttcatacagggtcatgaagaggagtgaggatggaaatggggaggagggagcgctgtttgttaatggtttgtaaacagctcaggcattaaattacttggttagtgaagaaaattctaccaaggcaaccaggctgaccacagactggagggctgaggggtcatcactgagtcatctctgccctggggccccaggcactggagctgctgcttgcagaaagttctggggctctggaagagaaatttttccttcggctcataaatgggtaaaaagacgttaacaaacaagcagactccactttggaaatgatagcccttctattgcagagtaatttgaagctctctgaagctcactctaatgacttcataaatcaaagctgcagcttgtaaaggtaagatatttttctgtagactttgtaggcagtggtgagactcggagtttcataaacattatgcatagagatgccagtgtctacatcattcctggatcccacagactcctgctgtgctaagtgggtcgttgtccagctggccaagggctcctgggattagaggcgggaagtgggatctcaaggccgcactggcttgtgatgtcactttgcaagaactgcttttttttccacagtccatcccatctttcagtacttaaaaacagaaaagataggtttttacaaaccatttctatttttagcactgatgacttagagaatggtgatggagatagcttagttttatatttcaaagcctgccattcagtcactatagtcttttttggcctagggcccatttcattataagcctttaagtctggataaactctaaaaacatgtagaactttgttgactaagtaaaatatttcagtttgcaccaccttagctcatatattagttaataggttacctggcattaagtatgtatctgctccttggaggggcggctgccagtgatgtgtgcgccttaaccttcacatgatactcacaccttgctgaatggcagttcttctacctggtgtcacataatgtcaatagccccttcctgtatttttctagcttgagtacagcagggccctgggagagacgcttgctctgttcactttctcatcacatctacctttgggggaaaaaaaatctaaaaaacaggacctggcttgctcctgatggaggaggaggctgcagtgttcagcctctgatgtttttctataggacatgctgccaaatagatgagggaggaggaggagtataaaaactaagggtttggcaaaaaacacagaagccacctgcaatatagtgaaggcttcagagagactttaggatgaaaatagactgaaaacaagattgtttctgtggccaggaaaatctccagctattcaggtgacatgatgccgcgtgatgatgagtgtgtccagtctgtctgtgctgttgttctgcacagcactgtcatcagccttcagcgtcccctttacccgttactcatagaatgtagcggagccacgactggaggaccgcagccttccagaagaaagttgagaaggctcagccttgacaaagacaaaggtggctggaaaaagagatgcagtgcaatctcacataggaagattgcactttgagatcatgaagtttagtttcaaatagagttccaatacacagtaacgcaataagagggttgctgaaatgtcctcaaagaaaagcagttcctttgtgttgttcccagcgaatacagtgcaaagtaatagagtcagctgaatttaagattcctatttcctgccggataaaacgtcttgcctgtttctaggtggcttgaaaaaggagaggagagaaggaagaggcaggagaaaagtcccactgaaaggacgtgggctacagtgtagtgagctaggctactgcctcactgcgctgggcggctccaacagttcactgtctcctagggaggttttcaaatgcaggacatttgctcacttttccaaggagagttattgtttttttgttttgttttgttttgttttgttttaaaaaattccagaatgtaaatgtataagataccggaagacaggcaaaataaaaataattggtttggggcagtgggtttataggtaacatttttctttactattrttttaaaattagatgtgatttaaaaaaatttccaaagccaaaaaacggtacaaatgctttaaaggatgaagatgttgtccccaagtgtcatcagacaaatttaggaggcccttcctcccaagcaaagcttcctgcagtccttccttcaactctgaattcaagcacattcctgcattgtgcacccaaatgatctcccgatttaagaccccctgtgtctcacagaagcttctggggctgaactttctccggccttggagggttggacgctttgaatgggaggagtggtggtgagtggagcatctctggcagcaggcatttgggagtctctggcaggaatcaatcagcgtagtctccaaaggtggcctttctctgacactaactagcccttgcaggggtcatacccataacctgcatctcattaacatcatctccttaccagtgcactgacctagtgagaaaaggaacaacaagcattcagcgactcctgcggtgctccaggggaagttagaattgcttggctggggcagaggcccctggtgatctggacctgcgtgcccccatttgcccaccttctgccctgcacaaccagtgcccctgccttgccagccagactgtttttcaggctcctgcacacctccctgtattgacaccctattttccttttattcagagtattaatcctgaggtctgacctaggaaattttcattggttcttcaagcagtcacctttctgtgggccttttctttcctctttgttctcgtaacaccctgggcataactctaccgaaccagaactccttggtgtctctgcagcgtgttctttgtgttttgctcatggcttaatctccagagcctaatacagtgcctgatgtgtattagatgctcaatagatgctcattaagttaaagtagaagacacctctcagcagagttctcttaaggtgttgtgaatagcattgggaaagaacatttattttttaattacattaaatacaaacagatataataaaataaatcatatgcccagtgctatgtcttaattttttaacatatcaataaagagactttaaaacacataacaccaccctctcccctccaaatttcctttccgggaaagtctccttttggaatcataggaagcacttactaagttgatttattgtaaaaaaaccaagatcctaataaatctcagaagatctcctgttaacctaaagagaccactgatgtggattctgtatttggttgtgctgacaaaagtttcccagtaattgtttattttaattggcgtagatgtggtactgtacctaatttaaggcacttgtccctctgagagtagagaccaagctatagaaaatcactggtgttgtagggaaagcctttccccaggatccctgcaaaaaaggtcttgatttttattctgaaagatgccctcattttttgttcagctataaaagttcatatattgaaaggaggtctaggaagtctcactgtgtaaaccactgaaacttcaaatttactttagagttttgtttctggaaatgtcatttctgtttaaaaatacatctttgttatagtattattttagatctttttattttctgtagtggggaattatacaggtagactacattttataaaccagatatttcagaggaatattcttcaattggcctgccttggtgtatgtaacacttaccctgaaaagctctgatttcaaagacacagttagttctctagtatatcttcccagcctcaacaaccagacttaagaaggaagtgaaggattcatctttcccactttcctgcggccaccctgagccatcagtagttgtgatgtttgtggaaagagtgtggaccctgagctgggtgggagaagcaggctgatctcagcgctggcatggcttagggctgcacccatctcagctcacatggttaattaagggttttgtggtggttacagaggatctcgagggctatcccagccagcgggctcctgggtctgtcatccctgcctgtgctttgttcagaaactacagggattcagtttcccatttgcacagcagcacccagtctttgcttttctgtttcttcgtggcttttaaatgttatcatattaaccatctagagaggcaccctgcaaggttattcctctcacctgcttttgctttccttgatttgatgaaatttacagcttctttctctcttccattatctttcagccaaaagaaacagagaaaagaaatactgacacttgcctccaattatatttctactctgatttttaaaattgtttttttcttatattattattctagttattaggtaacctgcctcagtttagtcaaccaataattagttatcgtsgctctgctttaaccccaggacatcagactctttttttccccagcagcttcaactctatgaggaaggtgagacagggctggggttgctgctcggccgcttgccttggccggtgccctccctcttattctgcagtctgtatagaagttgcatccatttgccagccactctaagaacaaaatatggccagaactaggaagtaaccttgacagagttcttgaactcctcagagggaaaaatgttctttattccattatcatgttaaaaatcagtaaacttgtatttaacaawgtacttctgcagttgtacagctgttgtacagtttttaaagatctttgaattctattccttgtttcaaaacagaggaaacagagacactttttcacttactctatcttaatttctgatgctttatctataaaaatcttttagtgtgacccataaaaacatgttttagtatctcctttaaaacccaggagcattcctggaaaaatagactaataaaacctttttccctttcccagtttaacttttgaagcatgtttgaattttattttcagagtaaaacataatttttaatgtttatgtacttttatttgcaatactgtcttgacaacactgtctgagatatcaggctcttaaaaatgaaataaagtttgcaatgtggggctatgtctcccacactcctgctctgtgatgtgtggaaaaggcaatggaatggtattgcgtgagaaactggtctggtttaactttctgcatttctgtgttttctcagatatgatttttcttcagggaacagaggtcatatttaaagtggctttaagtctgttgggaagccataagcccttgattctgcagcatgaaaacctagaaaccatagttgactttataaaaagcacgctacccaaccttggcttggtacagatggaaaagaccatcaatcaggtatgagtcagtccaaaccttgcaaatgcttaagccatcctagatatgtagaaacttaaatctctcttgagcaggaactgtttcctaccactttgtgttctgaacggcattctgcatgatgcctggcatggaggaggcatatcacaaacgtgtggaatgatcgtgagtgtgtgtgttgtgagcgtcatggtgaaatgccacatggaaacatggtggcaatgtttagctgtagaaaccagcacaggttattagtagtttcttacatttaagagacttcagctctagtagcttgttcttctgaaacatatatatatttatgcaatgatgcaatgtagggttttgtacattgagtgctttgatttgtgtgtgtgtatgttgaatggttttaattggaattttctccaaataattctttgataacaaagttatgatagggaacatatattctatgaatttgtttcatgatgtgtgtgtgtggtttttttttaactgaattcagttcaatatctgtggcttcattacctctggtttcagtatacaatagaaccattatcctctgaagtgttagaggctgagaggtgagttttactgggaattacaactaaactagatggtgaatgccctgggttgggcatgggagcagattttgatcctgtacagtttaaggaagaacctgccagtaatggctgctgatgatggaggactatgctccttagtagagaaaggggcatcattggaagtgtctagaggctgggtagctacgaaagcactggaagggatttcatcattgagtcactgcagagtcagcagtcaagcctctcataagcctagaatctgctccagatagtcatgtgtcacttaataatagcaatacgttttgagaactgcatcattaggtgatttcatcattgtgcaaacttcataaagtgtacttaaacccaagtggtatagcctactacacacctacacatgttgttcctaggccctacaaatctgtatgacatgtgactatactgaatactgttggtagttgtaacacaaggtggagtatttgtgtatctaaacataactaaacatagaaaaggtacaataaaaatatggtattctaatcttatgggaccaccatcatatatgcagttcgccattgaccaaaatgttgtaatgcaacacacaactgtgtaacgaaagcatagagcaatcaggcaaaaacaaatggtgaaataaagctatttttgaaaaatccttactctggagatttctgtagtccaaaaggaatccatgattccagtggattgcatccacatgcagtgtttgtgattttcatttgcagccacaccttaggtgttaagcacagaaaaagatgcaaatttggcctgcaaaagaaagaggtttcataccagttgttaactttagatttctgtttgcacattgcatatgcccttatgaaagaacagttcttgtctgttctgcactcatctttaattgagagcctctccatctcttttccttccctggaacactcttcttgatgtggatagctttggcttgttggggccttgctttttgccaggttgggtgggcatcgaaatatacgcatgatcccattcagtgcaggccgcactcctgagaggatataaagtggttcactgactgacccacactcactctgctgcaaagtggaaaggtaggggttcaaactcaagtccctcccacctcaaagtgctttagcagctctcctacactgccaagagcctctggaggtcatttaatttagagtttttccctattttaccaggattctaatactgacttctccacccttttgattctttgatttctggcattttcattcattctttctttcattcattctcttcttacagcttttgttgcatgtacttacttacatttacagcttctagggcagacccccgagagccttggttacctagactgagggctatatccactacctgacatgtcaccttgctcctgtccctcaggccatcccagctgacattgtttacctcctaagtattgagcctcagaaaaaaatcccattgtctcctattttctgtaaaaaacaaaaaataaaacgtattgagaatacttaggatacatcaggtgctgtttcagtgctggaagagtggaaatggacacagcatgggaagaaaatagctgtgcgtgtacctggtttgtttcaagccgctatctggctatttggaagttgccattcatttttccactgactttttttttttttttttttttgagacagagtcttgctctgctacccaggctggaatgcggtggtgtgatcttggctcactgcaacctccaccttctgggctcaagcattctcgtgcctcagcttcctgagtacctgggactataggcatgtgccaccacgctcagctaatttttgtatttttagtagaggtggggtttcgccatgttggccaggctggtcttgaactcctggcctcaagtgatccacctgcctcatcctcccaaagtgctgggattacatgcatgagccactgtgcccgacctccactgacttaataactctaggacataggtattataattcctatttttatagatgaagctgagcagagagtaacatgcccggccccctgtagaaaggcagggtctgtgggagccagggctgtgaggttggagctgaggtgtttgagtccagctggacttaaaagatgacctaagatcggctggcagacattttccaaagaaggccaggcagtatatattttaggcttcacgggtcataacgtcacaactactcacctctgcgacagctactcacctcagcctttgcagcacaaacaatccatgacagcatgtaaggggatgatgggtcatgttgcaaaaaactttattttaaaaactgtgcagcgtgatgggcttggtccacaggtggtggtttgctgatccctgaactaaaggatcacagcatgtgagaaggtacaggaatgagagcagagagcaattctcagaacctgaggtgttcaacattttgggggtatcgggagcacaaaaatttgcatttagggcccgggtttttatcagtggtccttatagaaagtagatctacccgcatctctcccctctttccctctggtgtttctatctgaacttgacatctgagtgttctctgtcaggctttctgctttcccactgccccctcccatccagagggtgctgtagtcttaagttctacacagaaagcagacatggggtccagcatgattcctctgcagccttagagatcccccaggcccaaatcttgggggtcttcagagtagatgtaatggatgcttccattgcagatggtggagcatactgccctgtgtacagatggggtggggcaggaagtgagggcatctgggtggtcctctgtgatagctgtggttccatttcattaaatgccctccctgtatagatagtctcagcctgagcaactgggacacagctggatcctgggtggaagaggtggccaaggacagggctctggagctctggctcaatgtggacaggctgaaagcagccagagagggcaattccaaaggtgacagagcccaggacagaagaccaagggtgtctgaagcctttgtggcagtgtacttaccaaaggacctgtccattgaataaaccatctacatcttctgacccaagaagaatggaaactttgggaataattagtaacaaaggaaaggaggtcagtgttgtattcttgtcacagtgggtgctctgtggctgtgaagtctcagctcagtttaaggaagaaaaaaaggaagggtggctgtgggaggcagggcaaatagcctattttcagcatcctttaggctccactcagagcatggcctcagcccagcattgtcatcatatcatgtcagagcttgttagaaactcagcatctggggccacacccgagacctgctgaatcagaatctgcatttcagtgagatcaccaggggattcacatacacacaaacagctgagaaaccctgctgtgggcaactctgttagaaacacaatgaacaaaggagcccctgttccagttgagcttgtaggttagaaaccagggttcctgtattcagaagacacacctcaaatcaggggcaaaggtgcctcttctgcctgtgggggagccgtcacttcttgggcagtttgcaccgtggaaaaggagtagttttgtacgaggacaactggtgccataccaggagggtggggcgtggcggggagaagtggtttaccactggcgttgttgaaaattgctcacatgcagtggtaataacaagcagagggacttttagtgggtttgatgttttttgtaattcactacagatagtgtgtgcccccttgttgctgataccaggccgactgttcccactctccagcccttggtatgacaatgggaccagcagattggagggcagggggttaggaaggcggaagctctgtggcgagttctgcaaaccrtcagggttcatgactttattaatcagtgtccatggactgtgaagagaaatgctgagtctacaatagcaaatgagccaagaacataaacagacaattcaccgaagaggagatatctagtaaacaaatatctgggaaagtatttggcttcatgtgtaatttaaacttatgtaacatataatgctttactctactagataatagaaagacatttcttgatgccagtacccagcaccaagggtatactgtatgcagaacattagcatgttgctgatggcagtgcacattgattagtggctgttgggagacaatttggcgaaacatatcccaagccagtaaaatattcataccctttgactcagtcatcccgtttcttggaatgtatcctcaggaaataatccaaaatatgagggaagccatatgtataaggatattctcctagacttgtcacttataataacagaaacttggaactagatgtctaacacttgatgactggattaatatgatgatggtaggttgagctggtagaatatcatgaagccagttatatatagcgacatgaaaaagctcttatttgatacaatgttaagtaaaggaaaagtgggataggaaattttatgttggttatgtttagaactagaaaaacatgcttttaggaaataggaaatatagctagatataaaagttgtatttggtgatttttcttttatttttcaagcttccaataatgtagctctattgcttcagtaacttaaaatagttttatcttttcggcaaaacatcgaaagtatggaaatagtcattcctactttggcaaacagaagagaaattttcttcagtaccaaaattctggaacttgactgaaaactatgaagaacctaagagccaggatgacaggaaggctctagatccccagtaattacaactctagtggaattgctctgagatgggccagcaagaaagaagatgagagccagtcccccttgcagaggggccaggtaccttgcagctttgtgtagtgaccagtgctcagggaacggcttaggcaagaccctgggggaggtgggcactgcacttgtccagcctcaggagtgactcagaccagaaatgaaaacaccttaaagtgtatatatcttgttttcctatcaacacctagtttttaatattcgtctgttttattcatctgagacaacataccaaaggattgggtttttaatgttaggccttcctgctctttctctggctgagaactgctcctggcagtggatcacttgtgctgtctaagtgtgcaaggacaggcgcccctcccaattcttttctttccccaagtaattagcccaagggctgaagccctcgtccagtgaccagggttctcctttgaccaccagcctcatattgccatggtttggggtaaattcaggggcataactgcagaatgaagggcctaggagtcttggcagtcaggagatcatcaggcaattaagcagagatgattgtgacccagggtggttcctagggattaatggaggcctggaagagtttatggctttgggtactgctgagagccattaacttaacacagaacatcaatccgtaggaaaagccagaggttttgttcccaggctttccaggttaggagatcacttaaatctttgtgaaagaaaaaaagtaatagtgtacatgacatttattcagcaccatatttataattatacatgagtgccaaacaatctcagttttaacatttgtggtttttactgttcagactattcagaagttccatgacgtggcatccattgatgatggttttgctgaggttgaaatgtgagggttttgtggcaggtgtgtggaagcagatcccttagctaagagagcgcctgctcaacctgccaggcgtgtgctgggaacttccaggagttgcctcgtttaattctcacagccatcctgggaggtaggcgctgtctgccccaggtgggaggccaggaactgtggctgagagaggttaagtaccgagctcaagtcattcatagagtcgccagtggagccgcaattttaaggctgactcaaagcctctgagccagttacctcggctgtcaaatggagataataatccctgctgacctcacggtcgctctgaaggacaaaagagaatcagttcaatccagtaaacaattctctctctccctcttactcccctcgcacacatgcacacacaccccacagatataatggattttagtttttaggcatcaaatgacttttgtgccttattaatattatccactgaatcaaaaacagcaagctgaaaaattcatctcaaggaagagaaaataagattgttgggaatggtgagaaaggaaacatggtttttgaaaattgattccagggaagataggctagtttgaatgccagtagggagccatcagaagaagtagttttacactgatttttaacaatattggagttgcttaaggcaatgcaatagagaggcagtttctgcccttttaaagcctgacttcactttctgaatgtgtgttctgatctagcagggttttttttttttttcttttaagatggtcccagcttgactgcattctcagatccatcagataaacgttagggcttcactgctgtgctgagaggccccagcccctggggttctctcatagaaacaactggaaagaaaggaaatgccttgggcagcagcagcagcagctgtcttctgattctgctttccgccctgccttccttaccaagagaaagtacagacacggacggcttgagtcacttaggcacttaggagttgtttttcacacgtgtggtgttttcgtcaccattactattgtgggaaagaagacaactcaggcatcgtttcgtattcactcatctgtgtgggtgacatgtgggttttggctcatttctgcatatttgtgtgcaaaggagagttttttagtaaacagtcccattacttagctgttcttgtaactctgaaaacccaactgaactataattaaactttgacttggtgactctgcaaacaggctatgattcttttgtttcttttctccttttaacccatagttgatgtatctaacctaacagaattttcagagaaaagaagtgaaataagaactaaaaataaatttttatgtctttaaaaatgagaggttttttttttttttttggcttttggaaggtgagtatcaaaaacctgtacttaatgttaccttggaattatttctagatgtttcttatatccttttgtcccaagtaaaattattaccttctcagtgcgtagtttttcttatttattacttctagtaccaagtgtagagctaagcgtagaggagacgcttcacaggtgcgcattgtcgtgattgcagacgcctgcctgtacttgtggggtttttctcagttttagtacgtgatgacttttctttctataacaggtatttgaaatggacatcgctaaacagttacaagcttatgaagttgagtaccacgtccttcaagaagaacttatcgattcctctcctctcagtgacaaccaaagaatggataaattagagaaaaccaacagcagcttacgcaaacagaaccttgacctccttgaacagttgcaggtagagcatatttataaagcagcttcctgaatcacaaatatatggtagttcattaactcaccaaaggcaacagcaggctgggctttcccatgaccagaggacctttcccaccctgatctgtttatagttgggatcaaaggtatcccgggagaatgggtcctttttattatggagcagacagattgtcctttgctaaggtcaggcagtcccagagctttctgagaggctgtttctgcacttaactcttttaggggacaggcccagagatgaacttggattcaggatgccgtggcctgttagctgaatgccagccgttgtcattactcaaagagaatctaagagcttttaacttctatgagcaaaaccagctaggtccacagagggatggtaaaggaggaaagtaacacagaaataaatataacaaaccagaagagatgataattctttgtgagtccttggtgcatatacaaagatttgattaatgaaggtctcagttctcccctctagaaacttccatttcaacacggatatactcaggtgaggacatacagaagaaagaccagttgagactgtgcacgcaggagggtgtgcagagcaagcactgaggtgcagcacggagaccagagctggccaggtccagcatcacccccacccccacatcacccaggcacactgcccaaaagaacacctaactgcggagtgcagctcttttgtcaatctgatggcatgaagcaaccatatgttctacttttttctactttttttaatgtcacaagtgtgtagcagtgctgtccctgttaaggagttgttttgagggtgtttttaaaagttgtttttgagtggctgtggataaaaatacatatttttgccgaaatttttatggtgttcctgggctgtcctgagaataagttccattctgatctaagcctctgatttttcttcatagaaagatgagctttgcagacacaagcttggcagcaaggtgagaaaggccagcctagtgagtcaagctatctgaaatgcattcctcccagcgggcattccatcccagcataccctatcagatatgtgaaagagaggaaccaagaccgaatgctattcctgcccagccctaataaccactcacattctgaaatttaacttctttttttcccctaagatagagatgtcctaactgaaaatatgcctgtatacaatttaccctggaagtctcagccatcactcaagggaagtctccagagggtgaagagcctgtctggcctgtaggggtacacagtgtaggtggtcattttaaatggcttccaagccaatgataggtccctgaaatataacatggtggaaacttctaataaagctcacatttgcattgaagtgtttagcttgttaagataggcagttctcaaataaaaggtttgttttattgggtaaatgaccttgtagttttttggtgacagagcatagaaagtaatttcatgctgctcctgtgctattgtttttgtgaagacagggagctgtgaaaaactgcttagctacctacattcctcaataaaggcatcagacagtaattggtgattacagatgttctccctggaatggtcgttctcttgaccaagtagtcctacacttctggaaggatcattcagaactgtggtctatgccaacccaccagtagttcctgagtccctgcagtgctgagtgctgggggccaccaagttgagtaagacactgcagctctcaaagagttggatctaggattgtattgtatcgatttgtggtgtttggatatagtttttccatgatcccctacgaaaatatgcctctcatatgtaagaatcatgcctcctccgtgtacacttttcagacactgacaaggaagggtgttcaatacagtactgaattttcatatagcttttctgggggggccaaaataccaaaatcaacccatttcctacctttattctgtccataaaattgttagaaatatcaaaatcccatttcattccctgttaaatacatgtgaacgttgtctagacgctggagagcaaattctaccacctcttttgttcagcagtacatcagacgattgcatagacgtgccagatggaaccaaataataatgcacatggatttgtcataatccgtacaagtcattgacgcccacactgagccaggtgctgtgggagacaggaccatgtgtgaaagagaagacatgcttgcttctataaaagcatcggtgttattgaggagacctgacattaatgcagaatagcaaatgaccatgcaaattaattcactactaactaagctgcaggttgcacctcggaatgcagaggggcttcaaagtgatgagggtaggccctgagccaggccctgatgatgggtggattttgaggatcagagagtacagcttagagagataccccaagtgggaccaccccttgcccagtaggctgacaaactaaggctcttggtccctttttcatattttgggtgttctagtggcccagccagagctagacttcgagtcatgaattttctggcacaagtgttgtcacattcaaaaaagtattttctttgtttgaaaaatgaaaaatatatatatatgtgtgtgtgtatatatatatgtgtgtgtgtgtgtatatatgtgtgtgtgtatatatacgtgtgtgtgtatatgtgtgtgtgtgtgtgtgtgtgtgtgtgtatatatgtgtatatatataccatttttcccacctaaaatggagcatggcaaatctggactggattagtgagatagaccaagtcacagagcactccaggatgcagctgtgagctggggaacaggtcagaaaggcctcagggacatcagcatacatgttggagtttctgcagttttcttagggaaccctttaatgtcactagagctaacacacttgtcacctgggaagcaagcctgccagagcaaattagagagacgagggacagttgctagaaagacacacctggaagttctatttaactagcattaattatgtgctaggtgctgaggatgtagactgagtgagatcctcattcctcctctgtagggtggaagagaggatattgcttgtctccatggctcgtagtgaacagtcagtgagaccaggcacataaacctcttagcagaacacttggcctttctaaggactccatatgtgttccggggtaaatgcctgtgtttcttgacgtagtgatgtcttgttcctctagacatcactaactttacacagtagctttagatggcgtggacgtgaataaatgcaacttaggttttcttgttggtttctttttgagtatcattgtgtttgtaaagaatttcagattagaggattgttaccacgtgggccttcaggaggaaactgttttgagtttttgtcagcccgaaatcgatttgtgcgtttaagtatatgtgctcatcaaaacaggccaggctctgctgcagtaacaaacttacaagtctccgaggcttggcacaacggaagtctttgtgtcactgacgcccacttcagctttgtgttgctgaagcattctttggccctatccgagctgtccctctggtggtggtgcctgggggtttgggttccctctgtgttgtgatccaccatctcagcatcggcttccacagcagccatagcaggagaagaaaatgctgggggctctcagggtgcttttaagggcctggccaccgacctgcaaggggtgcgagttgccttcctgtgtggccagaactgatgataaactgtagactcatccctgctgaaactcggctccagagtgctcccaaggctggacagcgtgtgggcactggatcccacctgtgttagcactggcaattgtattctcatttcttcttttattctccaggtggcaaatggtaggatccaaagccttgaggccaccattgagaagctcctgagcagtgagagcaagctgaagcaggccatgcttaccttagaactggagcggtcggccctgctgcagacggtggaggagctgcggcggcggagcgcagagcccagcgaccgggagcctgagtgcacgcagcccgagcccacgggcgactgacagctctgcaggagagattgcaacaccatcccacactgtccaggccttaactgagagggacagaagacgctggaaggagagaaggaagcgggaagtgtgcttctcagggaggaaaccggcttgccagcaagtagattcttacgaactccaacttgcaattcagggggcatgtcccagtgttttttttgttgtttttagatactaaatcgtcccttctccagtcctgattactgtacacagtagctttagatggcgtggacgtgaataaatgcaacttatgttttcttgttggttcctttttgagtgtcactgtgtttgtaaagagcattcacaatacggtggaatttcaaaagctggaagagctcgagatcatgcctcaggcaaaggcgtgggtccatcgttcttccgagagggtttgtgtggcgactacaccctcagcgtccctggcaaggtgcagttggctctcgcccattcttgttatggaaacctaagatgatcattgggaagatcagtgatcttgggtcattgatccctggctcagaggatagcggtttccatcataaaccaagatgatgagttcagcctttatccctcgtggttccactagatgtaacttaaaggagttaacatttgaggactttgttctacatcagattttactatttgaatgtttaagatcactttattgaatttgaagatcatcaaattaaataaaatgatttatttaatttggatatcctgatcactgtcaagtgaaatggatctctctctttggtatttaaggaagtttgtctttaaaaaaaaaatagagtgttttcatacatttttgcttatcccataagtacagttgatcaaagtcatagtaggtaaatgctttatgggacagctgacaccttttagaccctaccaggtattgctagcatgtgagctgcagttgtggggtctgagatatttctttgtggtagtttcatacccatactatagagtcatgtatttatttttgcctgttgtgtgatgtaatgcaatcatgttcctttgagtctccatcccttggaaatctgacttcttgcagaaggagtaggcacatcaagatattcaggggtgccccaagagtctgggactttcaaaaaaaaaagatcaggctnnaactgcagtcagatttatgacagctgacagtttttcagaggtcgcacacagtgactctcctctctcaggatgacgaggacctgtgccttcaacaagcaaaatgctgctcacggttgtcctgcttgcagccagtcactgtgtaaagcctctctgatgtgcacttaagagtgggttgctttctcacaaagatggggttctgtgcagtcacaggtcacttccttgacaacacaatcatttctgatctttatcactgtaaccacgtcttctattccataggagtttcttttgattctctcagttgcggggggcatctcttaatcctggggtaaaaggagagattgccatacttagactcactgtgagtctccccggccatttcacgaggagaccacagtgctgccaccagtgcctaaacaggtggctggcattcgagacttcctcctgttccctgggtcagaggatagcggtttccatcataaaccaagatgatgagttcagcctttatccctcgtggttccgctagatgtaacttataggagttaacatttgaggactttgttctgcatcagatcttactatttgaatgtttactgttggattttgggcatcttattactgttactcaaaaacattgactctgcatcaagaaagaaacaagaaagcaataaaacaagaaataattcatgctcacatttttatggtggttttttttttttttttaactttggatttttgcttttcagcccaggagtaaaggaatgccttatgaacacctgtggcctacgtgtggtcatgacccaaccatcagtgagattatttgagatattggtgtctgcatccagtgttgttatctgagtgtttattacgtaagttgtaacacctctacacagggtgtgagtttagcactgatgagaccagctccatcattgtatgtggcagtgagtcctgttacgagattgggttgggcagaaaggactgttgacatgagcctgtggatgtaggttggacagtctcagcctgtgactgactaggcaaggagcggagaggcaactgtgtgaggattctcagagccaaatttttaagccatgttttgggttatatttcccccaacactcatttgtgcacttggtggtgtcaa

SEQ ID NO: 3 - DNA, 3983 bp, Homo sapiens
  CDS 171..3725
  polyA_signal 3942..3947: AATAAA
ccaggccgtccccaggatgcccccaagcacctgcgngtcccggcccggccccgggctctgagcgcgccgcggcacaggtttctgcatatgaagtgtgtaaaatagattgcttgatccaaaacagaaaaacagtgataactgttttgctgagttcccagacccttcccaagatggaaccaataacattcacagcaaggaaacatctgcttcctaacgaggtctcggtggattttggcctgcagctggtgggctccctgcctgtgcattccctgaccaccatgcccatgctgccctgggttgtggctgaggtgcgaagactcagcaggcagtccaccagaaaggaacctgtaaccaagcaagtccggctttgcgtttcaccctctggactgagatgtgaacctgagccagggagaagtcaacagtgggatcccctgatctattccagcatctttgagtgcaagcctcagcgtgttcacaaactgattcacaacagtcatgacccaagttactttgcttgtctgattaaggaagacgctgtccaccggcagagtatctgctatgtgttcaaagccgatgatcaaacaaaagtgcctgagatcatcagctccatccgtcaggcggggaagatcgcccggcaggaggagctgcactgcccgtccgagttcgacgacacgttttccaagaagttcgaggtgctcttctgcggccgcgtgacggtggcgcacaagaaggctccgccggccctgatcgacgagtgcatcgagaagttcaatcacgtcagcggcagccgggggtccgagagcccccgccccaacccgccccatgccgcgcccacagggagccaggagcctgtgcgcaggcccatgcgcaagtccttctcccagcccggcctgcgctcgctggcctttaggaaggagctgcaggatgggggcctccgaagcagcggcttcttcagctccttcgaggagagcgacattgagaaccacctcattagcggacacaatattgtgcagcccacagatatcgaggaaaatcgaactatgctcttcacgattggccagtctgaagtttacctcatcagtcctgacaccaaaaaaatagcattggagaaaaattttaaggagatatccttttgctctcagggcatcagacacgtggaccactttgggtttatctgtcgggagtcttccggaggtggcggctttcattttgtctgttacgtgtttcagtgcacaaatgaggctctggttgatgaaattatgatgaccctgaaacaggccttcacggtggccgcagtgcagcagacagctaaggcgccagcccagctgtgtgagggctgccccctgcaaagcctgcacaagctctgtgagaggatagagggaatgaattcttccaaaacaaaactagaactgcaaaagcacctgacgacattaaccaatcaggagcaggcgactatttttgaagaggttcagaaattgagaccgagaaatgagcagcgagagaatgaattgattatttcttttctgagatgtttatatgaagagaaacagaaagaacacatccatattggggagatgaagcagacatcgcagatggcagcagagaatattggaagtgaattaccacccagtgccactcgatttaggctagatatgctgaaaaacaaagcaaagagatctttaacagagtctttagaaagtattttgtcccggggtaataaagccagaggcctgcaggaacactccatcagtgtggatctggatagctccctgtctagtacattaagtaacaccagcaaagagccatctgtgtgtgaaaaggaggccttgcccatctctgagagctcctttaagctcctcggctcctcggaggacctgtccagtgactcggagagtcatctcccagaagagccagctccgctgtcgccccagcaggccttcaggaggcgagcaaacaccctgagtcacttccccatcgaatgccaggaacctccacaacctgcccgggggtccccgggggtttcgcaaaggaaacttatgaggtatcactcagtgagcacagagacgcctcatgaacgaaaggactttgaatccaaagcaaaccatcttggtgattctggtgggactcctgtgaagacccggaggcattcctggaggcagcagatattcctccgagtagccaccccgcagaaggcgtgcgattcttccagcagatatgaagattattcagagctgggagagcttcccccacgatctcctttagaaccagtttgtgaagatgggccctttggccccccaccagaggaaaagaaaaggacatctcgtgagctccgagagctgtggcaaaaggctattcttcaacagatactgctgcttagaatggagaaggaaaatcagaagctccaagcctctgaaaatgatttgctgaacaagcgcctgaagctcgattatgaagaaattactccctgtcttaaagaagtaactacagtgtgggaaaagatgcttagcactccaggaagatcaaaaattaagtttgacatggaaaaaatgcactcggctgttgggcaaggtgtgccacgtcatcaccgaggtgaaatctggaaatttctagctgagcaattccaccttaaacaccagtttcccagcaaacagcagccaaaggatgtgccatacaaagaactcttaaagcagctgacttcccagcagcatgcgattcttattgaccttgggcgaacctttcctacacacccatacttctctgcccagcttggagcaggacagctatcgctttacaacattttgaaggcctactcacttctagaccaggaagtgggatattgccaaggtctcagctttgtagcaggcattttgcttcttcatatgagtgaggaagaggcgtttaaaatgctcaagtttctgatgtttgacatggggctgcggaaacagtatcggccagacatgattattttacagatccagatgtaccagctctcgaggttgcttcatgattaccacagagacctctacaatcacctggaggagcacgagatcggccccagcctctacgctgccccctggttcctcaccatgtttgcctcacagttcccgctgggattcgtagccagagtctttgatatgatttttcttcagggaacagaggtcatatttaaagtggctttaagtctgttgggaagccataagcccttgattctgcagcatgaaaacctagaaaccatagttgactttataaaaagcacgctacccaaccttggcttggtacagatggaaaagaccatcaatcaggtatttgaaatggacatcgctaaacagttacaagcttatgaagttgagtaccacgtccttcaagaagaacttatcgattcctctcctctcagtgacaaccaaagaatggataaattagagaaaaccaacagcagcttacgcaaacagaaccttgacctccttgaacagttgcaggtggcaaatggtaggatccaaagccttgaggccaccattgagaagctcctgagcagtgagagcaagctgaagcaggccatgcttaccttagaactggagcggtcggccctgctgcagacggtggaggagctgcggcggcggagcgcagagcccagcgaccgggagcctgagtgcacgcagcccgagcccacgggcgactgacagctctgcaggagagattgcaacaccatcccacactgtccaggccttaactgagagggacagaagacgctggaaggagagaaggaagcgggaagtgtgcttctcagggaggaaaccggcttgccagcaagtagattcttacgaactccaacttgcaattcagggggcatgtcccagtgttttttttgttgtttttagatactaaatcgtcccttctccagtcctgattactgtacacagtagctttagatggcgtggacgtgaataaatgcaacttatgttttaaaaaaaaaaaaaaaaaaaaaa

SEQ ID NO: 4 - DNA, 3988 bp, Homo sapiens
  CDS 176..3730
  polyA_signal 3947..3952: AATAAA
ataataggcactgaagacatgttaatggaaggtggatttgtgattcagaacctctagactacctgggcgagtcttttaaaatgtttctgcatatgaagtgtgtaaaatagattgcttgatccaaaacagaaaaacagtgataactgttttgctgagttcccagacccttcccaagatggaaccaataacattcacagcaaggaaacatctgcttcctaacgaggtctcggtggattttggcctgcagctggtgggctccctgcctgtgcattccctgaccaccatgcccatgctgccctgggttgtggctgaggtgcgaagactcagcaggcagtccaccagaaaggaacctgtaaccaagcaagtccggctttgcgtttcaccctctggactgagatgtgaacctgagccagggagaagtcaacagtgggatcccctgatctattccagcatctttgagtgcaagcctcagcgtgttcacaaactgattcacaacagtcatgacccaagttactttgcttgtctgattaaggaagacgctgtccaccggcagagtatctgctatgtgttcaaagccgatgatcaaacaaaagtgcctgagatcatcagctccatccgtcaggcggggaagatcgcccggcaggaggagctgcactgcccgtccgagttcgacgacacgttttccaagaagttcgaggtgctcttctgcggccgcgtgacggtggcgcacaagaaggctccgccggccctgatcgacgagtgcatcgagaagttcaatcacgtcagcggcagccgggggtccgagagcccccgccccaacccgccccatgccgcgcccacagggagccaggagcctgtgcgcaggcccatgcgcaagtccttctcccagcccggcctgcgctcgctggcctttaggaaggagctgcaggatgggggcctccgaagcagcggcttcttcagctccttcgaggagagcgacattgagaaccacctcattagcggacacaatattgtgcagcccacagatatcgaggaaaatcgaactatgctcttcacgattggccagtctgaagtttacctcatcagtcctgacaccaaaaaaatagcattggagaaaaattttaaggagatatccttttgctctcagggcatcagacacgtggaccactttgggtttatctgtcgggagtcttccggaggtggcggctttcattttgtctgttacgtgtttcagtgcacaaatgaggctctggttgatgaaattatgatgaccctgaaacaggccttcacggtggccgcagtgcagcagacagctaaggcgccagcccagctgtgtgagggctgccccctgcaaagcctgcacaagctctgtgagaggatagagggaatgaattcttccaaaacaaaactagaactgcaaaagcacctgacgacattaaccaatcaggagcaggcgactatttttgaagaggttcagaaattgagaccgagaaatgagcagcgagagaatgaattgattatttcttttctgagatgtttatatgaagagaaacagaaagaacacatccatattggggagatgaagcagacatcgcagatggcagcagagaatattggaagtgaattaccacccagtgccactcgatttaggctagatatgctgaaaaacaaagcaaagagatctttaacagagtctttagaaagtattttgtcccggggtaataaagccagaggcctgcaggaacactccatcagtgtggatctggatagctccctgtctagtacattaagtaacaccagcaaagagccatctgtgtgtgaaaaggaggccttgcccatctctgagagctcctttaagctcctcggctcctcggaggacctgtccagtgactcggagagtcatctcccagaagagccagctccgctgtcgccccagcaggccttcaggaggcgagcaaacaccctgagtcacttccccatcgaatgccaggaacctccacaacctgcccgggggtccccgggggtttcgcaaaggaaacttatgaggtatcactcagtgagcacagagacgcctcatgaacgaaaggactttgaatccaaagcaaaccatcttggtgattctggtgggactcctgtgaagacccggaggcattcctggaggcagcagatattcctccgagtagccaccccgcagaaggcgtgcgattcttccagcagatatgaagattattcagagctgggagagcttcccccacgatctcctttagaaccagtttgtgaagatgggccctttggccccccaccagaggaaaagaaaaggacatctcgtgagctccgagagctgtggcaaaaggctattcttcaacagatactgctgcttagaatggagaaggaaaatcagaagctccaagcctctgaaaatgatttgctgaacaagcgcctgaagctcgattatgaagaaattactccctgtcttaaagaagtaactacagtgtgggaaaagatgcttagcactccaggaagatcaaaaattaagtttgacatggaaaaaatgcactcggctgttgggcaaggtgtgccacgtcatcaccgaggtgaaatctggaaatttctagctgagcaattccaccttaaacaccagtttcccagcaaacagcagccaaaggatgtgccatacaaagaactcttaaagcagctgacttcccagcagcatgcgattcttattgaccttgggcgaacctttcctacacacccatacttctctgcccagcttggagcaggacagctatcgctttacaacattttgaaggcctactcacttctagaccaggaagtgggatattgccaaggtctcagctttgtagcaggcattttgcttcttcatatgagtgaggaagaggcgtttaaaatgctcaagtttctgatgtttgacatggggctgcggaaacagtatcggccagacatgattattttacagatccagatgtaccagctctcgaggttgcttcatgattaccacagagacctctacaatcacctggaggagcacgagatcggccccagcctctacgctgccccctggttcctcaccatgtttgcctcacagttcccgctgggattcgtagccagagtctttgatatgatttttcttcagggaacagaggtcatatttaaagtggctttaagtctgttgggaagccataagcccttgattctgcagcatgaaaacctagaaaccatagttgactttataaaaagcacgctacccaaccttggcttggtacagatggaaaagaccatcaatcaggtatttgaaatggacatcgctaaacagttacaagcttatgaagttgagtaccacgtccttcaagaagaacttatcgattcctctcctctcagtgacaaccaaagaatggataaattagagaaaaccaacagcagcttacgcaaacagaaccttgacctccttgaacagttgcaggtggcaaatggtaggatccaaagccttgaggccaccattgagaagctcctgagcagtgagagcaagctgaagcaggccatgcttaccttagaactggagcggtcggccctgctgcagacggtggaggagctgcggcggcggagcgcagagcccagcgaccgggagcctgagtgcacgcagcccgagcccacgggcgactgacagctctgcaggagagattgcaacaccatcccacactgtccaggccttaactgagagggacagaagacgctggaaggagagaaggaagcgggaagtgtgcttctcagggaggaaaccggcttgccagcaagtagattcttacgaactccaacttgcaattcagggggcatgtcccagtgttttttttgttgtttttagatactaaatcgtcccttctccagtcctgattactgtacacagtagctttagatggcgtggacgtgaataaatgcaacttatgttttaaaaaaaaaaaaaaaaaaaaaa

SEQ ID NO: 5 - protein, 1168 aa, Homo sapiens
MEPITFTARKHLLPNEVSVDFGLQLVGSLPVHSLTTMPMLPWVVAEVRRLSRQSTRKEPVTKQVRLCVSPSGLRCEPEPGRSQQWDPLIYSSIFECKPQRVHKLIHNSHDPSYFACLIKEDAVHRQSICYVFKADDQTKVPEIISSIRQAGKIARQEELHCPSEFDDTFSKKFEVLFCGRVTVAHKKAPPALIDECIEKFNHVSGSRGSESPRPNPPHAAPTGSQEPVRRPMRKSFSQPGLRSLAFRKELQDGGLRSSGFFSSFEESDIENHLISGHNIVQPTDIEENRTMLFTIGQSEVYLISPDTKKIALEKNFKEISFCSQGIRHVDHFGFICRESSGGGGFHFVCYVFQCTNEALVDEIMMTLKQAFTVAAVQQTAKAPAQLCEGCPLQSLHKLCERIEGMNSSKTKLELQKHLTTLTNQEQATIFEEVQKLRPRNEQRENELIISFLRCLYEEKQKEHIHIGEMKQTSQMAAENIGSELPPSATRFRLDMLKNKAKRSLTESLESILSRGNKARGLQEHSISVDLDSSLSSTLSNTSKEPSVCEKEALPISESSFKLLGSSEDLSSDSESHLPEEPAPLSPQQAFRRRANTLSHFPIECQEPPQPARGSPGVSQRKLMRYHSVSTETPHERKDFESKANHLGDSGGTPVKTRRHSWRQQIFLRVATPQKACDSSSRYEDYSELGELPPRSPLEPVCEDGPFGPPPEEKKRTSRELRELWQKAILQQILLLRMEKENQKLQASENDLLNKRLKLDYEEITPCLKEVTTVWEKMLSTPGRSKIKFDMEKMHSAVGQGVPRHHRGEIWKFLAEQFHLKHQFPSKQQPKDVPYKELLKQLTSQQHAILIDLGRTFPTHPYFSAQLGAGQLSLYNILKAYSLLDQEVGYCQGLSFVAGILLLHMSEEEAFKMLKFLMFDMGLRKQYRPDMIILQIQMYQLSRLLHDYHRDLYNHLEEHEIGPSLYAAPWFLTMFASQFPLGFVARVFDMIFLQGTEVIFKVALSLLGSHKPLILQHENLETIVDFIKSTLPNLGLVQMEKTINQVFEMDIAKQLQAYEVEYHVLQEELIDSSPLSDNQRMDKLEKTNSSLRKQNLDLLEQLQVANGRIQSLEATIEKLLSSESKLKQAMLTLELERSALLQTVEELRRRSAEPSDREPECTQPEPTGD

SEQ ID NO: 6 - DNA, 18 bp, Artificial Sequence
  misc_binding 1..18: sequencing oligonucleotide PrimerPU
tgtaaaacgacggccagt

SEQ ID NO: 7 - DNA, 18 bp, Artificial Sequence
  misc_binding 1..18: sequencing oligonucleotide PrimerRP
caggaaacagctatgacc

(19) United States
(12) Patent Application Publication
(10) Pub. No.: US 2006/0084073 A1
(43) Pub. Date: Apr. 20, 2006

(54) Nucleic acids encoding human TBC-1 protein and polymorphic markers thereof

(75) Inventors: Marta Blumenfeld, Paris (FR); Lydie Bougueleret, Petit-Lancy (CH); Ilya Chumakov, Vaux-le Penil (FR)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: Genset S.A., Evry (FR)

(21) Appl. No.: 10/970,750

(22) Filed: Oct. 20, 2004

(51) Int. Cl. C12Q 1/68; C07H 21/04; C07K 14/82

(52) U.S. Cl. 435/6; 435/69.3; 435/320.1; 435/325; 530/350; 536/23.2

(57) ABSTRACT

The invention concerns genomic and cDNA sequences of the human TBC-1 gene. The invention also concerns polypeptides encoded by the TBC-1 gene. The invention also deals with antibodies directed specifically against such polypeptides that are useful as diagnostic reagents. The invention further encompasses biallelic markers of the TBC-1 gene useful in genetic analysis.

### FIELD OF THE INVENTION

The invention concerns genomic and cDNA sequences of the human TBC-1 gene. The invention also concerns polypeptides encoded by the TBC-1 gene. The invention also deals with antibodies directed specifically against such polypeptides that are useful as diagnostic reagents. The invention further encompasses biallelic markers of the TBC-1 gene useful in genetic analysis.

### BACKGROUND OF THE INVENTION

The incidence of prostate cancer has dramatically increased over the last decades. It averages 30-50/100,000 males in Western European countries as well as within the US White male population. In these countries, it has recently become the most commonly diagnosed malignancy, being one of every four cancers diagnosed in American males. Prostate cancer's incidence is very much population specific, since it varies from 2/100,000 in China, to over 80/100,000 among African-American males.

In France, the incidence of prostate cancer is 35/100,000 males and it is increasing by 10/100,000 per decade. Mortality due to prostate cancer is also growing accordingly. It is the second cause of cancer death among French males, and the first one among French males aged over 70. This makes prostate cancer a serious burden in terms of public health.

Prostate cancer is a latent disease. Many men carry prostate cancer cells without overt signs of disease. Autopsies of individuals dying of other causes show prostate cancer cells in 30% of men at age 50 and in 60% of men at age 80. Furthermore, prostate cancer can take up to 10 years to kill a patient after the initial diagnosis.

The progression of the disease usually goes from a well-defined mass within the prostate to a breakdown and invasion of the lateral margins of the prostate, followed by metastasis to regional lymph nodes, and metastasis to the bone marrow. Cancer metastasis to bone is common and often associated with uncontrollable pain.

Unfortunately, in 80% of cases, diagnosis of prostate cancer is established when the disease has already metastasized to the bones. Of special interest is the observation that prostate cancers frequently grow more rapidly in sites of metastasis than within the prostate itself.

Early-stage diagnosis of prostate cancer mainly relies today on Prostate Specific Antigen (PSA) dosage, and allows the detection of prostate cancer seven years before clinical symptoms become apparent. The effectiveness of PSA dosage diagnosis is however limited, due to its inability to discriminate between malignant and non-malignant affections of the organ and because not all prostate cancers give rise to an elevated serum PSA concentration. Furthermore, PSA dosage and other currently available approaches such as physical examination, tissue biopsy and bone scans are of limited value in predicting disease progression.

Therefore, there is a strong need for a reliable diagnostic procedure which would enable a more systematic early-stage prostate cancer prognosis.

Although an early-stage prostate cancer prognosis is important, the possibility of measuring the period of time during which treatment can be deferred is also interesting as currently available medicaments are expensive and generate important adverse effects. However, the aggressiveness of prostate tumors varies widely. Some tumors are relatively aggressive, doubling every six months whereas others are slow-growing, doubling once every five years. In fact, the majority of prostate cancers grows relatively slowly and never becomes clinically manifest. Very often, affected patients are among the elderly and die from another disease before prostate cancer actually develops. Thus, a significant question in treating prostate carcinoma is how to discriminate between tumors that will progress and those that will not progress during the expected lifetime of the patient.

Hence, there is also a strong need for detection means which may be used to evaluate the aggressiveness or the development potential of prostate cancer tumors once diagnosed.

Furthermore, at the present time, there is no means to predict prostate cancer susceptibility. It would also be very beneficial to detect individual susceptibility to prostate cancer. This could allow preventive treatment and a careful follow up of the development of the tumor.

A further consequence of the slow growth rate of prostate cancer is that few cancer cells are actively dividing at any one time, rendering prostate cancer generally resistant to radiation and chemotherapy. Surgery is the mainstay of treatment but it is largely ineffective and removes the ejaculatory ducts, resulting in impotence. Oral oestrogens and luteinizing releasing hormone analogs are also used for treatment of prostate cancer. These hormonal treatments provide marked improvement for many patients, but they only provide temporary relief. Indeed, most of these cancers soon relapse with the development of hormone-resistant tumor cells and the oestrogen treatment can lead to serious cardiovascular complications. Consequently, there is a strong need for preventive and curative treatment of prostate cancer.

Efficacy/tolerance prognosis could be precious in prostate cancer therapy. Indeed, hormonal therapy, the main treatment currently available, presents important side effects. The use of chemotherapy is limited because of the small number of patients with chemosensitive tumors. Furthermore the age profile of the prostate cancer patient and intolerance to chemotherapy make the systematic use of this treatment very difficult.

Therefore, a valuable assessment of the eventual efficacy of a medicament to be administered to a prostate cancer patent as well as the patent's eventual tolerance to it may permit to enhance the benefit/risk ratio of prostate cancer treatment.

It is known today that there is a familial risk of prostate cancer. Clinical studies in the 1950s had already demonstrated a familial aggregation in prostate cancer. Control-case clinical studies have been conducted more recently to attempt to evaluate the incidence of the genetic risk factors in the disease. Thus Steinberg et al., 1990, and McWhorter et al., 1992 confirm that the risk of prostate cancer is increased in subjects having one or more relatives already affected by the disease and when forms of early diagnosis in the relatives exist.

It is now well established that cancer is a disease caused by the deregulation of the expression of certain genes. In fact, the development of a tumor necessitates an important succession of steps. Each of these steps comprises the deregulation of an important gene intervening in the normal metabolism of the cell and the emergence of an abnormal cellular sub-clone which overwhelms the other cell types because of a proliferative advantage. The genetic origin of this concept has found confirmation in the isolation and the characterization of genes which could be responsible. These genes, commonly called “cancer genes”, have an important role in the normal metabolism of the cell and are capable of intervening in carcinogenesis following a change.

Recent studies have identified three groups of genes which are frequently mutated in cancer. The first group of genes, called oncogenes, are genes whose products activate cell proliferation. The normal non-mutant versions are called protooncogenes. The mutated forms are excessively or inappropriately active in promoting cell proliferation, and act in the cell in a dominant way in that a single mutant allele is enough to affect the cell phenotype. Activated oncogenes are rarely transmitted as germline mutations since they may probably be lethal when expressed in all the cells. Therefore oncogenes can only be investigated in tumor tissues.

The second group of genes which are frequently mutated in cancer, called tumor suppressor genes, are genes whose products inhibit cell growth. Mutant versions in cancer cells have lost their normal function, and act in the cell in a recessive way in that both copies of the gene must be inactivated in order to change the cell phenotype. Most importantly, the tumor phenotype can be rescued by the wild type allele, as shown by cell fusion experiments first described by Harris and colleagues (1969). Germline mutations of tumor suppressor genes may be transmitted and thus studied in both constitutional and tumor DNA from familial or sporadic cases. The current family of tumor suppressors includes DNA-binding transcription factors (i.e., p53, WT1), transcription regulators (i.e., RB, APC, probably BRCA1), protein kinase inhibitors (i.e., p16), among others (for review, see Haber D & Harlow E, 1997).

The third group of genes which are frequently mutated in cancer, called mutator genes, are responsible for maintaining genome integrity and/or low mutation rates. Loss of function of both alleles increases cell mutation rates, and as a consequence, proto-oncogenes and tumor suppressor genes may be mutated. Mutator genes can also be classified as tumor suppressor genes, except for the fact that tumorigenesis caused by this class of genes cannot be suppressed simply by restoration of a wild-type allele, as described above. Genes whose inactivation may lead to a mutator henotype include mismatch repair genes (i.e., MLH1, MSH2), DNA helicases (i.e., BLM, WRN) or other genes involved in DNA repair and genomic stability (i.e., p53, possibly BRCA1 and BRCA2) (For review see Haber D & Harlow E, 1997; Fishel R & Wilson T. 1997; Ellis N A, 1997).

There is growing evidence that a critical event in the progression of a tumor cell from a non-metastatic to metastatic phenotype is the loss of function of metastasis-suppressor genes. These genes specifically suppress the ability of a cell to metastasize. Work from several groups has demonstrated that human chromosomes 8, 10, 11 and 17 encode prostate cancer metastasis suppressor activities. However, other human chromosomes such as chromosomes 1, 7, 13, 16, and 18 may also be associated to prostate cancer.

It thus remains to localize and to identify the genes specifically involved in the development and the progression of prostate cancers starting from the genetic analysis of the hereditary and the non-hereditary forms and to define their clinical implications in terms of prognosis and therapeutic innovations.

### SUMMARY OF THE INVENTION

The present invention pertains to nucleic acid molecules comprising the genomic sequence of a novel human gene which encodes a TBC-1 protein. The TBC-1 genomic sequences comprise regulatory sequence located upstream (5′-end) and downstream (3′-end) of the transcribed portion of said gene, these regulatory sequences being also part of the invention. The human TBC-1 genomic sequence is included in a previously unknown candidate region of prostate cancer located on chromosome 4.

The invention also deals with the two complete cDNA sequences encoding the TBC-1 protein, as well as with the corresponding translation product.

Oligonucleotide probes or primers hybridizing specifically with a TBC-1 genomic or cDNA sequence are also part of the present invention, as well as DNA amplification and detection methods using said primers and probes.

A further object of the invention consists of recombinant vectors comprising any of the nucleic acid sequences described above, and in particular of recombinant vectors comprising a TBC-1 regulatory sequence or a sequence encoding a TBC-1 protein, as well as of cell hosts and transgenic non human animals comprising said nucleic acid sequences or recombinant vectors.

The invention also concerns a TBC-1-related biallelic marker and the use thereof.

Finally, the invention is directed to methods for the screening of substances or molecules that inhibit the expression of TBC-1, as well as with methods for the screening of substances or molecules that interact with a TBC-1 polypeptide.

### BRIEF DESCRIPTION OF THE DRAWINGS

FIG. 1: An amino acid alignment of a portion of the amino acid sequence of the TBC-1 protein of SEQ ID No 5 with other proteins sharing amino acid homology with TBC-1. The amino acid numbering refers to the murine TBC-1.

### BRIEF DESCRIPTION OF THE SEQUENCES PROVIDED IN THE SEQUENCE LISTING

SEQ ID No 1 contains a first part of the TBC-1 genomic sequence comprising the 5′ regulatory sequence and the exons 1, 1bis, and 2.

SEQ ID No 2 contains a second part of the TBC-1 genomic sequence comprising the 12 last exons of the TBC-1 gene and the 3′ regulatory sequence.

SEQ ID No 3 contains a first cDNA sequence of the TBC-1 gene.

SEQ ID No 4 contains a second cDNA sequence of the TBC-1 gene.

SEQ ID No 5 contains the amino acid sequence encoded by the cDNAs of SEQ ID Nos 3 and 4.

SEQ ID No 6 contains a primer containing the additional PU 5′ sequence described further in Example 3.

SEQ ID No 7 contains a primer containing the additional RP 5′ sequence described further in Example 3.

In accordance with the regulations relating to Sequence Listings, the following codes have been used in the Sequence Listing to indicate the locations of biallelic markers within the sequences and to identify each of the alleles present at the polymorphic base. The code “r” in the sequences indicates that one allele of the polymorphic base is a guanine, while the other allele is an adenine. The code “y” in the sequences indicates that one allele of the polymorphic base is a thymine, while the other allele is a cytosine. The code “m” in the sequences indicates that one allele of the polymorphic base is an adenine, while the other allele is an cytosine. The code “k” in the sequences indicates that one allele of the polymorphic base is a guanine, while the other allele is a thymine. The code “s” in the sequences indicates that one allele of the polymorphic base is a guanine, while the other allele is a cytosine. The code “w” in the sequences indicates that one allele of the polymorphic base is an adenine, while the other allele is an thymine. The nucleotide code of the original allele for each biallelic marker is the following:

| Biallelic marker | Original allele |
|---|---|
| 99-430-352 | G |
| 99-20508-456 | C |
| 99-20469-213 | C |
| 5-254-227 | A |
| 5-257-353 | C |
| 99-20511-32 | T |
| 99-20511-221 | A |
| 99-20504-90 | G |
| 99-20493-238 | A |
| 99-20499-221 | G |
| 99-20499-364 | A |
| 99-20499-399 | A |
| 5-249-304 | G |
| 99-20485-269 | A |
| 99-20481-131 | G |
| 99-20481-419 | T |
| 99-20480-233 | A |

### DETAILED DESCRIPTION OF THE INVENTION

The present invention concerns polynucleotides and polypeptides related to the human TBC-1 gene (also termed “TBC-1 gene” throughout the present specification), which is potentially involved in the regulation of the differentiation of various cell types in mammals. A deregulation or an alteration of TBC-1 expression, or alternatively an alteration in the amino acid sequence of the TBC-1 protein may be involved in the generation of a pathological state related to cell differentiation in a patient, more particularly to abnormal cell proliferation leading to cancer states, such as prostate cancer.

### Definitions

Before describing the invention in greater detail, the following definitions are set forth to illustrate and define the meaning and scope of the terms used to describe the invention herein.

The term “TBC-1 gene”, when used herein, encompasses mRNA and cDNA sequences encoding the TBC-1 protein. In the case of a genomic sequence, the TBC-1 gene also includes native regulatory regions which control the expression of the coding sequence of the TBC-1 gene.

The term “functionally active fragment” of the TBC-1 protein is intended to designate a polypeptide carrying at least one of the structural features of the TBC-1 protein involved in at least one of the biological functions and/or activity of the TBC-1 protein.

A “heterologous” or “exogenous” polynucleotide designates a purified or isolated nucleic acid that has been placed, by genetic engineering techniques, in the environment of unrelated nucleotide sequences, such as the final polynucleotide construct does not occur naturally. An illustrative, but not limitative, embodiment of such a polynucleotide construct may be represented by a polynucleotide comprising (1) a regulatory polynucleotide derived from the TBC-1 gene sequence and (2) a polynucleotide encoding a cytokine, for example GM-CSF. The polypeptide encoded by the heterologous polynucleotide will be termed an heterologous polypeptide for the purpose of the present invention.

By a “biologically active fragment or variant” of a regulatory polynucleotide according to the present invention is intended a polynucleotide comprising or alternatively consisting in a fragment of said polynucleotide which is functional as a regulatory region for expressing a recombinant polypeptide or a recombinant polynucleotide in a recombinant cell host.

For the purpose of the invention, a nucleic acid or polynucleotide is “functional” as a regulatory region for expressing a recombinant polypeptide or a recombinant polynucleotide if said regulatory polynucleotide contains nucleotide sequences which contain transcriptional and translational regulatory information, and such sequences are “operatively linked” to nucleotide sequences which encode the desired polypeptide or the desired polynucleotide. An operable linkage is a linkage in which the regulatory nucleic acid and the DNA sequence sought to be expressed are linked in such a way as to permit gene expression.

A “promoter” refers to a DNA sequence recognized by the synthetic machinery of the cell required to initiate the specific transcription of a gene.

A sequence which is “operably linked” to a regulatory sequence such as a promoter means that said regulatory element is in the correct location and orientation in relation to the nucleic acid to control RNA polymerase initiation and expression of the nucleic acid of interest.

As used herein, the term “operably linked” refers to a linkage of polynucleotide elements in a functional relationship. For instance, a promoter or enhancer is operably linked to a coding sequence if it affects the transcription of the coding sequence. More precisely, two DNA molecules (such as a polynucleotide containing a promoter region and a polynucleotide encoding a desired polypeptide or polynucleotide) are said to be “operably linked” if the nature of the linkage between the two polynucleotides does not (1) result in the introduction of a frame-shift mutation or (2) interfere with the ability of the polynucleotide containing the promoter to direct the transcription of the coding polynucleotide. The promoter polynucleotide would be operably linked to a polynucleotide encoding a desired polypeptide or a desired polynucleotide if the promoter is capable of effecting transcription of the polynucleotide of interest.

The term “primer” denotes a specific oligonucleotide sequence which is complementary to a target nucleotide sequence and used to hybridize to the target nucleotide sequence. A primer serves as an initiation point for nucleotide polymerization catalyzed by either DNA polymerase, RNA polymerase or reverse transcriptase.

The term “probe” denotes a defined nucleic acid segment (or nucleotide analog segment, e.g., polynucleotide as defined hereinbelow) which can be used to identify a specific polynucleotide sequence present in samples, said nucleic acid segment comprising a nucleotide sequence complementary of the specific polynucleotide sequence to be identified.

The terms “sample” or “material sample” are used herein to designate a solid or a liquid material suspected to contain a polynucleotide or a polypeptide of the invention. A solid material may be, for example, a tissue slice or biopsy within which is searched the presence of a polynucleotide encoding a TBC-1 protein, either a DNA or RNA molecule or within which is searched the presence of a native or a mutated TBC-1 protein, or alternatively the presence of a desired protein of interest the expression of which has been placed under the control of a TBC-1 regulatory polynucleotide. A liquid material may be, for example, any body fluid like serum, urine etc., or a liquid solution resulting from the extraction of nucleic acid or protein material of interest from a cell suspension or from cells in a tissue slice or biopsy. The term “biological sample” is also used and is more precisely defined within the Section dealing with DNA extraction.

As used herein, the term “purified” does not require absolute purity; rather, it is intended as a relative definition. Purification if starting material or natural material to at least one order of magnitude, preferably two or three orders, and more preferably four or five orders of magnitude is expressly contemplated. As an example, purification from 0.1% concentration to 10% concentration is two orders of magnitude.

The term “isolated” requires that the material be removed from its original environment (e.g. the natural environment if it is naturally occurring). For example, a naturally-occurring polynucleotide or polypeptide present in a living animal is not isolated, but the same polynucleotide or DNA or polypeptide, separated from some or all of the coexisting materials in the natural system, is isolated. Such polynucleotide could be part of a vector and/or such polynucleotide or polypeptide could be part of a composition and still be isolated in that the vector or composition is not part of its natural environment.

The term “polypeptide” refers to a polymer of amino acids without regard to the length of the polymer; thus, peptides, oligopeptides, and proteins are included within the definition of polypeptide. This term also does not specify or exclude post-expression modifications of polypeptides, for example, polypeptides which include the covalent attachment of glycosyl groups, acetyl groups, phosphate groups, lipid groups and the like are expressly encompassed by the term polypeptide. Also included within the definition are polypeptides which contain one or more analogs of an amino acid (including, for example, non-naturally occurring amino acids, amino acids which only occur naturally in an unrelated biological system, modified amino acids from mammalian systems etc.), polypeptides with substituted linkages, as well as other modifications known in the art, both naturally occurring and non-naturally occurring.

The term “recombinant polypeptide” is used herein to refer to polypeptides that have been artificially designed and which comprise at least two polypeptide sequences that are not found as contiguous polypeptide sequences in their initial natural environment, or to refer to polypeptides which have been expressed from a recombinant polynucleotide.

The term “purified” is used herein to describe a polypeptide of the invention which has been separated from other compounds including, but not limited to nucleic acids, lipids, carbohydrates and other proteins. A polypeptide is substantially pure when at least about 50%, preferably 60 to 75% of a sample exhibits a single polypeptide sequence. A substantially pure polypeptide typically comprises about 50%, preferably 60 to 90% weight/weight of a protein sample, more usually about 95%, and preferably is over about 99% pure. Polypeptide purity or homogeneity is indicated by a number of means well known in the art, such as polyacrylamide gel electrophoresis of a sample, followed by visualizing a single polypeptide band upon staining the gel. For certain purposes higher resolution can be provided by using HPLC or other means well known in the art.

As used herein, the term “non-human animal” refers to any non-human vertebrate, birds and more usually mammals, preferably primates, farm animals-such as swine, goats, sheep, donkeys, and horses, rabbits or rodents, more preferably rats or mice. As used herein, the term “animal” is used to refer to any vertebrate, preferable a mammal. Both the terms “animal” and “mammal” expressly embrace human subjects unless preceded with the term “non-human”.

As used herein, the term “antibody” refers to a polypeptide or group of polypeptides which are comprised of at least one binding domain, where an antibody binding domain is formed from the folding of variable domains of an antibody molecule to form three-dimensional binding spaces with an internal surface shape and charge distribution complementary to the features of an antigenic determinant of an antigen, which allows an immunological reaction with the antigen. Antibodies include recombinant proteins comprising the binding domains, as wells as fragments, including Fab, Fab′, F(ab)₂, and F(ab′)₂ fragments.

As used herein, an “antigenic determinant” is the portion of an antigen molecule, in this case a TBC-1 polypeptide, that determines the specificity of the antigen-antibody reaction. An “epitope” refers to an antigenic determinant of a polypeptide. An epitope can comprise as few as 3 amino acids in a spatial conformation which is unique to the epitope. Generally an epitope consists of at least 6 such amino acids, and more usually at least 8-10 such amino acids. Methods for determining the amino acids which make up an epitope include x-ray crystallography, 2-dimensional nuclear magnetic resonance, and epitope mapping e.g. the Pepscan method described by Geysen et al. 1984; PCT Publication No. WO 84/03564; and PCT Publication No. WO 84/03506.

Throughout the present specification, the expression “nucleotide sequence” may be employed to designate indifferently a polynucleotide or an oligonucleotide or a nucleic acid. More precisely, the expression “nucleotide sequence” encompasses the nucleic material itself and is thus not restricted to the sequence information (i.e. the succession of letters chosen among the four base letters) that biochemically characterizes a specific DNA or RNA molecule.

As used interchangeably herein, the term “oligonucleotides”, and “polynucleotides” include RNA, DNA, or RNA/DNA hybrid sequences of more than one nucleotide in either single chain or duplex form. The term “nucleotide” as used herein as an adjective to describe molecules comprising RNA, DNA, or RNA/DNA hybrid sequences of any length in single-stranded or duplex form. The term “nucleotide” is also used herein as a noun to refer to individual nucleotides or varieties of nucleotides, meaning a molecule, or individual unit in a larger nucleic acid molecule, comprising a purine or pyrimidine, a ribose or deoxyribose sugar moiety, and a phosphate group, or phosphodiester linkage in the case of nucleotides within an oligonucleotide or polynucleotide. Although the term “nucleotide” is also used herein to encompass “modified nucleotides” which comprise at least one modification (a) an alternative linking group, (b) an analogous form of purine, (c) an analogous form of pyrimidine, or (d) an analogous sugar, for examples of analogous linking groups, purine, pyrimidines, and sugars see for example PCT publication No WO 95/04064. However, the polynucleotides of the invention are preferably comprised of greater than 50% conventional deoxyribose nucleotides, and most preferably greater than 90% conventional deoxyribose nucleotides. The polynucleotide sequences of the invention may be prepared by any known method, including synthetic, recombinant, ex vivo generation, or a combination thereof, as well as utilizing any purification methods known in the art.

The term “heterozygosity rate” is used herein to refer to the incidence of individuals in a population which are heterozygous at a particular allele. In a biallelic system, the heterozygosity rate is on average equal to 2Pₐ(1−Pₐ), where Pₐ is the frequency of the least common allele. In order to be useful in genetic studies, a genetic marker should have an adequate level of heterozygosity to allow a reasonable probability that a randomly selected person will be heterozygous.

The term “genotype” as used herein refers the identity of the alleles present in an individual or a sample. In the context of the present invention a genotype preferably refers to the description of the biallelic marker alleles present in an individual or a sample. The term “genotyping” a sample or an individual for a biallelic marker consists of determining the specific allele or the specific nucleotide carried by an individual at a biallelic marker.

The term “polymorphism” as used herein refers to the occurrence of two or more alternative genomic sequences or alleles between or among different genomes or individuals. “Polymorphic” refers to the condition in which two or more variants of a specific genomic sequence can be found in a population. A “polymorphic site” is the locus at which the variation occurs. A single nucleotide polymorphism is a single base pair change. Typically a single nucleotide polymorphism is the replacement of one nucleotide by another nucleotide at the polymorphic site. Deletion of a single nucleotide or insertion of a single nucleotide, also give rise to single nucleotide polymorphisms. In the context of the present invention “single nucleotide polymorphism” preferably refers to a single nucleotide substitution. However, the polymorphism can also involve an insertion or a deletion of at least one nucleotide, preferably between 1 and 5 nucleotides. Typically, between different genomes or between different individuals, the polymorphic site may be occupied by two different nucleotides.

The term “biallelic polymorphism” and “biallelic marker” are used interchangeably herein to refer to a single nucleotide polymorphism having two alleles at a fairly high frequency in the population. A “biallelic marker allele” refers to the nucleotide variants present at a biallelic marker site. Typically, the frequency of the less common allele of the biallelic markers of the present invention has been validated to be greater than 1%, preferably the frequency is greater than 10%, more preferably the frequency is at least 20% (i.e. heterozygosity rate of at least 0.32), even more preferably the frequency is at least 30% (i.e. heterozygosity rate of at least 0.42). A biallelic marker wherein the frequency of the less common allele is 30% or more is termed a “high quality biallelic marker”.

The location of nucleotides in a polynucleotide with respect to the center of the polynucleotide are described herein in the following manner. When a polynucleotide has an odd number of nucleotides, the nucleotide at an equal distance from the 3′ and 5′ ends of the polynucleotide is considered to be “at the center” of the polynucleotide, and any nucleotide immediately adjacent to the nucleotide at the center, or the nucleotide at the center itself is considered to be “within 1 nucleotide of the center.” With an odd number of nucleotides in a polynucleotide any of the five nucleotides positions in the middle of the polynucleotide would be considered to be within 2 nucleotides of the center, and so on. When a polynucleotide has an even number of nucleotides, there would be a bond and not a nucleotide at the center of the polynucleotide. Thus, either of the two central nucleotides would be considered to be “within 1 nucleotide of the center” and any of the four nucleotides in the middle of the polynucleotide would be considered to be “within 2 nucleotides of the center”, and so on. For polymorphisms which involve the substitution, insertion or deletion of 1 or more nucleotides, the polymorphism, allele or biallelic marker is “at the center” of a polynucleotide if the difference between the distance from the substituted, inserted, or deleted polynucleotides of the polymorphism and the 3′ end of the polynucleotide, and the distance from the substituted, inserted, or deleted polynucleotides of the polymorphism and the 5′ end of the polynucleotide is zero or one nucleotide. If this difference is 0 to 3, then the polymorphism is considered to be “within 1 nucleotide of the center.” If the difference is 0 to 5, the polymorphism is considered to be “within 2 nucleotides of the center.” If the difference is 0 to 7, the polymorphism is considered to be “within 3 nucleotides of the center,” and so on.

As used herein the terminology “defining a biallelic marker” means that a sequence includes a polymorphic base from a biallelic marker. The sequences defining a biallelic marker may be of any length consistent with their intended use, provided that they contain a polymorphic base from a biallelic marker. The sequence has between 1 and 500 nucleotides in length, preferably between 5, 10, 15, 20, 25, or 40 and 200 nucleotides and more preferably between 30 and 50 nucleotides in length. Each biallelic marker therefore corresponds to two forms of a polynucleotide sequence included in a gene, which, when compared with one another, present a nucleotide modification at one position. Preferably, the sequences defining a biallelic marker include a polymorphic base selected from the group consisting of the biallelic markers A1 to A19 and the complements thereof. In some embodiments the sequences defining a biallelic marker comprise one of the sequences selected from the group consisting of P1 to P7, P9 to P13, P15 to P19 and the complementary sequences thereto. Likewise, the term “marker” or “biallelic marker” requires that the sequence is of sufficient length to practically (although not necessarily unambiguously) identify the polymorphic allele, which usually implies a length of at least 4, 5, 6, 10, 15, 20, 25, or 40 nucleotides.

The term “upstream” is used herein to refer to a location which is toward the 5′ end of the polynucleotide from a specific reference point.

The terms “base paired” and “Watson & Crick base paired” are used interchangeably herein to refer to nucleotides which can be hydrogen bonded to one another be virtue of their sequence identities in a manner like that found in double-helical DNA with thymine or uracil residues linked to adenine residues by two hydrogen bonds and cytosine and guanine residues linked by three hydrogen bonds (See Stryer, L., Biochemistry, 4^{th} edition, 1995).

The terms “complementary” or “complement thereof” are used herein to refer to the sequences of polynucleotides which is capable of forming Watson & Crick base pairing with another specified polynucleotide throughout the entirety of the complementary region. For the purpose of the present invention, a first polynucleotide is deemed to be complementary to a second polynucleotide when each base in the first polynucleotide is paired with its complementary base. Complementary bases are, generally, A and T (or A and U), or C and G. “Complement” is used herein as a synonym from “complementary polynucleotide”, “complementary nucleic acid” and “complementary nucleotide sequence”. These terms are applied to pairs of polynucleotides based solely upon their sequences and not any particular set of conditions under which the two polynucleotides would actually bind.

Variants and Fragments

1. Polynucleotides

The invention also relates to variants and fragments of the polynucleotides described herein, particularly of a TBC-1 gene containing one or more biallelic markers according to the invention.

Variants of polynucleotides, as the term is used herein, are polynucleotides that differ from a reference polynucleotide. A variant of a polynucleotide may be a naturally occurring variant such as a naturally occurring allelic variant, or it may be a variant that is not known to occur naturally. Such non-naturally occurring variants of the polynucleotide may be made by mutagenesis techniques, including those applied to polynucleotides, cells or organisms. Generally, differences are limited so that the nucleotide sequences of the reference and the variant are closely similar overall and, in many regions, identical.

Variants of polynucleotides according to the invention include, without being limited to, nucleotide sequences that are at least 95% identical to any of SEQ ID Nos 1-4 or the sequences complementary thereto or to any polynucleotide fragment of at least 8 consecutive nucleotides of any of SEQ ID Nos 1-4 or the sequences complementary thereto, and preferably at least 98% identical, more particularly at least 99.5% identical, and most preferably at least 99.9% identical to any of SEQ ID Nos 1-4 or the sequences complementary thereto or to any polynucleotide fragment of at least 8 consecutive nucleotides of any of SEQ ID Nos 1-4 or the sequences complementary thereto.

Changes in the nucleotide of a variant may be silent, which means that they do not alter the amino acids encoded by the polynucleotide.

However, nucleotide changes may also result in amino acid substitutions, additions, deletions, fusions and truncations in the polypeptide encoded by the reference sequence. The substitutions, deletions or additions may involve one or more nucleotides. The variants may be altered in coding or non-coding regions or both. Alterations in the coding regions may produce conservative or non-conservative amino acid substitutions, deletions or additions.

In the context of the present invention, particularly preferred embodiments are those in which the polynucleotides encode polypeptides which retain substantially the same biological function or activity as the mature TBC-1 protein.

A polynucleotide fragment is a polynucleotide having a sequence that entirely is the same as part but not all of a given nucleotide sequence, preferably the nucleotide sequence of a TBC-1 gene, and variants thereof. The fragment can be a portion of an exon or of an intron of a TBC-1 gene. It can also be a portion of the regulatory sequences of the TBC-1 gene. Preferably, such fragments comprise the polymorphic base of a biallelic marker selected from the group consisting of the biallelic markers A1 to A19 and the complements thereof.

Such fragments may be “free-standing”, i.e. not part of or fused to other polynucleotides, or they may be comprised within a single larger polynucleotide of which they form a part or region. However, several fragments may be comprised within a single larger polynucleotide.

As representative examples of polynucleotide fragments of the invention, there may be mentioned those which have from about 4, 6, 8, 15, 20, 25, 40, 10 to 20, 10 to 30, 30 to 55, 50 to 100, 75 to 100 or 100 to 200 nucleotides in length. Preferred are those fragments having about 49 nucleotides in length, such as those of P1 to P7, P9 to P13, P15 to P19 or the sequences complementary thereto and containing at least one of the biallelic markers of a TBC-1 gene which are described herein.

2. Polypeptides.

The invention also relates to variants, fragments, analogs and derivatives of the polypeptides described herein, including mutated TBC-1 proteins.

The variant may be 1) one in which one or more of the amino acid residues are substituted with a conserved or non-conserved amino acid residue (preferably a conserved amino acid residue) and such substituted amino acid residue may or may not be one encoded by the genetic code, or 2) one in which one or more of the amino acid residues includes a substituent group, or 3) one in which the mutated TBC-1 is fused with another compound, such as a compound to increase the half-life of the polypeptide (for example, polyethylene glycol), or 4) one in which the additional amino acids are fused to the mutated TBC-1, such as a leader or secretory sequence or a sequence which is employed for purification of the mutated TBC-1 or a preprotein sequence. Such variants are deemed to be within the scope of those skilled in the art.

More particularly, a variant TBC-1 polypeptide comprises amino acid changes ranging from 1, 2, 3, 4, 5, 10 to 20 substitutions, additions or deletions of one aminoacid, preferably from 1 to 10, more preferably from 1 to 5 and most preferably from 1 to 3 substitutions, additions or deletions of one amino acid. The preferred amino acid changes are those which have little or no influence on the biological activity or the capacity of the variant TBC-1 polypeptide to be recognized by antibodies raised against a native TBC-1 protein.

By homologous peptide according to the present invention is meant a polypeptide containing one or several aminoacid additions, deletions and/or substitutions in the amino acid sequence of a TBC-1 polypeptide. In the case of an aminoacid substitution, one or several—consecutive or non-consecutive-aminoacids are replaced by <<equivalent >> aminoacids.

The expression “equivalent” amino acid is used herein to designate any amino acid that may be substituted for one of the amino acids having similar properties, such that one skilled in the art of peptide chemistry would expect the secondary structure and hydropathic nature of the polypeptide to be substantially unchanged. Generally, the following groups of amino acids represent equivalent changes: (1) Ala, Pro, Gly, Glu, Asp, Gln, Asn, Ser, Thr; (2) Cys, Ser, Tyr, Thr; (3) Val, Ile, Leu, Met, Ala, Phe; (4) Lys, Arg, His; (5) Phe, Tyr, Trp, His.

By an equivalent aminoacid according to the present invention is also meant the replacement of a residue in the L-form by a residue in the D form or the replacement of a Glutamic acid (E) residue by a Pyro-glutamic acid compound. The synthesis of peptides containing at least one residue in the D-form is, for example, described by Koch (1977).

A specific, but not restrictive, embodiment of a modified peptide molecule of interest according to the present invention, which consists in a peptide molecule which is resistant to proteolysis, is a peptide in which the —CONH— peptide bond is modified and replaced by a (CH₂NH) reduced bond, a (NHCO) retro inverso bond, a (CH₂—O) methylene-oxy bond, a (CH₂—S) thiomethylene bond, a (CH₂CH₂) carba bond, a (CO—CH₂) cetomethylene bond, a (CHOH—CH₂) hydroxyethylene bond), a (N—N) bound, a E-alcene bond or also a —CH═CH— bond.

The polypeptide accoding to the invention could have post-translational modifications. For example, it can present the following modifications: acylation, disulfide bond formation, prenylation, carboxymethylation and phosphorylation.

A polypeptide fragment is a polypeptide having a sequence that entirely is the same as part but not all of a given polypeptide sequence, preferably a polypeptide encoded by a TBC-1 gene and variants thereof. Preferred fragments include those regions possessing antigenic properties and which can be used to raise antibodies against the TBC-1 protein.

Such fragments may be “free-standing”, i.e. not part of or fused to other polypeptides, or they may be comprised within a single larger polypeptide of which they form a part or region. However, several fragments may be comprised within a single larger polypeptide.

As representative examples of polypeptide fragments of the invention, there may be mentioned those which comprise at least about 5, 6, 7, 8, 9 or 10 to 15, 10 to 20, 15 to 40, or 30 to 55 amino acids of the TBC-1. In some embodiments, the fragments contain at least one amino acid mutation in the TBC-1 protein.

Identity Between Nucleic Acids or Polypeptides

The terms “percentage of sequence identity” and “percentage homology” are used interchangeably herein to refer to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Homology is evaluated using any of the variety of sequence comparison algorithms and programs known in the art. Such algorithms and programs include, but are by no means limited to, TBLASTN, BLASTP, FASTA, TFASTA, and CLUSTALW (Pearson and Lipman, 1988; Altschul et al., 1990; Thompson et al., 1994; Higgins et al., 1996; Altschul et al., 1993). In a particularly preferred embodiment, protein and nucleic acid sequence homologies are evaluated using the Basic Local Alignment Search Tool (“BLAST”) which is well known in the art (see, e.g., Karlin and Altschul, 1990; Altschul et al., 1990, 1993, 1997). In particular, five specific BLAST programs are used to perform the following task:

(1) BLASTP and BLAST3 compare an amino acid query sequence against a protein sequence database;

(2) BLASTN compares a nucleotide query sequence against a nucleotide sequence database;

(3) BLASTX compares the six-frame conceptual translation products of a query nucleotide sequence (both strands) against a protein sequence database;

(4) TBLASTN compares a query protein sequence against a nucleotide sequence database translated in all six reading frames (both strands); and

(5) TBLASTX compares the six-frame translations of a nucleotide query sequence against the six-frame translations of a nucleotide sequence database.

The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. High-scoring segment pairs are preferably identified (i.e., aligned) by means of a scoring matrix, many of which are known in the art. Preferably, the scoring matrix used is the BLOSUM62 matrix (Gonnet et al., 1992; Henikoff and Henikoff, 1993). Less preferably, the PAM or PAM250 matrices may also be used (see, e.g., Schwartz and Dayhoff, eds., 1978). The BLAST programs evaluate the statistical significance of all high-scoring segment pairs identified, and preferably selects those segments which satisfy a user-specified threshold of significance, such as a user-specified percent homology. Preferably, the statistical significance of a high-scoring segment pair is evaluated using the statistical significance formula of Karlin (see, e.g., Karlin and Altschul, 1990).

Stringent Hybridization Conditions

By way of example and not limitation, procedures using conditions of high stringency are as follows: Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C., the preferred hybridization temperature, in prehybridization mixture containing 100 μg/ml denatured salmon sperm DNA and 5-20×10⁶ cpm of ³²P-labeled probe. Alternatively, the hybridization step can be performed at 65° C. in the presence of SSC buffer, 1×SSC corresponding to 0.15M NaCl and 0.05 M Na citrate. Subsequently, filter washes can be done at 37° C. for 1 h in a solution containing 2×SSC, 0.01% PVP, 0.01% Ficoll, and 0.01% BSA, followed by a wash in 0.1×SSC at 50° C. for 45 min. Alternatively, filter washes can be performed in a solution containing 2×SSC and 0.1% SDS, or 0.5×SSC and 0.1% SDS, or 0.1×SSC and 0.1% SDS at 68° C. for 15 minute intervals. Following the wash steps, the hybridized probes are detectable by autoradiography. Other conditions of high stringency which may be used are well known in the art and as cited in Sambrook et al., 1989; and Ausubel et al., 1989, are incorporated herein in their entirety. These hybridization conditions are suitable for a nucleic acid molecule of about 20 nucleotides in length. There is no need to say that the hybridization conditions described above are to be adapted according to the length of the desired nucleic acid, following techniques well known to the one skilled in the art. The suitable hybridization conditions may for example be adapted according to the teachings disclosed in the book of Hames and Higgins (1985) or in Sambrook et al. (1989).

### Candidate Region on the Chromosome 4 (Linkage Analysis)

In order to localize the prostate cancer gene(s) starting from families, a systematic familial study of genetic link research is carried out using markers of the microsatellite type described at the Genethon laboratory by the Jean Weissenbach team (Dib et al., 1996).

The studies of genetic link or of “linkage” are based on the principle according to which two neighboring sequences on a chromosome do not present (or very rarely present) recombinations by crossing-over during meiosis. To do this, microsatellite DNA sequences (chromosomal markers) constantly co-inherited with the disease studied are searched for in a family having a predisposition for this disease. These DNA sequences organized in the form of a repetition of di-, tri- or tetranucleotides are systematically present along the genome, and thus allow the identification of chromosomal fragments harboring them. More than 5000 microsatellite markers, have been localized with precision on the genome as a result of the first studies on the genetic map carried out at Genethon under the supervision of Jean Weissenbach, and on the physical map (using the “Yeast Artificial Chromosomes”), work conducted by Daniel Cohen at C.E.P.H. and at Genethon (Chumakov et al., 1995). Genetic link analysis calculates the probabilities of recombinations of the target gene with the chromosomal markers used, according to the genealogical tree, the transmission of the disease, and the transmission of the markers. Thus if a particular allele of a given marker is transmitted with the disease more often than chance would have it (recombination level of between 0 and 0.5), it is possible to deduce that the target gene in question is found in the neighborhood of the marker. Using this technique, it has been possible to localize several genes of genetic predisposition to familial cancers. In order to be able to be included in a genetic link study, the families affected by a hereditary form of the disease must satisfy the “informativeness” criteria: several affected subjects (and whose constitutional DNA is available) per generation, and at best having a large number of siblings.

By linkage analysis, the inventors have identified a candidate region for prostate cancer on chromosome 4. Indeed, the LOD scores at 2 points between the disease and the markers on a total population of approximately fifty families present a value of 2.49 for marker D4S398 which indicates a probable genetic link with this marker. The curve of the variation of the LOD score on a map of 5 markers is centered on D4S398 and the value higher than 3.3 indicates that a gene involved in familial prostate cancer is probably found in the region located between markers D4S2978 and D4S3018, or a space of approximately 9.7 cM.

Homologies of the Novel Human Gene Translation Product with a Known Murine Protein

A novel human gene was found in this candidate region. It presents a good probability to be involved in cancer. Database homology searches have allowed the inventors to determine that the translation product of this novel human gene has significant identity with a murine protein called tbc1. Therefore, the novel human gene of the invention has thus been called TBC-1 throughout the present specification. TBC-1 comprises an open Reading frame that encodes a novel protein, the TBC-1 protein. Based on sequence similarity, an alignment of a portion of the TBC-1 amino acid sequence with the known tbc1 murine protein, it is expected that TBC1 protein may play a role in the cell cycle and in differentiation of various tissues. Indeed, the TBC1 protein contains a 200 amino acid domain called the TBC domain that is homologous to regions in the tre2-oncogene and in the yeast regulators of mitosis BLB2 and cdc16.

The cDNA of the murine tbc1 gene has been described in U.S. Pat. No. 5,700,927 and it encodes a putative protein product of 1141 amino acids. The N-terminus of the murine tbc1 protein contains stretches of cysteines and histidines which may form zinc finger structures in the mature polypeptides. The N-terminus also comprises short stretches of basic amino acids which may be involved in a nuclear localization signal. The TBC domain of the murine tbc1 protein contains several tyrosine residues which are conserved in BUB2 and cdc16. The C-terminus of the murine tbc1 protein contains a long stretch of evenly spaced leucine residues which are susceptible to form a leucine zipper motif.

The murine tbc1 gene has been shown to be highly expressed in testis and kidney. However, lower levels of expression have also be identified in lung, spleen, brain, and heart. Moreover, murine tbc1 is a nuclear protein which is expressed in a cell- and stage-specific manner.

Studies of murine bone marrow have demonstrated that erythroid cells and megakaryocytes expressed substantial levels of the murine tbc1 protein, but none was detected in mature neutrophils. Similarly, spermatogonia do not express murine tbc1, but primary and secondary spermatocytes express abundant tbc1. Later in the differentiation of the germ cells, the tbc1 levels appear to decrease in spermatids and active sperm. The differentiation program of spermatogonia to spermatocytes therefore involves a significant upregulation of murine tbc1 expression.

The general distribution of murine tbc1 is not tissue-specific, but is cell-specific within individual tissues and intimately linked to tissue differentiation. The developmental expression of murine tbc1, particularly in hematopoietic and germ cells, suggests that this gene plays a role in the terminal differentiation program of several tissues.

Consequently, an alteration in the expression of the TBC-1 gene or in the amino acid sequence of the TBC-1 protein leading to an altered biological activity of the latter is likely to cause, directly or indirectly, cell proliferation disorders and thus diseases related to an abnormal cell proliferation such as cancer, particularly prostate cancer.

### Genomic Sequence of TBC-1

The present invention concerns the genomic sequence of TBC-1. The present invention encompasses the TBC-1 gene, or TBC-1 genomic sequences consisting of, consisting essentially of, or comprising a sequence selected from the group consisting of SEQ ID Nos 1 and 2, a sequence complementary thereto, as well as fragments and variants thereof. These polynucleotides may be purified, isolated, or recombinant.

The inventors have sequenced two portions of the TBC-1 genomic sequence. The first portion of the TBC-1 gene sequence contains the three first exons of the TBC-1 gene, designated as Exon 1, Exon 1bis and Exon 2, and the 5′ regulatory sequence located upstream of the transcribed sequences. The sequence of the first portion of the genomic sequence is disclosed in SEQ ID No 1. The second portion contains the twelve last exons of the TBC-1 gene, designated as exons A, B, C, D, E, F, G, H, I, J, K, and L, and the 3′ regulatory sequence which is located downstream of the transcribed sequences.

The exon positions in SEQ ID Nos 1 and 2 are detailed below in Table A.

TABLE A

| | Position in SEQ ID No 1 | | | Position in SEQ ID No 1 | |
|---|---|---|---|---|---|
| Exon | Beginning | End | Intron | Beginning | End |
| 1 | 2001 | 2077 | 1 | 2078 | 12739 |
| 1bis | 12292 | 12373 | 1bis | 12374 | 12739 |
| 2 | 12740 | 13249 | 2 | 13250 | at least |
| | | | | | 17590 |
| | Position in SEQ ID No 2 | | | Position in SEQ ID No 2 | |
| Exon | Beginning | End | Intron | Beginning | End |
| A | 4661 | 4789 | A | 4790 | 6115 |
| B | 6116 | 6202 | B | 6203 | 9918 |
| C | 9919 | 10199 | C | 10200 | 14520 |
| D | 14521 | 14660 | D | 14661 | 50256 |
| E | 50257 | 50442 | E | 50443 | 56255 |
| F | 56256 | 56417 | F | 56418 | 63325 |
| G | 63326 | 63484 | G | 63485 | 76035 |
| H | 76036 | 76280 | H | 76281 | 78363 |
| I | 78364 | 78523 | I | 78524 | 85294 |
| J | 85295 | 85464 | J | 85465 | 93416 |
| K | 93417 | 93590 | K | 93591 | 97475 |
| L | 97476 | 97960 | | | |

Intron 1 refers to the nucleotide sequence located between Exon 1 and Exon 2; Intron 1bis refers to the nucleotide sequence located between Exon 1bis and Exon 2; Intron A refers to the nucleotide sequence located between Exon A and Exon B; and so on. The position of the introns is detailed in Table A.

The TBC-1 introns defined hereinafter for the purpose of the present invention are not exactly what is generally understood as “introns” by the one skilled in the art and will consequently be further defined below.

Generally, an intron is defined as a nucleotide sequence that is present both in the genomic DNA and in the unspliced mRNA molecule, and which is absent from the mRNA molecule which has already gone through splicing events. In the case of the TBC-1 gene, the inventors have found that at least two different spliced mRNA molecules are produced when this gene is transcribed, as it will be described in detail in a further section of the specification. The first spliced mRNA molecule comprises Exons 1 and 2. Thus, the genomic nucleotide sequence comprised between Exon 1 and Exon 2 is an intronic sequence as regards to this first mRNA molecule, despite the fact that this intronic sequence contains Exon 1bis. In contrast, Exon 1bis is of course an exonic nucleotide sequence as regards to the second TBC-1 mRNA molecule.

For the purpose of the present invention and in order to make a clear and unambiguous designation of the different nucleic acids encompassed, it has been postulated that the polynucleotides contained both in any of the nucleotide sequences of SEQ ID Nos 1 or 2 and in any of the nucleotide sequences of SEQ ID Nos 3 or 4 are considered as exonic sequences. Conversely, the polynucleotides contained in any of the nucleotide sequences of SEQ ID Nos 1 or 2 but which are absent both from the nucleotide sequence of SEQ ID No 3 and from the nucleotide sequence of SEQ ID No 4 are considered as intronic sequences.

The nucleic acids defining the TBC-1 introns described above, as well as their fragments and variants, may be used as oligonucleotide primers or probes in order to detect the presence of a copy of the TBC-1 gene in a test sample, or alternatively in order to amplify a target nucleotide sequence within the TBC-1 intronic sequences.

Thus, the invention embodies purified, isolated, or recombinant polynucleotides comprising a nucleotide sequence selected from the group consisting of the 15 exons of the TBC-1 gene which are described in the present invention, or a sequence complementary thereto. The invention also deals with purified, isolated, or recombinant nucleic acids comprising a combination of at least two exons of the TBC-1 gene, wherein the polynucleotides are arranged within the nucleic acid, from the 5′-end to the 3′-end of said nucleic acid, in the same order as in SEQ ID Nos 1 and 2.

Thus, the invention embodies purified, isolated, or recombinant polynucleotides comprising a nucleotide sequence selected from the group consisting of the introns of the TBC-1 gene, or a sequence complementary thereto.

The invention also encompasses a purified, isolated, or recombinant polynucleotide comprising a nucleotide sequence having at least 70, 75, 80, 85, 90, or 95% nucleotide identity with a sequence selected from the group consisting of SEQ ID Nos 1 and 2 or a complementary sequence thereto or a fragment thereof. The nucleotide differences as regards to the nucleotide sequence of SEQ ID Nos 1 or 2 may be generally randomly distributed throughout the entire nucleic acid. Nevertheless, preferred nucleic acids are those wherein the nucleotide differences as regards to the nucleotide sequence of SEQ ID Nos 1 or 2 are predominantly located outside the coding sequences contained in the exons. These nucleic acids, as well as their fragments and variants, may be used as oligonucleotide primers or probes in order to detect the presence of a copy of the TBC-1 gene in a test sample, or alternatively in order to amplify a target nucleotide sequence within the TBC-1 sequences.

Another object of the invention consists of a purified, isolated, or recombinant nucleic acid that hybridizes with a sequence selected from the group consisting of SEQ ID Nos 1 and 2 or a complementary sequence thereto or a variant thereof, under the stringent hybridization conditions as defined above.

Particularly preferred nucleic acids of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of a nucleotide sequence selected from the group consisting of SEQ ID Nos 1 and 2, or the complements thereof. Additionally preferred nucleic acids of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID No 1 or the complements thereof, wherein said contiguous span comprises at least 1, 2, 3, 5, or 10 of the following nucleotide positions of SEQ ID No 1: 1-1000, 1001-2000, 2001-3000, 30014000, 4001-5000, 5001-6000, 6001-7000, 7001-8000, 8001-9000, 9001-10000, 10001-11000, 11001-12000, 12001-13000, 13001-14000, 14001-15000, 15001-16000, 16001-17000, and 17001-17590. Other preferred nucleic acids of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID No 2 or the complements thereof, wherein said contiguous span comprises at least 1, 2, 3, 5, or 10 of the following nucleotide positions of SEQ ID No 2: 1-5000, 5001-10000, 10001-15000, 15001-20000, 20001-25000, 25001-30000, 30001-35000, 35001-40000, 40001-45000, 45001-50000, 50001-55000, 55001-60000, 60001-65000, 65001-70000, 70001-75000, 75001-80000, 80001-85000, 85001-90000, 90001-95000, and 95001-99960.

While this section is entitled “Genomic Sequences of TBC-1,” it should be noted that nucleic acid fragments of any size and sequence may also be comprised by the polynucleotides described in this section, flanking the genomic sequences of TBC-1 on either side or between two or more such genomic sequences.

### TBC-1 cDNA Sequences

The inventors have discovered that the expression of the TBC-1 gene leads to the production of at least two mRNA molecules, respectively a first and a second TBC-1 transcription product, as the results of alternative splicing events. They result from two distinct first exons, namely Exon 1 and Exon 1bis.

The first transcription product comprises Exons 1, 2, A, B, C, D, E, F, G, H, I, J, K, and L. This cDNA of SEQ ID No 3 includes a 5′-UTR region, spanning the whole Exon 1 and part of Exon 2. This 5′-UTR region starts from the nucleotide at position 1 and ends at the nucleotide at position 170 of the nucleotide sequence of SEQ ID No 3. The cDNA of SEQ ID No 3 includes a 3′-UTR region starting from the nucleotide at position 3726 and ending at the nucleotide at position 3983 of the nucleotide sequence of SEQ ID No 3. This first transcription product harbors a polyadenylation signal located between the nucleotide at position 3942 and the nucleotide at position 3947 of the nucleotide sequence of SEQ ID No 3.

The second TBC-1 transcription product comprises Exons 1bis, 2, A, B, C, D, E, F, G, H, I, J, K, and L. This cDNA of SEQ ID No 4 includes a 5′-UTR region starting from the nucleotide at position 1 and ending at the nucleotide at position 175 of the nucleotide sequence of SEQ ID No 4. This second cDNA also includes a 3′-UTR region starting from the nucleotide at position 3731 and ending at the nucleotide at position 3988 of the nucleotide sequence of SEQ ID No 4. This second transcription product harbors a polyadenylation signal located between the nucleotide at position 3947 and the nucleotide at position 3952 of the nucleotide sequence of SEQ ID No 4.

The 5′-end sequence of this second TBC-1 mRNA, more particularly the nucleotide sequence comprised between the nucleotide in position 1 and the nucleotide in position 458 of the nucleic acid of SEQ ID No 4 molecule corresponds to the nucleotide sequence of a 5′-EST that has been obtained from a human pancreas cDNA library and characterized following the teachings of the PCT Application No WO 96/34981. This 5′-EST is also part of the invention.

Another object of the invention consists of a purified or isolated nucleic acid comprising a polynucleotide selected from the group consisting of the nucleotide sequences of SEQ ID Nos 3 and 4 and to nucleic acid fragments thereof.

Preferred nucleic acid fragments of the nucleotide sequences of SEQ ID Nos 3 and 4 consist in polynucleotides comprising their respective Open Reading Frames encoding the TBC-1 protein.

Other preferred nucleic acid fragments of the nucleotide sequences of SEQ ID Nos 3 and 4 consist in polynucleotides comprising at least a part of their respective 5′-UTR or 3′-UTR regions.

The invention also pertains to a purified or isolated nucleic acid having at least a 95% of nucleotide identity with any one of the nucleotide sequences of SEQ ID Nos 3 and 4, or a fragment thereof.

Another object of the invention consists of purified, isolated or recombinant nucleic acids comprising a polynucleotide that hybridizes, under the stringent hybridization conditions defined herein, with any one of the nucleotide sequences of SEQ ID Nos 3 and 4, or a sequence complementary thereto or a fragment thereof.

The invention also relates to isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of a nucleotide sequence selected from the group consisting of SEQ ID Nos 3 and 4, or the complements thereof. Particularly preferred nucleic acids of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID No 3 or the complements thereof, wherein said contiguous span comprises at least 1, 2, 3, 5, or 10 of the following nucleotide positions of SEQ ID No 3: 1-500, 501-1000, 1001-1500, 1501-2000, 2001-2500, 2501-3000, 3001-3500, and 3501-3983. Additionally preferred nucleic acids of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID No 4 or the complements thereof, wherein said contiguous span comprises at least 1, 2, 3, 5, or 10 of the following nucleotide positions of SEQ ID No 4: 1-500, 501-1000, 1001-1500, 1501-2000, 2001-2500, 2501-3000, 3001-3500, and 3501-3988. Such a nucleic acid is notably useful as polynucleotide probe or primer specific for the TBC-1 gene or the TBC-1 mRNAs and cDNAs.

While this section is entitled “TBC-1 cDNA Sequences,” it should be noted that nucleic acid fragments of any size and sequence may also be comprised by the polynucleotides described in this section, flanking the genomic sequences of TBC-1 on either side or between two or more such genomic sequences.

### Coding Regions

The TBC-1 open reading frame is contained in the two TBC-1 mRNA molecules of about 4 kilobases isolated by the inventors.

More precisely, the effective TBC-1 coding sequence is comprised between the nucleotide at position 171 and the nucleotide at position 3725 of SEQ ID No 3, and between the nucleotide at position 176 and the nucleotide at position 3730 of the nucleotide sequence of SEQ ID No 4.

The invention further provides a purified or isolated nucleic acid comprising a polynucleotide selected from the group consisting of a polynucleotide comprising a nucleic acid sequence located between the nucleotide at position 171 and the nucleotide at position 3725 of SEQ ID No 3, and a polynucleotide comprising a nucleic acid sequence located between the nucleotide at position 176 and the nucleotide at position 3730 of SEQ ID No 4 or a variant or fragment thereof or a sequence complementary thereto.

The present invention concerns a purified or isolated nucleic acid encoding a human TBC-1 protein, wherein said TBC-1 protein comprises an amino acid sequence of SEQ ID No 5, a nucleotide sequence complementary thereto, a fragment or a variant thereof. The present invention also embodies isolated, purified, and recombinant polynucleotides which encode a polypeptides comprising a contiguous span of at least 6 amino acids, preferably at least 8 or 10 amino acids, more preferably at least 12, 15, 20, 25, 30, 40, 50, or 100 amino acids of SEQ ID No 5. In a preferred embodiment, the present invention embodies isolated, purified, and recombinant polynucleotides which encode a polypeptides comprising a contiguous span of at least 6 amino acids, preferably at least 8 or 10 amino acids, more preferably at least 12, 15, 20, 25, 30, 40, 50, or 100 amino acids of SEQ ID No 5 wherein said contiguous span includes at least 1, 2, 3, 5 or 10 of the following amino acid positions in SEQ ID No 5: 1-300, 301-600, 601-900, and 901-1168.

The above disclosed polynucleotide that contains only coding sequences derived from the TBC-1 ORF may be expressed in a desired host cell or a desired host organism, when said polynucleotide is placed under the control of suitable expression signals. Such a polynucleotide, when placed under the suitable expression signals, may be inserted in a vector for its expression.

### Regulatory Sequences of TBC-1

The invention further deals with a purified or isolated nucleic acid comprising the nucleotide sequence of a regulatory region which is located either upstream of the first exon of the TBC-1 gene and which is contained in the TBC-1 genomic sequence of SEQ ID No 1, or downstream of the last exon of the TBC-1 gene and which is contained in the TBC-1 genomic sequence of SEQ ID No 2.

The 5′-regulatory sequence of the TBC-1 gene is localized between the nucleotide in position 1 and the nucleotide in position 2000 of the nucleotide sequence of SEQ ID No 1. The 3′-regulatory sequence of the TBC-1 gene is localized between nucleotide position 97961 and nucleotide position 99960 of SEQ ID No 2.

Polynucleotides derived from the 5′ and 3′ regulatory regions are useful in order to detect the presence of at least a copy of a nucleotide sequence of SEQ ID Nos 1 or 2 or a fragment thereof in a test sample.

The promoter activity of the 5′ regulatory regions contained in TBC-1 can be assessed as described below.

Genomic sequences lying upstream of the TBC-1 Exons are cloned into a suitable promoter reporter vector, such as the pSEAP-Basic, pSEAP-Enhancer, pβgal-Basic, pβgal-Enhancer, or pEGFP-1 Promoter Reporter vectors available from Clontech. Briefly, each of these promoter reporter vectors include multiple cloning sites positioned upstream of a reporter gene encoding a readily assayable protein such as secreted alkaline phosphatase, beta galactosidase, or green fluorescent protein. The sequences upstream of the TBC-1 coding region are inserted into the cloning sites upstream of the reporter gene in both orientations and introduced into an appropriate host cell. The level of reporter protein is assayed and compared to the level obtained from a vector which lacks an insert in the cloning site. The presence of an elevated expression level in the vector containing the insert with respect to the control vector indicates the presence of a promoter in the insert. If necessary, the upstream sequences can be cloned into vectors which contain an enhancer for increasing transcription levels from weak promoter sequences. A significant level of expression above that observed with the vector lacking an insert indicates that a promoter sequence is present in the inserted upstream sequence.

Promoter sequences within the upstream genomic DNA may be further defined by constructing nested deletions in the upstream DNA using conventional techniques such as Exonuclease III digestion. The resulting deletion fragments can be inserted into the promoter reporter vector to determine whether the deletion has reduced or obliterated promoter activity. In this way, the boundaries of the promoters may be defined. If desired, potential individual regulatory sites within the promoter may be identified using site directed mutagenesis or linker scanning to obliterate potential transcription factor binding sites within the promoter, individually or in combination. The effects of these mutations on transcription levels may be determined by inserting the mutations into the cloning sites in the promoter reporter vectors.

Thus, the minimal size of the promoter of the TBC-1 gene can be determined through the measurement of TBC-1 expression levels. For this assay, an expression vector comprising decreasing sizes from the promoter generally ranging from 2 kb to 100 bp, with a 3′ end which is constant, operably linked to TBC-1 coding sequence or to a reporter gene is used. Cells, which are preferably prostate cells and more preferably prostate cancer cells, are transfected with this vector and the expression level of the gene is assessed.

The strength and the specificity of the promoter of the TBC-1 gene can be assessed through the expression levels of the gene operably linked to this promoter in different types of cells and tissues. In one embodiment, the efficacy of the promoter of the TBC-1 gene is assessed in normal and cancer cells. In a preferred embodiment, the efficacy of the promoter of the TBC-1 gene is assessed in normal prostate cells and in prostate cancer cells which can present different degrees of malignancy.

Polynucleotides carrying the regulatory elements located both at the 5′ end and at the 3′ end of the TBC-1 cDNAs may be advantageously used to control the transcriptional and translational activity of an heterologous polynucleotide of interest.

Thus, the present invention also concerns a purified or isolated nucleic acid comprising a polynucleotide which is selected from the group consisting of the 5′ and 3′ regulatory regions, or a sequence complementary thereto or a biologically active fragment or variant thereof. “5′ regulatory region” refers to the nucleotide sequence located between positions 1 and 2000 of SEQ ID No 1. “3′ regulatory region” refers to the nucleotide sequence located between positions 97961 and 99960 of SEQ ID No 2.

The invention also pertains to a purified or isolated nucleic acid comprising a polynucleotide having at least 95% nucleotide identity with a polynucleotide selected from the group consisting of the 5′ and 3′ regulatory regions, advantageously 99% nucleotide identity, preferably 99.5% nucleotide identity and most preferably 99.8% nucleotide identity with a polynucleotide selected from the group consisting of the 5′ and 3′ regulatory regions, or a sequence complementary thereto or a variant thereof or a biologically active fragment thereof.

Another object of the invention consists of purified, isolated or recombinant nucleic acids comprising a polynucleotide that hybridizes, under the stringent hybridization conditions defined herein, with a polynucleotide selected from the group consisting of the nucleotide sequences of the 5′- and 3′ regulatory regions, or a sequence complementary thereto or a variant thereof or a biologically active fragment thereof.

The 5′ UTR and 3′ UTR regions of a gene are of particular importance in that they often comprise regulatory elements which can play a role in providing appropriate expression levels, particularly through the control of mRNA stability.

A 5′ regulatory polynucleotide of the invention may include the 5′-UTR located between the nucleotide at position 1 and the nucleotide at position 170 of SEQ ID No 3, or a biologically active fragment or variant thereof.

Alternatively, a 5′-regulatory polynucleotide of the invention may include the 5′-UTR located between the nucleotide at position 1 and the nucleotide at position 175 of SEQ ID No 4, or a biologically active fragment or variant thereof.

A 3′ regulatory polynucleotide of the invention may include the 3′-UTR located between the nucleotide at position 3726 and the nucleotide at position 3983 of SEQ ID No 4, or a biologically active fragment or variant thereof.

Thus, the invention also pertains to a purified or isolated nucleic acid which is selected from the group consisting of:

a) a nucleic acid comprising the nucleotide sequence of the 5′ regulatory region;

b) a nucleic acid comprising a biologically active fragment or variant of the nucleic acid of the 5′ regulatory region.

Preferred fragments of the nucleic acid of the 5′ regulatory region have a length of about 1000 nucleotides, more particularly of about 400 nucleotides, more preferably of about 200 nucleotides and most preferably about 100 nucleotides. More particularly, the invention further includes specific elements within this regulatory region, these elements preferably including the promoter region.

Preferred fragments of the 3′ regulatory region are at least 50, 100, 150, 200, 300 or 400 bases in length.

By a “biologically active fragment or variant” of a TBC-1 regulatory polynucleotide according to the present invention is intended a polynucleotide comprising or alternatively consisting in a fragment of said polynucleotide which is functional as a regulatory region for expressing a recombinant polypeptide or a recombinant polynucleotide in a recombinant cell host.

For the purpose of the invention, a nucleic acid or polynucleotide is “functional” as a regulatory region for expressing a recombinant polypeptide or a recombinant polynucleotide if said regulatory polynucleotide contains nucleotide sequences which contain transcriptional and translational regulatory information, and if such sequences are “operatively linked” to nucleotide sequences which encode the desired polypeptide or the desired polynucleotide. An operable linkage is a linkage in which the regulatory nucleic acid and the DNA sequence sought to be expressed are linked in such a way as to permit gene expression.

In order, to identify the relevant biologically active polynucleotide derivatives of the 5′ or 3′ regulatory region, the one skill in the art will refer to the book of Sambrook et al. (Sambrook, 1989) in order to use a recombinant vector carrying a marker gene (i.e. beta galactosidase, chloramphenicol acetyl transferase, etc.) the expression of which will be detected when placed under the control of a biologically active derivative polynucleotide of the 5′ or 3′ regulatory region.

Regulatory polynucleotides of the invention may be prepared from any of the nucleotide sequences of SEQ ID Nos 1 or 2 by cleavage using the suitable restriction enzymes, the one skill in the art being guided by the book of Sambrook et al. (1989). Regulatory polynucleotides may also be prepared by digestion of any of the nucleotide sequences of SEQ ID Nos 1 or 2 by an exonuclease enzyme, such as Bal31 (Wabiko et al., 1986). These regulatory polynucleotides can also be prepared by chemical synthesis, as described elsewhere in the specification, when the synthesis of oligonucleotide probes or primers is disclosed.

The regulatory polynucleotides according to the invention may be advantageously part of a recombinant expression vector that may be used to express a coding sequence in a desired host cell or host organism. The recombinant expression vectors according to the invention are described elsewhere in the specification.

The invention also encompasses a polynucleotide comprising

a) a nucleic acid comprising a regulatory nucleotide sequence of the 5′ regulatory region, or a biologically active fragment or variant thereof,

b) a polynucleotide encoding a desired polypeptide or nucleic acid, operably linked to the nucleic acid comprising a regulatory nucleotide sequence of the 5′ regulatory region, or its biologically active fragment or variant.

c) Optionally, a nucleic acid comprising a 3′ regulatory polynucleotide, preferably a 3′ regulatory polynucleotide of the invention.

The desired polypeptide encoded by the above described nucleic acid may be of various nature or origin, encompassing proteins of prokaryotic or eukaryotic origin. Among the polypeptides expressed under the control of a TBC-1 regulatory region, it may be cited bacterial, fungal or viral antigens. Are also encompassed eukaryotic proteins such as intracellular proteins, such as “house keeping” proteins, membrane-bound proteins, like receptors, and secreted proteins like the numerous endogenous mediators such as cytokines.

The desired nucleic acid encoded by the above described polynucleotide, usually a RNA molecule, may be complementary to a TBC-1 coding sequence and thus useful as an antisense polynucleotide.

Such a polynucleotide may be included in a recombinant expression vector in order to express a desired polypeptide or a desired polynucleotide in host cell or in a host organism. Suitable recombinant vectors that contain a polynucleotide such as described hereinbefore are disclosed elsewhere in the specification.

### TBC-1 Polypeptide and Peptide Fragments Thereof

It is now easy to produce proteins in high amounts by genetic engineering techniques through expression vectors such as plasmids, phages or phagemids. The polynucleotide that code for one the polypeptides of the present invention is inserted in an appropriate expression vector in order to produce the polypeptide of interest in vitro.

Thus, the present invention also concerns a method for producing one of the polypeptides described herein, and especially a polypeptide of SEQ ID No 5 or a fragment or a variant thereof, wherein said method comprises the steps of:

a) culturing, in an appropriate culture medium, a cell host previously transformed or transfected with the recombinant vector comprising a nucleic acid encoding a TBC-1 polypeptide, or a fragment or a variant thereof;

b) harvesting the culture medium thus conditioned or lyse the cell host, for example by sonication or by an osmotic shock;

c) separating or purifying, from the said culture medium, or from the pellet of the resultant host cell lysate the thus produced polypeptide of interest.

d) Optionally characterizing the produced polypeptide of interest.

In a specific embodiment of the above method, step a) is preceded by a step wherein the nucleic acid coding for a TBC-1 polypeptide, or a fragment or a variant thereof, is inserted in an appropriate vector, optionally after an appropriate cleavage of this amplified nucleic acid with one or several restriction endonucleases. The nucleic acid coding for a TBC-1 polypeptide or a fragment or a variant thereof may be the resulting product of an amplification reaction using a pair of primers according to the invention (by SDA, TAS, 3SR NASBA, TMA etc.).

The polypeptides according to the invention may be characterized by binding onto an immunoaffinity chromatography column on which polyclonal or monoclonal antibodies directed to a polypeptide of SEQ ID No 5, or a fragment or a variant thereof, have previously been immobilized.

Purification of the recombinant proteins or peptides according to the present invention may be carried out by passage onto a Nickel or Cupper affinity chromatography column. The Nickel chromatography column may contain the Ni-NTA resin (Porath et al., 1975).

The polypeptides or peptides thus obtained may be purified, for example by high performance liquid chromatography, such as reverse phase and/or cationic exchange HPLC, as described by Rougeot et al. (1994). The reason to prefer this kind of peptide or protein purification is the lack of byproducts found in the elution samples which renders the resultant purified protein or peptide more suitable for a therapeutic use.

Another object of the present invention consists in a purified or isolated TBC-1 polypeptide or a fragment or a variant thereof.

In a preferred embodiment, the TBC-1 polypeptide comprises an amino acid sequence of SEQ ID No 5 or a fragment or a variant thereof. The present invention also embodies isolated, purified, and recombinant polypeptides comprising a contiguous span of at least 6 amino acids, preferably at least 8 to 10 amino acids, more preferably at least 12, 15, 20, 25, 30, 40, 50, 100, 150 or 200 amino acids of SEQ ID No 5. The present invention also embodies isolated, purified, and recombinant polypeptides comprising a contiguous span of at least 6 amino acids, preferably at least 8 to 10 amino acids, more preferably at least 12, 15, 20, 25, 30, 40, 50, 100, 150 or 200 amino acids of SEQ ID No 5, wherein said contiguous span includes at least 1, 2, 3, 5 or 10 of the following amino acid positions: 1-200, 201-400, 401-600, 601-800, 801-1000, 1001-1168.

The invention also encompasses a purified, isolated, or recombinant polypeptides comprising an amino acid sequence having at least 90, 95, 98 or 99% amino acid identity with the amino acid sequence of SEQ ID No 5 or a fragment thereof.

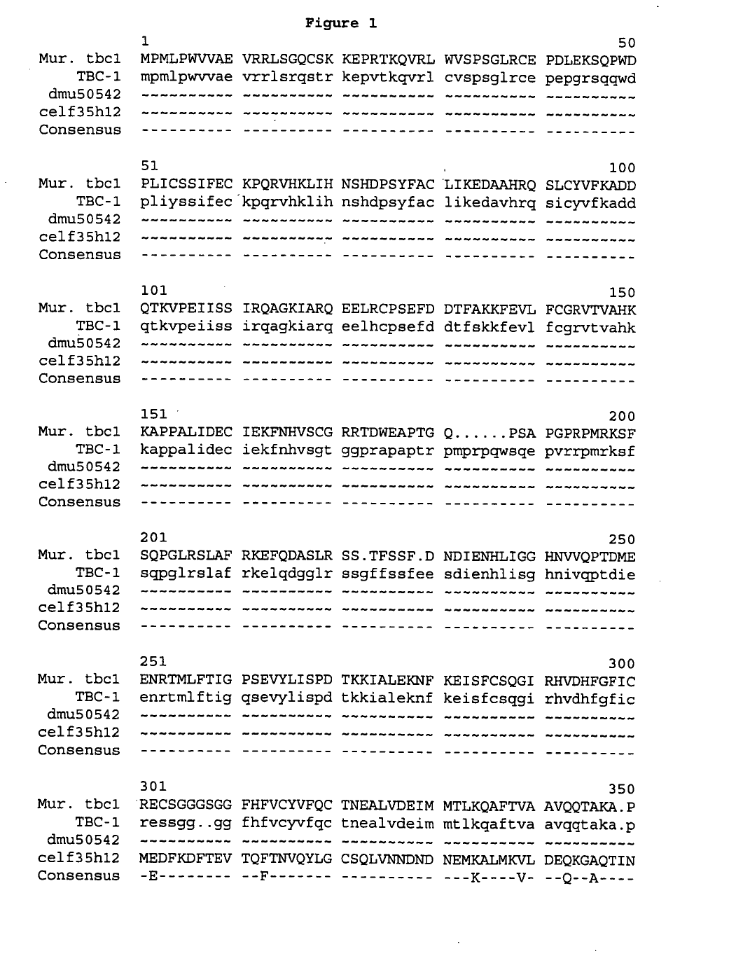

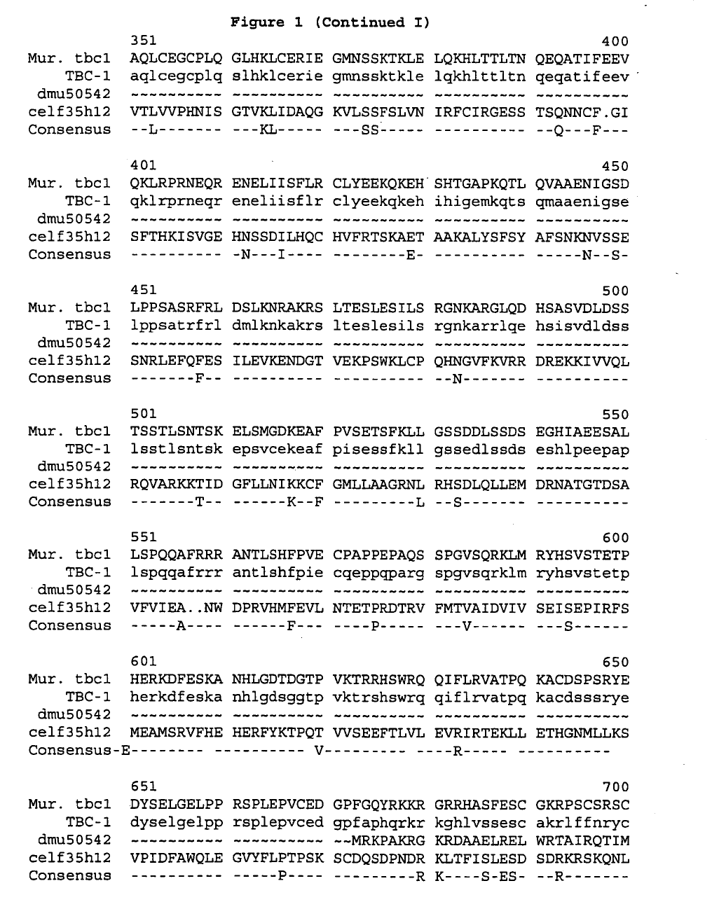

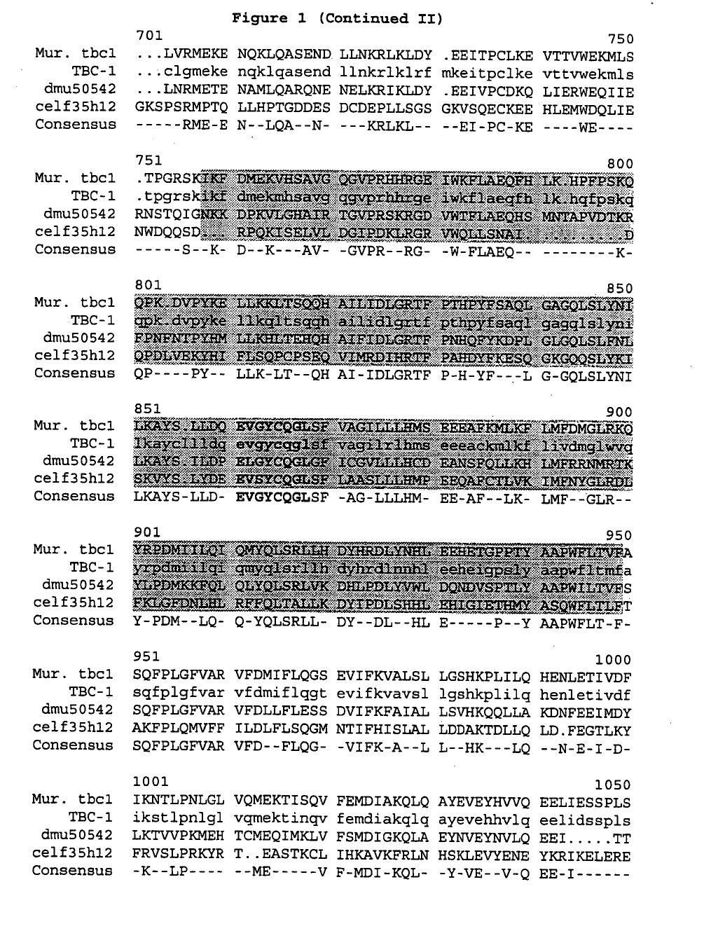

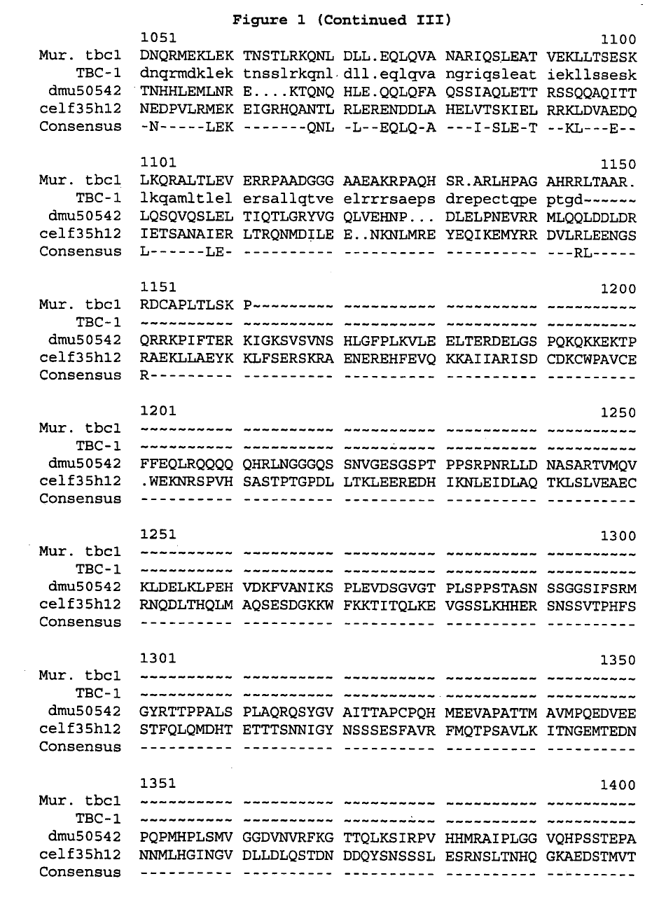

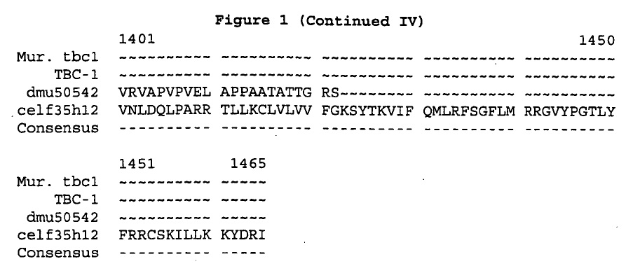

The TBC-1 polypeptide of the invention possesses amino acid homologies as regards to the murine TBC-1 protein of 1141 amino acids in length which is described in U.S. Pat. No. 5,700,927. The TBC-1 protein of the invention also possesses some homologies with two other proteins: the Pollux drosophila protein (Zhang et al., 1996) and the CDC16 protein from Caenorhabditis elegans (Wilson et al., 1994). FIG. 1 represents an amino acid alignment of a portion of the amino acid sequence of the TBC-1 protein of SEQ ID No 5 with other proteins' sharing amino acid homology with TBC-1. The upper line shows the whole amino acid sequence of the murine tbc-1 protein described in U.S. Pat. No. 5,700,927; the second line represents part of the amino acid sequence of the TBC-1 protein of SEQ ID No 5; the third line (Genbank access No dmu50542) depicts the amino acid sequence of the Pollux protein mentioned above; the fourth line (Genbank access No: celf35h12) shows the amino acid sequence of the C. elegans protein mentioned above; the fifth line presents positions in which consensus amino acids are identified, i.e. amino acids shared by the sequences presented in the four upper lines, when present.

The TBC-1 polypeptide of the amino acid sequence of SEQ ID No 5 has 1168 amino acids in length. The TBC-1 polypeptide includes a “TBC domain” which is spanning from the amino acid in position 786 to the amino acid in position 974 of the amino acid sequence of SEQ ID No 5. This TBC domain is represented in FIG. 1 as a grey area spanning from the amino acid numbered 758 to the amino acid numbered 949. This TBC domain is likely to regulate protein-protein interactions. Moreover, the TBC-1 TBC domain includes the amino acid sequence EVGYCQGL, spanning from the amino acid in position 886 to the amino acid in position 893 of the amino acid sequence of SEQ ID No 5. The EVGYCQGL amino acid sequence spans from the amino acid numbered 861 to the amino acid numbered 868 of FIG. 1. This site may interact with a kinase. Based on the structural similarity to cdc16, a yeast regulator of mitosis, TBC-1 is likely to regulate mitosis and cytokinesis by interacting with other proteins which also participate with the regulation of mitosis, cytokinesis and septum formation.

Preferred polypeptides of the invention comprise the TBC domain of TBC-1, or alternatively at least the EVGYCQGL amino acid sequence motif.

A further object of the present invention concerns a purified or isolated polypeptide which is encoded by a nucleic acid comprising a nucleotide sequence selected from the group consisting of SEQ ID Nos 1, 2, 3, and 4 or fragments or variants thereof.

A single variant molecule of the TBC-1 protein is explicitly excluded from the scope of the present invention, which is a polypeptide having the same amino acid sequence than the murine tbc1 protein described in the U.S. Pat. No. 5,700,927.

Amino acid deletions, additions or substitutions in the TBC-1 protein are preferably located outside of the TBC domain as defined above. Most preferably, a mutated TBC-1 protein has an intact “EVGYCQGL” amino acid motif.

Such a mutated TBC-1 protein may be the target of diagnostic tools, such as specific monoclonal or polyclonal antibodies, useful for detecting the mutated TBC-1 protein in a sample.

The invention also encompasses a TBC-1 polypeptide or a fragment or a variant thereof in which at least one peptide bound has been modified as described in the “Definitions” section.

### Antibodies that Bind TBC-1 Polypeptides of the Invention

Any TBC-1 polypeptide or whole protein may be used to generate antibodies capable of specifically binding to an expressed TBC-1 protein or fragments thereof as described.

One antibody composition of the invention is capable of specifically binding or specifically bind to the variant of the TBC-1 protein of SEQ ID No 5. For an antibody composition to specifically bind to TBC-1, it must demonstrate at least a 5%, 10%, 15%, 20%, 25%, 50%, or 100% greater binding affinity for TBC-1 protein than for another protein in an ELISA, RIA, or other antibody-based binding assay.

In a preferred embodiment, the invention concerns antibody compositions, either polyclonal or monoclonal, capable of selectively binding, or selectively bind to an epitope-containing a polypeptide comprising a contiguous span of at least 6 amino acids, preferably at least 8 to 10 amino acids, more preferably at least 12, 15, 20, 25, 30, 40, 50, 100, 150 or 200 amino acids of SEQ ID No 5; Optionally said epitope comprises at least 1, 2, 3, 5 or 10 of the following amino acid positions: 1-200,201-400, 401-600, 601-800, 801-1000, 1001-1168.

The invention also concerns a purified or isolated antibody capable of specifically binding to a mutated TBC-1 protein or to a fragment or variant thereof comprising an epitope of the mutated TBC-1 protein. In another preferred embodiment, the present invention concerns an antibody capable of binding to a polypeptide comprising at least 10 consecutive amino acids of a TBC-1 protein and including at least one of the amino acids which can be encoded by the trait causing mutations.

In a preferred embodiment, the invention concerns the use in the manufacture of antibodies of a polypeptide comprising a contiguous span of at least 6 amino acids, preferably at least 8 to 10 amino acids, more preferably at least 12, 15, 20, 25, 30, 40, 50, 100, 150 or 200 amino acids of SEQ ID No 5; Optionally said polypeptide comprises at least 1, 2, 3, 5 or 10 of the following amino acid positions: 1-200, 201-400, 401-600, 601-800, 801-1000, 1001-1168.

The antibodies of the invention may be labeled by any one of the radioactive, fluorescent or enzymatic labels known in the art.

The TBC-1 polypeptide of SEQ ID No 5 or a fragment thereof can be used for the preparation of polyclonal or monoclonal antibodies.

The TBC-1 polypeptide expressed from a DNA sequence comprising at least one of the nucleic acid sequences of SEQ ID Nos 1, 2, 3 and 4 may also be used to generate antibodies capable of specifically binding to the TBC-1 polypeptide of SEQ ID No 5 or a fragment thereof.

Preferred antibodies according to the invention are prepared using TBC-1 peptide fragments that do not comprise the EVGYCQGL amino acid motif.

Other preferred antibodies of the invention are prepared using TBC-1 peptide fragments that do not comprise the TBC domain defined elsewhere in the specification.

The antibodies may be prepared from hybridomas according to the technique described by Kohler and Milstein in 1975. The polyclonal antibodies may be prepared by immunization of a mammal, especially a mouse or a rabbit, with a polypeptide according to the invention that is combined with an adjuvant of immunity, and then by purifying of the specific antibodies contained in the serum of the immunized animal on a affinity chromatography column on which has previously been immobilized the polypeptide that has been used as the antigen.

The present invention also includes, chimeric single chain Fv antibody fragments (Martineau et al., 1998), antibody fragments obtained through phage display libraries (Ridder et al., 1995; Vaughan et al., 1995) and humanized antibodies (Reinmann et al., 1997; Leger et al., 1997).

Antibody preparations prepared according to either protocol are useful in quantitative immunoassays which determine concentrations of antigen-bearing substances in biological samples; they are also used semi-quantitatively or qualitatively to identify the presence of antigen in a biological sample. The antibodies may also be used in therapeutic compositions for killing cells expressing the protein or reducing the levels of the protein in the body.

Consequently, the invention is also directed to a method for detecting specifically the presence of a TBC-1 polypeptide according to the invention in a biological sample, said method comprising the following steps:

a) bringing into contact the biological sample with a polyclonal or monoclonal antibody that specifically binds a TBC-1 polypeptide comprising an amino acid sequence of SEQ ID No 5, or to a peptide fragment or variant thereof; and

b) detecting the antigen-antibody complex formed.

The invention also concerns a diagnostic kit for detecting in vitro the presence of a TBC-1 polypeptide according to the present invention in a biological sample, wherein said kit comprises:

a) a polyclonal or monoclonal antibody that specifically binds a TBC-1 polypeptide comprising an amino acid sequence of SEQ ID No 5, or to a peptide fragment or variant thereof, optionally labeled;

b) a reagent allowing the detection of the antigen-antibody complexes formed, said reagent carrying optionally a label, or being able to be recognized itself by a labeled reagent, more particularly in the case when the above-mentioned monoclonal or polyclonal antibody is not labeled by itself.

### TBC-1-Related Biallelic Markers

The inventors have discovered nucleotide polymorphisms located within the genomic DNA containing the TBC-1 gene, and among them SNP that are also termed biallelic markers. The biallelic markers of the invention can be used for example for the generation of genetic map, the linkage analysis, the association studies.

A—Identification Of TBC-1-Related Biallelic Markers

There are two preferred methods through which the biallelic markers of the present invention can be generated. In a first method, DNA samples from unrelated individuals are pooled together, following which the genomic DNA of interest is amplified and sequenced. The nucleotide sequences thus obtained are then analyzed to identify significant polymorphisms.

One of the major advantages of this method resides in the fact that the pooling of the DNA samples substantially reduces the number of DNA amplification reactions and sequencing which must be carried out. Moreover, this method is sufficiently sensitive so that a biallelic marker obtained therewith usually shows a sufficient degree of informativeness for conducting association studies.

In a second method for generating biallelic markers, the DNA samples are not pooled and are therefore amplified and sequenced individually. The resulting nucleotide sequences obtained are then also analyzed to identify significant polymorphisms.

It will readily be appreciated that when this second method is used, a substantially higher number of DNA amplification reactions must be carried out. It will further be appreciated that including such potentially less informative biallelic markers in association studies to identify potential genetic associations with a trait may allow in some cases the direct identification of causal mutations, which may, depending on their penetrance, be rare mutations. This method is usually preferred when biallelic markers need to be identified in order to perform association studies within candidate genes.

In both methods, the genomic DNA samples from which the biallelic markers of the present invention are generated are preferably obtained from unrelated individuals corresponding to a heterogeneous population of known ethnic background, or from familial cases.

The number of individuals from whom DNA samples are obtained can vary substantially, preferably from about 10 to about 1000, preferably from about 50 to about 200 individuals. It is usually preferred to collect DNA samples from at least about 100 individuals in order to have sufficient polymorphic diversity in a given population to generate as many markers as possible and to generate statistically significant results.

As for the source of the genomic DNA to be subjected to analysis, any test sample can be foreseen without any particular limitation. The preferred source of genomic DNA used in the context of the present invention is the peripheral venous blood of each donor.

The techniques of DNA extraction are well-known to the skilled technician. Details of a preferred embodiment are provided in Example 2.

DNA samples can be pooled or unpooled for the amplification step. DNA amplification techniques are well-known to those skilled in the art.

Amplification techniques that can be used in the context of the present invention include, but are not limited to, the ligase chain reaction (LCR) described in EP-A-320 308, WO 9320227 and EP-A-439 182, the polymerase chain reaction (PCR, RT-PCR) and techniques such as the nucleic acid sequence based amplification (NASBA) described in Guatelli J. C., et al. (1990) and in Compton J. (1991), Q-beta amplification as described in European Patent Application No 4544610, strand displacement amplification as described in Walker et al. (1996) and EP A 684 315 and, target mediated amplification as described in PCT Publication WO 9322461.

LCR and Gap LCR are exponential amplification techniques, both depend on DNA ligase to join adjacent primers annealed to a DNA molecule. In Ligase Chain Reaction (LCR), probe pairs are used which include two primary (first and second) and two secondary (third and fourth) probes, all of which are employed in molar excess to target. The first probe hybridizes to a first segment of the target strand and the second probe hybridizes to a second segment of the target strand, the first and second segments being contiguous so that the primary probes abut one another in 5′ phosphate-3′hydroxyl relationship, and so that a ligase can covalently fuse or ligate the two probes into a fused product. In addition, a third (secondary) probe can hybridize to a portion of the first probe and a fourth (secondary) probe can hybridize to a portion of the second probe in a similar abutting fashion. Of course, if the target is initially double stranded, the secondary probes also will hybridize to the target complement in the first instance. Once the ligated strand of primary probes is separated from the target strand, it will hybridize with the third and fourth probes, which can be ligated to form a complementary, secondary ligated product. It is important to realize that the ligated products are functionally equivalent to either the target or its complement. By repeated cycles of hybridization and ligation, amplification of the target sequence is achieved. A method for multiplex LCR has also been described (WO 9320227). Gap LCR (GLCR) is a version of LCR where the probes are not adjacent but are separated by 2 to 3 bases.

For amplification of mRNAs, it is within the scope of the present invention to reverse transcribe mRNA into cDNA followed by polymerase chain reaction (RT-PCR); or, to use a single enzyme for both steps as described in U.S. Pat. No. 5,322,770 or, to use Asymmetric Gap LCR (RT-AGLCR) as described by Marshall et al. (1994). AGLCR is a modification of GLCR that allows the amplification of RNA.

The PCR technology is the preferred amplification technique used in the present invention. A variety of PCR techniques are familiar to those skilled in the art. For a review of PCR technology, see White (1997) and the publication entitled “PCR Methods and Applications” (1991, Cold Spring Harbor Laboratory Press). In each of these PCR procedures, PCR primers on either side of the nucleic acid sequences to be amplified are added to a suitably prepared nucleic acid sample along with dNTPs and a thermostable polymerase such as Taq polymerase, Pfu polymerase, or Vent polymerase. The nucleic acid in the sample is denatured and the PCR primers are specifically hybridized to complementary nucleic acid sequences in the sample. The hybridized primers are extended. Thereafter, another cycle of denaturation, hybridization, and extension is initiated. The cycles are repeated multiple times to produce an amplified fragment containing the nucleic acid sequence between the primer sites. PCR has further been described in several patents including U.S. Pat. Nos. 4,683,195; 4,683,202; and 4,965,188.

The PCR technology is the preferred amplification technique used to identify new biallelic markers. A typical example of a PCR reaction suitable for the purposes of the present invention is provided in Example 3.

One of the aspects of the present invention is a method for the amplification of a TBC-1 gene, particularly the genomic sequences of SEQ ID Nos 1 and 2 or of the cDNA sequence of SEQ ID Nos 3 or 4 or a fragment or variant thereof in a test sample, preferably using the PCR technology. The method comprises the steps of contacting a test sample suspected of containing the target TBC-1 sequence or portion thereof with amplification reaction reagents comprising a pair of amplification primers.

Thus, the present invention also relates to a method for the amplification of a TBC-1 gene sequence, particularly of a fragment of the genomic sequence of SEQ ID No 1 or of the cDNA sequence of SEQ ID No 2 or 3, or a fragment or a variant thereof in a test sample, said method comprising the steps of:

a) contacting a test sample suspected of containing the targeted TBC-1 gene sequence or portion thereof with amplification reaction reagents comprising a pair of amplification primers located on either side of the TBC-1 region to be amplified, and

b) optionally, detecting the amplification products.

The invention also concerns a kit for the amplification of a TBC-1 gene sequence, particularly of a portion of the genomic sequence of SEQ ID Nos 1 or 2, or of the cDNA sequence of SEQ ID Nos 3 or 4, or a variant thereof in a test sample, wherein said kit comprises:
a) a pair of oligonucleotide primers located on either side of the TBC-1 region to be amplified;
b) optionally, the reagents necessary for performing the amplification reaction.

In one embodiment of the above amplification method and kit, the amplification product is detected by hybridization with a labeled probe having a sequence which is complementary to the amplified region. In another embodiment of the above amplification method and kit, primers comprise a sequence which is selected from the group consisting of B1 to B15, C1 to C15, D1 to D19, and E1 to E19.

In a first embodiment of the present invention, biallelic markers are identified using genomic sequence information generated by the inventors. Sequenced genomic DNA fragments are used to design primers for the amplification of 500 bp fragments. These 500 bp fragments are amplified from genomic DNA and are scanned for biallelic markers. Primers may be designed using the OSP software (Hillier L. and Green P., 1991). All primers may contain, upstream of the specific target bases, a common oligonucleotide tail that serves as a sequencing primer. Those skilled in the art are familiar with primer extensions, which can be used for these purposes.

Preferred primers, useful for the amplification of genomic sequences encoding the candidate genes, focus on promoters, exons and splice sites of the genes. A biallelic marker presents a higher probability to be an eventual causal mutation if it is located in these functional regions of the gene. Preferred amplification primers of the invention include the nucleotide sequences of B1 to B15 and C1 to C15 further detailed in Example 3.

The amplification products generated as described above with the primers of the invention are then sequenced using methods known and available to the skilled technician. Preferably, the amplified DNA is subjected to automated dideoxy terminator sequencing reactions using a dye-primer cycle sequencing protocol. Following gel image analysis and DNA sequence extraction, sequence data are automatically processed with adequate software to assess sequence quality.

A polymorphism analysis software is used that detects the presence of biallelic sites among individual or pooled amplified fragment sequences. Polymorphism search is based on the presence of superimposed peaks in the electrophoresis pattern. These peaks which present distinct colors correspond to two different nucleotides at the same position on the sequence. The polymorphism has to be detected on both strands for validation.

19 biallelic markers were found in the TBC-1 gene. They are detailed in the Table 2. They are located in intronic regions.

B—Genotyping of TBC-1-Related Biallelic Markers

The polymorphisms identified above can be further confirmed and their respective frequencies can be determined through various methods using the previously described primers and probes. These methods can also be useful for genotyping either new populations in association studies or linkage analysis or individuals in the context of detection of alleles of biallelic markers which are known to be associated with a given trait. The genotyping of the biallelic markers is also important for the mapping. Those skilled in the art should note that the methods described below can be equally performed on individual or pooled DNA samples.

Once a given polymorphic site has been found and characterized as a biallelic marker as described above, several methods can be used in order to determine the specific allele carried by an individual at the given polymorphic base.

The identification of biallelic markers described previously allows the design of appropriate oligonucleotides, which can be used as probes and primers, to amplify a TBC-1 gene containing the polymorphic site of interest and for the detection of such polymorphisms.

The biallelic markers according to the present invention may be used in methods for the identification and characterization of an association between alleles for one or several biallelic markers of the sequence of the TBC-1 gene and a trait.

The identified polymorphisms, and consequently the biallelic markers of the invention, may be used in methods for the detection in an individual of TBC-1 alleles associated with a trait, more particularly a trait related to a cell differentiation or abnormal cell proliferation disorders, and most particularly a trait related to cancer diseases, specifically prostate cancer.

In one embodiment the invention encompasses methods of genotyping comprising determining the identity of a nucleotide at a TBC-1-related biallelic marker or the complement thereof in a biological sample; optionally, wherein said TBC-1-related biallelic marker is selected from the group consisting of A1 to A19, and the complements thereof, or optionally the biallelic markers in linkage disequilibrium therewith; optionally, wherein said biological sample is derived from a single subject; optionally, wherein the identity of the nucleotides at said biallelic marker is determined for both copies of said biallelic marker present in said individual's genome; optionally, wherein said biological sample is derived from multiple subjects; Optionally, the genotyping methods of the invention encompass methods with any further limitation described in this disclosure, or those following, specified alone or in any combination; Optionally, said method is performed in vitro; optionally, further comprising amplifying a portion of said sequence comprising the biallelic marker prior to said determining step; Optionally, wherein said amplifying is performed by PCR, LCR, or replication of a recombinant vector comprising an origin of replication and said fragment in a host cell; optionally, wherein said determining is performed by a hybridization assay, a sequencing assay, a microsequencing assay, or an enzyme-based mismatch detection assay.

Source of Nucleic Acids for Genotyping

Any source of nucleic acids, in purified or non-purified form, can be utilized as the starting nucleic acid, provided it contains or is suspected of containing the specific nucleic acid sequence desired. DNA or RNA may be extracted from cells, tissues, body fluids and the like as described above. While nucleic acids for use in the genotyping methods of the invention can be derived from any mammalian source, the test subjects and individuals from which nucleic acid samples are taken are generally understood to be human.

Amplification of DNA Fragments Comprising Biallelic Markers

Methods and polynucleotides are provided to amplify a segment of nucleotides comprising one or more biallelic marker of the present invention. It will be appreciated that amplification of DNA fragments comprising biallelic markers may be used in various methods and for various purposes and is not restricted to genotyping. Nevertheless, many genotyping methods, although not all, require the previous amplification of the DNA region carrying the biallelic marker of interest. Such methods specifically increase the concentration or total number of sequences that span the biallelic marker or include that site and sequences located either distal or proximal to it. Diagnostic assays may also rely on amplification of DNA segments carrying a biallelic marker of the present invention. Amplification of DNA may be achieved by any method known in the art. Amplification techniques are described above in the section entitled, “Identification of TBC-1-related biallelic markers.”

Some of these amplification methods are particularly suited for the detection of single nucleotide polymorphisms and allow the simultaneous amplification of a target sequence and the identification of the polymorphic nucleotide as it is further described below.

The identification of biallelic markers as described above allows the design of appropriate oligonucleotides, which can be used as primers to amplify DNA fragments comprising the biallelic markers of the present invention. Amplification can be performed using the primers initially used to discover new biallelic markers which are described herein or any set of primers allowing the amplification of a DNA fragment comprising a biallelic marker of the present invention.

In some embodiments the present invention provides primers for amplifying a DNA fragment containing one or more biallelic markers of the present invention. Preferred amplification primers are listed in Example 2. It will be appreciated that the primers listed are merely exemplary and that any other set of primers which produce amplification products containing one or more biallelic markers of the present invention are also of use.

The spacing of the primers determines the length of the segment to be amplified. In the context of the present invention, amplified segments carrying biallelic markers can range in size from at least about 25 bp to 35 kbp. Amplification fragments from 25-3000 bp are typical, fragments from 50-1000 bp are preferred and fragments from 100-600 bp are highly preferred. It will be appreciated that amplification primers for the biallelic markers may be any sequence which allow the specific amplification of any DNA fragment carrying the markers. Amplification primers may be labeled or immobilized on a solid support as described in “Oligonucleotide probes and primers”.

Methods of Genotyping DNA Samples for Biallelic Markers

Any method known in the art can be used to identify the nucleotide present at a biallelic marker site. Since the biallelic marker allele to be detected has been identified and specified in the present invention, detection will prove simple for one of ordinary skill in the art by employing any of a number of techniques. Many genotyping methods require the previous amplification of the DNA region carrying the biallelic marker of interest. While the amplification of target or signal is often preferred at present, ultrasensitive detection methods which do not require amplification are also encompassed by the present genotyping methods. Methods well-known to those skilled in the art that can be used to detect biallelic polymorphisms include methods such as, conventional dot blot analyzes, single strand conformational polymorphism analysis (SSCP) described by Orita et al. (1989), denaturing gradient gel electrophoresis (DGGE), heteroduplex analysis, mismatch cleavage detection, and other conventional techniques as described in Sheffield et al. (1991), White et al. (1992), Grompe et al. (1989 and 1993). Another method for determining the identity of the nucleotide present at a particular polymorphic site employs a specialized exonuclease-resistant nucleotide derivative as described in U.S. Pat. No. 4,656,127.

Preferred methods involve directly determining the identity of the nucleotide present at a biallelic marker site by sequencing assay, enzyme-based mismatch detection assay, or hybridization assay. The following is a description of some preferred methods. A highly preferred method is the microsequencing technique. The term “sequencing” is generally used herein to refer to polymerase extension of duplex primer/template complexes and includes both traditional sequencing and microsequencing.

### 1) Sequencing Assays

The nucleotide present at a polymorphic site can be determined by sequencing methods. In a preferred embodiment, DNA samples are subjected to PCR amplification before sequencing as described above. DNA sequencing methods are described in “Sequencing Of Amplified Genomic DNA And Identification Of Single Nucleotide Polymorphisms”.

Preferably, the amplified DNA is subjected to automated dideoxy terminator sequencing reactions using a dye-primer cycle sequencing protocol. Sequence analysis allows the identification of the base present at the biallelic marker site.

### 2) Microsequencing Assays

In microsequencing methods, the nucleotide at a polymorphic site in a target DNA is detected by a single nucleotide primer extension reaction. This method involves appropriate microsequencing primers which, hybridize just upstream of the polymorphic base of interest in the target nucleic acid. A polymerase is used to specifically extend the 3′ end of the primer with one single ddNTP (chain terminator) complementary to the nucleotide at the polymorphic site. Next the identity of the incorporated nucleotide is determined in any suitable way.

Typically, microsequencing reactions are carried out using fluorescent ddNTPs and the extended microsequencing primers are analyzed by electrophoresis on ABI 377 sequencing machines to determine the identity of the incorporated nucleotide as described in EP 412 883, the disclosure of which is incorporated herein by reference in its entirety. Alternatively capillary electrophoresis can be used in order to process a higher number of assays simultaneously. An example of a typical microsequencing procedure that can be used in the context of the present invention is provided in Example 4.

Different approaches can be used for the labeling and detection of ddNTPs. A homogeneous phase detection method based on fluorescence resonance energy transfer has been described by Chen and Kwok (1997) and Chen et al. (1997). In this method, amplified genomic DNA fragments containing polymorphic sites are incubated with a 5′-fluorescein-labeled primer in the presence of allelic dye-labeled dideoxyribonucleoside triphosphates and a modified Taq polymerase. The dye-labeled primer is extended one base by the dye-terminator specific for the allele present on the template. At the end of the genotyping reaction, the fluorescence intensities of the two dyes in the reaction mixture are analyzed directly without separation or purification. All these steps can be performed in the same tube and the fluorescence changes can be monitored in real time. Alternatively, the extended primer may be analyzed by MALDI-TOF Mass Spectrometry. The base at the polymorphic site is identified by the mass added onto the microsequencing primer (see Haff and Smirnov, 1997).

Microsequencing may be achieved by the established microsequencing method or by developments or derivatives thereof. Alternative methods include several solid-phase microsequencing techniques. The basic microsequencing protocol is the same as described previously, except that the method is conducted as a heterogeneous phase assay, in which the primer or the target molecule is immobilized or captured onto a solid support. To simplify the primer separation and the terminal nucleotide addition analysis, oligonucleotides are attached to solid supports or are modified in such ways that permit affinity separation as well as polymerase extension. The 5′ ends and internal nucleotides of synthetic oligonucleotides can be modified in a number of different ways to permit different affinity separation approaches, e.g., biotinylation. If a single affinity group is used on the oligonucleotides, the oligonucleotides can be separated from the incorporated terminator regent. This eliminates the need of physical or size separation. More than one oligonucleotide can be separated from the terminator reagent and analyzed simultaneously if more than one affinity group is used. This permits the analysis of several nucleic acid species or more nucleic acid sequence information per extension reaction. The affinity group need not be on the priming oligonucleotide but could alternatively be present on the template. For example, immobilization can be carried out via an interaction between biotinylated DNA and streptavidin-coated microtitration wells or avidin-coated polystyrene particles. In the same manner, oligonucleotides or templates may be attached to a solid support in a high-density format. In such solid phase microsequencing reactions, incorporated ddNTPs can be radiolabeled (Syvanen, 1994) or linked to fluorescein (Livak and Hainer, 1994). The detection of radiolabeled ddNTPs can be achieved through scintillation-based techniques. The detection of fluorescein-linked ddNTPs can be based on the binding of antifluorescein antibody conjugated with alkaline phosphatase, followed by incubation with a chromogenic substrate (such asp-nitrophenyl phosphate). Other possible reporter-detection pairs include: ddNTP linked to dinitrophenyl (DNP) and anti-DNP alkaline phosphatase conjugate (Harju et al., 1993) or biotinylated ddNTP and horseradish peroxidase-conjugated streptavidin with o-phenylenediamine as a substrate (WO 92/15712). As yet another alternative solid-phase microsequencing procedure, Nyren et al. (1993) described a method relying on the detection of DNA polymerase activity by an enzymatic luminometric inorganic pyrophosphate detection assay (ELIDA).

Pastinen et al. (1997) describe a method for multiplex detection of single nucleotide polymorphism in which the solid phase minisequencing principle is applied to an oligonucleotide array format. High-density arrays of DNA probes attached to a solid support (DNA chips) are further described below.

In one aspect the present invention provides polynucleotides and methods to genotype one or more biallelic markers of the present invention by performing a microsequencing assay. Preferred microsequencing primers include the nucleotide sequences D1 to D15 and E1 to E15. It will be appreciated that the microsequencing primers listed in Example 5 are merely exemplary and that, any primer having a 3′ end immediately adjacent to the polymorphic nucleotide may be used. Similarly, it will be appreciated that microsequencing analysis may be performed for any biallelic marker or any combination of biallelic markers of the present invention. One aspect of the present invention is a solid support which includes one or more microsequencing primers listed in Example 5, or fragments comprising at least 8, 12, 15, 20, 25, 30, 40, or 50 consecutive nucleotides thereof, to the extent that such lengths are consistent with the primer described, and having a 3′ terminus immediately upstream of the corresponding biallelic marker, for determining the identity of a nucleotide at a biallelic marker site.

### 3) Mismatch Detection Assays Based on Polymerases and Ligases

In one aspect the present invention provides polynucleotides and methods to determine the allele of one or more biallelic markers of the present invention in a biological sample, by mismatch detection assays based on polymerases and/or ligases. These assays are based on the specificity of polymerases and ligases. Polymerization reactions places particularly stringent requirements on correct base pairing of the 3′ end of the amplification primer and the joining of two oligonucleotides hybridized to a target DNA sequence is quite sensitive to mismatches close to the ligation site, especially at the 3′ end. Methods, primers and various parameters to amplify DNA fragments comprising biallelic markers of the present invention are further described above in “Amplification Of DNA Fragments Comprising Biallelic Markers”.

### Allele Specific Amplification Primers

Discrimination between the two alleles of a biallelic marker can also be achieved by allele specific amplification, a selective strategy, whereby one of the alleles is amplified without amplification of the other allele. For allele specific amplification, at least one member of the pair of primers is sufficiently complementary with a region of a TBC-1 gene comprising the polymorphic base of a biallelic marker of the present invention to hybridize therewith and to initiate the amplification. Such primers are able to discriminate between the two alleles of a biallelic marker.

This is accomplished by placing the polymorphic base at the 3′ end of one of the amplification primers. Because the extension forms from the 3′ end of the primer, a mismatch at or near this position has an inhibitory effect on amplification. Therefore, under appropriate amplification conditions, these primers only direct amplification on their complementary allele. Determining the precise location of the mismatch and the corresponding assay conditions are well within the ordinary skill in the art.

### Ligation/Amplification Based Methods

The “Oligonucleotide Ligation Assay” (OLA) uses two oligonucleotides which are designed to be capable of hybridizing to abutting sequences of a single strand of a target molecules. One of the oligonucleotides is biotinylated, and the other is detectably labeled. If the precise complementary sequence is found in a target molecule, the oligonucleotides will hybridize such that their termini abut, and create a ligation substrate that can be captured and detected. OLA is capable of detecting single nucleotide polymorphisms and may be advantageously combined with PCR as described by Nickerson et al. (1990). In this method, PCR is used to achieve the exponential amplification of target DNA, which is then detected using OLA.

Other amplification methods which are particularly suited for the detection of single nucleotide polymorphism include LCR (ligase chain reaction), Gap LCR (GLCR) which are described above in “DNA Amplification”. LCR uses two pairs of probes to exponentially amplify a specific target. The sequences of each pair of oligonucleotides, is selected to permit the pair to hybridize to abutting sequences of the same strand of the target. Such hybridization forms a substrate for a template-dependant ligase. In accordance with the present invention, LCR can be performed with oligonucleotides having the proximal and distal sequences of the same strand of a biallelic marker site. In one embodiment, either oligonucleotide will be designed to include the biallelic marker site. In such an embodiment, the reaction conditions are selected such that the oligonucleotides can be ligated together only if the target molecule either contains or lacks the specific nucleotide that is complementary to the biallelic marker on the oligonucleotide. In an alternative embodiment, the oligonucleotides will not include the biallelic marker, such that when they hybridize to the target molecule, a “gap” is created as described in WO 90/01069. This gap is then “filled” with complementary dNTPs (as mediated by DNA polymerase), or by an additional pair of oligonucleotides. Thus at the end of each cycle, each single strand has a complement capable of serving as a target during the next cycle and exponential allele-specific amplification of the desired sequence is obtained.

Ligase/Polymerase-mediated Genetic Bit Analysis™ is another method for determining the identity of a nucleotide at a preselected site in a nucleic acid molecule (WO 95/21271). This method involves the incorporation of a nucleoside triphosphate that is complementary to the nucleotide present at the preselected site onto the terminus of a primer molecule, and their subsequent ligation to a second oligonucleotide. The reaction is monitored by detecting a specific label attached to the reaction's solid phase or by detection in solution.

### 4) Hybridization Assay Methods

A preferred method of determining the identity of the nucleotide present at a biallelic marker site involves nucleic acid hybridization. The hybridization probes, which can be conveniently used in such reactions, preferably include the probes defined herein. Any hybridization assay may be used including Southern hybridization, Northern hybridization, dot blot hybridization and solid-phase hybridization (see Sambrook et al., 1989).

Hybridization refers to the formation of a duplex structure by two single stranded nucleic acids due to complementary base pairing. Hybridization can occur between exactly complementary nucleic acid strands or between nucleic acid strands that contain minor regions of mismatch. Specific probes can be designed that hybridize to one form of a biallelic marker and not to the other and therefore are able to discriminate between different allelic forms. Allele-specific probes are often used in pairs, one member of a pair showing perfect match to a target sequence containing the original allele and the other showing a perfect match to the target sequence containing the alternative allele. Hybridization conditions should be sufficiently stringent that there is a significant difference in hybridization intensity between alleles, and preferably an essentially binary response, whereby a probe hybridizes to only one of the alleles. Stringent, sequence specific hybridization conditions, under which a probe will hybridize only to the exactly complementary target sequence are well known in the art (Sambrook et al., 1989). Stringent conditions are sequence dependent and will be different in different circumstances. Generally, stringent conditions are selected to be about 5° C. lower than the thermal melting point (Tm) for the specific sequence at a defined ionic strength and pH. Although such hybridization can be performed in solution, it is preferred to employ a solid-phase hybridization assay. The target DNA comprising a biallelic marker of the present invention may be amplified prior to the hybridization reaction. The presence of a specific allele in the sample is determined by detecting the presence or the absence of stable hybrid duplexes formed between the probe and the target DNA. The detection of hybrid duplexes can be carried out by a number of methods. Various detection assay formats are well known which utilize detectable labels bound to either the target or the probe to enable detection of the hybrid duplexes. Typically, hybridization duplexes are separated from unhybridized nucleic acids and the labels bound to the duplexes are then detected. Those skilled in the art will recognize that wash steps may be employed to wash away excess target DNA or probe as well as unbound conjugate. Further, standard heterogeneous assay formats are suitable for detecting the hybrids using the labels present on the primers and probes.

Two recently developed assays allow hybridization-based allele discrimination with no need for separations or washes (see Landegren U. et al., 1998). The TaqMan assay takes advantage of the 5′ nuclease activity of Taq DNA polymerase to digest a DNA probe annealed specifically to the accumulating amplification product. TaqMan probes are labeled with a donor-acceptor dye pair that interacts via fluorescence energy transfer. Cleavage of the TaqMan probe by the advancing polymerase during amplification dissociates the donor dye from the quenching acceptor dye, greatly increasing the donor fluorescence. All reagents necessary to detect two allelic variants can be assembled at the beginning of the reaction and the results are monitored in real time (see Livak et al., 1995). In an alternative homogeneous hybridization based procedure, molecular beacons are used for allele discriminations. Molecular beacons are hairpin-shaped oligonucleotide probes that report the presence of specific nucleic acids in homogeneous solutions. When they bind to their targets they undergo a conformational reorganization that restores the fluorescence of an internally quenched fluorophore (Tyagi et al., 1998).

The polynucleotides provided herein can be used to produce probes which can be used in hybridization assays for the detection of biallelic marker alleles in biological samples. These probes are characterized in that they preferably comprise between 8 and 50 nucleotides, and in that they are sufficiently complementary to a sequence comprising a biallelic marker of the present invention to hybridize thereto and preferably sufficiently specific to be able to discriminate the targeted sequence for only one nucleotide variation. A particularly preferred probe is 25 nucleotides in length. Preferably the biallelic marker is within 4 nucleotides of the center of the polynucleotide probe. In particularly preferred probes, the biallelic marker is at the center of said polynucleotide. Preferred probes comprise a nucleotide sequence selected from the group consisting of amplicons listed in Table 1 and the sequences complementary thereto, or a fragment thereof, said fragment comprising at least about 8 consecutive nucleotides, preferably 10, 15, 20, more preferably 25, 30, 40, 47, or 50 consecutive nucleotides and containing a polymorphic base. Preferred probes comprise a nucleotide sequence selected from the group consisting of P1 to P7, P9 to P13, P15 to P19 and the sequences complementary thereto. In preferred embodiments the polymorphic base(s) are within 5, 4, 3, 2, 1, nucleotides of the center of the said polynucleotide, more preferably at the center of said polynucleotide.

Preferably the probes of the present invention are labeled or immobilized on a solid support. Labels and solid supports are further described in “Oligonucleotide Probes and Primers”. The probes can be non-extendable as described in “Oligonucleotide Probes and Primers”.

By assaying the hybridization to an allele specific probe, one can detect the presence or absence of a biallelic marker allele in a given sample. High-Throughput parallel hybridization in array format is specifically encompassed within “hybridization assays” and are described below.

### 5) Hybridization to Addressable Arrays of Oligonucleotides

Hybridization assays based on oligonucleotide arrays rely on the differences in hybridization stability of short oligonucleotides to perfectly matched and mismatched target sequence variants. Efficient access to polymorphism information is obtained through a basic structure comprising high-density arrays of oligonucleotide probes attached to a solid support (e.g., the chip) at selected positions. Each DNA chip can contain thousands to millions of individual synthetic DNA probes arranged in a grid-like pattern and miniaturized to the size of a dime.

The chip technology has already been applied with success in numerous cases. For example, the screening of mutations has been undertaken in the BRCA1 gene, in S. cerevisiae mutant strains, and in the protease gene of HIV-1 virus (Hacia et al., 1996; Shoemaker et al., 1996; Kozal et al., 1996). Chips of various formats for use in detecting biallelic polymorphisms can be produced on a customized basis by Affymetrix (GeneChip™), Hyseq (HyChip and HyGnostics), and Protogene Laboratories.

In general, these methods employ arrays of oligonucleotide probes that are complementary to target nucleic acid sequence segments from an individual which, target sequences include a polymorphic marker. EP 785280 describes a tiling strategy for the detection of single nucleotide polymorphisms. Briefly, arrays may generally be “tiled” for a large number of specific polymorphisms. By “tiling” is generally meant the synthesis of a defined set of oligonucleotide probes which is made up of a sequence complementary to the target sequence of interest, as well as preselected variations of that sequence, e.g., substitution of one or more given positions with one or more members of the basis set of nucleotides. Tiling strategies are further described in PCT application No. WO 95/11995. In a particular aspect, arrays are tiled for a number of specific, identified biallelic marker sequences. In particular, the array is tiled to include a number of detection blocks, each detection block being specific for a specific biallelic marker or a set of biallelic markers. For example, a detection block may be tiled to include a number of probes, which span the sequence segment that includes a specific polymorphism. To ensure probes that are complementary to each allele, the probes are synthesized in pairs differing at the biallelic marker. In addition to the probes differing at the polymorphic base, monosubstituted probes are also generally tiled within the detection block. These monosubstituted probes have bases at and up to a certain number of bases in either direction from the polymorphism, substituted with the remaining nucleotides (selected from A, T, G, C and U). Typically the probes in a tiled detection block will include substitutions of the sequence positions up to and including those that are 5 bases away from the biallelic marker. The monosubstituted probes provide internal controls for the tiled array, to distinguish actual hybridization from artefactual cross-hybridization. Upon completion of hybridization with the target sequence and washing of the array, the array is scanned to determine the position on the array to which the target sequence hybridizes. The hybridization data from the scanned array is then analyzed to identify which allele or alleles of the biallelic marker are present in the sample. Hybridization and scanning may be carried out as described in PCT application No. WO 92/10092 and WO 95/11995 and U.S. Pat. No. 5,424,186.

Thus, in some embodiments, the chips may comprise an array of nucleic acid sequences of fragments of about 15 nucleotides in length. In further embodiments, the chip may comprise an array including at least one of the sequences selected from the group consisting of amplicons listed in table 1 and the sequences complementary thereto, or a fragment thereof, said fragment comprising at least about 8 consecutive nucleotides, preferably 10, 15, 20, more preferably 25, 30, 40, 47, or 50 consecutive nucleotides and containing a polymorphic base. In preferred embodiments the polymorphic base is within 5, 4, 3, 2, 1, nucleotides of the center of the said polynucleotide, more preferably at the center of said polynucleotide. In some embodiments, the chip may comprise an array of at least 2, 3, 4, 5, 6, 7, 8 or more of these polynucleotides of the invention. Solid supports and polynucleotides of the present invention attached to solid supports are further described in “Oligonucleotide Probes And Primers”.

### 6) Integrated Systems

Another technique, which may be used to analyze polymorphisms, includes multicomponent integrated systems, which miniaturize and compartmentalize processes such as PCR and capillary electrophoresis reactions in a single functional device. An example of such technique is disclosed in U.S. Pat. No. 5,589,136, which describes the integration of PCR amplification and capillary electrophoresis in chips.

Integrated systems can be envisaged mainly when microfluidic systems are used. These systems comprise a pattern of microchannels designed onto a glass, silicon, quartz, or plastic wafer included on a microchip. The movements of the samples are controlled by electric, electroosmotic or hydrostatic forces applied across different areas of the microchip to create functional microscopic valves and pumps with no moving parts.

For genotyping biallelic markers, the microfluidic system may integrate nucleic acid amplification, microsequencing, capillary electrophoresis and a detection method such as laser-induced fluorescence detection.

### Association Studies with the Biallelic Markers of the TBC-1 Gene

The identification of genes involved in suspected heterogeneous, polygenic and multifactorial traits such as cancer can be carried out through two main strategies currently used for genetic mapping: linkage analysis and association studies. Association studies examine the frequency of marker alleles in unrelated trait positive (T+) individuals compared with trait negative (T−) controls, and are generally employed in the detection of polygenic inheritance. Association studies as a method of mapping genetic traits rely on the phenomenon of linkage disequilibrium.

If two genetic loci lie on the same chromosome, then sets of alleles of these loci on the same chromosomal segment (called haplotypes) tend to be transmitted as a block from generation to generation. When not broken up by recombination, haplotypes can be tracked not only through pedigrees but also through populations. The resulting phenomenon at the population level is that the occurrence of pairs of specific alleles at different loci on the same chromosome is not random, and the deviation from random is called linkage disequilibrium (LD).

If a specific allele in a given gene is directly involved in causing a particular trait T, its frequency will be statistically increased in a trait positive population when compared to the frequency in a trait negative population. As a consequence of the existence of linkage disequilibrium, the frequency of all other alleles present in the haplotype carrying the trait-causing allele (TCA) will also be increased in trait positive individuals compared to trait negative individuals. Therefore, association between the trait and any allele in linkage disequilibrium with the trait-causing allele will suffice to suggest the presence of a trait-related gene in that particular allele's region. Linkage disequilibrium allows the relative frequencies in trait positive and trait negative populations of a limited number of genetic polymorphisms (specifically biallelic markers) to be analyzed as an alternative to screening all possible functional polymorphisms in order to find trait-causing alleles.

The general strategy to perform association studies using biallelic markers derived from a candidate region is to scan two groups of individuals (trait positive and trait negative control individuals which are characterized by a well defined phenotype as described below) in order to measure and statistically compare the allele frequencies of such biallelic markers in both groups.

If a statistically significant association with a trait is identified for at least one or more of the analyzed biallelic markers, one can assume that: either the associated allele is directly responsible for causing the trait (associated allele is the trait-causing allele), or the associated allele is in linkage disequilibrium with the trait-causing allele. If the evidence indicates that the associated allele within the candidate region is most probably not the trait-causing allele but is in linkage disequilibrium with the real trait-causing allele, then the trait-causing allele, and by consequence the gene carrying the trait-causing allele, can be found by sequencing the vicinity of the associated marker.

Collection of DNA Samples from Trait Positive (Trait+) and Trait Negative (Trait− Individuals (Inclusion Criteria)

In order to perform efficient and significant association studies such as those described herein, the trait under study should preferably follow a bimodal distribution in the population under study, presenting two clear non-overlapping phenotypes, trait positive and trait negative.

Nevertheless, even in the absence of such a bimodal distribution (as may in fact be the case for more complex genetic traits), any genetic trait may still be analyzed by the association method proposed here by carefully selecting the individuals to be included in the trait positive and trait negative phenotypic groups. The selection procedure involves to select individuals at opposite ends of the non-bimodal phenotype spectra of the trait under study, so as to include in these trait positive and trait negative populations individuals which clearly represent extreme, preferably non-overlapping phenotypes.

The definition of the inclusion criteria for the trait positive and trait negative populations is an important aspect of the present invention. The selection of drastically different but relatively uniform phenotypes enables efficient comparisons in association studies and the possible detection of marked differences at the genetic level, provided that the sample sizes of the populations under study are significant enough.

Generally, trait positive and trait negative populations to be included in association studies such as proposed in the present invention consist of phenotypically homogenous populations of individuals each representing 100% of the corresponding trait if the trait distribution is bimodal.

A first group of between 50 and 300 trait positive individuals, preferably about 100 individuals, can be recruited according to clinical-inclusion criteria.

In each case, a similar number of trait negative individuals, preferably more than 100 individuals, are included in such studies who are preferably both ethnically- and age-matched to the trait positive cases. They are checked for the absence of the clinical criteria defined above. Both trait positive and trait negative individuals should correspond to unrelated cases.

Genotyping of Trait Positive and Trait Negative Individuals

Allelic frequencies of the biallelic markers in each of the above described population can be determined using one of the methods described above under the heading “Methods of Genotyping DNA samples for biallelic markers”. Analyses are preferably performed on amplified fragments obtained by genomic PCR performed on the DNA samples from each individual in similar conditions as those described above for the generation of biallelic markers.

In a preferred embodiment, amplified DNA samples are subjected to automated microsequencing reactions using fluorescent ddNTPs (specific fluorescence for each ddNTP) and the appropriate microsequencing oligonucleotides which hybridize just upstream of the polymorphic base.

Genotyping is further described in Example 5.

Associations studies can be carried out by the skilled technician using the biallelic markers of the invention defined above, with different trait positive and trait negative populations. Suitable examples of association studies using biallelic markers of the TBC-1 gene, including the biallelic markers A1 to A19, involve studies on the following populations:

a trait positive population suffering from a cancer, preferably prostate cancer and a healthy unaffected population; or

a trait positive population suffering from prostate cancer treated with agents acting against prostate cancer and suffering from side-effects resulting from this treatment and an trait negative population suffering from prostate cancer treated with same agents without any substantial side-effects, or

a trait positive population suffering from prostate cancer treated with agents acting against prostate cancer showing a beneficial response and a trait negative population suffering from prostate cancer treated with same agents without any beneficial response, or

a trait positive population suffering from prostate cancer presenting highly aggressive prostate cancer tumors and a trait negative population suffering from prostate cancer with prostate cancer tumors devoid of aggressiveness.

It is another object of the present invention to provide a method for the identification and characterization of an association between an allele of one or more biallelic markers of a TBC-1 gene and a trait. The method comprises the steps of:

genotyping a marker or a group of biallelic markers according to the invention in trait positive;

genotyping a marker or a group of biallelic markers according to the invention in and trait negative individuals; and

establishing a statistically significant association between one allele of at least one marker and the trait.

Preferably, the trait positive and trait negative individuals are selected from non-overlapping phenotypes as regards to the trait under study. In one embodiment, the biallelic marker are selected from the group consisting of the biallelic markers A1 to A19.

In a preferred embodiment, the trait is cancer, prostate cancer, an early onset of prostate cancer, a susceptibility to prostate cancer, the level of aggressiveness of prostate cancer tumors, a modified expression of the TBC-1 gene, a modified production of the TBC-1 protein, or the production of a modified TBC-1 protein.

In a further embodiment, the trait negative population can be replaced in the association studies by a random control population.

The step of testing for and detecting the presence of DNA comprising specific alleles of a biallelic marker or a group of biallelic markers of the present invention can be carried out as described further below.

### Oligonucleotide Probes and Primers

The invention relates also to oligonucleotide molecules useful as probes or primers, wherein said oligonucleotide molecules hybridize specifically with a nucleotide sequence comprised in the TBC-1 gene, particularly the TBC-1 genomic sequence of SEQ ID Nos 1 and 2 or the TBC-1 cDNAs sequences of SEQ ID Nos 3 and 4. More particularly, the present invention also concerns oligonucleotides for the detection of alleles of biallelic markers of the TBC-1 gene. These oligonucleotides are useful either as primers for use in various processes such as DNA amplification and microsequencing or as probes for DNA recognition in hybridization analyses. Polynucleotides derived from the TBC-1 gene are useful in order to detect the presence of at least a copy of a nucleotide sequence of SEQ ID Nos 1-4, or a fragment, complement, or variant thereof in a test sample.

Particularly preferred probes and primers of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of a nucleotide sequence selected from the group consisting of SEQ ID Nos 1 and 2, or the complements thereof. Additionally preferred probes and primers of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID No 1 or the complements thereof, wherein said contiguous span comprises at least 1, 2, 3, 5, or 10 of the following nucleotide positions of SEQ ID No 1: 1-1000, 1001-2000, 2001-3000, 3001-4000, 4001-5000, 5001-6000, 6001-7000, 7001-8000, 8001-9000, 9001-10000, 10001-11000, 11001-12000, 12001-13000, 13001-14000, 14001-15000, 15001-16000, 16001-17000, and 17001-17590. Other preferred probes and primers of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID No 2 or the complements thereof, wherein said contiguous span comprises at least 1, 2, 3, 5, or 10 of the following nucleotide positions of SEQ ID No 2: 1-5000, 5001-10000, 10001-15000, 15001-20000, 20001-25000, 25001-30000, 30001-35000, 35001-40000, 40001-45000, 45001-50000, 50001-55000, 55001-60000, 60001-65000, 65001-70000, 70001-75000, 75001-80000, 80001-85000, 85001-90000, 90001-95000, and 95001-99960.

Moreover, preferred probes and primers of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of a nucleotide sequence selected from the group consisting of SEQ ID Nos 3 and 4, or the complements thereof. Particularly preferred probes and primers of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID No 3 or the complements thereof, wherein said contiguous span comprises at least 1, 2, 3, 5, or 10 of the following nucleotide positions of SEQ ID No 3: 1-500, 501-1000, 1001-1500, 1501-2000, 2001-2500, 2501-3000, 3001-3500, and 3501-3983. Additional preferred probes and primers of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID No 4 or the complements thereof, wherein said contiguous span comprises at least 1, 2, 3, 5, or 10 of the following nucleotide positions of SEQ ID No 4: 1-500, 501-1000, 1001-1500, 1501-2000, 2001-2500, 2501-3000, 3001-3500, and 3501-3988.

Thus, the invention also relates to nucleic acid probes characterized in that they hybridize specifically, under the stringent hybridization conditions defined above, with a nucleic acid selected from the group consisting of the nucleotide sequences of SEQ ID Nos 1-4 or a variant thereof or a sequence complementary thereto.

In one embodiment the invention encompasses isolated, purified, and recombinant polynucleotides consisting of, or consisting essentially of a contiguous span of 8 to 50 nucleotides of any one of SEQ ID Nos 1 and 2 and the complement thereof, wherein said span includes a TBC-1-related biallelic marker in said sequence; optionally, wherein said TBC-1-related biallelic marker is selected from the group consisting of A1 to A19, and the complements thereof, or optionally the biallelic markers in linkage disequilibrium therewith; optionally, wherein said contiguous span is 18 to 35 nucleotides in length and said biallelic marker is within 4 nucleotides of the center of said polynucleotide; optionally, wherein said polynucleotide consists of said contiguous span and said contiguous span is 25 nucleotides in length and said biallelic marker is at the center of said polynucleotide; optionally, wherein the 3′ end of said contiguous span is present at the 3′ end of said polynucleotide; and optionally, wherein the 3′ end of said contiguous span is located at the 3′ end of said polynucleotide and said biallelic marker is present at the 3′ end of said polynucleotide. In a preferred embodiment, said probes comprises, consists of, or consists essentially of a sequence selected from the following sequences: P1 to P7, P9 to P13, P15 to P19 and the complementary sequences thereto.

In another embodiment the invention encompasses isolated, purified and recombinant polynucleotides comprising, consisting of, or consisting essentially of a contiguous span of 8 to 50 nucleotides of SEQ ID Nos 1 and 2, or the complements thereof, wherein the 3′ end of said contiguous span is located at the 3′ end of said polynucleotide, and wherein the 3′ end of said polynucleotide is located within 20 nucleotides upstream of a TBC-1-related biallelic marker in said sequence; optionally, wherein said TBC-1-related biallelic marker is selected from the group consisting of A1 to A19, and the complements thereof, or optionally the biallelic markers in linkage disequilibrium therewith; optionally, wherein the 3′ end of said polynucleotide is located 1 nucleotide upstream of said TBC-1-related biallelic marker in said sequence; and optionally, wherein said polynucleotide consists essentially of a sequence selected from the following sequences: D1 to D19 and E1 to E19.

In a further embodiment, the invention encompasses isolated, purified, or recombinant polynucleotides comprising, consisting of, or consisting essentially of a sequence selected from the following sequences: B1 to B15 and C1 to C15.

In an additional embodiment, the invention encompasses polynucleotides for use in hybridization assays, sequencing assays, and enzyme-based mismatch detection assays for determining the identity of the nucleotide at a TBC-1-related biallelic marker in SEQ ID Nos 1 and 2, or the complements thereof, as well as polynucleotides for use in amplifying segments of nucleotides comprising a TBC-1-related biallelic marker in SEQ ID Nos 1 and 2, or the complements thereof; optionally, wherein said TBC-1-related biallelic marker is selected from the group consisting of A1 to A19, and the complements thereof, or optionally the biallelic markers in linkage disequilibrium therewith.

A probe or a primer according to the invention has between 8 and 1000 nucleotides in length, or is specified to be at least 12, 15, 18, 20, 25, 35, 40, 50, 60, 70, 80, 100, 250, 500 or 1000 nucleotides in length. More particularly, the length of these probes and primers can range from 8, 10, 15, 20, or 30 to 100 nucleotides, preferably from 10 to 50, more preferably from 15 to 30 nucleotides. Shorter probes and primers tend to lack specificity for a target nucleic acid sequence and generally require cooler temperatures to form sufficiently stable hybrid complexes with the template. Longer probes and primers are expensive to produce and can sometimes self-hybridize to form hairpin structures. The appropriate length for primers and probes under a particular set of assay conditions may be empirically determined by one of skill in the art. A preferred probe or primer consists of a nucleic acid comprising a polynucleotide selected from the group of the nucleotide sequences of P1 to P7, P9 to P13, P15 to P19 and the complementary sequence thereto, B1 to B15, C1 to C15, D1 to D19, E1 to E19, for which the respective locations in the sequence listing are provided in Tables 2, 3 and 4.

The formation of stable hybrids depends on the melting temperature (Tm) of the DNA. The Tm depends on the length of the primer or probe, the ionic strength of the solution and the G+C content. The higher the G+C content of the primer or probe, the higher is the melting temperature because G:C pairs are held by three H bonds whereas A:T pairs have only two. The GC content in the probes of the invention usually ranges between 10 and 75%, preferably between 35 and 60%, and more preferably between 40 and 55%.

The primers and probes can be prepared by any suitable method, including, for example, cloning and restriction of appropriate sequences and direct chemical synthesis by a method such as the phosphodiester method of Narang et al. (1979), the phosphodiester method of Brown et al. (1979), the diethylphosphoramidite method of Beaucage et al. (1981) and the solid support method described in EP 0 707 592.

Detection probes are generally nucleic acid sequences or uncharged nucleic acid analogs such as, for example peptide nucleic acids which are disclosed in International Patent Application WO 92/20702, morpholino analogs which are described in U.S. Pat. Nos. 5,185,444; 5,034,506 and 5,142,047. The probe may have to be rendered “non-extendable” in that additional dNTPs cannot be added to the probe. In and of themselves analogs usually are non-extendable and nucleic acid probes can be rendered non-extendable by modifying the 3′ end of the probe such that the hydroxyl group is no longer capable of participating in elongation. For example, the 3′ end of the probe can be functionalized with the capture or detection label to thereby consume or otherwise block the hydroxyl group. Alternatively, the 3′ hydroxyl group simply can be cleaved, replaced or modified, U.S. patent application Ser. No. 07/049,061 filed Apr. 19, 1993 describes modifications, which can be used to render a probe non-extendable.

Any of the polynucleotides of the present invention can be labeled, if desired, by incorporating any label known in the art to be detectable by spectroscopic, photochemical, biochemical, immunochemical, or chemical means. For example, useful labels include radioactive substances (including, ³²P, ³⁵S, ³H, ¹²⁵I), fluorescent dyes (including, 5-bromodesoxyuridin, fluorescein, acetylaminofluorene, digoxigenin) or biotin. Preferably, polynucleotides are labeled at their 3′ and 5′ ends. Examples of non-radioactive labeling of nucleic acid fragments are described in the French patent No. FR-7810975 or by Urdea et al (1988) or Sanchez-Pescador et al (1988). In addition, the probes according to the present invention may have structural characteristics such that they allow the signal amplification, such structural characteristics being, for example, branched DNA probes as those described by Urdea et al. in 1991 or in the European patent No. EP 0 225 807 (Chiron).

A label can also be used to capture the primer, so as to facilitate the immobilization of either the primer or a primer extension product, such as amplified DNA, on a solid support. A capture label is attached to the primers or probes and can be a specific binding member which forms a binding pair with the solid's phase reagent's specific binding member (e.g. biotin and streptavidin). Therefore depending upon the type of label carried by a polynucleotide or a probe, it may be employed to capture or to detect the target DNA. Further, it will be understood that the polynucleotides, primers or probes provided herein, may, themselves, serve as the capture label. For example, in the case where a solid phase reagent's binding member is a nucleic acid sequence, it may be selected such that it binds a complementary portion of a primer or probe to thereby immobilize the primer or probe to the solid phase. In cases where a polynucleotide probe itself serves as the binding member, those skilled in the art will recognize that the probe will contain a sequence or “tail” that is not complementary to the target. In the case where a polynucleotide primer itself serves as the capture label, at least a portion of the primer will be free to hybridize with a nucleic acid on a solid phase. DNA Labeling techniques are well known to the skilled technician.

The probes of the present invention are useful for a number of purposes. They can be notably used in Southern hybridization to genomic DNA. The probes can also be used to detect PCR amplification products. They may also be used to detect mismatches in the TBC-1 gene or mRNA using other techniques.

Any of the polynucleotides, primers and probes of the present invention can be conveniently immobilized on a solid support. Solid supports are known to those skilled in the art and include the walls of wells of a reaction tray, test tubes, polystyrene beads, magnetic beads, nitrocellulose strips, membranes, microparticles such as latex particles, sheep (or other animal) red blood cells, duracytes and others. The solid support is not critical and can be selected by one skilled in the art. Thus, latex particles, microparticles, magnetic or non-magnetic beads, membranes, plastic tubes, walls of microtiter wells, glass or silicon chips, sheep (or other suitable animal's) red blood cells and duracytes are all suitable examples. Suitable methods for immobilizing nucleic acids on solid phases include ionic, hydrophobic, covalent interactions and the like. A solid support, as used herein, refers to any material which is insoluble, or can be made insoluble by a subsequent reaction. The solid support can be chosen for its intrinsic ability to attract and immobilize the capture reagent. Alternatively, the solid phase can retain an additional receptor which has the ability to attract and immobilize the capture reagent. The additional receptor can include a charged substance that is oppositely charged with respect to the capture reagent itself or to a charged substance conjugated to the capture reagent. As yet another alternative, the receptor molecule can be any specific binding member which is immobilized upon (attached to) the solid support and which has the ability to immobilize the capture reagent through a specific binding reaction. The receptor molecule enables the indirect binding of the capture reagent to a solid support material before the performance of the assay or during the performance of the assay. The solid phase thus can be a plastic, derivatized plastic, magnetic or non-magnetic metal, glass or silicon surface of a test tube, microtiter well, sheet, bead, microparticle, chip, sheep (or other suitable animal's) red blood cells, duracytes® and other configurations known to those of ordinary skill in the art. The polynucleotides of the invention can be attached to or immobilized on a solid support individually or in groups of at least 2, 5, 8, 10, 12, 15, 20, or 25 distinct polynucleotides of the invention to a single solid support. In addition, polynucleotides other than those of the invention may be attached to the same solid support as one or more polynucleotides of the invention.

Consequently, the invention also deals with a method for detecting the presence of a nucleic acid comprising a nucleotide sequence selected from a group consisting of SEQ ID Nos 1-4, a fragment or a variant thereof and a complementary sequence thereto in a sample, said method comprising the following steps of:

a) bringing into contact a nucleic acid probe or a plurality of nucleic acid probes which can hybridize with a nucleotide sequence included in a nucleic acid selected form the group consisting of the nucleotide sequences of SEQ ID Nos 1-4, a fragment or a variant thereof and a complementary sequence thereto and the sample to be assayed; and

b) detecting the hybrid complex formed between the probe and a nucleic acid in the sample.

The invention further concerns a kit for detecting the presence of a nucleic acid comprising a nucleotide sequence selected from a group consisting of SEQ ID Nos 1-4, a fragment or a variant thereof and a complementary sequence thereto in a sample, said kit comprising:

a) a nucleic acid probe or a plurality of nucleic acid probes which can hybridize with a nucleotide sequence included in a nucleic acid selected form the group consisting of the nucleotide sequences of SEQ ID Nos 1-4, a fragment or a variant thereof and a complementary sequence thereto; and

b) optionally, the reagents necessary for performing the hybridization reaction.

In a first preferred embodiment of this detection method and kit, said nucleic acid probe or the plurality of nucleic acid probes are labeled with a detectable molecule. In a second preferred embodiment of said method and kit, said nucleic acid probe or the plurality of nucleic acid probes has been immobilized on a substrate. In a third preferred embodiment, the nucleic acid probe or the plurality of nucleic acid probes comprise either a sequence which is selected from the group consisting of the nucleotide sequences of P1 to P7, P9 to P13, P15 to P19 and the complementary sequence thereto, B1 to B15, C1 to C15, D1 to D19, E1 to E19 or a biallelic marker selected from the group consisting of A1 to A19 and the complements thereto.

Oligonucleotide Arrays

A substrate comprising a plurality of oligonucleotide primers or probes of the invention may be used either for detecting or amplifying targeted sequences in the TBC-1 gene and may also be used for detecting mutations in the coding or in the non-coding sequences of the TBC-1 gene.

Any polynucleotide provided herein may be attached in overlapping areas or at random locations on the solid support. Alternatively the polynucleotides of the invention may be attached in an ordered array wherein each polynucleotide is attached to a distinct region of the solid support which does not overlap with the attachment site of any other polynucleotide. Preferably, such an ordered array of polynucleotides is designed to be “addressable” where the distinct locations are recorded and can be accessed as part of an assay procedure. Addressable polynucleotide arrays typically comprise a plurality of different oligonucleotide probes that are coupled to a surface of a substrate in different known locations. The knowledge of the precise location of each polynucleotides location makes these “addressable” arrays particularly useful in hybridization assays. Any addressable array technology known in the art can be employed with the polynucleotides of the invention. One particular embodiment of these polynucleotide arrays is known as the Genechips™, and has been generally described in U.S. Pat. No. 5,143,854; PCT publications WO 90/15070 and 92/10092. These arrays may generally be produced using mechanical synthesis methods or light directed synthesis methods which incorporate a combination of photolithographic methods and solid phase oligonucleotide synthesis (Fodor et al., 1991). The immobilization of arrays of oligonucleotides on solid supports has been rendered possible by the development of a technology generally identified as “Very Large Scale Immobilized Polymer Synthesis” (VLSIPS™) in which, typically, probes are immobilized in a high density array on a solid surface of a chip. Examples of VLSIPS™ technologies are provided in U.S. Pat. Nos. 5,143,854; and 5,412,087 and in PCT Publications WO 90/15070, WO 92/10092 and WO 95/11995, which describe methods for forming oligonucleotide arrays through techniques such as light-directed synthesis techniques. In designing strategies aimed at providing arrays of nucleotides immobilized on solid supports, further presentation strategies were developed to order and display the oligonucleotide arrays on the chips in an attempt to maximize hybridization patterns and sequence information. Examples of such presentation strategies are disclosed in PCT Publications WO 94/12305, WO 94/11530, WO 97/29212 and WO 97/31256.

In another embodiment of the oligonucleotide arrays of the invention, an oligonucleotide probe matrix may advantageously be used to detect mutations occurring in the TBC-1 gene and preferably in its regulatory region. For this particular purpose, probes are specifically designed to have a nucleotide sequence allowing their hybridization to the genes that carry known mutations (either by deletion, insertion or substitution of one or several nucleotides). By known mutations, it is meant, mutations on the TBC-1 gene that have been identified according, for example to the technique used by Huang et al. (1996) or Samson et al. (1996).

Another technique that is used to detect mutations in the TBC-1 gene is the use of a high-density DNA array. Each oligonucleotide probe constituting a unit element of the high density DNA array is designed to match a specific subsequence of the TBC-1 genomic DNA or cDNA. Thus, an array consisting of oligonucleotides complementary to subsequences of the target gene sequence is used to determine the identity of the target sequence with the wild gene sequence, measure its amount, and detect differences between the target sequence and the reference wild gene sequence of the TBC-1 gene. In one such design, termed 4L tiled array, is implemented a set of four probes (A, C, G, T), preferably 15-nucleotide oligomers. In each set of four probes, the perfect complement will hybridize more strongly than mismatched probes. Consequently, a nucleic acid target of length L is scanned for mutations with a tiled array containing 4L probes, the whole probe set containing all the possible mutations in the known wild reference sequence. The hybridization signals of the 15-mer probe set tiled array are perturbed by a single base change in the target sequence. As a consequence, there is a characteristic loss of signal or a “footprint” for the probes flanking a mutation position. This technique was described by Chee et al. in 1996.

Consequently, the invention concerns an array of nucleic acid molecules comprising at least one polynucleotide described above as probes and primers. Preferably, the invention concerns an array of nucleic acid comprising at least two polynucleotides described above as probes and primers.

A further object of the invention consists of an array of nucleic acid sequences comprising either at least one of the sequences selected from the group consisting of P1 to P7, P9 to P13, P15 to P19, B1 to B15, C1 to C15, D1 to D19, E1 to E19, the sequences complementary thereto, a fragment thereof of at least 8, 10, 12, 15, 18, 20, 25, 30, or 40 consecutive nucleotides thereof, and at least one sequence comprising a biallelic marker selected from the group consisting of A1 to A19 and the complements thereto.

The invention also pertains to an array of nucleic acid sequences comprising either at least two of the sequences selected from the group consisting of P1 to P7, P9 to P13, P15 to P19, B1 to B15, C1 to C15, D1 to D19, E1 to E19, the sequences complementary thereto, a fragment thereof of at least 8 consecutive nucleotides thereof, and at least two sequences comprising a biallelic marker selected from the group consisting of A1 to A19 and the complements thereof.

### Vectors for the Expression of a Regulatory or a Coding Polynucleotide of TBC-1

Any of the regulatory polynucleotides or the coding polynucleotides of the invention may be inserted into recombinant vectors for expression in a recombinant host cell or a recombinant host organism.

Thus, the present invention also encompasses a family of recombinant vectors that contains either a regulatory polynucleotide selected from the group consisting of any one of the regulatory polynucleotides derived from the TBC-1 genomic sequences of SEQ ID Nos 1 and 2, or a polynucleotide comprising the TBC-1 coding sequence, or both.

In a first preferred embodiment, a recombinant vector of the invention is used as an expression vector: (a) the TBC-1 regulatory sequence comprised therein drives the expression of a coding polynucleotide operably linked thereto; (b) the TBC-1 coding sequence is operably linked to regulation sequences allowing its expression in a suitable cell host and/or host organism.

In a second preferred embodiment, a recombinant vector of the invention is used to amplify the inserted polynucleotide derived from the TBC-1 genomic sequences of SEQ ID Nos 1 and 2 or TBC-1 cDNAs in a suitable cell host, this polynucleotide being amplified at every time that the recombinant vector replicates.

More particularly, the present invention relates to expression vectors which include nucleic acids encoding a TBC-1 protein, preferably the TBC-1 protein of the amino acid sequence of SEQ ID No 5 described therein, under the control of a regulatory sequence selected among the TBC-1 regulatory polynucleotides, or alternatively under the control of an exogenous regulatory sequence.

A recombinant expression vector comprising a nucleic acid selected from the group consisting of 5′ and 3′ regulatory regions, or biologically active fragments or variants thereof, is also part of the present invention.

The invention also encompasses a recombinant expression vector comprising:

a) a nucleic acid comprising the 5′ regulatory polynucleotide of the nucleotide sequence SEQ ID No 1, or a biologically active fragment or variant thereof;

b) a polynucleotide encoding a polypeptide or a polynucleotide of interest operably linked with said nucleic acid.

c) optionally, a nucleic acid comprising a 3′-regulatory polynucleotide, preferably a 3′-regulatory polynucleotide of the invention, or a biologically active fragment or variant thereof.

The nucleic acid comprising the 5′ regulatory polynucleotide or a biologically active fragment or variant thereof may also comprises the 5′-UTR sequence from any of the two cDNA of the invention or a biologically active fragment or variant thereof.

The invention also pertains to a recombinant expression vector useful for the expression of the TBC-1 coding sequence, wherein said vector comprises a nucleic acid selected from the group consisting of SEQ ID Nos 3 and 4 or a nucleic acid having at least 95% nucleotide identity with a polynucleotide selected from the group consisting of the nucleotide sequences of SEQ ID Nos 3 and 4.

Another recombinant expression vector of the invention consists in a recombinant vector comprising a nucleic acid comprising the nucleotide sequence beginning at the nucleotide in position 176 and ending in position 3730 of the polynucleotide of SEQ ID No 4.

Generally, a recombinant vector of the invention may comprise any of the polynucleotides described herein, including regulatory sequences, and coding sequences, as well as any TBC-1 primer or probe as defined above. More particularly, the recombinant vectors of the present invention can comprise any of the polynucleotides described in the “TBC-1 cDNA Sequences” section, the “Coding Regions” section, “Genomic sequence of TBC-1” section and the “Oligonucleotide Probes And Primers” section.

Some of the elements which can be found in the vectors of the present invention are described in further detail in the following sections.

a) Vectors

A recombinant vector according to the invention comprises, but is not limited to, a YAC (Yeast Artificial Chromosome), a BAC (Bacterial Artificial Chromosome), a phage, a phagemid, a cosmid, a plasmid or even a linear DNA molecule which may consist of a chromosomal, non-chromosomal and synthetic DNA. Such a recombinant vector can comprise a transcriptional unit comprising an assembly of:

(1) a genetic element or elements having a regulatory role in gene expression, for example promoters or enhancers. Enhancers are cis-acting elements of DNA, usually from about 10 to 300 bp in length that act on the promoter to increase the transcription.

(2) a structural or coding sequence which is transcribed into mRNA and eventually translated into a polypeptide, and

(3) appropriate transcription initiation and termination sequences. Structural units intended for use in yeast or eukaryotic expression systems preferably include a leader sequence enabling extracellular secretion of translated protein by a host cell. Alternatively, where a recombinant protein is expressed without a leader or transport sequence, it may include an N-terminal residue. This residue may or may not be subsequently cleaved from the expressed recombinant protein to provide a final product.

Generally, recombinant expression vectors will include origins of replication, selectable markers permitting transformation of the host cell, and a promoter derived from a highly expressed gene to direct transcription of a downstream structural sequence. The heterologous structural sequence is assembled in appropriate phase with translation initiation and termination sequences, and preferably a leader sequence capable of directing secretion of the translated protein into the periplasmic space or the extracellular medium.

The selectable marker genes for selection of transformed host cells are preferably dihydrofolate reductase or neomycin resistance for eukaryotic cell culture, TRP1 for S. cerevisiae or tetracycline, rifampicin or ampicillin resistance in E. coli, or levan saccharase for mycobacteria.

As a representative but non-limiting example, useful expression vectors for bacterial use can comprise a selectable marker and a bacterial origin of replication derived from commercially available plasmids comprising genetic elements of pBR322 (ATCC 37017). Such commercial vectors include, for example, pKK223-3 (Pharmacia, Uppsala, Sweden), and GEM1 (Promega Biotec, Madison, Wis., USA).

Large numbers of suitable vectors and promoters are known to those of skill in the art, and commercially available, such as bacterial vectors: pQE70, pQE60, pQE-9 (Qiagen), pbs, pD10, phagescript, psiX174, pbluescript SK, pbsks, pNH8A, pNH16A, pNH18A, pNH46A (Stratagene); ptrc99a, pKK223-3, pKK233-3, pDR540, pRIT5 (Pharmacia); or eukaryotic vectors: pWLNEO, pSV2CAT, pOG44, pXT1, pSG (Stratagene); pSVK3, pBPV, pMSG, pSVL (Pharmacia); baculovirus transfer vector pVL1392/1393 (Pharmingen); pQE-30 (QIAexpress).

A suitable vector for the expression of the TBC-1 polypeptide of SEQ ID No 5 is a baculovirus vector that can be propagated in insect cells and in insect cell lines. A specific suitable host vector system is the pVL1392/1393 baculovirus transfer vector (Pharmingen) that is used to transfect the SF9 cell line (ATCC N^{o}CRL 1711) which is derived from Spodoptera frugiperda.

Other suitable vectors for the expression of the TBC-1 polypeptide of SEQ ID No 5 in a baculovirus expression system include those described by Chai et al. (1993), Vlasak et al. (1983) and Lenhard et al. (1996).

Mammalian expression vectors will comprise an origin of replication, a suitable promoter and enhancer, and also any necessary ribosome binding sites, polyadenylation site, splice donor and acceptor sites, transcriptional termination sequences, and 5′ flanking nontranscribed sequences. DNA sequences derived from the SV40 viral genome, for example SV40 origin, early promoter, enhancer, splice and polyadenylation sites may be used to provide the required nontranscribed genetic elements.

b) Promoters

The suitable promoter regions used in the expression vectors according to the present invention are chosen taking into account the cell host in which the heterologous gene has to be expressed.

A suitable promoter may be heterologous with respect to the nucleic acid for which it controls the expression or alternatively can be endogenous to the native polynucleotide containing the coding sequence to be expressed. Additionally, the promoter is generally heterologous with respect to the recombinant vector sequences within which the construct promoter/coding sequence has been inserted.

Preferred bacterial promoters are the LacI, LacZ, the T3 or T7 bacteriophage RNA polymerase promoters, the polyhedrin promoter, or the p10 protein promoter from baculovirus (Kit Novagen) (Smith et al., 1983; O'Reilly et al., 1992), the lambda P_{R} promoter or also the trc promoter.

Promoter regions can be selected from any desired gene using, for example, CAT (chloramphenicol transferase) vectors and more preferably pKK232-8 and pCM7 vectors. Particularly preferred bacterial promoters include lacI, lacZ, T3, T7, gpt, lambda PR, PL and trp. Eukaryotic promoters include CMV immediate early, HSV thymidine kinase, early and late SV40, LTRs from retrovirus, and mouse metallothionein-L. Selection of a convenient vector and promoter is well within the level of ordinary skill in the art.

The choice of a promoter is well within the ability of a person skilled in the field of genetic egineering. For example, one may refer to the book of Sambrook et al. (1989) or also to the procedures described by Fuller et al. (1996).

The vector containing the appropriate DNA sequence as described above, more preferably a TBC-1 gene regulatory polynucleotide, a polynucleotide encoding the TBC-1 polypeptide of SEQ ID No 5 or both of them, can be utilized to transform an appropriate host to allow the expression of the desired polypeptide or polynucleotide.

c) Other Types of Vectors

The in vivo expression of a TBC-1 polypeptide of SEQ ID No 5 may be useful in order to correct a genetic defect related to the expression of the native gene in a host organism or to the production of a biologically inactive TBC-1 protein.

Consequently, the present invention also deals with recombinant expression vectors mainly designed for the in vivo production of the TBC-1 polypeptide of SEQ ID No 5 by the introduction of the appropriate genetic material in the organism of the patient to be treated. This genetic material may be introduced in vitro in a cell that has been previously extracted from the organism, the modified cell being subsequently reintroduced in the said organism, directly in vivo into the appropriate tissue.

By <<vector >> according to this specific embodiment of the invention is intended either a circular or a linear DNA molecule.

One specific embodiment for a method for delivering a protein or peptide to the interior of a cell of a vertebrate in vivo comprises the step of introducing a preparation comprising a physiologically acceptable carrier and a naked polynucleotide operatively coding for the polypeptide of interest into the interstitial space of a tissue comprising the cell, whereby the naked polynucleotide is taken up into the interior of the cell and has a physiological effect.

In a specific embodiment, the invention provides a composition for the in vivo production of the TBC-1 protein or polypeptide described herein. It comprises a naked polynucleotide operatively coding for this polypeptide, in solution in a physiologically acceptable carrier, and suitable for introduction into a tissue to cause cells of the tissue to express the said protein or polypeptide.

Compositions comprising a polynucleotide are described in PCT application N^{o} WO 90/11092 (Vical Inc.) and also in PCT application N^{o} WO 95/11307 (Institut Pasteur, INSERM, Université d'Ottawa) as well as in the articles of Tacson et al. (1996) and of Huygen et al. (1996).

The amount of vector to be injected to the desired host organism varies according to the site of injection. As an indicative dose, it will be injected between 0.1 and 100 μg of the vector in an animal body, preferably a mammal body, for example a mouse body.

In another embodiment of the vector according to the invention, it may be introduced in vitro in a host cell, preferably in a host cell previously harvested from the animal to be treated and more preferably a somatic cell such as a muscle cell. In a subsequent step, the cell that has been transformed with the vector coding for the desired TBC-1 polypeptide or the desired fragment thereof is reintroduced into the animal body in order to deliver the recombinant protein within the body either locally or systemically.

In one specific embodiment, the vector is derived from an adenovirus. Preferred adenovirus vectors according to the invention are those described by Feldman and Steg (1996) or Ohno et al. (1994). Another preferred recombinant adenovirus according to this specific embodiment of the present invention is the human adenovirus type 2 or 5 (Ad 2 or Ad 5) or an adenovirus of animal origin (French patent application N^{o} FR-93.05954).

Retrovirus vectors and adeno-associated virus vectors are generally understood to be the recombinant gene delivery systems of choice for the transfer of exogenous polynucleotides in vivo, particularly to mammals, including humans. These vectors provide efficient delivery of genes into cells, and the transferred nucleic acids are stably integrated into the chromosomal DNA of the host

Particularly preferred retroviruses for the preparation or construction of retroviral in vitro or in vitro gene delivery vehicles of the present invention include retroviruses selected from the group consisting of Mink-Cell Focus Inducing Virus, Murine Sarcoma Virus, Reticuloendotheliosis virus and Rous Sarcoma virus. Particularly preferred Murine Leukemia Viruses include the 4070A and the 1504A viruses, Abelson (ATCC No VR-999), Friend (ATCC No VR-245), Gross (ATCC No VR-590), Rauscher (ATCC No VR-998) and Moloney Murine Leukemia Virus (ATCC No VR-190; PCT Application No WO 94/24298). Particularly preferred Rous Sarcoma Viruses include Bryan high titer (ATCC Nos VR-334, VR-657, VR-726, VR-659 and VR-728). Other preferred retroviral vectors are those described in Roth et al. (Roth J. A. et al., 1996), PCT Application No WO 93/25234, PCT Application No WO 94/06920, Roux et al., 1989, Julan et al., 1992 and Neda et al., 1991.

Yet another viral vector system that is contemplated by the invention consists in the adeno-associated virus (AAV). The adeno-associated virus is a naturally occurring defective virus that requires another virus, such as an adenovirus or a herpes virus, as a helper virus for efficient replication and a productive life cycle (Muzyczka et al., 1992). It is also one of the few viruses that may integrate its DNA into non-dividing cells, and exhibits a high frequency of stable integration (Flotte et al., 1992; Samulski et al., 1989; McLaughlin et al., 1989). One advantageous feature of AAV derives from its reduced efficacy for transducing primary cells relative to transformed cells.

Other compositions containing a vector of the invention advantageously comprise an oligonucleotide fragment of a nucleic sequence selected from the group consisting of SEQ ID Nos 3 or 4 as an antisense tool that inhibits the expression of the corresponding TBC-1 gene. Preferred methods using antisense polynucleotide according to the present invention are the procedures described by Sczakiel et al. (1995) or those described in PCT Application No WO 95/24223.

### Host Cells

Another object of the invention consists in host cell that have been transformed or transfected with one of the polynucleotides described therein, and more precisely a polynucleotide either comprising a TBC-1 regulatory polynucleotide or the coding sequence of the TBC-1 polypeptide having the amino acid sequence of SEQ ID No 5. Are included host cells that are transformed (prokaryotic cells) or that are transfected (eukaryotic cells) with a recombinant vector such as one of those described above.

A recombinant host cell of the invention comprises any one of the polynucleotides or the recombinant vectors described therein. More particularly, the cell hosts of the present invention can comprise any of the polynucleotides described in “TBC-1 cDNA Sequences” section, the “Coding Regions” section, “Genomic sequence of TBC-1” section and the “Oligonucleotide Probes And Primers” section.

Another preferred recombinant cell host according to the present invention is characterized in that its genome or genetic background (including chromosome, plasmids) is modified by the nucleic acid coding for the TBC-1 polypeptide of SEQ ID No 5.

Preferred host cells used as recipients for the expression vectors of the invention are the following:

a) Prokaryotic host cells: Escherichia coli strains (I.E. DH5-α strain) or Bacillus subtilis.

b) Eukaryotic host cells: HeLa cells (ATCC N^{o}CCL2; N^{o}CCL2.1; N^{o}CCL2.2), Cv 1 cells (ATCC N^{o}CCL70), COS cells (ATCC N^{o}CRL1650; N^{o}CRL1651), Sf-9 cells (ATCC N^{o}CRL1711).

The constructs in the host cells can be used in a conventional manner to produce the gene product encoded by the recombinant sequence.

Following transformation of a suitable host and growth of the host to an appropriate cell density, the selected promoter is induced by appropriate means, such as temperature shift or chemical induction, and cells are cultivated for an additional period.

Cells are typically harvested by centrifugation, disrupted by physical or chemical means, and the resulting crude extract retained for further purification.

Microbial cells employed in the expression of proteins can be disrupted by any convenient method, including freeze-thaw cycling, sonication, mechanical disruption, or use of cell lysing agents. Such methods are well known by the skill artisan.

### Transgenic Animals

The terms “transgenic animals” or “host animals” are used herein to designate animals that have their genome genetically and artificially manipulated so as to include one of the nucleic acids according to the invention. Preferred animals are non-human mammals and include those belonging to a genus selected from Mus (e.g. mice), Rattus (e.g. rats) and Oryctogalus (e.g. rabbits) which have their genome artificially and genetically altered by the insertion of a nucleic acid according to the invention.

The transgenic animals of the invention all include within a plurality of their cells a cloned recombinant or synthetic DNA sequence, more specifically one of the purified or isolated nucleic acids comprising a TBC-1 coding sequence, a TBC-1 regulatory polynucleotide or a DNA sequence encoding an antisense polynucleotide such as described in the present specification.

More particularly, transgenic animals according to the invention contain in their somatic cells and/or in their germ line cells any of the polynucleotides described in “TBC-1 cDNA Sequences” section, the “Coding Regions” section, “Genomic sequence of TBC-1” section, the “Oligonucleotide Probes And Primers” section and the “Vectors for the expression of a regulatory or coding polynucleotide of TBC-1” section.

The transgenic animals of the invention thus contain specific sequences of exogenous genetic material such as the nucleotide sequences described above in detail.

In a first preferred embodiment, these transgenic animals may be good experimental models in order to study the diverse pathologies related to cell differentiation, in particular concerning the transgenic animals within the genome of which has been inserted one or several copies of a polynucleotide encoding a native TBC-1 protein, or alternatively a mutant TBC-1 protein.

In a second preferred embodiment, these transgenic animals may express a desired polypeptide of interest under the control of the regulatory polynucleotides of the TBC-1 gene, leading to good yields in the synthesis of this protein of interest, and eventually a tissue specific expression of this protein of interest.

Since it is possible to produce transgenic animals of the invention using a variety of different sequences, a general description will be given of the production of transgenic animals by referring generally to exogenous genetic material. This general description can be adapted by those skilled in the art in order to incorporate the DNA sequences into animals. For more details regarding the production of transgenic animals, and specifically transgenic mice, it may be referred to Sandou et al. (1994) and also to U.S. Pat. No. 4,873,191, issued Oct. 10, 1989, U.S. Pat. No. 5,968,766, issued Dec. 16, 1997 and U.S. Pat. No. 5,387,742, issued Feb. 28, 1995, these documents being herein incorporated by reference to disclose methods for producing transgenic mice.

Transgenic animals of the present invention are produced by the application of procedures which result in an animal with a genome that incorporates exogenous genetic material which is integrated into the genome. The procedure involves obtaining the genetic material, or a portion thereof, which encodes either a TBC-1 coding sequence, a TBC-1 regulatory polynucleotide or a DNA sequence encoding an antisense polynucleotide such as described in the present specification.

A recombinant polynucleotide of the invention is inserted into an embryonic or ES stem cell line. The insertion is made using electroporation. The cells subjected to electroporation are screened (e.g. Southern blot analysis) to find positive cells which have integrated the exogenous recombinant polynucleotide into their genome. An illustrative positive-negative selection procedure that may be used according to the invention is described by Mansour et al. (1988). Then, the positive cells are isolated, cloned and injected into 3.5 days old blastocysts from mice. The blastocysts are then inserted into a female host animal and allowed to grow to term. The offsprings of the female host are tested to determine which animals are transgenic e.g. include the inserted exogenous DNA sequence and which are wild-type.

### Screening of Agents Interacting with TBC-1

In a further embodiment, the present invention also concerns a method for the screening of new agents, or candidate substances interacting with TBC-1. These new agents could be useful against cancer.

In a preferred embodiment, the invention relates to a method for the screening of candidate substances comprising the following steps:

providing a cell line, an organ, or a mammal expressing a TBC-1 gene or a fragment thereof, preferably the regulatory region or the promoter region of the TBC-1 gene.

obtaining a candidate substance preferably a candidate substance capable of inhibiting the binding of a transcription factor to the TBC-1 regulatory region,

testing the ability of the candidate substance to decrease the symptoms of prostate cancer and/or to modulate the expression levels of TBC-1.

In some embodiments, the cell line, organ or mammal expresses a heterologous protein, the coding sequence of which is operably linked to the TBC-1 regulatory or promoter sequence. In other embodiments, they express a TBC-1 gene comprising alleles of one or more TBC-1-related biallelic markers.

A candidate substance is a substance which can interact with or modulate, by binding or other intramolecular interactions, expression, stability, and function of TBC-1. Such substances may be potentially interesting for patients who are not responsive to existing drugs or develop side effects to them. Screening may be effected using either in vitro methods or in vivo methods.

Such methods can be carried out in numerous ways such as on transformed cells which express the considered alleles of the TBC-1 gene, on tumors induced by said transformed cells, for example in mice, or on a TBC-1 protein encoded by the considered allelic variant of TBC-1.

Screening assays of the present invention generally involve determining the ability of a candidate substance to present a cytotoxic effect, to change the characteristics of transformed cells such as proliferative and invasive capacity, to affect the tumor growth, or to modify the expression level of TBC-1.

Typically, this method includes preparing transformed cells with different forms of TBC-1 sequences containing particular alleles of one or more biallelic markers and/or trait causing mutations described above. This is followed by testing the cells expressing the TBC-1 with a candidate substance to determine the ability of the substance to present cytotoxic effect, to affect the characteristics of transformed cells, the tumor growth, or to modify the expression level of TBC-1.

Typical examples of such drug screening assays are provided below. It is to be understood that the parameters set forth in these examples can be modified by the skilled person without undue experimentation.

Methods for Screening Substances Interacting with a TBC-1 Polypeptide

A method for the screening of a candidate substance according to the invention comprises the following steps:

a) providing a polypeptide comprising the amino acid sequence SEQ ID No 5, or a peptide fragment or a variant thereof;

b) obtaining a candidate substance;

c) bringing into contact said polypeptide with said candidate substance;

d) detecting the complexes formed between said polypeptide and said candidate substance.

For the purpose of the present invention, a ligand means a molecule, such as a protein, a peptide, an antibody or any synthetic chemical compound capable of binding to the TBC-1 protein or one of its fragments or variants or to modulate the expression of the polynucleotide coding for TBC-1 or a fragment or variant thereof.

In the ligand screening method according to the present invention, a biological sample or a defined molecule to be tested as a putative ligand of the TBC-1 protein is brought into contact with a purified TBC-1 protein, for example a purified recombinant TBC-1 protein produced by a recombinant cell host as described hereinbefore, in order to form a complex between the TBC-1 protein and the putative ligand molecule to be tested.

A. Candidate Ligands Obtained Form Random Peptide Libraries

In a particular embodiment of the screening method, the putative ligand is the expression product of a DNA insert contained in a phage vector (Parmley and Smith, 1988). Specifically, random peptide phages libraries are used. The random DNA inserts encode peptides of 8 to 20 aminoacids in length (Oldenburg K. R. et al., 1992; Valadon P., et al., 1996; Lucas A. H., 1994; Westerink M. A. J., 1995; Castagnoli L. et al., 1991). According to this particular embodiment, the recombinant phages expressing a protein that binds to the immobilized TBC-1 protein are retained and the complex formed between the TBC-1 protein and the recombinant phage may be subsequently immunoprecipitated by a polyclonal or a monoclonal antibody directed against the TBC-1 protein.

Once the ligand library in recombinant phages has been constructed, the phage population is brought into contact with the immobilized TBC-1 protein. Then the preparation of complexes is washed in order to remove the non-specifically bound recombinant phages. The phages that bind specifically to the TBC-1 protein are then eluted by a buffer (acid pH) or immunoprecipitated by the anti-TBC-1 monoclonal antibody produced by a hybridoma, and this phage population is subsequently amplified by an over-infection of bacteria (for example E. coli). The selection step may be repeated several times, preferably 2-4 times, in order to select the more specific recombinant phage clones. The last step consists in characterizing the peptide produced by the selected recombinant phage clones either by expression in infected bacteria and isolation, expressing the phage insert in another host-vector system, or sequencing the insert contained in the selected recombinant phages.

B. Candidate Ligands Obtained Through a Two-Hybrid Screening Assay.

The yeast two-hybrid system is designed to study protein-protein interactions in vivo (Fields and Song, 1989), and relies upon the fusion of a bait protein to the DNA binding domain of the yeast Gal4 protein. This technique is also described in U.S. Pat. No. 5,667,973 and U.S. Pat. No. 5,283,173 (Fields et al.) the technical teachings of both patents being herein incorporated by reference.

The general procedure of library screening by the two-hybrid assay may be performed as described by Harper et al. (Harper J W et al., 1993) or as described by Cho et al. (1998) or also Fromont-Racine et al. (1997).

The bait protein or polypeptide consists of a TBC-1 polypeptide or a fragment or variant thereof.

More precisely, the nucleotide sequence encoding the TBC-1 polypeptide or a fragment or variant thereof is fused to a polynucleotide encoding the DNA binding domain of the GAL4 protein, the fused nucleotide sequence being inserted in a suitable expression vector, for example pAS2 or pM3.

Then, a human cDNA library is constructed in a specially designed vector, such that the human cDNA insert is fused to a nucleotide sequence in the vector that encodes the transcriptional domain of the GAL4 protein. Preferably, the vector used is the pACT vector. The polypeptides encoded by the nucleotide inserts of the human cDNA library are termed “pray” polypeptides.

A third vector contains a detectable marker gene, such as beta galactosidase gene or CAT gene that is placed under the control of a regulation sequence that is responsive to the binding of a complete Gal4 protein containing both the transcriptional activation domain and the DNA binding domain. For example, the vector pG5EC may be used.

Two different yeast strains are also used. As an illustrative but non limiting example the two different yeast strains may be the following:
Y190, the phenotype of which is (MATa, Leu2-3, 112 ura3-12, trp1-901, his3-D200, ade2-101, gal4Dgal180D URA3 GAL-LacZ, LYS GAL-HIS3, cyh′);
Y187, the phenotype of which is (MATa gal4 gal80 his3 trp1-901 ade2-101 ura3-52 leu2-3, -112 URA3 GAL-lacZmet⁻), which is the opposite mating type of Y190.

Briefly, 20 μg of pAS2/TBC-1 and 20 μg of pACT-cDNA library are co-transformed into yeast strain Y190. The transformants are selected for growth on minimal media lacking histidine, leucine and tryptophan, but containing the histidine synthesis inhibitor 3-AT (50 mM). Positive colonies are screened for beta galactosidase by filter lift assay. The double positive colonies (His⁺, beta-gal⁺) are then grown on plates lacking histidine, leucine, but containing tryptophan and cycloheximide (10 mg/ml) to select for loss of pAS2/TBC-1 plasmids but retention of pACT-cDNA library plasmids. The resulting Y190 strains are mated with Y187 strains expressing TBC-1 or non-related control proteins; such as cyclophilin B, lamin, or SNF1, as Gal4 fusions as described by Harper et al. (1993) and by Bram et al. (1993), and screened for beta galactosidase by filter lift assay. Yeast clones that are beta gal− after mating with the control Gal4 fusions are considered false positives.

In another embodiment of the two-hybrid method according to the invention, the interaction between TBC-1 or a fragment or variant thereof with cellular proteins may be assessed using the Matchmaker Two Hybrid System 2 (Catalog No. K1604-1, Clontech).). As described in the manual accompanying the Matchmaker Two Hybrid System 2 (Catalog No. K1604-1, Clontech), the disclosure of which is incorporated herein by reference, nucleic acids encoding the TBC-1 protein or a portion thereof, are inserted into an expression vector such that they are in frame with DNA encoding the DNA binding domain of the yeast transcriptional activator GAL4. A desired cDNA, preferably human cDNA, is inserted into a second expression vector such that they are in frame with DNA encoding the activation domain of GAL4. The two expression plasmids are transformed into the yeast cells and the yeast cells are plated on selection medium which selects for expression of selectable markers on each of the expression vectors as well as GALA dependent expression of the HIS3 gene. Transformants capable of growing on medium lacking histidine are screened for GAL4 dependent lacZ expression. Those cells which are positive in both the histidine selection and the lacZ assay are those in which an interaction between TBC-1 and the protein or peptide encoded by the initially selected cDNA insert has taken place.

Method for Screening Ligands that Modulate the Expression of the TBC-1 Gene

Another subject of the present invention is a method for screening molecules that modulate the expression of the TBC-1 protein. Such a screening method comprises the steps of:

a) cultivating a prokaryotic or an eukaryotic cell that has been transfected with a nucleotide sequence encoding the TBC-1 protein, operably linked to a TBC-1 5′-regulatory sequence;

b) bringing into contact the cultivated cell with a molecule to be tested;

c) quantifying the expression of the TBC-1 protein.

Using DNA recombination techniques well known by the one skill in the art, the TBC-1 protein encoding DNA sequence is inserted into an expression vector, downstream from a TBC-1 5′-regulatory sequence that contains a TBC-1 promoter sequence.

The quantification of the expression of the TBC-1 protein may be realized either at the mRNA level or at the protein level. In the latter case, polyclonal or monoclonal antibodies may be used to quantify the amounts of the TBC-1 protein that have been produced, for example in an ELISA or a RIA assay.

In a preferred embodiment, the quantification of the TBC-1 mRNAs is realized by a quantitative PCR amplification of the cDNAs obtained by a reverse transcription of the total mRNA of the cultivated TBC-1-transfected host cell, using a pair of primers specific for TBC-1.

Expression levels and patterns of TBC-1 may be analyzed by solution hybridization with long probes as described in International Patent Application No. WO 97/05277, the entire contents of which are incorporated herein by reference. Briefly, the TBC-1 cDNA or the TBC-1 genomic DNA described above, or fragments thereof, is inserted at a cloning site immediately downstream of a bacteriophage (T3, T7 or SP6) RNA polymerase promoter to produce antisense RNA. Preferably, the TBC-1 insert comprises at least 100 or more consecutive nucleotides of the genomic DNA sequence or the cDNA sequences, particularly those comprising one of the nuceotide sequences of SEQ ID Nos 3, 4 and 6-8 or those encoding a mutated TBC-1. The plasmid is linearized and transcribed in the presence of ribonucleotides comprising modified ribonucleotides (i.e. biotin-UTP and DIG-UTP). An excess of this doubly labeled RNA is hybridized in solution with mRNA isolated from cells or tissues of interest. The hybridizations are performed under standard stringent conditions (40-50° C. for 16 hours in an 80% formamide, 0.4 M NaCl buffer, pH 7-8). The unhybridized probe is removed by digestion with ribonucleases specific for single-stranded RNA (i.e. RNases CL3, T1, Phy M, U2 or A). The presence of the biotin-UTP modification enables capture of the hybrid on a microtitration plate coated with streptavidin. The presence of the DIG modification enables the hybrid to be detected and quantified by ELISA using an anti-DIG antibody coupled to alkaline phosphatase.

Quantitative analysis of TBC-1 gene expression may also be performed using arrays. As used herein, the term array means a one dimensional, two dimensional, or multidimensional arrangement of a plurality of nucleic acids of sufficient length to permit specific detection of expression of mRNAs capable of hybridizing thereto. For example, the arrays may contain a plurality of nucleic acids derived from genes whose expression levels are to be assessed. The arrays may include the TBC-1 genomic DNA, the TBC-1 cDNA sequences or the sequences complementary thereto or fragments thereof, particularly those comprising at least one of the biallelic markers according the present invention. Preferably, the fragments are at least 15 nucleotides in length. In other embodiments, the fragments are at least 25 nucleotides in length. In some embodiments, the fragments are at least 50 nucleotides in length. More preferably, the fragments are at least 100 nucleotides in length. In another preferred embodiment, the fragments are more than 100 nucleotides in length. In some embodiments the fragments may be more than 500 nucleotides in length.

For example, quantitative analysis of TBC-1 gene expression may be performed with a complementary DNA microarray as described by Schena et al. (1995). Full length TBC-1 cDNAs or fragments thereof are amplified by PCR and arrayed from a 96-well microtiter plate onto silylated microscope slides using high-speed robotics. Printed arrays are incubated in a humid chamber to allow rehydration of the array elements and rinsed, once in 0.2% SDS for 1 min, twice in water for 1 min and once for 5 min in sodium borohydride solution. The arrays are submerged in water for 2 min at 95° C., transferred into 0.2% SDS for 1 min, rinsed twice with water, air dried and stored in the dark at 25° C.

Cell or tissue mRNA is isolated or commercially obtained and probes are prepared by a single round of reverse transcription. Probes are hybridized to 1 cm² microarrays under a 14×14 mm glass coverslip for 6-12 hours at 60° C. Arrays are washed for 5 min at 25° C. in low stringency wash buffer (1×SSC/0.2% SDS), then for 10 min at room temperature in high stringency wash buffer (0.1×SSC/0.2% SDS). Arrays are scanned in 0.1×SSC using a fluorescence laser scanning device fitted with a custom filter set. Accurate differential expression measurements are obtained by taking the average of the ratios of two independent hybridizations.

Quantitative analysis of TBC-1 gene expression may also be performed with full length TBC-1 cDNAs or fragments thereof in complementary DNA arrays as described by Pietu et al. (1996). The full length TBC-1 cDNA or fragments thereof is PCR amplified and spotted on membranes. Then, mRNAs originating from various tissues or cells are labeled with radioactive nucleotides. After hybridization and washing in controlled conditions, the hybridized mRNAs are detected by phospho-imaging or autoradiography. Duplicate experiments are performed and a quantitative analysis of differentially expressed mRNAs is then performed.

Alternatively, expression analysis using the TBC-1 genomic DNA, the TBC-1 cDNAs, or fragments thereof can be done through high density nucleotide arrays or chips as described by Lockhart et al. (1996) and Sosnowsky et al. (1997). Oligonucleotides of 15-50 nucleotides from the sequences of the TBC-1 genomic DNA, the TBC-1 cDNA sequences particularly those comprising at least one of biallelic markers according the present invention, preferably at least one of SEQ ID No 7-8 or those comprising the trait causing mutation, or the sequences complementary thereto, are synthesized directly on the chip (Lockhart et al., supra) or synthesized and then addressed to the chip (Sosnowski et al., supra). Preferably, the oligonucleotides are about 20 nucleotides in length.

TBC-1 cDNA probes labeled with an appropriate compound, such as biotin, digoxigenin or fluorescent dye, are synthesized from the appropriate mRNA population and then randomly fragmented to an average size of 50 to 100 nucleotides. The said probes are then hybridized to the chip. After washing as described in Lockhart et al., supra and application of different electric fields (Sosnowsky et al., 1997), the dyes or labeling compounds are detected and quantified. Duplicate hybridizations are performed. Comparative analysis of the intensity of the signal originating from cDNA probes on the same target oligonucleotide in different cDNA samples indicates a differential expression of TBC-1 mRNAs.

Thus, is also part of the present invention a method for screening of a candidate substance or molecule that modulates the expression of the TBC-1 gene according to the invention, wherein this method comprises the following steps:

a) providing a recombinant cell host containing a nucleic acid, wherein said nucleic acid comprises the 5′ regulatory region sequence or a biologically active fragment or variant thereof, the 5′ regulatory region or its biologically active fragment or variant being operably linked to a polynucleotide encoding a detectable protein;

b) obtaining a candidate substance, and

c) determining the ability of the candidate substance to modulate the expression levels of the polynucleotide encoding the detectable protein.

In a preferred embodiment of the above screening method, the nucleic acid comprising the 5′ regulatory region sequence or a biologically active fragment or variant thereof also includes a 5′ UTR region of one of the TBC-1 cDNAs of SEQ ID Nos 3 and 4, or one of their biologically active fragments or variants thereof.

A second method for the screening of a candidate substance or molecule that modulates the expression of the TBC-1 gene comprises the following steps

a) providing a recombinant cell host containing a nucleic acid, wherein said nucleic acid comprises a 5′ UTR sequence of one of the TBC-1 cDNAs of SEQ ID Nos 3 and 4, or one of their biologically active fragments or variants, the 5′ UTR sequence or its biologically active fragment or variant being operably linked to a polynucleotide encoding a detectable protein;

b) obtaining a candidate substance, and

c) determining the ability of the candidate substance to modulate the expression levels of the polynucleotide encoding the detectable protein.

In a preferred embodiment of the screening method described above, the nucleic acid that comprises a nucleotide sequence selected from the group consisting of the 5′ UTR sequence of one of the TBC-1 cDNAs of SEQ ID Nos 3 and 4 or one of their biologically active fragments or variants, includes a promoter sequence, wherein said promoter sequence can be either endogenous, or in contrast exogenous with respect to the TBC-1 5′ UTR sequences defined therein.

Among the preferred polynucleotides encoding a detectable protein, there may be cited polynucleotides encoding beta galactosidase, green fluorescent protein (GFP) and chloramphenicol acetyl transferase (CAT).

For the design of suitable recombinant vectors useful for performing the screening methods described above, it will be referred to the section of the present specification wherein the preferred recombinant vectors of the invention are detailed.

### Screening Using Transgenic Animals

In vivo methods can utilize transgenic animals for drug screening. Nucleic acids including at least one of the biallelic polymorphisms of interest can be used to generate genetically modified non-human animals or to generate site specific gene modifications in cell lines. The term “transgenic” is intended to encompass genetically modified animals having a deletion or other knock-out of TBC-1 gene activity, having an exogenous TBC-1 gene that is stably transmitted in the host cells, or having an exogenous TBC-1 promoter operably linked to a reporter gene. Transgenic animals may be made through homologous recombination, where the TBC-1 locus is altered. Alternatively, a nucleic acid construct is randomly integrated into the genome. Vectors for stable integration include for example plasmids, retroviruses and other animal viruses, and YACs. Of interest are transgenic mammals e.g. cows, pigs, goats, horses, and particularly rodents such as rats and mice. Transgenic animals allow to study both efficacy and toxicity of the candidate drug.

Methods for Inhibiting the Expression of a TBC-1 Gene

Other therapeutic compositions according to the present invention comprise advantageously an oligonucleotide fragment of the nucleic sequence of TBC-1 as an antisense tool that inhibits the expression of the corresponding TBC-1 gene. Preferred methods using antisense polynucleotide according to the present invention are the procedures described by Sczakiel et al. (1995).

Preferably, the antisense tools are chosen among the polynucleotides (15-200 bp long) that are complementary to the 5′ end of the TBC-1 mRNA. In another embodiment, a combination of different antisense polynucleotides complementary to different parts of the desired targetted gene are used.

Preferred antisense polynucleotides according to the present invention are complementary to a sequence of the mRNAs of TBC-1 that contains the translation initiation codon ATG.

The antisense nucleic acid molecules to be used in gene therapy may be either DNA or RNA sequences. They comprise a nucleotide sequence complementary to the targeted sequence of the PTCA-1 genomic DNA, the sequence of which can be determined using one of the detection methods of the present invention. The targeted DNA or RNA sequence preferably comprises at least one of the biallelic markers according to the present invention. The antisense nucleic acids should have a length and melting temperature sufficient to permit formation of an intracellular duplex having sufficient stability to inhibit the expression of the TBC-1 mRNA in the duplex. Strategies for designing antisense nucleic acids suitable for use in gene therapy are disclosed in Green et al., (1986) and Izant and Weintraub, (1984), the disclosures of which are incorporated herein by reference.

In some strategies, antisense molecules are obtained by reversing the orientation of the TBC-1 coding region with respect to a promoter so as to transcribe the opposite strand from that which is normally transcribed in the cell. The antisense molecules may be transcribed using in vitro transcription systems such as those which employ T7 or SP6 polymerase to generate the transcript. Another approach involves transcription of TBC-1 antisense nucleic acids in vivo by operably linking DNA containing the antisense sequence to a promoter in a suitable expression vector.

Alternatively, suitable antisense strategies are those described by Rossi et al. (1991), in the International Applications Nos. WO 94/23026, WO 95/04141, WO 92/18522 and in the European Patent Application No. EP 0 572 287 A2

An alternative to the antisense technology that is used according to the present invention consists in using ribozymes that will bind to a target sequence via their complementary polynucleotide tail and that will cleave the corresponding RNA by hydrolyzing its target site (namely <<hammerhead ribozymes >>). Briefly, the simplified cycle of a hammerhead ribozyme consists of (1) sequence specific binding to the target RNA via complementary antisense sequences; (2) site-specific hydrolysis of the cleavable motif of the target strand; and (3) release of cleavage products, which gives rise to another catalytic cycle. Indeed, the use of long-chain antisense polynucleotide (at least 30 bases long) or ribozymes with long antisense arms are advantageous. A preferred delivery system for antisense ribozyme is achieved by covalently linking these antisense ribozymes to lipophilic groups or to use liposomes as a convenient vector. Preferred antisense ribozymes according to the present invention are prepared as described by Sczakiel et al. (1995), the specific preparation procedures being referred to in said article being herein incorporated by reference.

Throughout this application, various publications, patents and published patent applications are cited. The disclosures of these publications, patents and published patent specification referenced in this application are hereby incorporated by reference into the present disclosure to more fully describe the sate of the art to which this invention pertains.

### EXAMPLES

### Example 1

### Analysis of the First mRNA Encoding a TBC-1 Polypeptide Synthesized by the Cells

TBC-1 cDNA was obtained as follows: 411 of ethanol suspension containing 1 mg of human prostate total RNA (Clontech laboratories, Inc., Palo Alto, USA; Catalogue N. 64038-1) was centrifuged, and the resulting pellet was air dried for 30 minutes at room temperature.

First strand cDNA synthesis was performed using the Advantage™ RT-for-PCR kit (Clontech laboratories Inc., catalogue N. K1402-1). 1 μl of 20 mM solution of a specific oligo dT primer was added to 12.5 μl of RNA solution in water, heated at 74° C. for 2.5 min and rapidly quenched in an ice bath. 10 μl of 5×RT buffer (50 mM Tris-HCl, pH 8.3, 75 mM KCl, 3 mM MgCl₂), 2.5 μl of dNTP mix (10 mM each), 1.25 μl of human recombinant placental RNA inhibitor were mixed with 1 ml of MMLV reverse transcriptase (200 units). 6.5 μl of this solution were added to RNA-primer mix and incubated at 42° C. for one hour. 80 μl of water were added and the solution was incubated at 94° C. for 5 minutes.

5 μl of the resulting solution were used in a Long Range PCR reaction with hot start, in 50 μl final volume, using 2 units of rtTHXL, 20 pmol/μl of each of 5′-TGACCACCATGCCCATGCT-3′ (271-289 in SEQ ID No 3) and 5′-GCATTTATTCACGTCCACGCC-3′ (3929-3949 in SEQ ID No 3) primers with 35 cycles of elongation for 6 minutes at 67° C. in thermocycler.

The amplification products corresponding to both cDNA strands were partially sequenced in order to ensure the specificity of the amplification reaction.

Results of Nothern blot analysis of prostate mRNAs supported the existence of the first TBC-1 cDNA having about 4 kb in length, which is the nucleotide sequence of SEQ ID No 3.

### Example 2

### Detection of TBC-1 Biallelic Markers: DNA Extraction

Donors were unrelated and healthy. They presented a sufficient diversity for being representative of a French heterogeneous population. The DNA from 100 individuals was extracted and tested for the detection of the biallelic markers.

30 ml of peripheral venous blood were taken from each donor in the presence of EDTA. Cells (pellet) were collected after centrifugation for 10 minutes at 2000 rpm. Red cells were lysed by a lysis solution (50 ml final volume: 10 mM Tris pH7.6; 5 mM MgCl₂; 10 mM NaCl). The solution was centrifuged (10 minutes, 2000 rpm) as many times as necessary to eliminate the residual red cells present in the supernatant, after resuspension of the pellet in the lysis solution.

The pellet of white cells was lysed overnight at 42° C. with 3.7 ml of lysis solution composed of:
3 ml TE 10-2 (Tris-HCl 10 mM, EDTA 2 mM)/NaCl 0.4 M
200 μl SDS 10%
500 μl K-proteinase (2 mg K-proteinase in TE 10-2/NaCl 0.4 M).

For the extraction of proteins, 1 ml saturated NaCl (6M) (1/3.5 v/v) was added. After vigorous agitation, the solution was centrifuged for 20 minutes at 10000 rpm.

For the precipitation of DNA, 2 to 3 volumes of 100% ethanol were added to the previous supernatant, and the solution was centrifuged for 30 minutes at 2000 rpm. The DNA solution was rinsed three times with 70% ethanol to eliminate salts, and centrifuged for 20 minutes at 2000 rpm. The pellet was dried at 37° C., and resuspended in 1 ml TE 10-1 or 1 ml water. The DNA concentration was evaluated by measuring the OD at 260 nm (1 unit OD=50 μg/ml DNA).

To determine the presence of proteins in the DNA solution, the OD 260/OD 280 ratio was determined. Only DNA preparations having a OD 260/OD 280 ratio between 1.8 and 2 were used in the subsequent examples described below.

The pool was constituted by mixing equivalent quantities of DNA from each individual.

### Example 3

### Detection of the Biallelic Markers: Amplification of Genomic DNA by PCR

The amplification of specific genomic sequences of the DNA samples of example 2 was carried out on the pool of DNA obtained previously. In addition, 50 individual samples were similarly amplified.

PCR assays were performed using the following protocol:

| | | |
|---|---|---|
| Final volume | 25 | μl |
| DNA | 2 | ng/μl |
| MgCl₂ | 2 | mM |
| dNTP (each) | 200 | μM |
| primer (each) | 2.9 | ng/μl |
| Ampli Taq Gold DNA polymerase | 0.05 | unit/μl |
| PCR buffer (10× = 0.1 M TrisHCl pH 8.3 0.5 M KCl | 1× | |

Each pair of first primers was designed using the sequence information of the TBC-1 gene disclosed herein and the OSP software (Hillier & Green, 1991). This first pair of primers was about 20 nucleotides in length and had the sequences disclosed in Table 1 in the columns labeled PU and RP.

TABLE 1

| | Position | | | | | | Complementary | |
|---|---|---|---|---|---|---|---|---|
| | range of the | | | Position range of | | | position range of | |
| | amplicon in | | Primer | amplification primer | | Primer | amplification primer | |
| Amplicon | SEQ ID 1 | | name | in SEQ ID No 1 | | name | in SEQ ID No 1 | |
| 99-430 | 9391 | 9845 | B1 | 9391 | 9408 | C1 | 9828 | 9845 |
| | Position | | | | | | Complementary | |
| | range of the | | | Position range of | | | position range of | |
| | amplicon in | | Primer | amplification primer | | Primer | amplification primer | |
| Amplicon | SEQ ID 2 | | name | in SEQ ID No 2 | | name | in SEQ ID No 2 | |
| 99-20508 | 988 | 1529 | B2 | 988 | 1006 | C2 | 1509 | 1529 |
| 99-20469 | 5039 | 5554 | B3 | 5039 | 5056 | C3 | 5534 | 5554 |
| 5-254 | 5997 | 6350 | B4 | 5997 | 6015 | C4 | 6332 | 6350 |
| 5-257 | 14371 | 14817 | B5 | 14371 | 14390 | C5 | 14798 | 14817 |
| 99-20511 | 18751 | 19217 | B6 | 18751 | 18771 | C6 | 19198 | 19217 |
| 99-20510 | 19605 | 20005 | B7 | 19605 | 19625 | C7 | 19986 | 20005 |
| 99-20504 | 29529 | 30061 | B8 | 29529 | 29547 | C8 | 30041 | 30061 |
| 99-20493 | 42268 | 42752 | B9 | 42268 | 42287 | C9 | 42732 | 42752 |
| 99-20499 | 69026 | 69543 | B10 | 69026 | 69046 | C10 | 69525 | 69543 |
| 99-20473 | 76323 | 76790 | B11 | 76323 | 76343 | C11 | 76771 | 76790 |
| 5-249 | 78292 | 78721 | B12 | 78292 | 78309 | C12 | 78704 | 78721 |
| 99-20485 | 81893 | 82372 | B13 | 81893 | 81912 | C13 | 82353 | 82372 |
| 99-20481 | 84392 | 84929 | B14 | 84392 | 84412 | C14 | 84909 | 84929 |
| 99-20480 | 89746 | 90198 | B15 | 89746 | 89765 | C15 | 90179 | 90198 |

Preferably, the primers contained a common oligonucleotide tail upstream of the specific bases targeted for amplification which was useful for sequencing.

Primers PU contain the following additional PU 5′ sequence: TGTAAAACGACGGCCAGT (SEQ ID No 6); primers RP contain the following RP 5′ sequence: CAGGAAACAGCTATGACC (SEQ ID No 7).

The synthesis of these primers was performed following the phosphoramidite method, on a GENSET UFPS 24.1 synthesizer.

DNA amplification was performed on a Genius II thermocycler. After heating at 95° C. for 10 min, 40 cycles were performed. Each cycle comprised: 30 sec at 95° C., 54° C. for 1 min, and 30 sec at 72° C. For final elongation, 10 min at 72° C. ended the amplification. The quantities of the amplification products obtained were determined on 96-well microtiter plates, using a fluorometer and Picogreen as intercalant agent (Molecular Probes).

### Example 4

### Detection of the Biallelic Markers: Sequencing of Amplified Genomic DNA and Identification of Polymorphisms

The sequencing of the amplified DNA obtained in example 3 was carried out on ABI 377 sequencers. The sequences of the amplification products were determined using automated dideoxy terminator sequencing reactions with a dye terminator cycle sequencing protocol. The products of the sequencing reactions were run on sequencing gels and the sequences were determined using gel image analysis [ABI Prism DNA Sequencing Analysis software (2.1.2 version)].

The sequence data were further evaluated to detect the presence of biallelic markers among the pooled amplified fragments. The polymorphism search was based on the presence of superimposed peaks in the electrophoresis pattern resulting from different bases occurring at the same position as described previously.

15 fragments of amplification was analyzed. In this segment, 19 biallelic markers were detected. The localization of the biallelic marker is as shown in Table 2.

TABLE 2

| | | Marker | Localization | Polymorphism | | BM position in |
|---|---|---|---|---|---|---|
| Amplicon | BM | Name | in TBC-1 gene | Allele 1 | allele 2 | SEQ ID No 1 |
| 99-430 | A1 | 99-430-352 | Intron 1 | A | G | 9494 |
| | | Marker | Localization | Polymorphism | | BM position in |
| Amplicon | BM | Name | in TBC-1 gene | allele 1 | allele 2 | SEQ ID No 2 |
| 99-20508 | A2 | 99-20508-456 | Intron upstream | C | T | 1443 |
| | | | to Exon A | | | |
| 99-20469 | A3 | 99-20469-213 | Intron A | C | T | 5247 |
| 5-254 | A4 | 5-254-227 | Intron B | A | G | 6223 |
| 5-257 | A5 | 5-257-353 | Intron D | C | T | 14723 |
| 99-20511 | A6 | 99-20511-32 | Intron D | C | T | 19186 |
| 99-20511 | A7 | 99-20511-221 | Intron D | A | G | 18997 |
| 99-20510 | A8 | 99-20510-115 | Intron D | deletion of | | 19891 |
| | | | | TCT | | |
| 99-20504 | A9 | 99-20504-90 | Intron D | A | G | 29617 |
| 99-20493 | A10 | 99-20493-238 | Intron D | A | C | 42519 |
| 99-20499 | A11 | 99-20499-221 | Intron G | A | G | 69324 |
| 99-20499 | A12 | 99-20499-364 | Intron G | A | T | 69181 |
| 99-20499 | A13 | 99-20499-399 | Intron G | A | G | 69146 |
| 99-20473 | A14 | 99-20473-138 | Intron H | deletion of | | 76458 |
| | | | | TAACA | | |
| 5-249 | A15 | 5-249-304 | Intron I | A | G | 78595 |
| 99-20485 | A16 | 99-20485-269 | Intron I | A | G | 82159 |
| 99-20481 | A17 | 99-20481-131 | Intron I | G | C | 84522 |
| 99-20481 | A18 | 99-20481-419 | Intron I | A | T | 84810 |
| 99-20480 | A19 | 99-20480-233 | Intron J | A | G | 89967 |

BM refers to “biallelic marker”. All1 and all2 refer respectively to allele 1 and allele 2 of the biallelic marker.

TABLE 3

| | | | |
|---|---|---|---|
| | | Position range | |
| | | of probes in | |
| BM | Marker Name | SEQ ID No 1 | Probes |

| | | | | |
|---|---|---|---|---|
| A1 | 99-430-352 | 9482 | 9506 | P1 |

| | | | |
|---|---|---|---|
| | | Position range | |
| | | of probes in | |
| BM | Marker Name | SEQ ID No 2 | Probes |

| | | | | |
|---|---|---|---|---|
| A2 | 99-20508-456 | 1431 | 1455 | P2 |
| A3 | 99-20469-213 | 5235 | 5259 | P3 |
| A4 | 5-254-227 | 6211 | 6235 | P4 |
| A5 | 5-257-353 | 14711 | 14735 | P5 |
| A6 | 99-20511-32 | 19174 | 19198 | P6 |
| A7 | 99-20511-221 | 18985 | 19009 | P7 |
| A9 | 99-20504-90 | 29605 | 29629 | P9 |
| A10 | 99-20493-238 | 42507 | 42531 | P10 |
| A11 | 99-20499-221 | 69312 | 69336 | P11 |
| A12 | 99-20499-364 | 69169 | 69193 | P12 |
| A13 | 99-20499-399 | 69134 | 69158 | P13 |
| A15 | 5-249-304 | 78583 | 78607 | P15 |
| A16 | 99-20485-269 | 82147 | 82171 | P16 |
| A17 | 99-20481-131 | 84510 | 84534 | P17 |
| A18 | 99-20481-419 | 84798 | 84822 | P18 |
| A19 | 99-20480-233 | 89955 | 89979 | P19 |

### Example 5

### Validation of the Polymorphisms Through Microsequencing

The biallelic markers identified in example 4 were further confirmed and their respective frequencies were determined through microsequencing. Microsequencing was carried out for each individual DNA sample described in Example 2.

Amplification from genomic DNA of individuals was performed by PCR as described above for the detection of the biallelic markers with the same set of PCR primers (Table 1).

The preferred primers used in microsequencing were about 19 nucleotides in length and hybridized just upstream of the considered polymorphic base. According to the invention, the primers used in microsequencing are detailed in Table 4.

TABLE 4

| | | | Position range of | | | Complementary position | |
|---|---|---|---|---|---|---|---|
| | | | microsequencing | | | range of | |
| | Biallelic | | primer mis 1 in | | | microsequencing primer | |
| Marker Name | Marker | Mis. 1 | SEQ ID No 1 | | Mis. 2 | mis. 2 in SEQ ID No 1 | |
| 99-430-352 | A1 | D1 | 9475 | 9493 | E1 | 9495 | 9513 |
| | | | Position range of | | | Complementary position | |
| | | | microsequencing | | | range of | |
| | Biallelic | | primer mis 1 in | | | microsequencing primer | |
| Marker Name | Marker | Mis. 1 | SEQ ID No 2 | | Mis. 2 | mis. 2 in SEQ ID No 2 | |
| 99-20508-456 | A2 | D2 | 1424 | 1442 | E2 | 1444 | 1462 |
| 99-20469-213 | A3 | D3 | 5228 | 5246 | E3 | 5248 | 5266 |
| 5-254-227 | A4 | D4 | 6204 | 6222 | E4 | 6224 | 6242 |
| 5-257-353 | A5 | D5 | 14704 | 14722 | E5 | 14724 | 14742 |
| 99-20511-32 | A6 | D6 | 19167 | 19185 | E6 | 19187 | 19205 |
| 99-20511-221 | A7 | D7 | 18978 | 18996 | E7 | 18998 | 19016 |
| 99-20510-115 | A8 | D8 | 19872 | 19890 | E8 | 19892 | 19910 |
| 99-20504-90 | A9 | D9 | 29598 | 29616 | E9 | 29618 | 29636 |
| 99-20493-238 | A10 | D10 | 42500 | 42518 | E10 | 42520 | 42538 |
| 99-20499-221 | A11 | D11 | 69305 | 69323 | E11 | 69325 | 69343 |
| 99-20499-364 | A12 | D12 | 69162 | 69180 | E12 | 69182 | 69200 |
| 99-20499-399 | A13 | D13 | 69127 | 69145 | E13 | 69147 | 69165 |
| 99-20473-138 | A14 | D14 | 76439 | 76457 | E14 | 76459 | 76477 |
| 5-249-304 | A15 | D15 | 78576 | 78594 | E15 | 78596 | 78614 |
| 99-20485-269 | A16 | D16 | 82140 | 82158 | E16 | 82160 | 82178 |
| 99-20481-131 | A17 | D17 | 84503 | 84521 | E17 | 84523 | 84541 |
| 99-20481-419 | A18 | D18 | 84791 | 84809 | E18 | 84811 | 84829 |
| 99-20480-233 | A19 | D19 | 89948 | 89966 | E19 | 89968 | 89986 |

The microsequencing reaction was performed as follows

After purification of the amplification products, the microsequencing reaction mixture was prepared by adding, in a 20 μl final volume: 10 pmol microsequencing oligonucleotide, 1 U Thermosequenase (Amersham E79000G), 1.25 μl Thermosequenase buffer (260 mM Tris HCl pH 9.5, 65 mM MgCl₂), and the two appropriate fluorescent ddNTPs (Perkin Elmer, Dye Terminator Set 401095) complementary to the nucleotides at the polymorphic site of each biallelic marker tested, following the manufacturer's recommendations. After 4 minutes at 94° C., 20 PCR cycles of 15 sec at 55° C., 5 sec at 72° C., and 10 sec at 94° C. were carried out in a Tetrad PTC-225 thermocycler (MJ Research). The unincorporated dye terminators were then removed by ethanol precipitation. Samples were finally resuspended in formamide-EDTA loading buffer and heated for 2 min at 95° C. before being loaded on a polyacrylamide sequencing gel. The data were collected by an ABI PRISM 377 DNA sequencer and processed using the GENESCAN software (Perkin Elmer).

Following gel analysis, data were automatically processed with software that allows the determination of the alleles of biallelic markers present in each amplified fragment.

The software evaluates such factors as whether the intensities of the signals resulting from the above microsequencing procedures are weak, normal, or saturated, or whether the signals are ambiguous. In addition, the software identifies significant peaks (according to shape and height criteria). Among the significant peaks, peaks corresponding to the targeted site are identified based on their position. When two significant peaks are detected for the same position, each sample is categorized classification as homozygous or heterozygous type based on the height ratio.

### REFERENCES

Altschul et al., 1990, J. Mol. Biol. 215(3):403-410/Altschul et al., 1993, Nature Genetics 3:266-272/Altschul et al., 1997, Nuc. Acids Res. 25:3389-3402/Ausubel et al. (1989) Current Protocols in Molecular Biology, Green Publishing Associates and Wiley Interscience, N.Y./Beaucage et al., Tetrahedron Lett 1981, 22: 1859-1862/Bram R J et al., 1993, Mol. Cell Biol., 13: 4760-4769./Brown E L, Belagaje R, Ryan M J, Khorana H G, Methods Enzymol 1979; 68:109-151/Castagnoli L. et al. (Felici F.), 1991, J. Mol. Biol., 222:301-310./Chai H. et al., 1993, Biotechnol. Appl. Biochem., 18:259-273/Chee et al. (1996) Science. 274:610-614./Chen and Kwok Nucleic Acids Research 25:347-353 1997/Chen et al. Proc. Natl. Acad. Sci. USA 94/20 10756-10761, 1997/Cho R J et al., 1998, Proc. Natl. Acad. Sci. USA, 95(7): 3752-3757./Chumakov I. et al., 1995, Nature, 377(6547 Suppl): 175-297./Compton J. (1991) Nature. 350(6313):91-92./Dib et al., 1996, Nature, 380: III-V./Ellis N A, 1997 Curr. Op. Genet. Dev., 7: 354-363/Feldman and Steg, 1996, Medecine/Sciences, synthese, 12:47-55/Fields and Song, 1989, Nature, Vol. 340: 245-246./Fishel R & Wilson T. 1997, Curr. Op. Genet. Dev. 7: 105-113/Flotte et al., 1992, Am. J. Respir. Cell Mol. Biol., 7: 349-356. /Fodor et al. (1991) Science 251:767-777./Fromont-Racine M. et al., 1997, Nature Genetics, 16(3): 277-282./Fuller S. A. et al., 1996, Immunology in Current Protocols in Molecular Biology, Ausubel et al. Eds, John Wiley & Sons, Inc., USA/Geysen H. Mario et al. 1984. Proc. Natl. Acad. Sci. U.S.A. 81:3998-4002/Gonnet et al., 1992, Science 256:1443-1445/Green et al., Ann. Rev. Biochem. 55:569-597 (1986)/Grompe, M. et al., Proc. Natl. Acad. Sci. U.S.A 1989; 86:5855-5892/Grompe, M. Nature Genetics 1993; 5:111-117/Guatelli J C et al. Proc. Natl. Acad. Sci. USA. 35:273-286./Haber D & Harlow E, 1997, Nature Genet. 16:320-322./Hacia J G, Brody L C, Chee M S, Fodor S P, Collins F S, Nat Genet 1996; 14(4):441-447/Haff L. A. and Smirnov I. P. (1997) Genome Research, 7:378-388./Hames B. D. and Higgins S. J. (1985) Nucleic Acid Hybridization: A Practical Approach. Hames and Higgins Ed., IRL Press, Oxford./Haju L, et al., Clin Chem 1993; 39(11Pt 1):2282-2287/Harper J W et al., 1993, Cell, Vol. 75: 805-816./Harris H et al., 1969, Nature 223:363-368./Henikoff and Henikoff, 1993, Proteins 17:49-61/Higgins et al., 1996, Methods Enzymol. 266:383-402/Hillier L. and Green P. Methods Appl., 1991, 1: 124-8./Huang L. et al. (1996) Cancer Res 56(5):1137-1141./Huygen et al., 1996, Nature Medicine, 2(8):893-898/Izant and Weintraub, Cell 36:1007-1015 (1984)/Julan et al., 1992, J. Gen. Virol., 73: 3251-3255./Karlin and Altschul, 1990, Proc. Natl. Acad. Sci. USA 87:2267-2268/Koch Y., 1977, Biochem. Biophys. Res. Commun., 74:488-491/Kohler G. and Milstein C., 1975, Nature, 256: 495./Kozal M J, et al., Nat Med 1996; 2(7):753-759/Landegren U. et al. (1998) Genome Research, 8:769-776./Leger O J, et al., 1997, Hum Antibodies, 8(1): 3-16/Lenhard T. et al., 1996, Gene, 169:187-190/Livak et al., Nature Genetics, 9:341-342, 1995/Livak K J, and Hainer J W., 1994, Hum Mutat., 3(4): 379-385./Lockhart et al. Nature Biotechnology 14: 1675-1680, 1996/Lucas A. H., 1994, In: Development and Clinical Uses of Haempophilus b Conjugate./Mansour S L et al., 1988, Nature, 336: 348-352./Marshall R. L. et al. (1994) PCR Methods and Applications. 4:80-84./Martineau P, Jones P, Winter G, 1998, J Mol Biol, 280(1):117-127/Mc Whorter W. P., et al. A screening study of prostate cancer in high risk families. J Urol 1992; 148:826-828./McLaughlin et al., 1989, J. Virol., 62: 1963-1973./Muzyczka et al., 1992, Cuur. Topics in Micro. and Immunol., 158: 97-129. /Narang S A, Hsiung H M, Brousseau R, Methods Enzymol 1979; 68:90-98/Neda et al., 1991, J. Biol. Chem., 266: 14143-14146./Nickerson D. A. et al. (1990) Proc. Natl. Acad. Sci. U.S.A. 87:8923-8927./Nyren P, Pettersson B, Uhlen M, Anal Biochem 1993; 208(1):171-175/O'Reilly et al., 1992, Baculovirus expression vectors: a Laboratory Manual. W.H. Freeman and Co., New York/Ohno et al., 1994, Sciences, 265:781-784/Oldenburg K. R. et al., 1992, Proc. Natl. Acad. Sci., 89:5393-5397./Orita et al., Proc. Natl. Acad. Sci. U.S.A. 1989; 86: 2776-2770/Parmley and Smith, Gene, 1988, 73:305-318./Pastinen et al., Genome Research 1997; 7:606-614/PCR Methods and Applications”, 1991, Cold Spring Harbor Laboratory Press./Pearson and Lipman, 1988, Proc. Natl. Acad. Sci. USA 85(8):2444-2448/Pietu et al. Genome Research 6:492-503, 1996/Porath J et al., 1975, Nature, 258(5536): 598-599./Reimann K A, et al., 1997, AIDS Res Hum Retroviruses. 13(11): 933-943/Ridder R, et al., 1995, Biotechnology (NY), 13(3):255-260/Rossi et al., Pharmacol. Ther. 50:245-254, (1991)/Roth J. A. et al., 1996, Nature Medicine, 2(9):985-991/Rougeot, C. et al.,. Eur. J. Biochem. 219 (3): 765-773, 1994/Roux et al., 1989, Proc. Natl. Acad. Sci. USA, 86: 9079-9083./Sambrook, et al. 1989. Molecular cloning: a laboratory manual. 2ed. Cold Spring Harbor Laboratory, Cold spring Harbor, N.Y./Samson M, et al. (1996) Nature, 382(6593):722-725./Samulski et al., 1989, J. Virol., 63: 3822-3828./Sanchez-Pescador R., 1988, J. Clin. Microbiol., 26(10):1934-1938/Sandou et al., 1994, Science, 265: 1875-1878./Schena et al. Science 270:467-470, 1995/Schwartz and Dayhoff, eds., 1978, Matrices for Detecting Distance Relationships: Atlas of Protein Sequence and Structure, Washington: National Biomedical Research Foundation/Sczakiel G. et al., 1995, Trends Microbiol., 1995, 3(6):213-217/Sheffield, V. C. et al, Proc. Natl. Acad. Sci. U.S.A 1991; 49:699-706/Shoemaker D D, et al., Nat Genet 1996; 14(4):450-456/Smith et al., 1983, Mol. Cell. Biol., 3:2156-2165./Sosnowski R G, et al., Proc Natl Acad Sci USA 1997; 94:1119-1123/Steinberg G. D., et al. Family history and the risk of prostate cancer, The prostate 1990; 17, 337-347. /Stryer, L., Biochemistry, 4th edition, 1995/Syvanen A C, et al., 1994, Hum Mutat., 3(3): 172-179./Tacson et al., 1996, Nature Medicine, 2(8):888-892./Thompson et al., 1994, Nucleic Acids Res. 22(2):4673-4680/Tyagi et al. (1998) Nature Biotechnology. 16:49-53./Urdea M. S., 1988, Nucleic Acids Research, 11: 4937-4957/Urdea M S et al., 1991, Nucleic Acids Symp Ser., 24: 197-200./Valadon P., et al., 1996, J. Mol. Biol., Vol. 261:11-22./Vaughan T J, et al., 1996, Nat Biotechnol. 14(3): 309-314/Vlasak R. et al., 1983, Eur. J. Biochem., 135:123-126/Wabiko et al., 1986, DNA, 5(4):305-314./Walker et al. (1996) Clin. Chem. 42:9-13./Westerink M. A. J., 1995, Proc. Natl. Acad. Sci., 92:4021-4025./White, M. B. et al. (1992) Genomics. 12:301-306./White, M. B. et al. (1997) Genomics. 12:301-306./Wilson R. et al., 1994, Nature, 368(6466): 32-38./Zhang S D et al., 1996, Genes and development, 10: 1108-1119.

### SEQUENCE LISTING FREE TEXT

The following free text appears in the accompanying sequence listing:
5′ regulatory region
polymorphic base
complement
3′ regulatory region
deletion of
or
probe
homology with Genset 5′ EST in ref
sequencing oligonucleotide primerPU
sequencing oligonucleotide primerRP

We claim:

1. An isolated, purified, or recombinant polynucleotide comprising a contiguous span of at least 60 nucleotides of SEQ ID NO:1, 2, 3, 4 and the complements thereof.

2. The isolated, purified, or recombinant polynucleotide according to claim 1, wherein said contiguous span of any one of SEQ ID NOs:1, 2, or the complements thereof, includes a biallelic marker that is selected from the group consisting of the biallelic markers at position 9494 of SEQ ID NO:1, position 1443 of SEQ ID NO:2, position 5247 of SEQ ID NO:2, position 6223 of SEQ ID NO:2, position 14723 of SEQ ID NO:2, position 19186 of SEQ ID NO:2, position 18997 of SEQ ID NO:2, position 19891 of SEQ ID NO:2, position 29617 of SEQ ID NO:2, position 42519 of SEQ ID NO:2, position 69324 of SEQ ID NO:2, position 69181 of SEQ ID NO:2, position 69146 of SEQ ID NO:2, position 76458 of SEQ ID NO:2, position 78595 of SEQ ID NO:2, position 82159, position 84522 of SEQ ID NO:2, position 84810 of SEQ ID NO:2, and position 89967 of SEQ ID NO:2.

3. A composition comprising a recombinant vector comprising the isolated, purified, or recombinant polynucleotide of claim 1.

4. The composition according to claim 3, wherein said composition comprises a host cell transformed with said recombinant vector.

5. An isolated polypeptide comprising SEQ ID NO:5.

6. A composition comprising an isolated or purified antibody composition capable of selectively binding to an epitope-containing fragment of a polypeptide comprising SEQ ID NO:5.

7. A method of genotyping comprising the steps of:
(a) obtaining a nucleic acid sample from an individual; and
(b) determining the identity of a polymorphic base at a TBC-1-related biallelic marker or the complement thereof in said nucleic acid sample, wherein the identity of the polymorphic base determines the genotype of the individual at said TBC-1-related biallelic marker and, wherein said TBC-1-related biallelic marker is positioned in SEQ ID NO:1 or SEQ ID NO:2.

8. The method according to claim 7, further comprising amplifying a portion of said sequence comprising the biallelic marker prior to said determining step.

9. The method according to claim 7, wherein said determining is performed by a hybridization assay, sequencing assay, microsequencing assay, or enzyme-based mismatch detection assay.

10. The method according to claim 7, wherein said TBC-1-related biallelic marker is selected from the group consisting of the biallelic markers in positions 9494 of the SEQ ID NO:1, and 1443, 5247, 6223, 14723, 19186, 18997, 19891, 29617, 42519, 69324, 69181, 69146, 76458, 78595, 82159, 84522, 84810, and 89967 of the SEQ ID NO:2.
